(19) United States
(12) Patent Application Publication
(10) Pub. No.: US 2017/0317290 A1
(43) Pub. Date: Nov. 2, 2017

(54) ORGANIC ELECTRONIC DEVICE AND DISPLAY APPARATUS USING COMPOSITION FOR ORGANIC ELECTRONIC DEVICE

(71) Applicant: DUK SAN NEOLUX CO., LTD., Cheonan-si, Chungcheongnam-do (KR)

(72) Inventors: Mun Jae LEE, Cheonan-si, Chungcheongnam-do (KR); Jung Cheol PARK, Suwon-si, Gyeonggi-do (KR); Soung Yun MUN, Cheonan-si, Chungcheongnam-do (KR); Jae Taek KWON, Jeonju-si, Jeollabuk-do (KR); Bum Sung LEE, Cheonan-si, Chungcheongnam-do (KR); Sun Pil HWANG, Ansan-si, Gyeonggi-do (KR); Jin Ho YUN, Cheonan-si, Chungcheongnam-do (KR); Sun Hee LEE, Hwaseong-si, Gyeonggi-do (KR); Seok Hyun KIM, Seongnam-si, Gyeonggi-do (KR)

(73) Assignee: Duk San Neolux Co., Ltd., Cheonan-si, Chungcheongnam-do (KR)

(21) Appl. No.: 15/521,505

(22) PCT Filed: Oct. 6, 2015
(86) PCT No.: PCT/KR2015/010542

§ 371 Date: Apr. 24, 2017

(30) Foreign Application Priority Data
Oct. 24, 2014 (KR) 10-2014-0145507
Oct. 24, 2014 (KR) 10-2014-0145511

(51) Int. Cl. H01L 51/00; C07D 307/91; C07D 333/76; C07D 405/12; C07D 409/12; C07D 495/04; G09G 3/3208; C07C 211/54; C07D 409/14; C07D 209/86; H01L 51/50; H01L 51/52

(52) U.S. Cl. CPC H01L 51/0059; C07D 209/86; C07D 307/91; C07D 333/76; C07D 405/12; C07D 409/12; H01L 51/0061; C07D 495/04; G09G 3/3208; H01L 51/0067; C07C 211/54; H01L 51/006; C07D 409/14; G09G 2330/021; G09G 2320/045; H01L 51/0052; H01L 51/0054; H01L 51/0056; H01L 51/0058; H01L 51/0072; H01L 51/0073; H01L 51/0074; H01L 51/5008; H01L 51/5012; H01L 51/5056; H01L 51/5206; H01L 51/5262; H01L 51/5221

(57) ABSTRACT

The present invention relates to an organic electric element and a display device using the same as a hole transport layer comprising a composition composed of two or more compounds having similar structures to improve luminous efficiency, stability and life span of an electric element, and an electronic device including the same.

### CROSS-REFERENCE TO RELATED APPLICATION

This patent application claims priority under US Patent Law Section 119(a) (35 U.S.C §119(a)) to U.S. Patent Application No. 10-2014-0145033 filed on Oct. 24, 2014, all of which are incorporated herein by reference. In addition, this patent application claims priority to countries other than the United States for the same reasons as above, the entire contents of which are incorporated herein by reference.

### BACKGROUND

### Technical Field

The present invention relates to organic electric element, display device and electronic device using composition composed of compound for organic electric element, and more specifically, display device and organic electric element comprising the organic material layer using two or more different hole transport material in the hole transport layer.

### Background Art

In general, an organic luminescence phenomenon refers to a phenomenon in which electric energy is converted into light energy by means of an organic material. The organic electric element using the organic luminescence phenomenon is, by applying current, self-luminous element using luminescence principle of luminescent material by recombination energy of holes injected from the anode and electron injected from the cathode.

The organic electric element may have a structure in which an anode is formed on a substrate, on which the organic electric element may have a structure formed sequentially a hole injection layer, a hole transport layer, an emitting layer, an electron transport layer, an electron injection layer, and a cathode. Here, the hole injection layer, the hole transport layer, the emitting layer, the electron transport layer, and the electron injection layer are organic thin films made of organic compounds.

Currently, the portable display market is a trend growing in size with large display, which requires larger power consumption than traditional portable displays. Accordingly, power consumption has become an important factor for a portable display having a limited power supply such as battery, and high efficiency, life span and the driving voltage are important factors to be solved.

In particular, because life span and driving voltage are very relevant to thermal degradation of a hole injection material and a hole transport material, a number of methods have been studied in order to overcome this. For example, a method of constituting the hole transport layer in multiple layers (U.S. Pat. No. 5,256,945) and methods of using materials having a high glass transition temperature (U.S. Pat. No. 5,061,569) and so on are proposed.

In addition, when a material having a good hole transport function is used in order to reduce driving voltage, the driving voltage reduction of the element is large, but the charge is excessively injected, and the efficiency and life span of the element are lowered, and therefore there have been many attempts to solve these problems.

However, there is a problem that the rise in the progressive driving voltage of the blue organic electric element among the red, green, and blue increases the power consumption and the shortened life span of the organic electric element, and in order to solve such a problem, a technique forming a buffer layer between the anode and the hole transport layer has been proposed (Korean Patent Publication No. 2006-0032099).

### SUMMARY

### Technical Challenge

An object of the present invention is to increase life span by reducing thermal degradation occurring at the interface between the hole injection layer and the hole transport layer and at the interface between the hole transport layer and the emitting layer by mixing two or more hole transport materials having different band gaps in the hole transport layer, and to provide an organic electric element having excellent luminous efficiency by efficiently controlling the injection amount of the charge in the emitting layer.

### Technical Solution

The present invention relates to the organic electric element and the electronic device thereof using a composition comprising mixture of compounds for an organic electric element represented by the Formula 1 and 2, or Formula 1 and 2′, more specially provides an organic electric element and the electronic device including the same using a composition comprising mixture of two or more hole transport materials having different chemical structures in the hole transport layer composed of the composition.

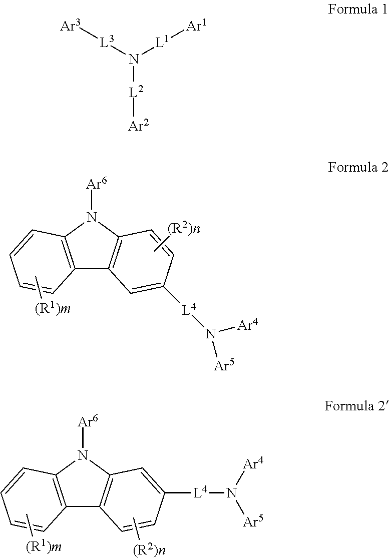

### Effects of the Invention

The organic electric element of the present invention and the display device including the same has a long life span by reducing the thermal degradation occurring at the interface between the hole injection layer and the hole transport layer and at the interface between the hole transport layer and the emitting layer, and has an excellent emitting efficiency by efficiently controlling the injection amount of the charge in the emitting layer.

### BRIEF DESCRIPTION OF THE DRAWINGS

FIG. 1 illustrates an example of an organic electric element according to an embodiment of the present invention.

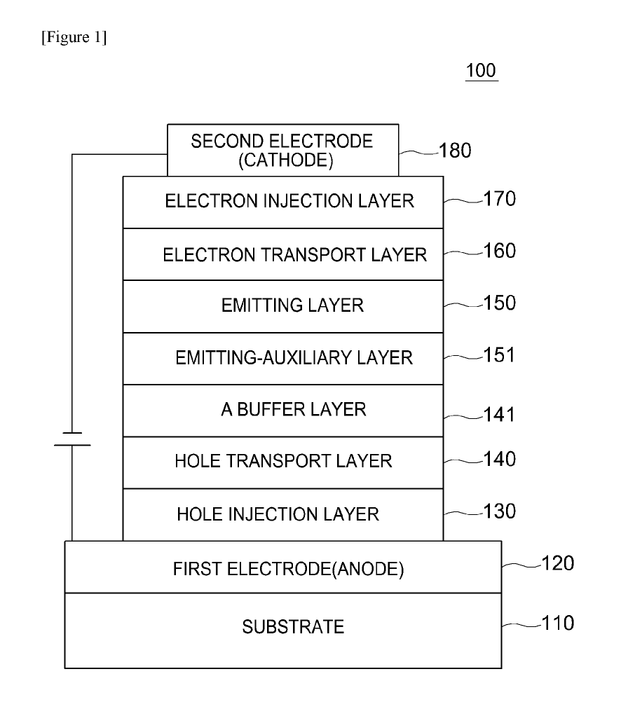

### DETAILED DESCRIPTION

Hereinafter, some embodiments of the present invention will be described in detail. Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “connected” to another component, the component may be directly connected or connected to the other component, but another component may be “connected”, “coupled” or “connected” between each component.

As used in the specification and the accompanying claims, unless otherwise stated, the following is the meaning of the term as follows.

Unless otherwise stated, the term “halo” or “halogen” as used herein includes fluorine, bromine, chlorine, or iodine.

Unless otherwise stated, the term “alkyl” or “alkyl group” as used herein has a single bond of 1 to 60 carbon atoms, and means saturated aliphatic functional radicals including a linear alkyl group, a branched chain alkyl group, a cycloalkyl group (alicyclic), a cycloalkyl group substituted with an alkyl, or an alkyl group substituted with a cycloalkyl.

Unless otherwise stated, the term “haloalkyl” or “halogen alkyl” as used herein means an alkyl group substituted with a halogen.

Unless otherwise stated, the term “heteroalkyl” as used herein means alkyl substituted one or more carbon atoms with heteroatom.

Unless otherwise stated, the term “alkenyl” or “alkynyl” as used herein has, but not limited to, double or triple bonds of 2 to 60 carbon atoms, and includes a linear or a branched chain group.

Unless otherwise stated, the term “cycloalkyl” as used herein means, but not limited to, alkyl forming a ring having 3 to 60 carbon atoms.

Unless otherwise stated, the term “alkoxyl group”, “alkoxy group” or “alkyloxy group” as used herein means an oxygen radical attached to an alkyl group, but not limited to, and has 1 to 60 carbon atoms.

Unless otherwise stated, the term “alkenoxyl group”, “alkenoxy group”, “alkenyloxyl group” or “alkenyloxy group” as used herein means an oxygen radical attached to an alkenyl group, but not limited to, and has 2 to 60 carbon atoms.

Unless otherwise stated, the term “aryloxyl group” or “aryloxy group” as used herein means an oxygen radical attached to an aryl group, but not limited to, and has 6 to 60 carbon atoms.

Unless otherwise stated, the term “aryl group” or “arylene group” as used herein has, but not limited to, 6 to 60 carbon atoms. Herein, the aryl group or the arylene group means a monocyclic or polycyclic aromatic group, and may include the aromatic ring formed in conjunction or reaction with an adjacent substituent. For examples, the aryl group may include a phenyl group, a biphenyl group, a fluorene group, or a spirofluorene group.

The prefix “aryl” or “ar” means a radical substituted with an aryl group. For example, an arylalkyl may be an alkyl substituted with an aryl, and an arylalkenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heteroalkyl” as used herein means alkyl containing one or more heteroatoms. Unless otherwise stated, the term “heteroaryl group” or “heteroarylene group” as used herein means, but not limited to, a C₂ to C₆₀ aryl or arylene group containing one or more heteroatoms, and includes at least one of monocyclic or polycyclic rings, and may also be formed in conjunction with an adjacent group.

Unless otherwise stated, the term “heterocyclic group” as used herein contains one or more heteroatoms, but not limited to, has 2 to 60 carbon atoms, includes any one of monocyclic or polycyclic rings, and may include heteroaliphadic ring and/or heteroaromatic ring. Also, the heterocyclic group may also be formed in conjunction with an adjacent group.

Unless otherwise stated, the term “heteroatom” as used herein represents at least one of N, O, S, P, or Si.

Also, the term “heterocyclic group” may include a ring comprising SO₂ instead of carbon consisting of cycle. For example, “heterocyclic group” includes compound below.

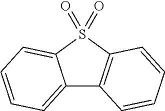 [SMILES: O=S1(=O)c2ccccc2-c2ccccc21]

Unless otherwise stated, the term “aliphatic” as used herein means an aliphatic hydrocarbon having 1 to 60 carbon atoms, and the term “aliphatic ring” as used herein means an aliphatic hydrocarbon ring having 3 to 60 carbon atoms.

Unless otherwise stated, the term “ring” means an aliphatic ring having 3 to 60 carbon atoms, or an aromatic ring having 6 to 60 carbon atoms, or a hetero ring having 2 to 60 carbon atoms, or a fused ring formed by the combination of them, and includes a saturated or unsaturated ring.

Other hetero compounds or hetero radicals other than the above-mentioned hetero compounds contain, but are not limited to, one or more heteroatoms.

Unless otherwise stated, the term “carbonyl” as used herein is represented by —COR′, wherein R′ may be hydrogen, an alkyl having 1 to 20 carbon atoms, an aryl having 6 to 30 carbon atoms, a cycloalkyl having 3 to 30 carbon atoms, an alkenyl having 2 to 20 carbon atoms, an alkynyl having 2 to 20 carbon atoms, or the combination of these.

Unless otherwise stated, the term “ether” as used herein is represented by —R—O—R′, wherein R or R′ may be independently hydrogen, an alkyl having 1 to 20 carbon atoms, an aryl having 6 to 30 carbon atoms, a cycloalkyl having 3 to 30 carbon atoms, an alkenyl having 2 to 20 carbon atoms, an alkynl having 2 to 20 carbon atoms, or the combination of these.

Unless otherwise stated, the term “substituted or unsubstituted” as used herein means that “substitution” is substituted with at least one substituent selected from the group consisting of, but not limited to, deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiophene group, a C₆-C₂₀ arylthiophene group, a C₂-C₂₀ alkenyl group, a C₂-C₂₀ alkynyl group, a C₃-C₂₀ cycloalkyl group, a C₆-C₆₀ aryl group, a C₆-C₂₀ aryl group substituted by deuterium, a C₈-C₂₀ arylalkenyl group, a silane group, a boron group, a germanium group, and a C₂-C₂₀ heterocyclic group.

Unless otherwise stated, the Formula used in the present invention is applied in the same manner as the substituent definition according to the definition of the exponent of the following Formula.

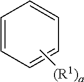

Wherein, when a is an integer of zero, the substituent R¹ is absent, when a is an integer of 1, the sole R¹ is linked to any one of the carbon atoms constituting the benzene ring, when a is an integer of 2 or 3, it is combined as follows and wherein R¹ is the same or differ from each other. When a is an integer of 4 to 6, the substituent R¹ is are combined to carbon of the benzene ring in a similar manner. Meanwhile, the indication of hydrogen bound to the carbon forming the benzene ring is omitted.

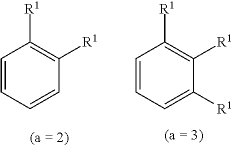

Further, the organic electronic element according to the present invention may be any one of an organic light emitting diode(OLED), an organic solar cell, an organic photo conductor(OPC), an organic transistor(organic TFT), and an element for monochromatic or white illumination.

Another embodiment of the present invention may include an electronic device including the display device which includes the described organic electronic element of the present invention, and a control unit for controlling the display device. Here, the electronic device may be a wired/wireless communication terminal which is currently used or will be used in the future, and covers all kinds of electronic devices including a mobile communication terminal such as a cellular phone, a personal digital assistant(PDA), an electronic dictionary, a point-to-multipoint(PMP), a remote controller, a navigation unit, a game player, various kinds of TVs, and various kinds of computers.

Hereinafter, a display device and an organic electric element according to an aspect of the present invention will be described.

The present invention provides an organic electric element and a display device including the same characterized by comprising a first electrode; a second electrode; and an organic material layer; disposed between the first electrode and the second electrode, and comprising at least one hole transport layer and emitting layer comprising of emitting compounds, wherein the hole transport layer is composed of composition comprising mixture of a compound represented by Formula 1 and a compound represented by Formula 2, or composition comprising mixture of a compound represented by Formula 1 and a compound represented by Formula 2′.

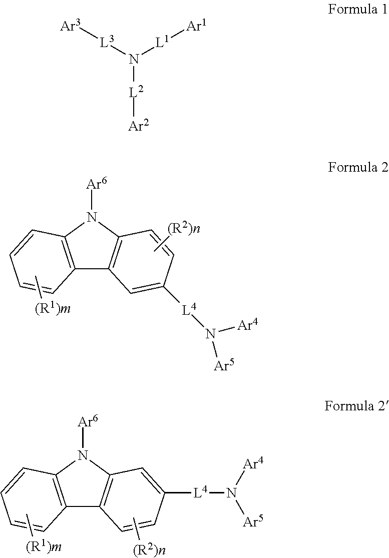

{In the Formula 1, Formula 2 or Formula 2′ wherein Ar¹, Ar², Ar³ is each independently selected from the group consisting of a C₆-C₆₀ aryl group, a C₂-C₆₀ hetero aryl group; a fluorenyl group;

L¹, L², L³ is each independently selected from the group consisting of a single bond, a C₆-C₆₀ arylene group, a divalent of C₂-C₆₀ heterocyclic group, a fluorenylene group, a divalent fused ring group of a C₃-C₆₀ aliphatic ring and a C₆-C₆₀ aromatic ring;
Ar⁴, Ar⁵ and Ar⁶ are each independently selected from the group consisting of a C₆-C₆₀ aryl group; a C₂-C₆₀ heterocyclic group; a fluorenyl group;
L⁴ may be selected from the group consisting of a C₆-C₆₀ arylene group, a divalent of C₂-C₆₀ heterocyclic group, a fluorenylene group, a fused ring group of a C₃-C₆₀ aliphatic ring and a C₆-C₆₀ aromatic ring;
m is integer of 0 to 4, n is an integer of 0 to 3;
R¹ and R² are the same or different, and are each independently selected from the group consisting of deuterium; halogen; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; and -L′-N(Rₐ)(R_{b}); where, L′ may be selected from the group consisting of a single bond; a C₆-C₆₀ arylene group; a fluorenylene group; a fused ring group of a C₃-C₆₀ aliphatic ring and a C₆-C₆₀ aromatic ring; and a C₂-C₆₀ heterocyclic, and the Rₐ and R_{b} may be independently selected from the group consisting of a C₆-C₆₀ aryl group, a fluorenyl group; a fused ring group of a C₃-C₆₀ aliphatic ring and a C₆-C₆₀ aromatic ring; and a C₂-C₆₀ heterocyclic group containing at least one heteroatom of O, N, S, Si, or P, or in case m, n are 2 or more, R¹, R² are each in plural and are the same or different, and a plurality of R¹ or a plurality of R² may combine to each other to form a ring, (where, Aryl group, heteroaryl group, fluorenyl group, arylene group, heterocyclic group and fused ring group may be substituted with one or more substituents selected from deuterium; halogen; a silane group; siloxane group; boron group; germanium group; cyano group; nitro group; -L′-N(Rₐ)(R_{b}); a C₁-C₂₀ alkylthio group; C₁-C₂₀ alkoxyl group; C₁-C₂₀ alkyl group; C₂-C₂₀ alkenyl group; C₂-C₂₀ alkynyl group; C₆-C₆₀ aryl group; C₆-C₆₀ aryl group substituted with deuterium; a fluorenyl group; C₂-C₂₀ heterocyclic group; C₃-C₂₀ cycloalkyl group; the group consisting of C₇-C₂₀ arylalkyl group and C₈-C₂₀ arylalkenyl group, and also, these substituents may combine each other and form a ring, wherein the term ‘ring’ means C₃-C₆₀ aliphatic ring or C₆-C₆₀ aromatic ring or a C₂-C₆₀ heterocyclic ring or a fused ring formed by the combination of them, and includes a saturated or unsaturated ring.)}

In another embodiment of the present invention, at least any one of the two kinds of compounds represented by the Formula 1 is one of the following Formulas 1-2, 1-3 and 1-4.

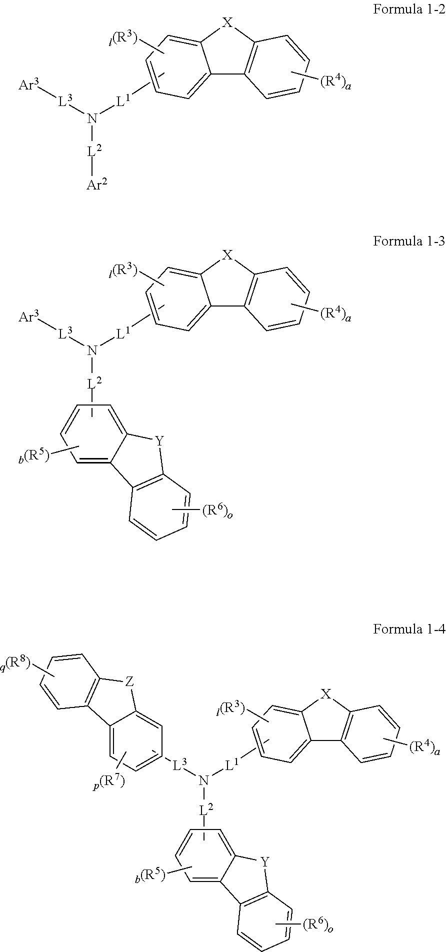

(In the Formula 1-2, 1-3 and 1-4, wherein Ar², Ar³, L¹, L² and L³ are the same as defined above, and X, Y and Z are each independently S, O or CR′R″, and R′ and R″ are each independently selected from the group consisting of a C₆-C₂₄ aryl group, C₁-C₂₀ alkyl group, C₂-C₂₀ alkenyl group and C₁-C₂₀ alkoxy group and R′ and R″ are may combine each other and form a spiro, and R³, R⁴, R⁵, R⁶, R⁷ and R⁸ are each independently selected from the group consisting of deuterium; tritium; a cyano group; nitro group; halogen; aryl group; alkenyl group; alkylene group; alkoxy group; and hetrocyclic group, and a plurality of R³ or plurality of R⁴ or plurality of R⁵ or plurality of R⁶ or plurality of R⁷ or plurality of R⁸ may combine to each other to form a ring, and 1, b and p are an integer of 0 to 3, and a, o and q are an integer of 0 to 4.)

In another embodiment of the present invention, the compound represented by Formula 2 above is represented by compounds of Formula 2-2 or Formula 2-3 below.

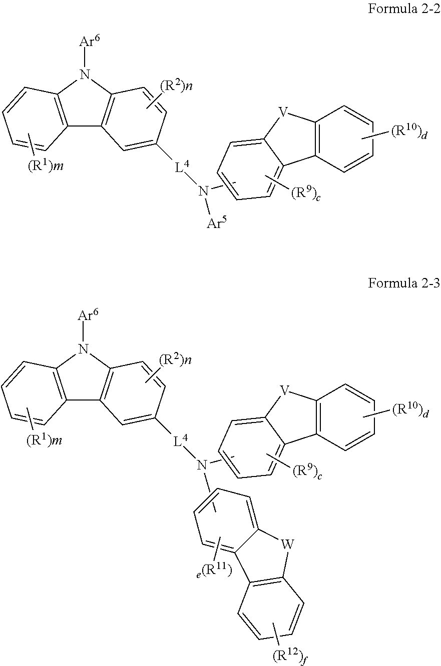

In another embodiment of the present invention, the compound represented by Formula 2′ above is represented by compounds of Formula 2′-2, 2′-3 below.

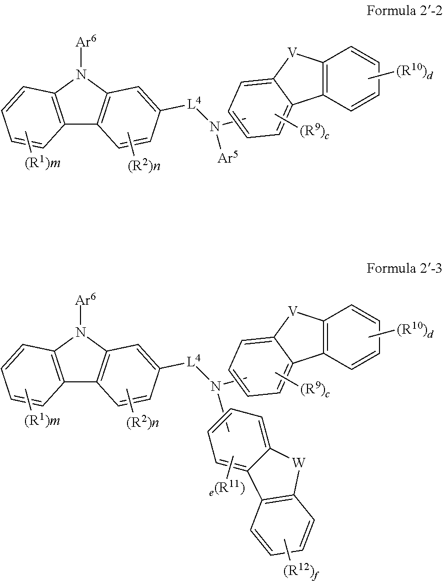

In the Formulae 2-2, 2-3, 2′-2 and 2′-3, wherein R¹, R², Ar⁵, Ar⁶, L⁴, m and n are the same as defined above, and V and W are each independently S, O or CR′R″, and R′ and R″ are each independently selected from the group consisting of a C₆-C₂₄ aryl group; C₁-C₂₀ alkyl group; C₂-C₂₀ alkenyl group; and C₁-C₂₀ alkoxy group, and R′ and R″ are may combine each other and form a spiro, and R⁹, R¹⁰, R¹¹, and R¹² are each independently selected from the group consisting of deuterium; tritium; a cyano group; nitro group; halogen; aryl group; alkenyl group; alkylene group; alkoxy group; and hetrocyclic group, and a plurality of R¹ or plurality of R² or plurality of R³ or plurality of R⁴ or plurality of R⁵ or plurality of R⁶ may combine to each other to form a ring, and c and e are integer of 0 to 3, and d and f are an integer of 0 to 4.)

In another embodiment of the present invention, the compound of Formula 1 includes a compound represented by the following formulae, and provides an organic electric element containing such a compound.

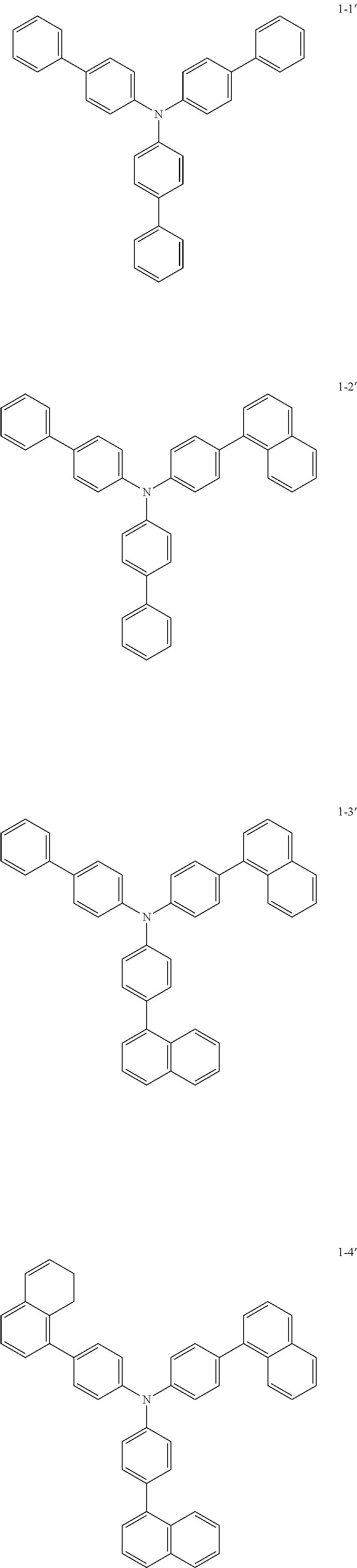 [SMILES: c1ccc(-c2ccc(N(c3ccc(-c4ccccc4)cc3)c3ccc(-c4ccccc4)cc3)cc2)cc1 ; c1ccc(-c2ccc(N(c3ccc(-c4ccccc4)cc3)c3ccc(-c4cccc5ccccc45)cc3)cc2)cc1 ; c1ccc(-c2ccc(N(c3ccc(-c4cccc5ccccc45)cc3)c3ccc(-c4cccc5ccccc45)cc3)cc2)cc1 ; C1=Cc2cccc(-c3ccc(N(c4ccc(-c5cccc6ccccc56)cc4)c4ccc(-c5cccc6ccccc56)cc4)cc3)c2CC1]

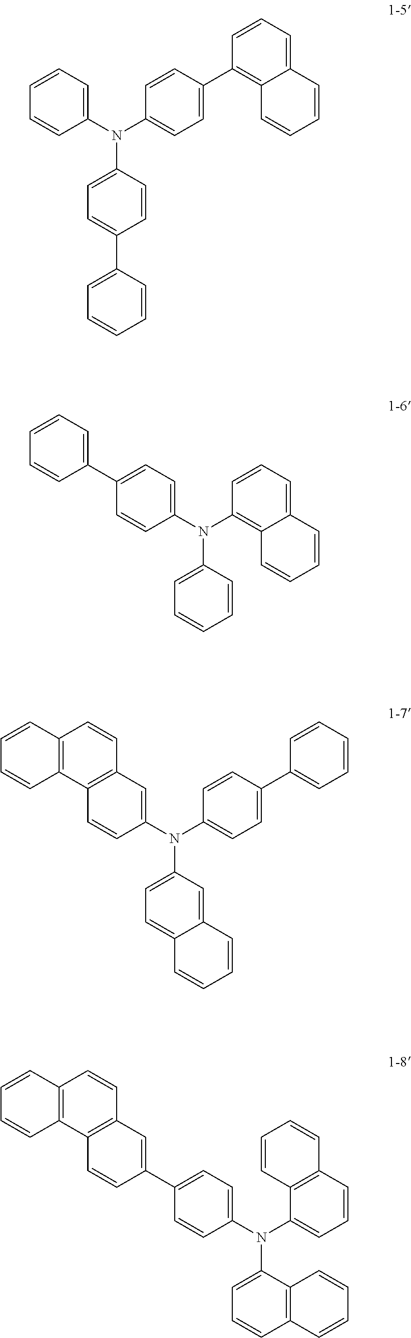 [SMILES: c1ccc(-c2ccc(N(c3ccccc3)c3ccc(-c4cccc5ccccc45)cc3)cc2)cc1 ; c1ccc(-c2ccc(N(c3ccccc3)c3cccc4ccccc34)cc2)cc1 ; c1ccc(-c2ccc(N(c3ccc4ccccc4c3)c3ccc4c(ccc5ccccc54)c3)cc2)cc1 ; c1ccc2c(N(c3ccc(-c4ccc5c(ccc6ccccc65)c4)cc3)c3cccc4ccccc34)cccc2c1]

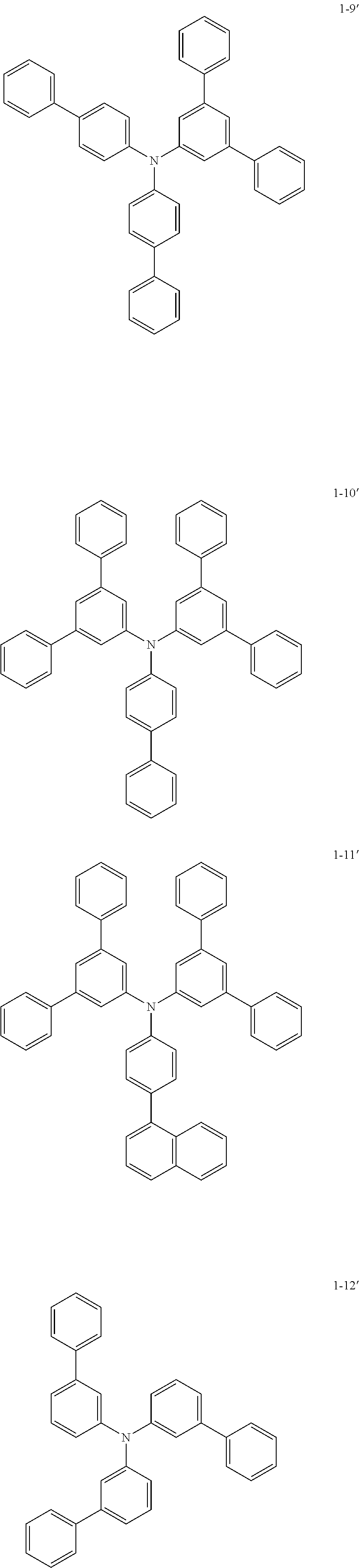 [SMILES: c1ccc(-c2ccc(N(c3ccc(-c4ccccc4)cc3)c3cc(-c4ccccc4)cc(-c4ccccc4)c3)cc2)cc1 ; c1ccc(-c2ccc(N(c3cc(-c4ccccc4)cc(-c4ccccc4)c3)c3cc(-c4ccccc4)cc(-c4ccccc4)c3)cc2)cc1 ; c1ccc(-c2cc(-c3ccccc3)cc(N(c3ccc(-c4cccc5ccccc45)cc3)c3cc(-c4ccccc4)cc(-c4ccccc4)c3)c2)cc1 ; c1ccc(-c2cccc(N(c3cccc(-c4ccccc4)c3)c3cccc(-c4ccccc4)c3)c2)cc1]

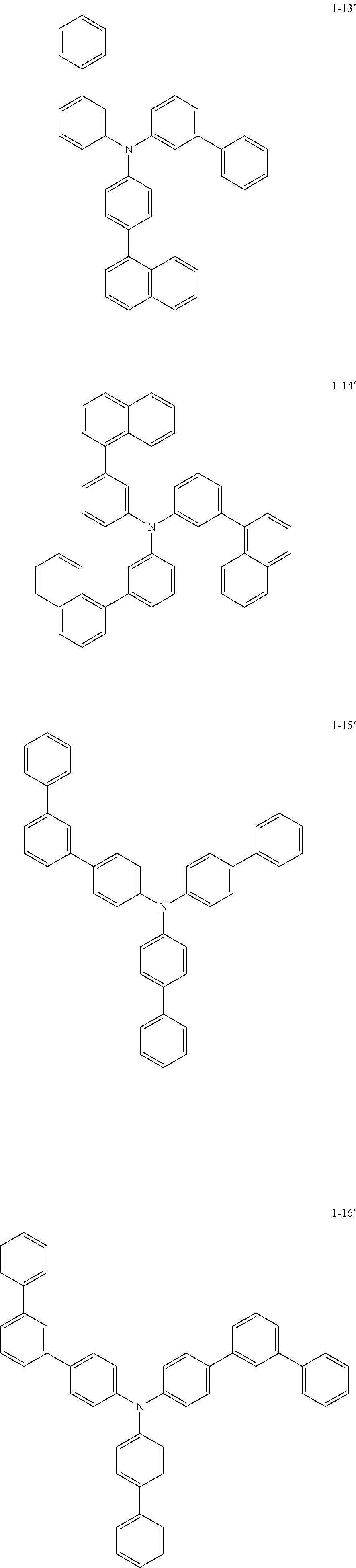 [SMILES: c1ccc(-c2cccc(N(c3ccc(-c4cccc5ccccc45)cc3)c3cccc(-c4ccccc4)c3)c2)cc1 ; c1cc(-c2cccc3ccccc23)cc(N(c2cccc(-c3cccc4ccccc34)c2)c2cccc(-c3cccc4ccccc34)c2)c1 ; c1ccc(-c2ccc(N(c3ccc(-c4ccccc4)cc3)c3ccc(-c4cccc(-c5ccccc5)c4)cc3)cc2)cc1 ; c1ccc(-c2ccc(N(c3ccc(-c4cccc(-c5ccccc5)c4)cc3)c3ccc(-c4cccc(-c5ccccc5)c4)cc3)cc2)cc1]

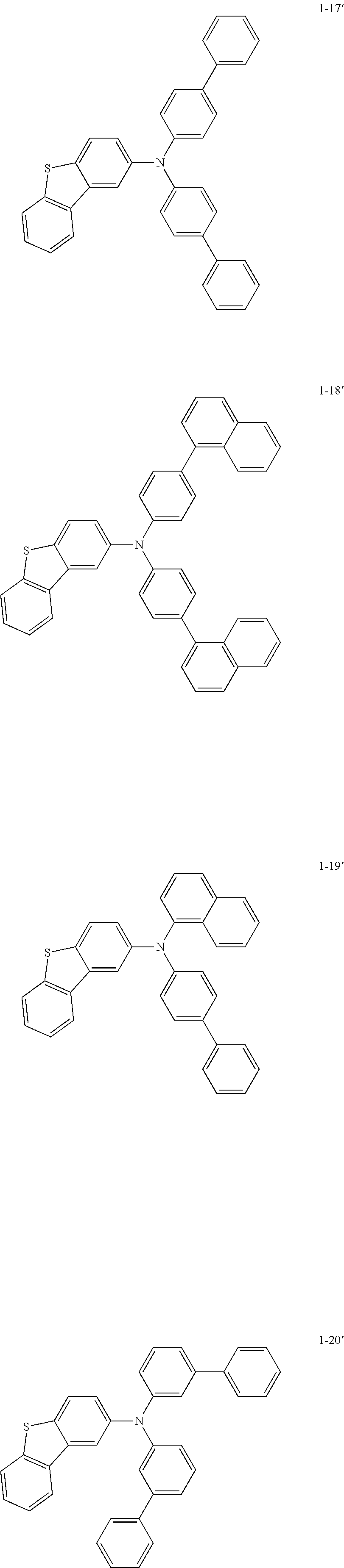 [SMILES: c1ccc(-c2ccc(N(c3ccc(-c4ccccc4)cc3)c3ccc4sc5ccccc5c4c3)cc2)cc1 ; c1ccc2c(-c3ccc(N(c4ccc(-c5cccc6ccccc56)cc4)c4ccc5sc6ccccc6c5c4)cc3)cccc2c1 ; c1ccc(-c2ccc(N(c3ccc4sc5ccccc5c4c3)c3cccc4ccccc34)cc2)cc1 ; c1ccc(-c2cccc(N(c3cccc(-c4ccccc4)c3)c3ccc4sc5ccccc5c4c3)c2)cc1]

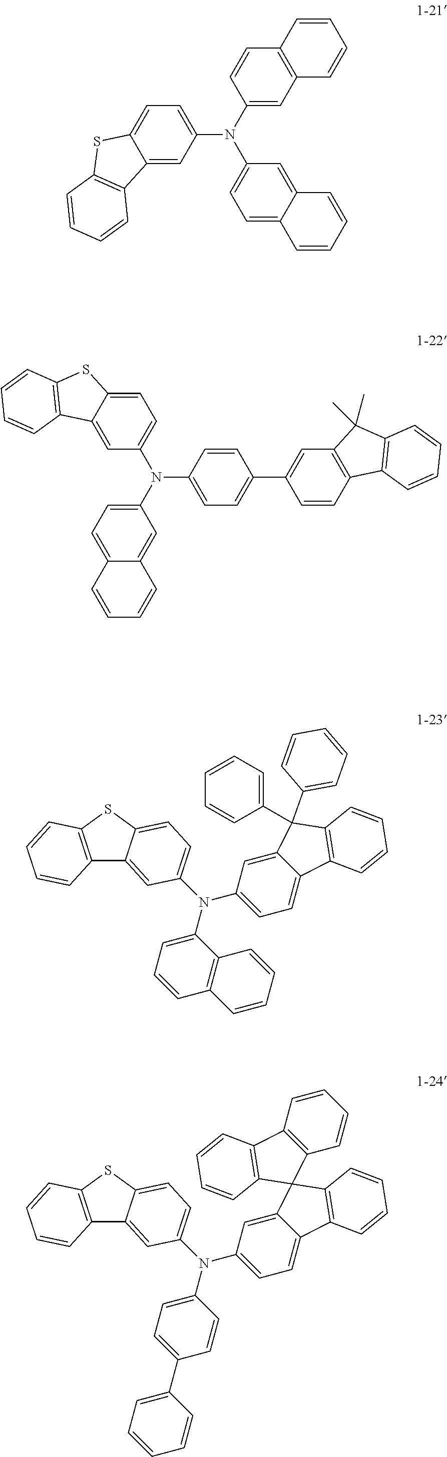 [SMILES: c1ccc2cc(N(c3ccc4ccccc4c3)c3ccc4sc5ccccc5c4c3)ccc2c1 ; CC1(C)c2ccccc2-c2ccc(-c3ccc(N(c4ccc5ccccc5c4)c4ccc5sc6ccccc6c5c4)cc3)cc21 ; c1ccc(C2(c3ccccc3)c3ccccc3-c3ccc(N(c4ccc5sc6ccccc6c5c4)c4cccc5ccccc45)cc32)cc1 ; c1ccc(-c2ccc(N(c3ccc4c(c3)C3(c5ccccc5-c5ccccc53)c3ccccc3-4)c3ccc4sc5ccccc5c4c3)cc2)cc1]

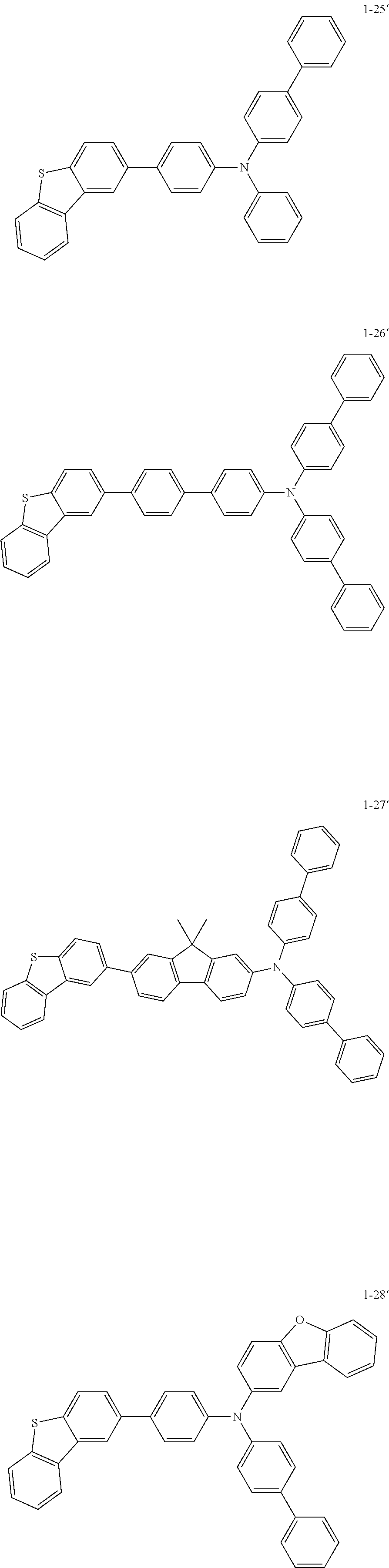 [SMILES: c1ccc(-c2ccc(N(c3ccccc3)c3ccc(-c4ccc5sc6ccccc6c5c4)cc3)cc2)cc1 ; c1ccc(-c2ccc(N(c3ccc(-c4ccccc4)cc3)c3ccc(-c4ccc(-c5ccc6sc7ccccc7c6c5)cc4)cc3)cc2)cc1 ; CC1(C)c2cc(-c3ccc4sc5ccccc5c4c3)ccc2-c2ccc(N(c3ccc(-c4ccccc4)cc3)c3ccc(-c4ccccc4)cc3)cc21 ; c1ccc(-c2ccc(N(c3ccc(-c4ccc5sc6ccccc6c5c4)cc3)c3ccc4oc5ccccc5c4c3)cc2)cc1]

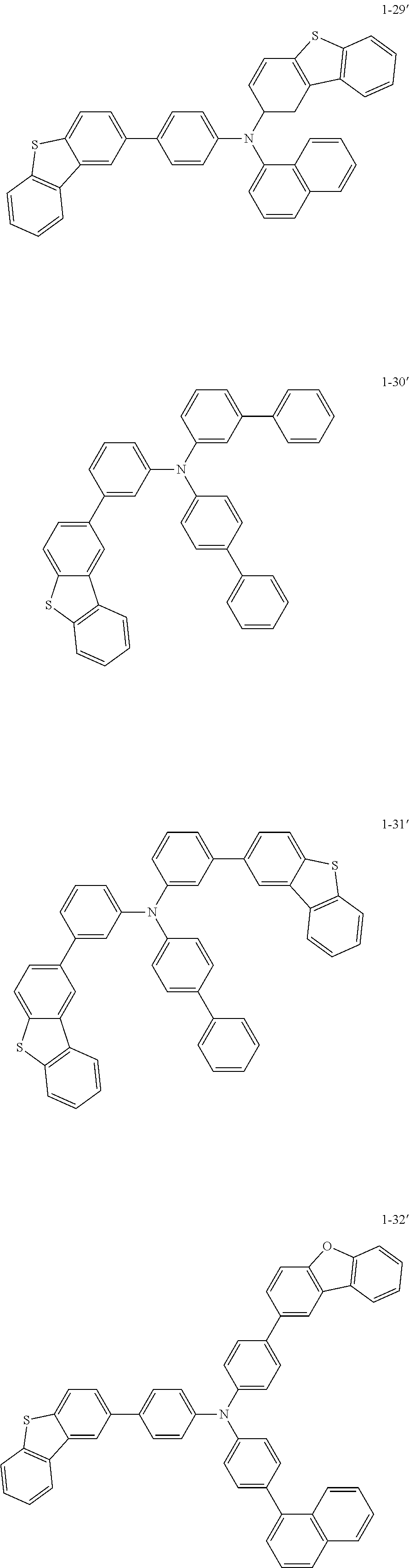 [SMILES: C1=CC(N(c2ccc(-c3ccc4sc5ccccc5c4c3)cc2)c2cccc3ccccc23)Cc2c1sc1ccccc21 ; c1ccc(-c2ccc(N(c3cccc(-c4ccccc4)c3)c3cccc(-c4ccc5sc6ccccc6c5c4)c3)cc2)cc1 ; c1ccc(-c2ccc(N(c3cccc(-c4ccc5sc6ccccc6c5c4)c3)c3cccc(-c4ccc5sc6ccccc6c5c4)c3)cc2)cc1 ; c1ccc2c(-c3ccc(N(c4ccc(-c5ccc6oc7ccccc7c6c5)cc4)c4ccc(-c5ccc6sc7ccccc7c6c5)cc4)cc3)cccc2c1]

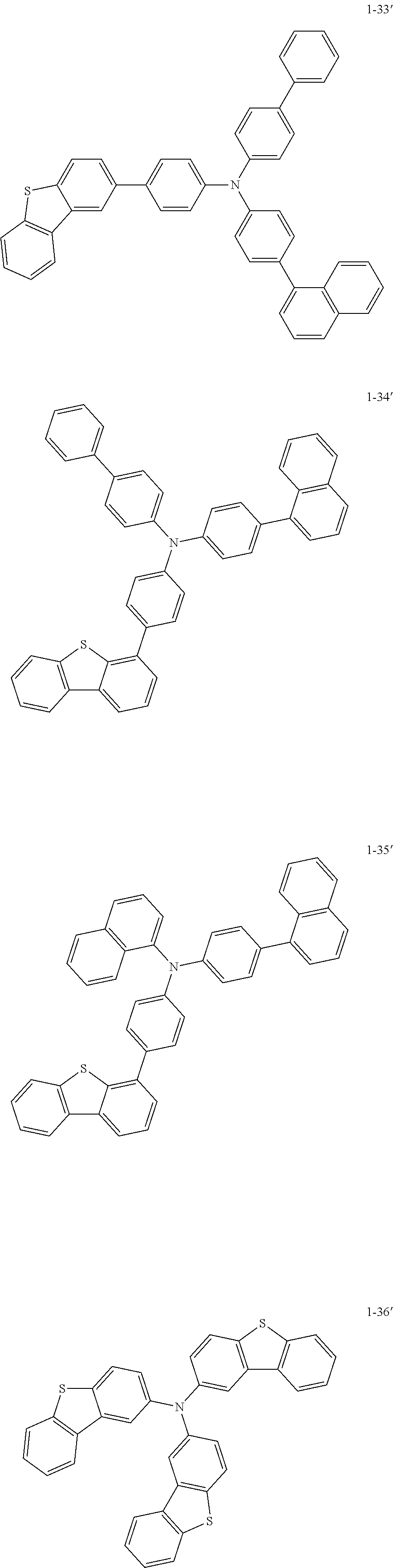 [SMILES: c1ccc(-c2ccc(N(c3ccc(-c4ccc5sc6ccccc6c5c4)cc3)c3ccc(-c4cccc5ccccc45)cc3)cc2)cc1 ; c1ccc(-c2ccc(N(c3ccc(-c4cccc5ccccc45)cc3)c3ccc(-c4cccc5c4sc4ccccc45)cc3)cc2)cc1 ; c1ccc2c(-c3ccc(N(c4ccc(-c5cccc6c5sc5ccccc56)cc4)c4cccc5ccccc45)cc3)cccc2c1 ; c1ccc2c(c1)sc1ccc(N(c3ccc4sc5ccccc5c4c3)c3ccc4sc5ccccc5c4c3)cc12]

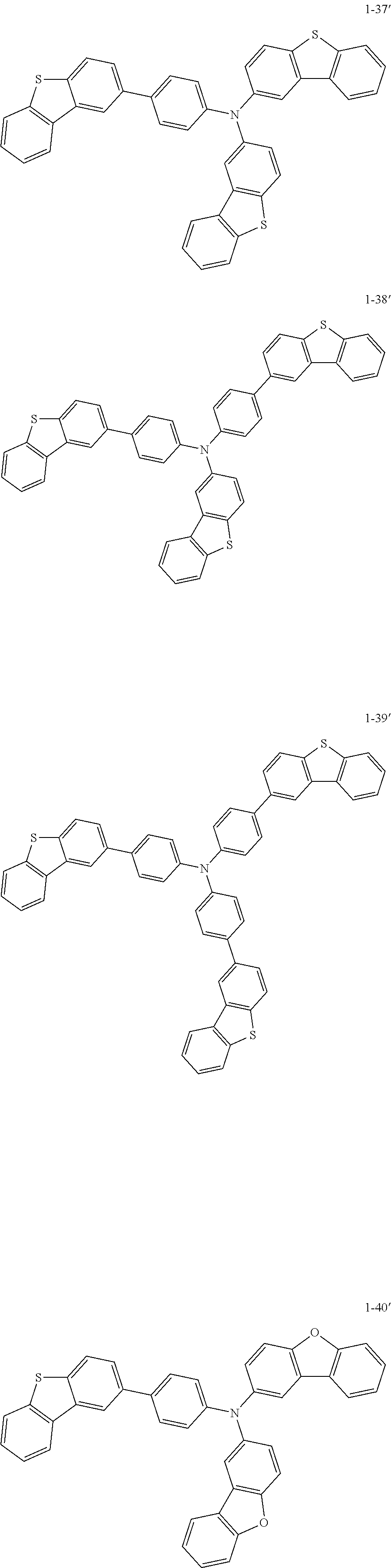 [SMILES: c1ccc2c(c1)oc1ccc(N(c3ccc(-c4ccc5sc6ccccc6c5c4)cc3)c3ccc4sc5ccccc5c4c3)cc12 ; c1ccc2c(c1)sc1ccc(-c3ccc(N(c4ccc(-c5ccc6sc7ccccc7c6c5)cc4)c4ccc5sc6ccccc6c5c4)cc3)cc12 ; c1ccc2c(c1)sc1ccc(-c3ccc(N(c4ccc(-c5ccc6sc7ccccc7c6c5)cc4)c4ccc(-c5ccc6sc7ccccc7c6c5)cc4)cc3)cc12 ; c1ccc2c(c1)oc1ccc(N(c3ccc(-c4ccc5sc6ccccc6c5c4)cc3)c3ccc4oc5ccccc5c4c3)cc12]

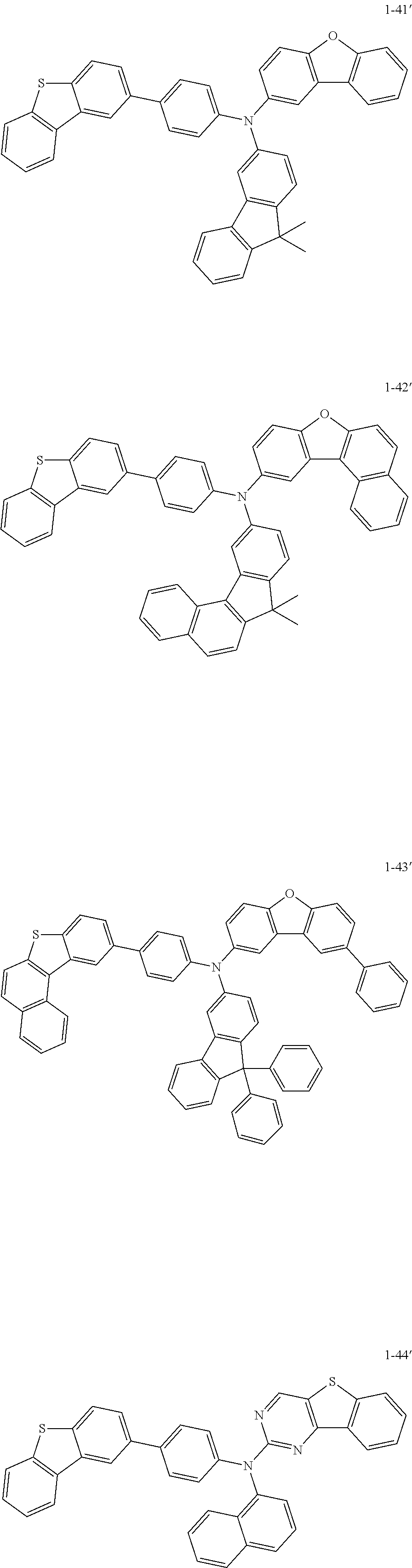 [SMILES: CC1(C)c2ccccc2-c2cc(N(c3ccc(-c4ccc5sc6ccccc6c5c4)cc3)c3ccc4oc5ccccc5c4c3)ccc21 ; CC1(C)c2ccc(N(c3ccc(-c4ccc5sc6ccccc6c5c4)cc3)c3ccc4oc5ccc6ccccc6c5c4c3)cc2-c2c1ccc1ccccc21 ; c1ccc(-c2ccc3oc4ccc(N(c5ccc(-c6ccc7sc8ccc9ccccc9c8c7c6)cc5)c5ccc6c(c5)-c5ccccc5C6(c5ccccc5)c5ccccc5)cc4c3c2)cc1 ; c1ccc2c(N(c3ccc(-c4ccc5sc6ccccc6c5c4)cc3)c3ncc4sc5ccccc5c4n3)cccc2c1]

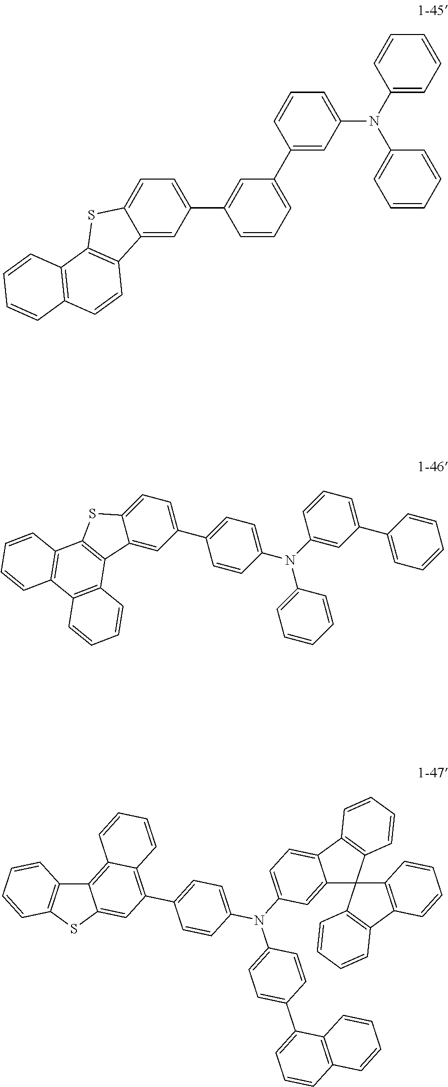 [SMILES: c1ccc(N(c2ccccc2)c2cccc(-c3cccc(-c4ccc5sc6c7ccccc7ccc6c5c4)c3)c2)cc1 ; c1ccc(-c2cccc(N(c3ccccc3)c3ccc(-c4ccc5sc6c7ccccc7c7ccccc7c6c5c4)cc3)c2)cc1 ; c1ccc2c(c1)-c1ccccc1C21c2ccccc2-c2ccc(N(c3ccc(-c4cccc5ccccc45)cc3)c3ccc(-c4cc5sc6ccccc6c5c5ccccc45)cc3)cc21]

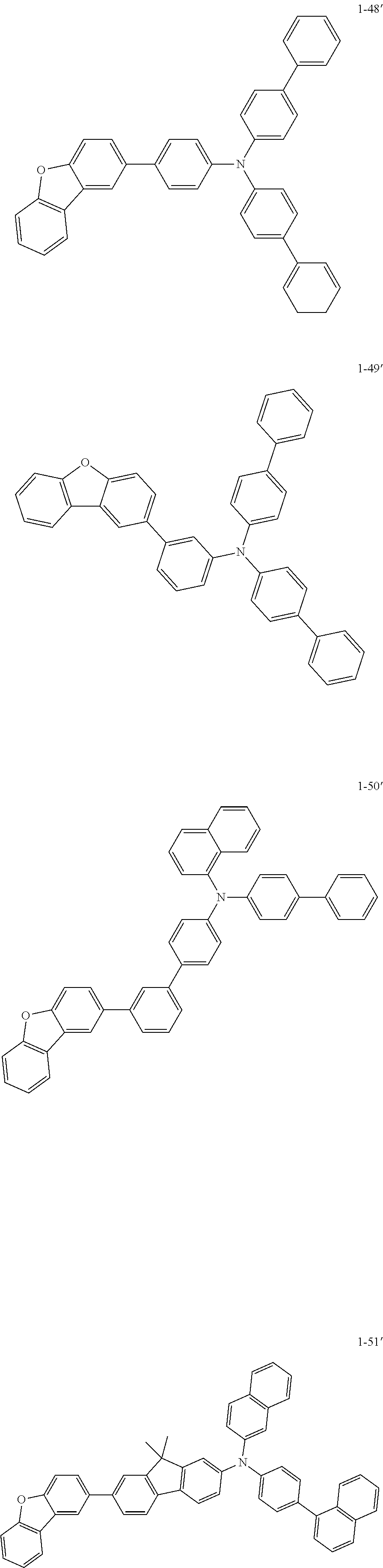 [SMILES: C1=CC(c2ccc(N(c3ccc(-c4ccccc4)cc3)c3ccc(-c4ccc5oc6ccccc6c5c4)cc3)cc2)=CCC1 ; c1ccc(-c2ccc(N(c3ccc(-c4ccccc4)cc3)c3cccc(-c4ccc5oc6ccccc6c5c4)c3)cc2)cc1 ; c1ccc(-c2ccc(N(c3ccc(-c4cccc(-c5ccc6oc7ccccc7c6c5)c4)cc3)c3cccc4ccccc34)cc2)cc1 ; CC1(C)c2cc(-c3ccc4oc5ccccc5c4c3)ccc2-c2ccc(N(c3ccc(-c4cccc5ccccc45)cc3)c3ccc4ccccc4c3)cc21]

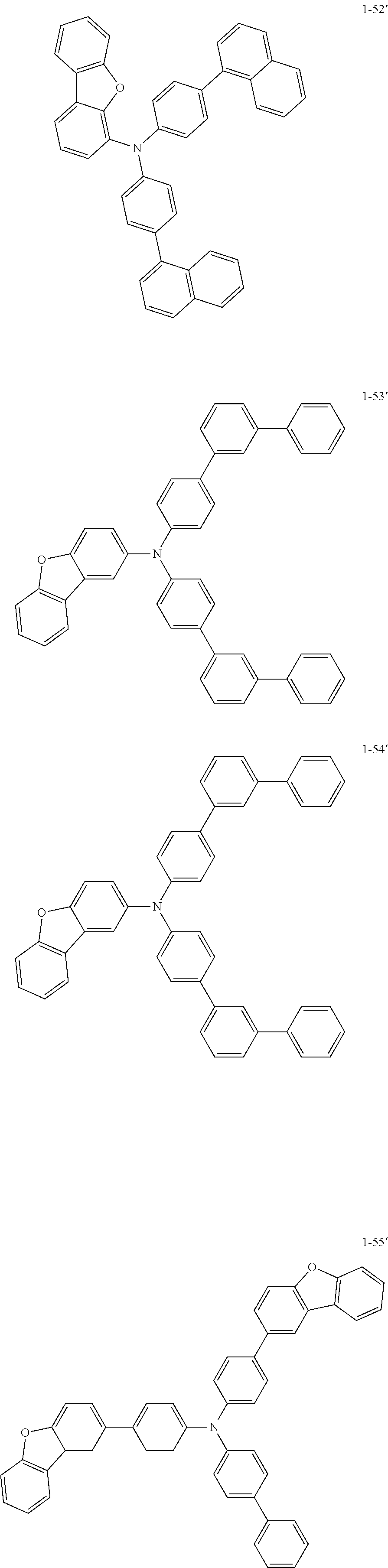 [SMILES: c1ccc2c(-c3ccc(N(c4ccc(-c5cccc6ccccc56)cc4)c4cccc5c4oc4ccccc45)cc3)cccc2c1 ; c1ccc(-c2cccc(-c3ccc(N(c4ccc(-c5cccc(-c6ccccc6)c5)cc4)c4ccc5oc6ccccc6c5c4)cc3)c2)cc1 ; c1ccc(-c2cccc(-c3ccc(N(c4ccc(-c5cccc(-c6ccccc6)c5)cc4)c4ccc5oc6ccccc6c5c4)cc3)c2)cc1 ; C1=C(C2=CC=C3Oc4ccccc4C3C2)CCC(N(c2ccc(-c3ccccc3)cc2)c2ccc(-c3ccc4oc5ccccc5c4c3)cc2)=C1]

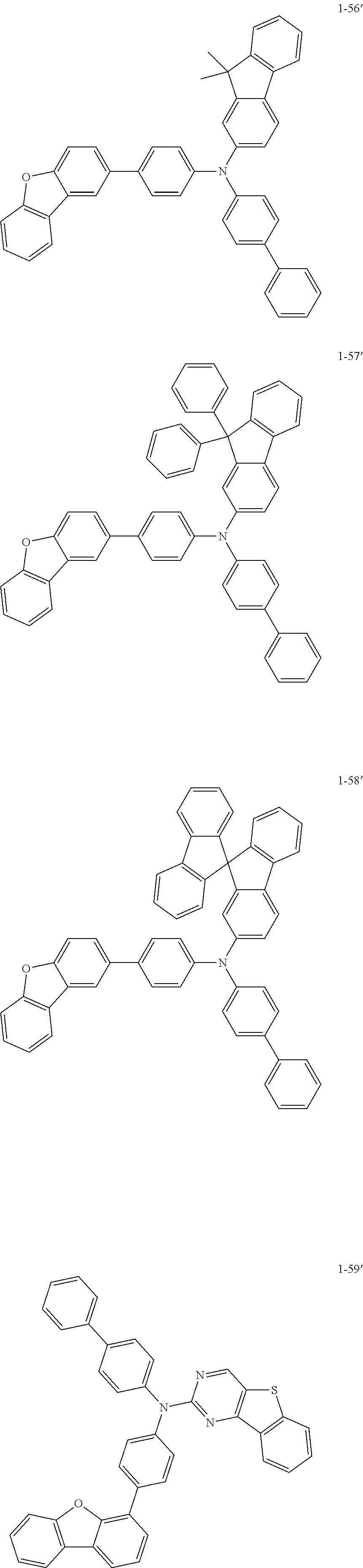 [SMILES: CC1(C)c2ccccc2-c2ccc(N(c3ccc(-c4ccccc4)cc3)c3ccc(-c4ccc5oc6ccccc6c5c4)cc3)cc21 ; c1ccc(-c2ccc(N(c3ccc(-c4ccc5oc6ccccc6c5c4)cc3)c3ccc4c(c3)C(c3ccccc3)(c3ccccc3)c3ccccc3-4)cc2)cc1 ; c1ccc(-c2ccc(N(c3ccc(-c4ccc5oc6ccccc6c5c4)cc3)c3ccc4c(c3)C3(c5ccccc5-c5ccccc53)c3ccccc3-4)cc2)cc1 ; c1ccc(-c2ccc(N(c3ccc(-c4cccc5c4oc4ccccc45)cc3)c3ncc4sc5ccccc5c4n3)cc2)cc1]

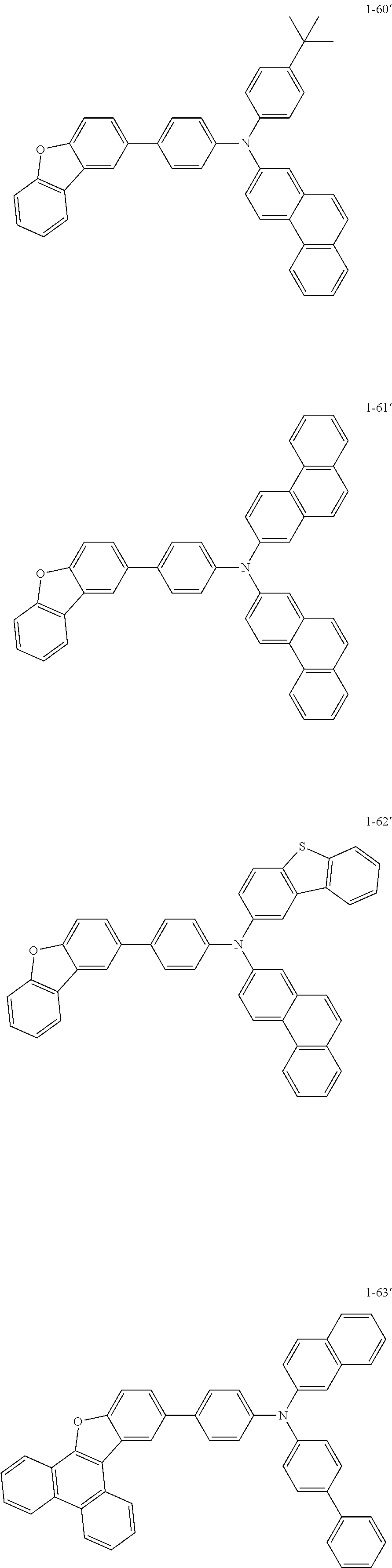 [SMILES: CC(C)(C)c1ccc(N(c2ccc(-c3ccc4oc5ccccc5c4c3)cc2)c2ccc3c(ccc4ccccc43)c2)cc1 ; c1ccc2c(c1)ccc1cc(N(c3ccc(-c4ccc5oc6ccccc6c5c4)cc3)c3ccc4c(ccc5ccccc54)c3)ccc12 ; c1ccc2c(c1)ccc1cc(N(c3ccc(-c4ccc5oc6ccccc6c5c4)cc3)c3ccc4sc5ccccc5c4c3)ccc12 ; c1ccc(-c2ccc(N(c3ccc(-c4ccc5oc6c7ccccc7c7ccccc7c6c5c4)cc3)c3ccc4ccccc4c3)cc2)cc1]

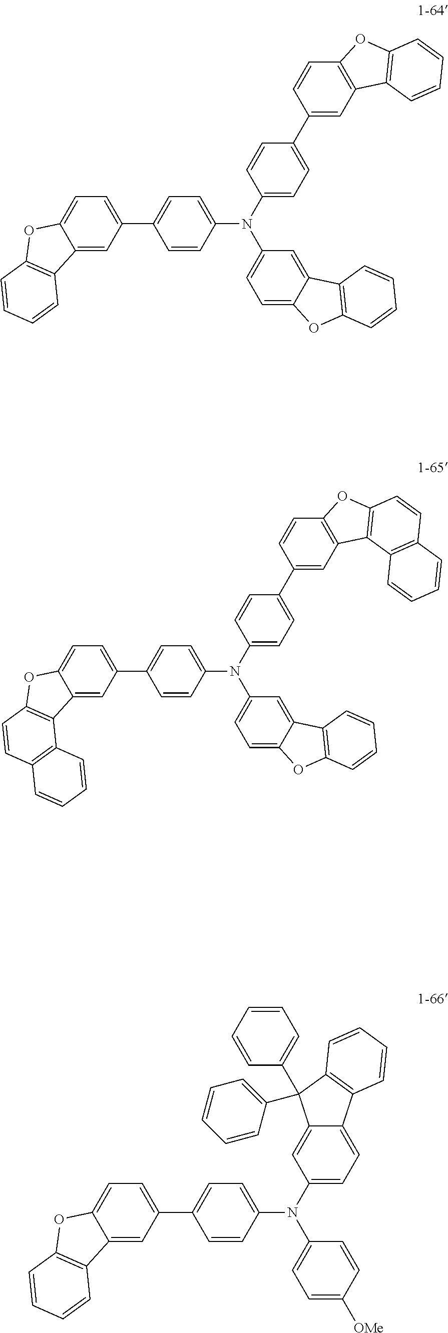 [SMILES: c1ccc2c(c1)oc1ccc(-c3ccc(N(c4ccc(-c5ccc6oc7ccccc7c6c5)cc4)c4ccc5oc6ccccc6c5c4)cc3)cc12 ; c1ccc2c(c1)ccc1oc3ccc(-c4ccc(N(c5ccc(-c6ccc7oc8ccc9ccccc9c8c7c6)cc5)c5ccc6oc7ccccc7c6c5)cc4)cc3c12 ; COc1ccc(N(c2ccc(-c3ccc4oc5ccccc5c4c3)cc2)c2ccc3c(c2)C(c2ccccc2)(c2ccccc2)c2ccccc2-3)cc1]

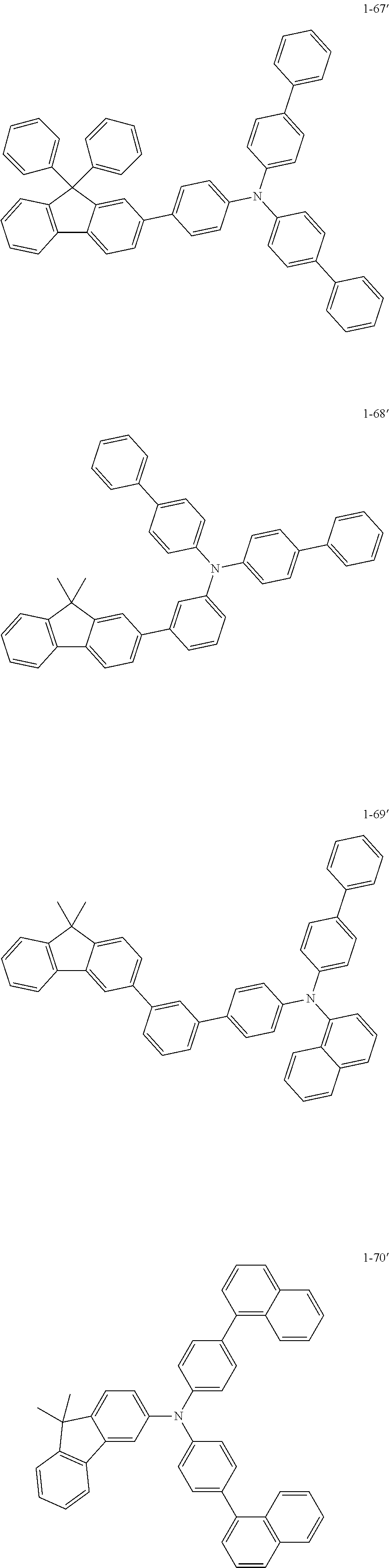 [SMILES: c1ccc(-c2ccc(N(c3ccc(-c4ccccc4)cc3)c3ccc(-c4ccc5c(c4)C(c4ccccc4)(c4ccccc4)c4ccccc4-5)cc3)cc2)cc1 ; CC1(C)c2ccccc2-c2ccc(-c3cccc(N(c4ccc(-c5ccccc5)cc4)c4ccc(-c5ccccc5)cc4)c3)cc21 ; CC1(C)c2ccccc2-c2cc(-c3cccc(-c4ccc(N(c5ccc(-c6ccccc6)cc5)c5cccc6ccccc56)cc4)c3)ccc21 ; CC1(C)c2ccccc2-c2cc(N(c3ccc(-c4cccc5ccccc45)cc3)c3ccc(-c4cccc5ccccc45)cc3)ccc21]

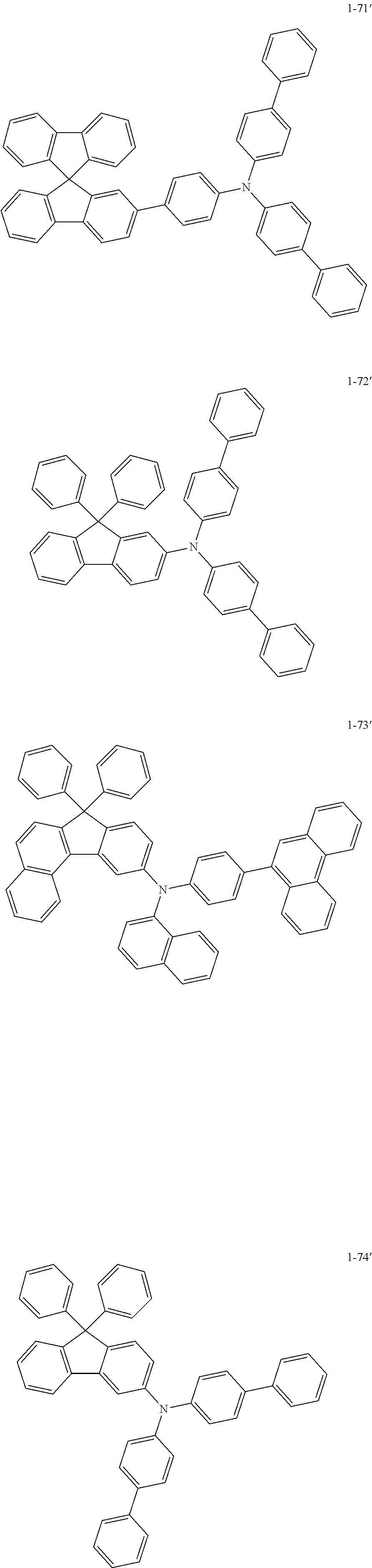 [SMILES: c1ccc(-c2ccc(N(c3ccc(-c4ccccc4)cc3)c3ccc(-c4ccc5c(c4)C4(c6ccccc6-c6ccccc64)c4ccccc4-5)cc3)cc2)cc1 ; c1ccc(-c2ccc(N(c3ccc(-c4ccccc4)cc3)c3ccc4c(c3)C(c3ccccc3)(c3ccccc3)c3ccccc3-4)cc2)cc1 ; c1ccc(C2(c3ccccc3)c3ccc(N(c4ccc(-c5cc6ccccc6c6ccccc56)cc4)c4cccc5ccccc45)cc3-c3c2ccc2ccccc32)cc1 ; c1ccc(-c2ccc(N(c3ccc(-c4ccccc4)cc3)c3ccc4c(c3)-c3ccccc3C4(c3ccccc3)c3ccccc3)cc2)cc1]

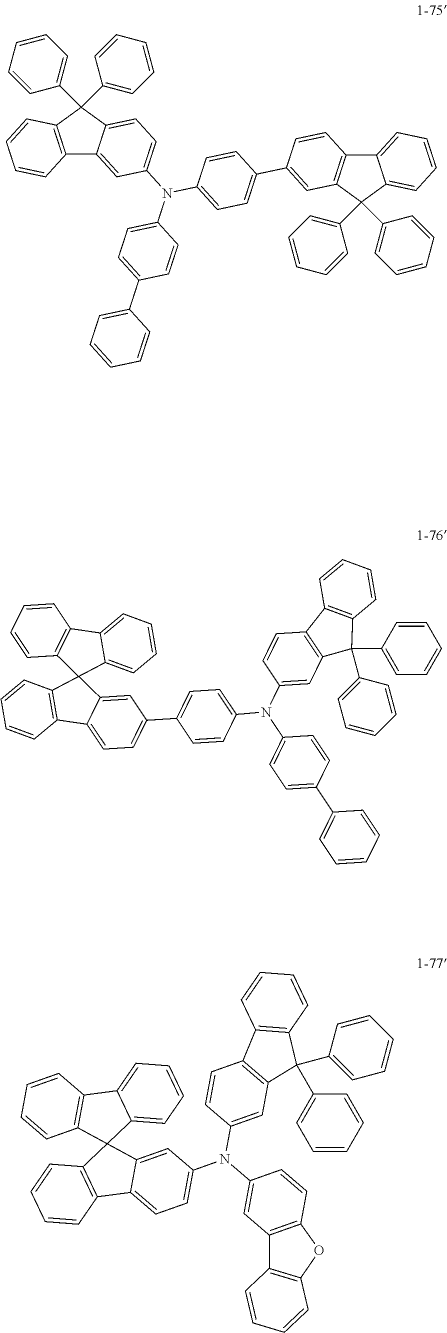 [SMILES: c1ccc(-c2ccc(N(c3ccc(-c4ccc5c(c4)C(c4ccccc4)(c4ccccc4)c4ccccc4-5)cc3)c3ccc4c(c3)-c3ccccc3C4(c3ccccc3)c3ccccc3)cc2)cc1 ; c1ccc(-c2ccc(N(c3ccc(-c4ccc5c(c4)C4(c6ccccc6-c6ccccc64)c4ccccc4-5)cc3)c3ccc4c(c3)C(c3ccccc3)(c3ccccc3)c3ccccc3-4)cc2)cc1 ; c1ccc(C2(c3ccccc3)c3ccccc3-c3ccc(N(c4ccc5c(c4)C4(c6ccccc6-c6ccccc64)c4ccccc4-5)c4ccc5oc6ccccc6c5c4)cc32)cc1]

In another embodiment of the present invention, the organic electric element includes a composition, wherein the Formula 2 is represented compounds by the followings.

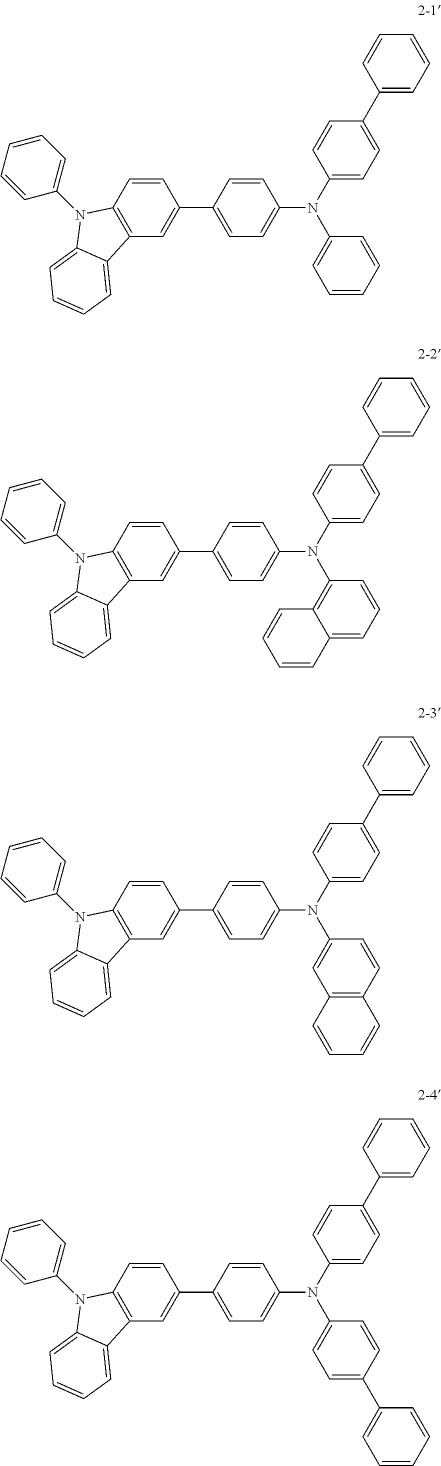 [SMILES: c1ccc(-c2ccc(N(c3ccccc3)c3ccc(-c4ccc5c(c4)c4ccccc4n5-c4ccccc4)cc3)cc2)cc1 ; c1ccc(-c2ccc(N(c3ccc(-c4ccc5c(c4)c4ccccc4n5-c4ccccc4)cc3)c3cccc4ccccc34)cc2)cc1 ; c1ccc(-c2ccc(N(c3ccc(-c4ccc5c(c4)c4ccccc4n5-c4ccccc4)cc3)c3ccc4ccccc4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1]

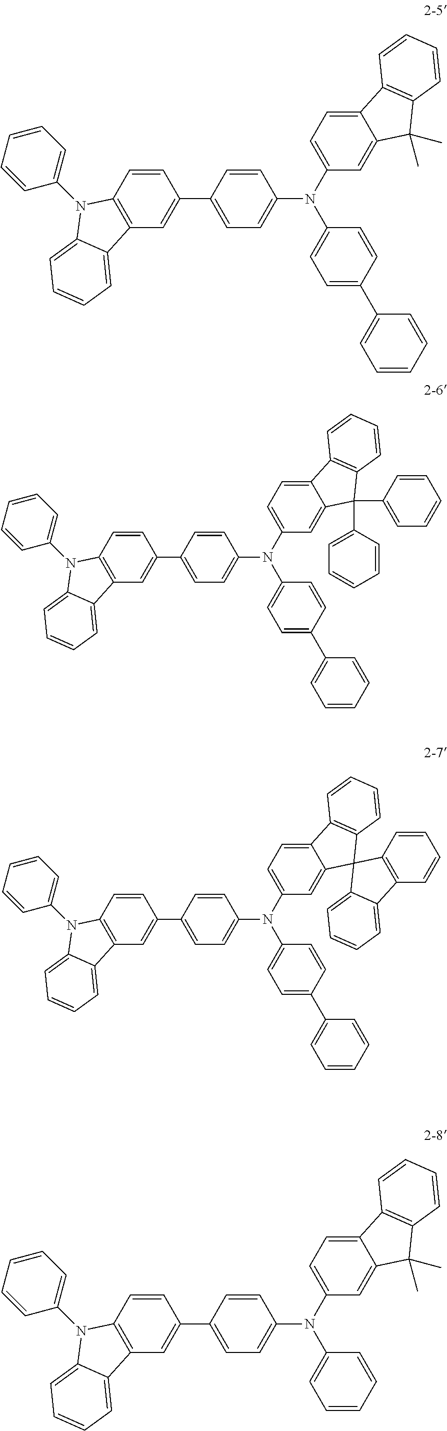 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; CC1(C)c2ccccc2-c2ccc(N(c3ccccc3)c3ccc(-c4ccc5c(c4)c4ccccc4n5-c4ccccc4)cc3)cc21]

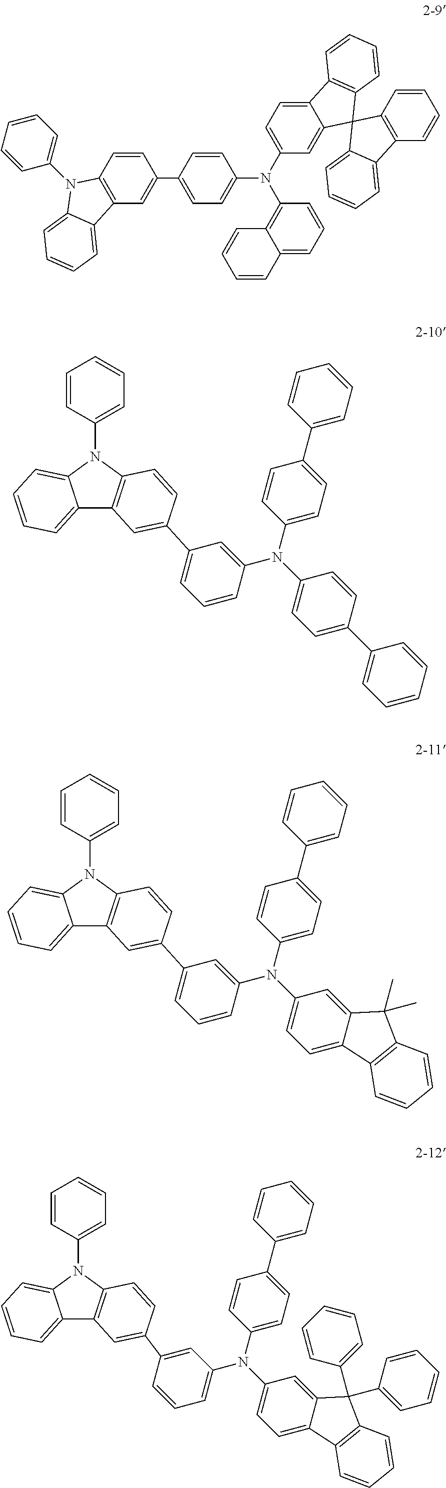 [SMILES: c1ccc(-n2c3ccccc3c3cc(-c4ccc(N(c5ccc6c(c5)C5(c7ccccc7-c7ccccc75)c5ccccc5-6)c5cccc6ccccc56)cc4)ccc32)cc1 ; c1ccc(-c2ccc(N(c3ccc(-c4ccccc4)cc3)c3cccc(-c4ccc5c(c4)c4ccccc4n5-c4ccccc4)c3)cc2)cc1 ; CC1(C)c2ccccc2-c2ccc(N(c3ccc(-c4ccccc4)cc3)c3cccc(-c4ccc5c(c4)c4ccccc4n5-c4ccccc4)c3)cc21 ; c1ccc(-c2ccc(N(c3cccc(-c4ccc5c(c4)c4ccccc4n5-c4ccccc4)c3)c3ccc4c(c3)C(c3ccccc3)(c3ccccc3)c3ccccc3-4)cc2)cc1]

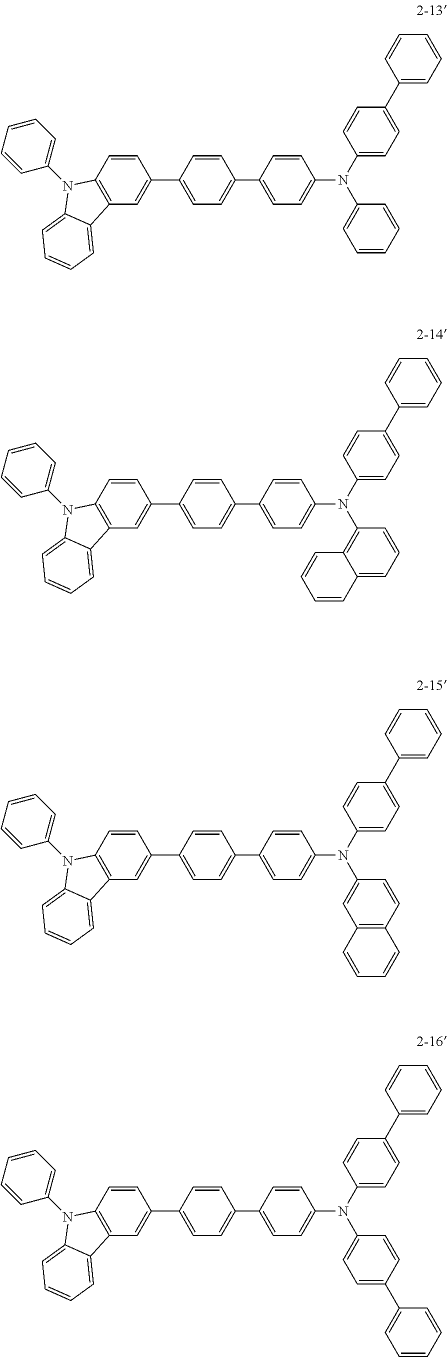 [SMILES: c1ccc(-c2ccc(N(c3ccccc3)c3ccc(-c4ccc(-c5ccc6c(c5)c5ccccc5n6-c5ccccc5)cc4)cc3)cc2)cc1 ; c1ccc(-c2ccc(N(c3ccc(-c4ccc(-c5ccc6c(c5)c5ccccc5n6-c5ccccc5)cc4)cc3)c3cccc4ccccc34)cc2)cc1 ; c1ccc(-c2ccc(N(c3ccc(-c4ccc(-c5ccc6c(c5)c5ccccc5n6-c5ccccc5)cc4)cc3)c3ccc4ccccc4c3)cc2)cc1 ; c1ccc(-c2ccc(N(c3ccc(-c4ccccc4)cc3)c3ccc(-c4ccc(-c5ccc6c(c5)c5ccccc5n6-c5ccccc5)cc4)cc3)cc2)cc1]

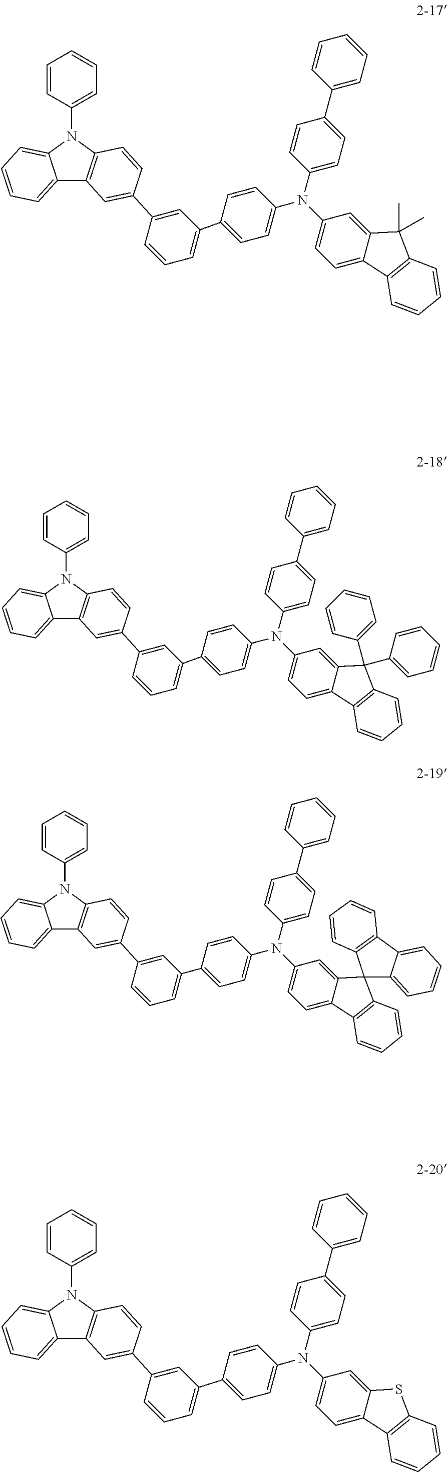 [SMILES: CC1(C)c2ccccc2-c2ccc(N(c3ccc(-c4ccccc4)cc3)c3ccc(-c4cccc(-c5ccc6c(c5)c5ccccc5n6-c5ccccc5)c4)cc3)cc21 ; c1ccc(-c2ccc(N(c3ccc(-c4cccc(-c5ccc6c(c5)c5ccccc5n6-c5ccccc5)c4)cc3)c3ccc4c(c3)C(c3ccccc3)(c3ccccc3)c3ccccc3-4)cc2)cc1 ; c1ccc(-c2ccc(N(c3ccc(-c4cccc(-c5ccc6c(c5)c5ccccc5n6-c5ccccc5)c4)cc3)c3ccc4c(c3)C3(c5ccccc5-c5ccccc53)c3ccccc3-4)cc2)cc1 ; c1ccc(-c2ccc(N(c3ccc(-c4cccc(-c5ccc6c(c5)c5ccccc5n6-c5ccccc5)c4)cc3)c3ccc4c(c3)sc3ccccc34)cc2)cc1]

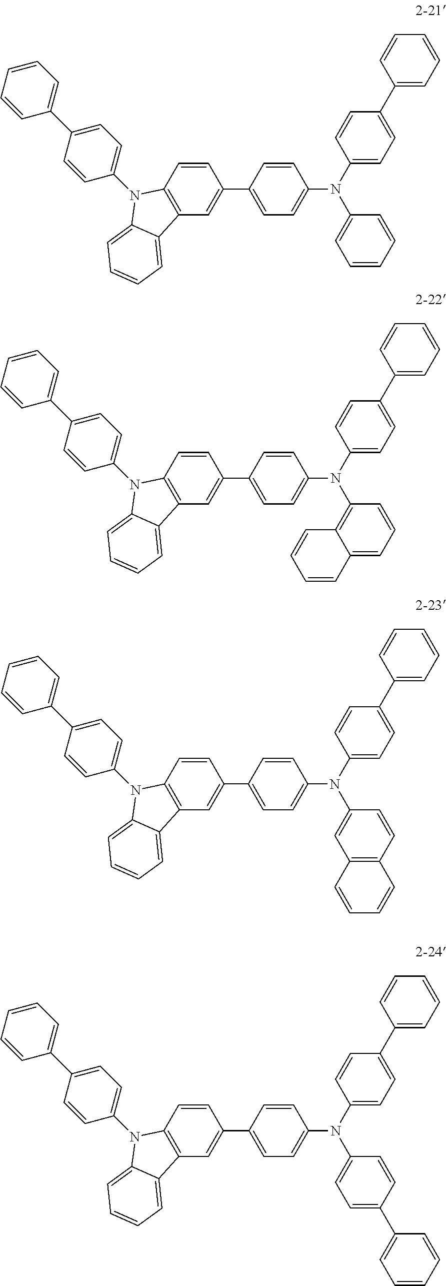 [SMILES: c1ccc(-c2ccc(N(c3ccccc3)c3ccc(-c4ccc5c(c4)c4ccccc4n5-c4ccc(-c5ccccc5)cc4)cc3)cc2)cc1 ; c1ccc(-c2ccc(N(c3ccc(-c4ccc5c(c4)c4ccccc4n5-c4ccc(-c5ccccc5)cc4)cc3)c3cccc4ccccc34)cc2)cc1 ; c1ccc(-c2ccc(N(c3ccc(-c4ccc5c(c4)c4ccccc4n5-c4ccc(-c5ccccc5)cc4)cc3)c3ccc4ccccc4c3)cc2)cc1 ; c1ccc(-c2ccc(N(c3ccc(-c4ccccc4)cc3)c3ccc(-c4ccc5c(c4)c4ccccc4n5-c4ccc(-c5ccccc5)cc4)cc3)cc2)cc1]

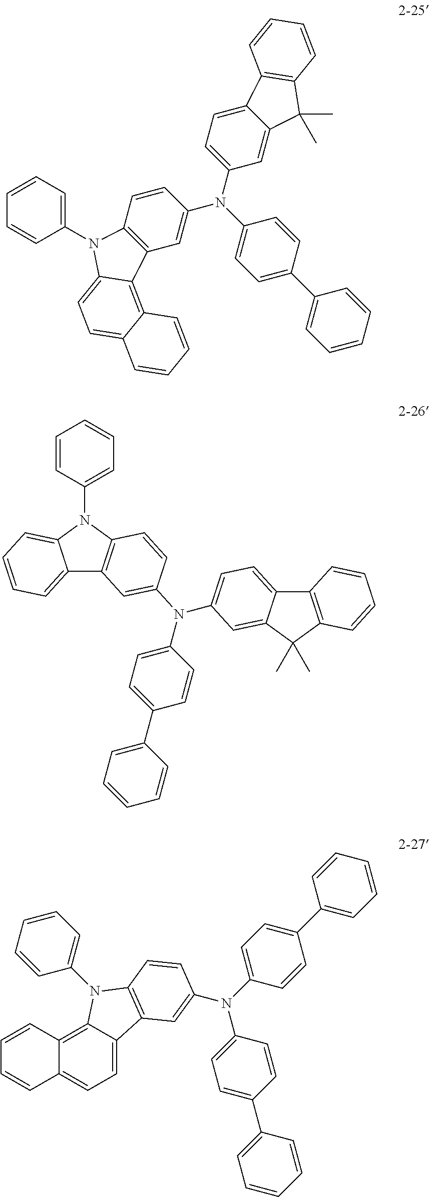 [SMILES: CC1(C)c2ccccc2-c2ccc(N(c3ccc(-c4ccccc4)cc3)c3ccc4c(c3)c3c5ccccc5ccc3n4-c3ccccc3)cc21 ; CC1(C)c2ccccc2-c2ccc(N(c3ccc(-c4ccccc4)cc3)c3ccc4c(c3)c3ccccc3n4-c3ccccc3)cc21 ; c1ccc(-c2ccc(N(c3ccc(-c4ccccc4)cc3)c3ccc4c(c3)c3ccc5ccccc5c3n4-c3ccccc3)cc2)cc1]

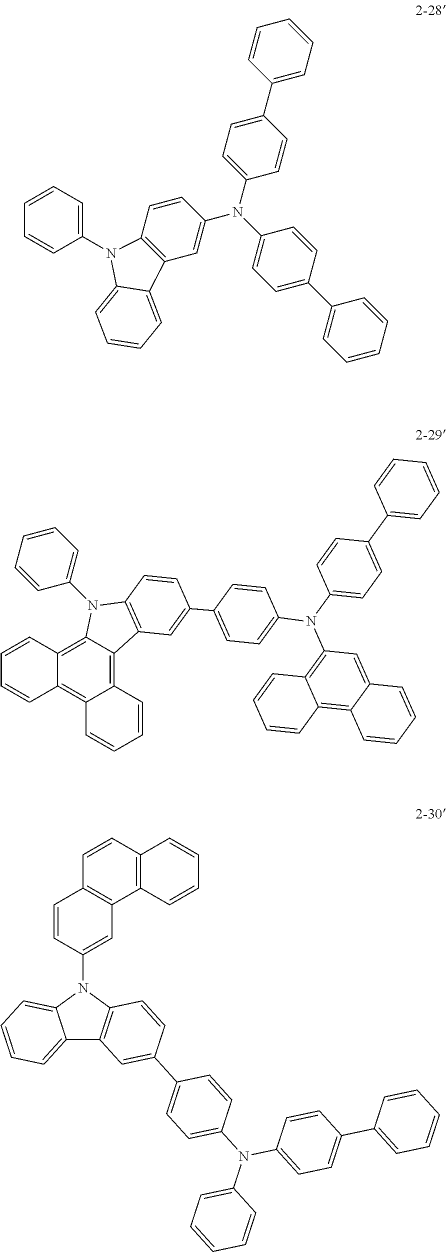 [SMILES: c1ccc(-c2ccc(N(c3ccc(-c4ccccc4)cc3)c3ccc4c(c3)c3ccccc3n4-c3ccccc3)cc2)cc1 ; c1ccc(-c2ccc(N(c3ccc(-c4ccc5c(c4)c4c6ccccc6c6ccccc6c4n5-c4ccccc4)cc3)c3cc4ccccc4c4ccccc34)cc2)cc1 ; c1ccc(-c2ccc(N(c3ccccc3)c3ccc(-c4ccc5c(c4)c4ccccc4n5-c4ccc5ccc6ccccc6c5c4)cc3)cc2)cc1]

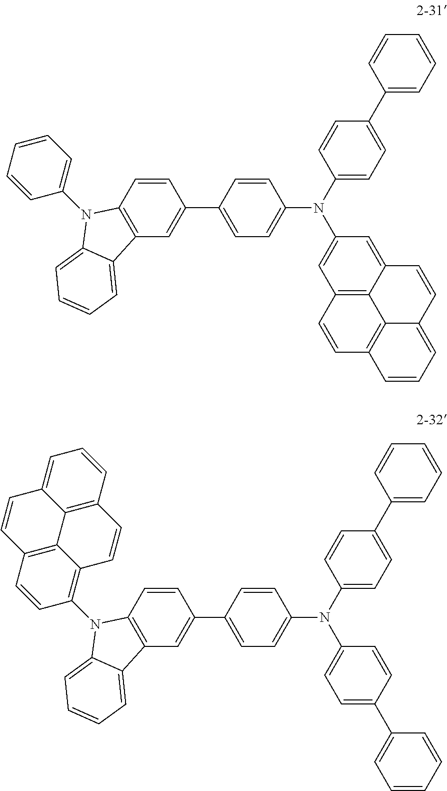 [SMILES: c1ccc(-c2ccc(N(c3ccc(-c4ccc5c(c4)c4ccccc4n5-c4ccccc4)cc3)c3cc4ccc5cccc6ccc(c3)c4c56)cc2)cc1 ; c1ccc(-c2ccc(N(c3ccc(-c4ccccc4)cc3)c3ccc(-c4ccc5c(c4)c4ccccc4n5-c4ccc5ccc6cccc7ccc4c5c67)cc3)cc2)cc1]

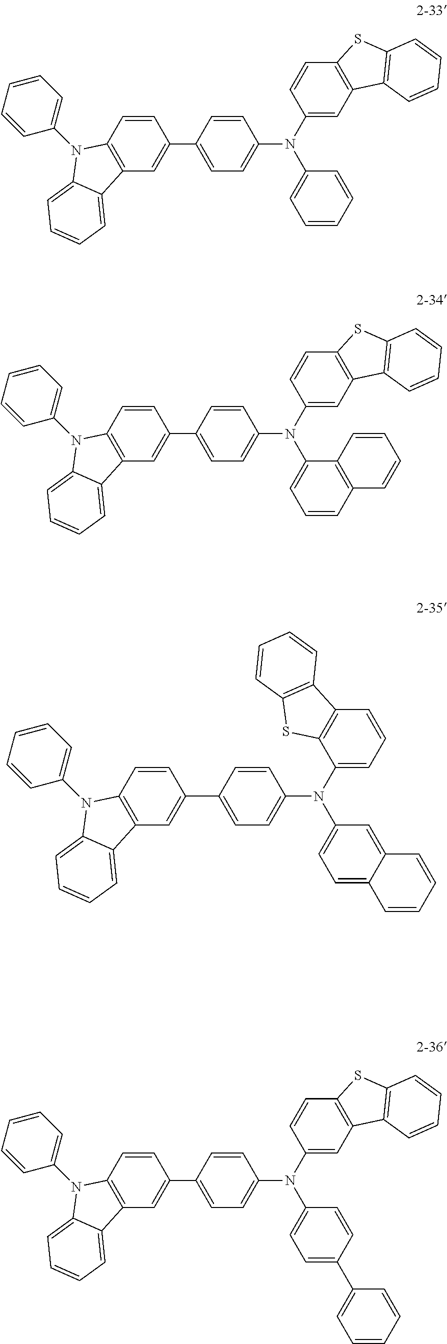 [SMILES: c1ccc(N(c2ccc(-c3ccc4c(c3)c3ccccc3n4-c3ccccc3)cc2)c2ccc3sc4ccccc4c3c2)cc1 ; c1ccc(-n2c3ccccc3c3cc(-c4ccc(N(c5ccc6sc7ccccc7c6c5)c5cccc6ccccc56)cc4)ccc32)cc1 ; c1ccc(-n2c3ccccc3c3cc(-c4ccc(N(c5ccc6ccccc6c5)c5cccc6c5sc5ccccc56)cc4)ccc32)cc1 ; c1ccc(-c2ccc(N(c3ccc(-c4ccc5c(c4)c4ccccc4n5-c4ccccc4)cc3)c3ccc4sc5ccccc5c4c3)cc2)cc1]

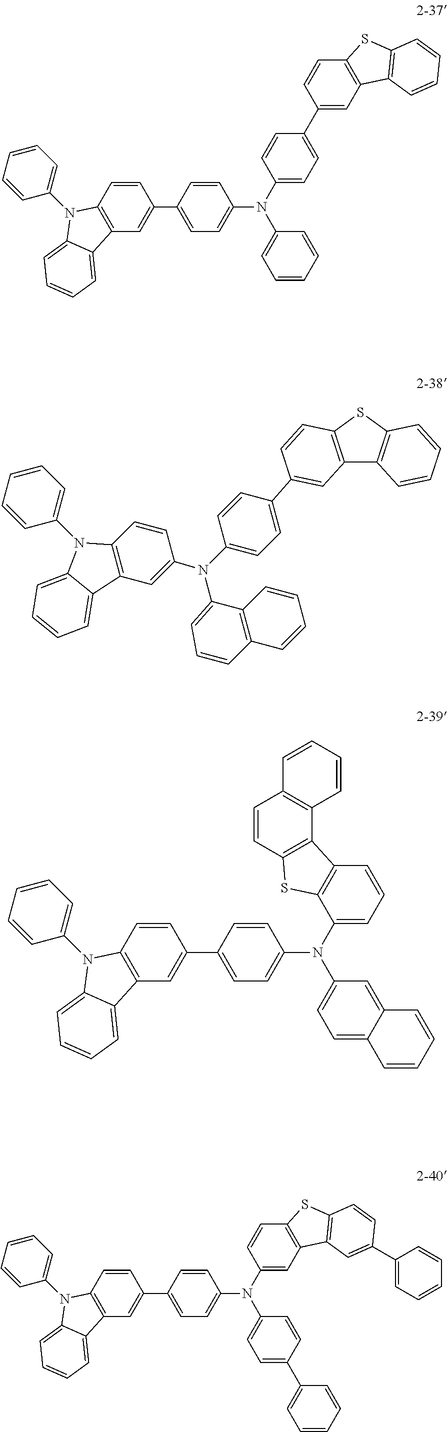 [SMILES: c1ccc(N(c2ccc(-c3ccc4sc5ccccc5c4c3)cc2)c2ccc(-c3ccc4c(c3)c3ccccc3n4-c3ccccc3)cc2)cc1 ; c1ccc(-n2c3ccccc3c3cc(N(c4ccc(-c5ccc6sc7ccccc7c6c5)cc4)c4cccc5ccccc45)ccc32)cc1 ; c1ccc(-n2c3ccccc3c3cc(-c4ccc(N(c5ccc6ccccc6c5)c5cccc6c5sc5ccc7ccccc7c56)cc4)ccc32)cc1 ; c1ccc(-c2ccc(N(c3ccc(-c4ccc5c(c4)c4ccccc4n5-c4ccccc4)cc3)c3ccc4sc5ccc(-c6ccccc6)cc5c4c3)cc2)cc1]

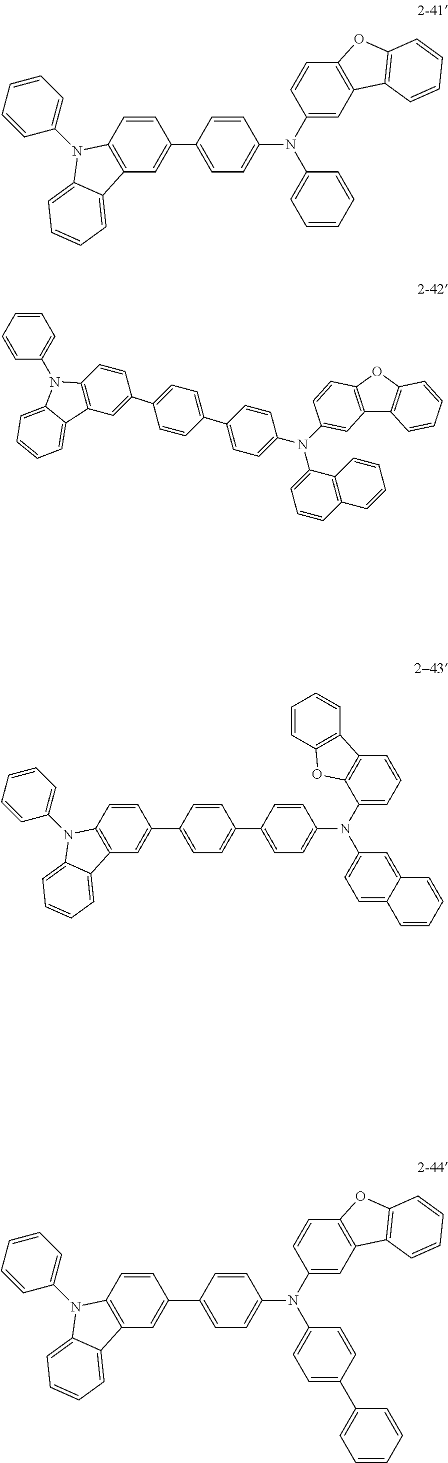 [SMILES: c1ccc(N(c2ccc(-c3ccc4c(c3)c3ccccc3n4-c3ccccc3)cc2)c2ccc3oc4ccccc4c3c2)cc1 ; c1ccc(-n2c3ccccc3c3cc(-c4ccc(-c5ccc(N(c6ccc7oc8ccccc8c7c6)c6cccc7ccccc67)cc5)cc4)ccc32)cc1 ; c1ccc(-n2c3ccccc3c3cc(-c4ccc(-c5ccc(N(c6ccc7ccccc7c6)c6cccc7c6oc6ccccc67)cc5)cc4)ccc32)cc1 ; c1ccc(-c2ccc(N(c3ccc(-c4ccc5c(c4)c4ccccc4n5-c4ccccc4)cc3)c3ccc4oc5ccccc5c4c3)cc2)cc1]

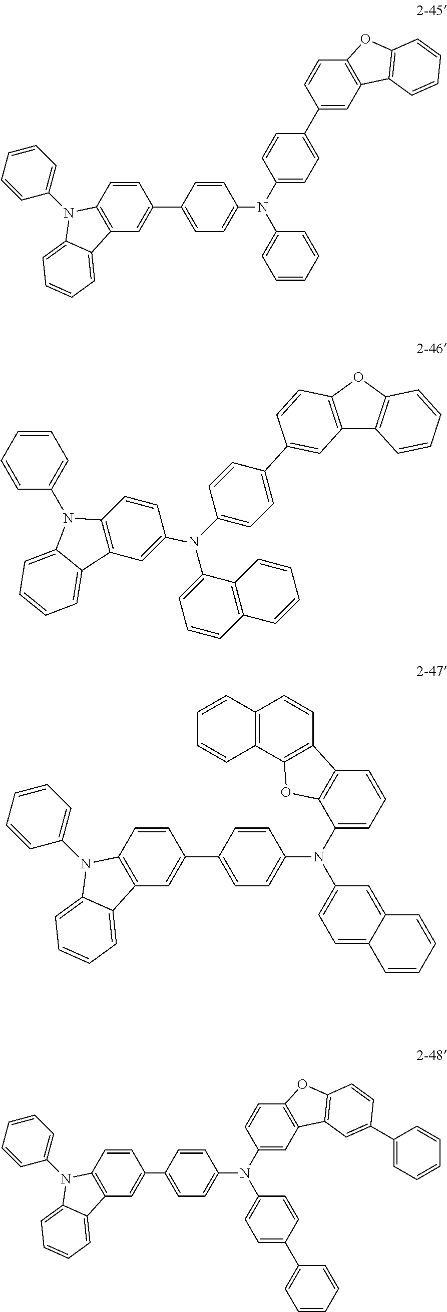 [SMILES: c1ccc(N(c2ccc(-c3ccc4oc5ccccc5c4c3)cc2)c2ccc(-c3ccc4c(c3)c3ccccc3n4-c3ccccc3)cc2)cc1 ; c1ccc(-n2c3ccccc3c3cc(N(c4ccc(-c5ccc6oc7ccccc7c6c5)cc4)c4cccc5ccccc45)ccc32)cc1 ; c1ccc(-n2c3ccccc3c3cc(-c4ccc(N(c5ccc6ccccc6c5)c5cccc6c5oc5c7ccccc7ccc65)cc4)ccc32)cc1 ; c1ccc(-c2ccc(N(c3ccc(-c4ccc5c(c4)c4ccccc4n5-c4ccccc4)cc3)c3ccc4oc5ccc(-c6ccccc6)cc5c4c3)cc2)cc1]

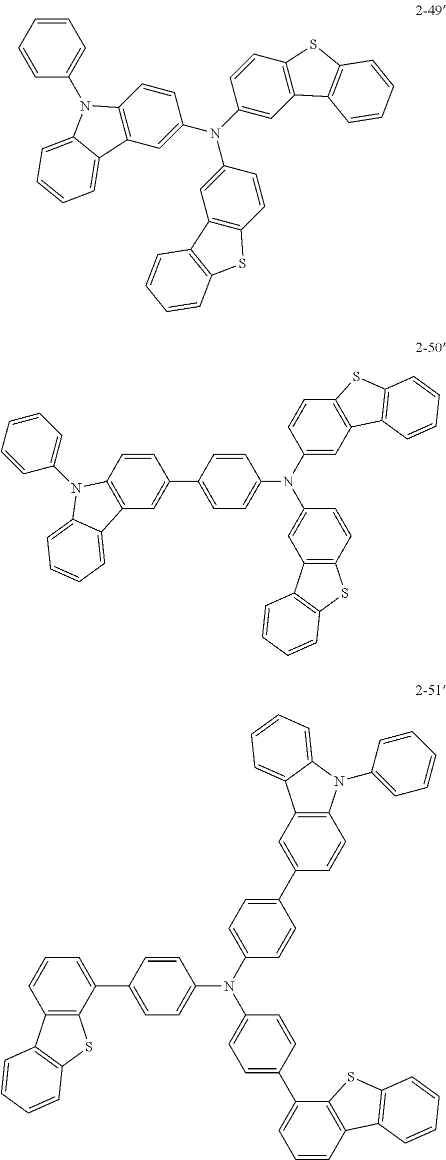 [SMILES: c1ccc(-n2c3ccccc3c3cc(N(c4ccc5sc6ccccc6c5c4)c4ccc5sc6ccccc6c5c4)ccc32)cc1 ; c1ccc(-n2c3ccccc3c3cc(-c4ccc(N(c5ccc6sc7ccccc7c6c5)c5ccc6sc7ccccc7c6c5)cc4)ccc32)cc1 ; c1ccc(-n2c3ccccc3c3cc(-c4ccc(N(c5ccc(-c6cccc7c6sc6ccccc67)cc5)c5ccc(-c6cccc7c6sc6ccccc67)cc5)cc4)ccc32)cc1]

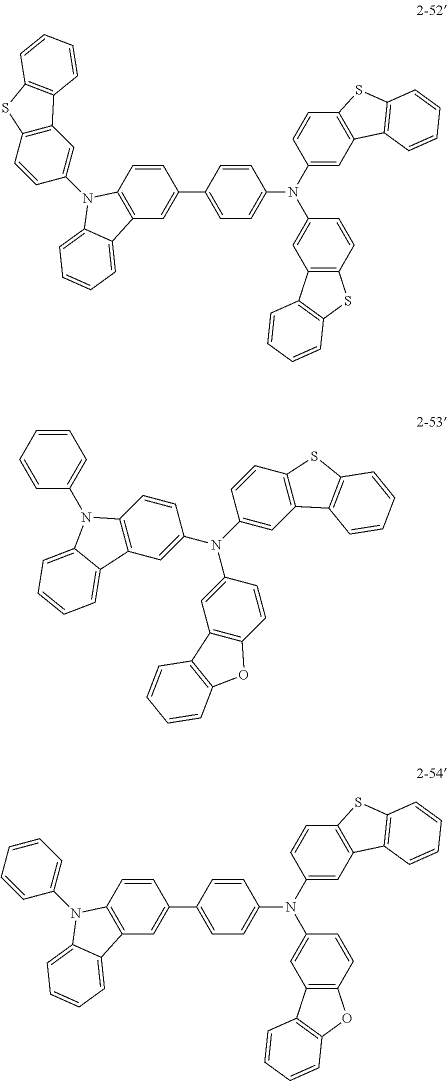 [SMILES: c1ccc2c(c1)sc1ccc(N(c3ccc(-c4ccc5c(c4)c4ccccc4n5-c4ccc5sc6ccccc6c5c4)cc3)c3ccc4sc5ccccc5c4c3)cc12 ; c1ccc(-n2c3ccccc3c3cc(N(c4ccc5oc6ccccc6c5c4)c4ccc5sc6ccccc6c5c4)ccc32)cc1 ; c1ccc(-n2c3ccccc3c3cc(-c4ccc(N(c5ccc6oc7ccccc7c6c5)c5ccc6sc7ccccc7c6c5)cc4)ccc32)cc1]

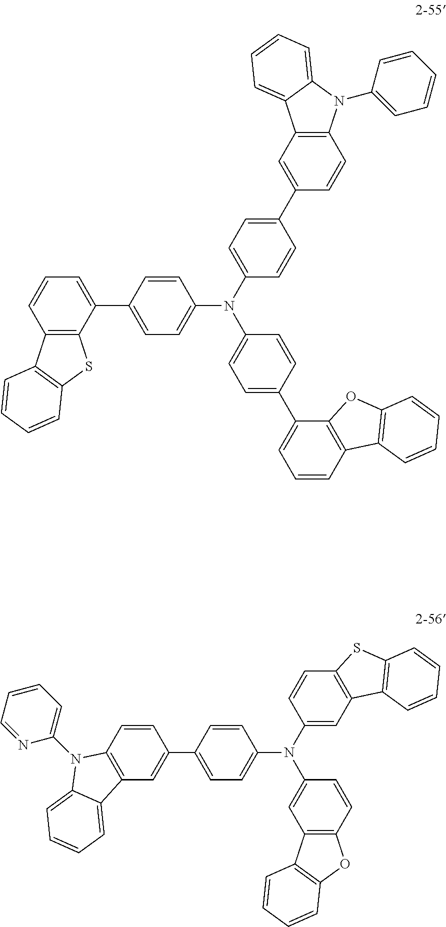 [SMILES: c1ccc(-n2c3ccccc3c3cc(-c4ccc(N(c5ccc(-c6cccc7c6oc6ccccc67)cc5)c5ccc(-c6cccc7c6sc6ccccc67)cc5)cc4)ccc32)cc1 ; c1ccc(-n2c3ccccc3c3cc(-c4ccc(N(c5ccc6oc7ccccc7c6c5)c5ccc6sc7ccccc7c6c5)cc4)ccc32)nc1]

In another embodiment of the present invention, the organic electric element includes a composition, wherein the Formula 2′ is represented compounds by the followings.

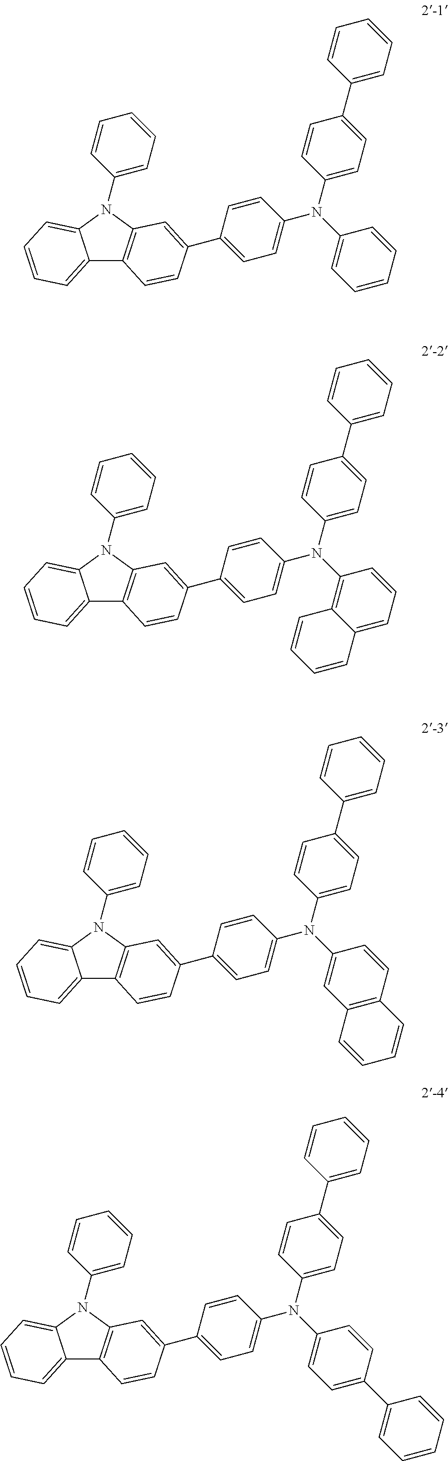 [SMILES: c1ccc(-c2ccc(N(c3ccccc3)c3ccc(-c4ccc5c6ccccc6n(-c6ccccc6)c5c4)cc3)cc2)cc1 ; c1ccc(-c2ccc(N(c3ccc(-c4ccc5c6ccccc6n(-c6ccccc6)c5c4)cc3)c3cccc4ccccc34)cc2)cc1 ; c1ccc(-c2ccc(N(c3ccc(-c4ccc5c6ccccc6n(-c6ccccc6)c5c4)cc3)c3ccc4ccccc4c3)cc2)cc1 ; c1ccc(-c2ccc(N(c3ccc(-c4ccccc4)cc3)c3ccc(-c4ccc5c6ccccc6n(-c6ccccc6)c5c4)cc3)cc2)cc1]

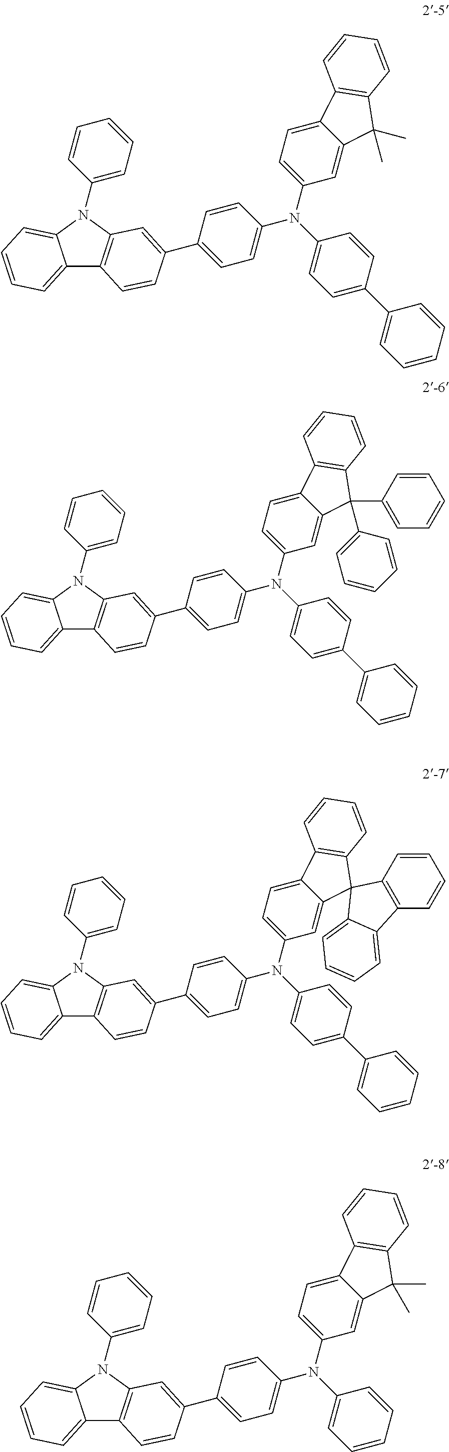 [SMILES: CC1(C)c2ccccc2-c2ccc(N(c3ccc(-c4ccccc4)cc3)c3ccc(-c4ccc5c6ccccc6n(-c6ccccc6)c5c4)cc3)cc21 ; c1ccc(-c2ccc(N(c3ccc(-c4ccc5c6ccccc6n(-c6ccccc6)c5c4)cc3)c3ccc4c(c3)C(c3ccccc3)(c3ccccc3)c3ccccc3-4)cc2)cc1 ; c1ccc(-c2ccc(N(c3ccc(-c4ccc5c6ccccc6n(-c6ccccc6)c5c4)cc3)c3ccc4c(c3)C3(c5ccccc5-c5ccccc53)c3ccccc3-4)cc2)cc1 ; CC1(C)c2ccccc2-c2ccc(N(c3ccccc3)c3ccc(-c4ccc5c6ccccc6n(-c6ccccc6)c5c4)cc3)cc21]

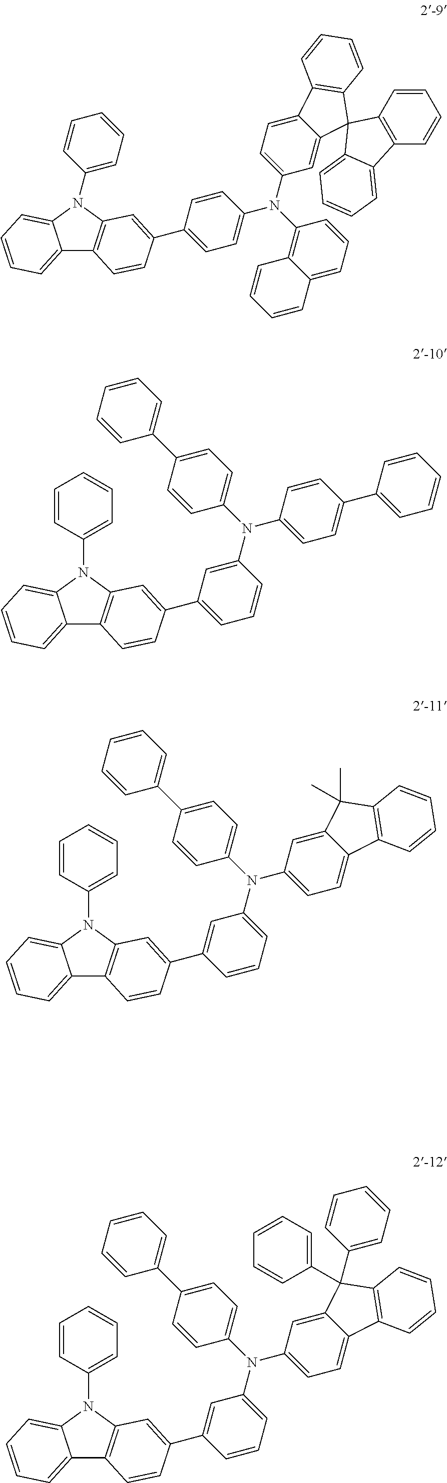 [SMILES: c1ccc(-n2c3ccccc3c3ccc(-c4ccc(N(c5ccc6c(c5)C5(c7ccccc7-c7ccccc75)c5ccccc5-6)c5cccc6ccccc56)cc4)cc32)cc1 ; c1ccc(-c2ccc(N(c3ccc(-c4ccccc4)cc3)c3cccc(-c4ccc5c6ccccc6n(-c6ccccc6)c5c4)c3)cc2)cc1 ; CC1(C)c2ccccc2-c2ccc(N(c3ccc(-c4ccccc4)cc3)c3cccc(-c4ccc5c6ccccc6n(-c6ccccc6)c5c4)c3)cc21 ; c1ccc(-c2ccc(N(c3cccc(-c4ccc5c6ccccc6n(-c6ccccc6)c5c4)c3)c3ccc4c(c3)C(c3ccccc3)(c3ccccc3)c3ccccc3-4)cc2)cc1]

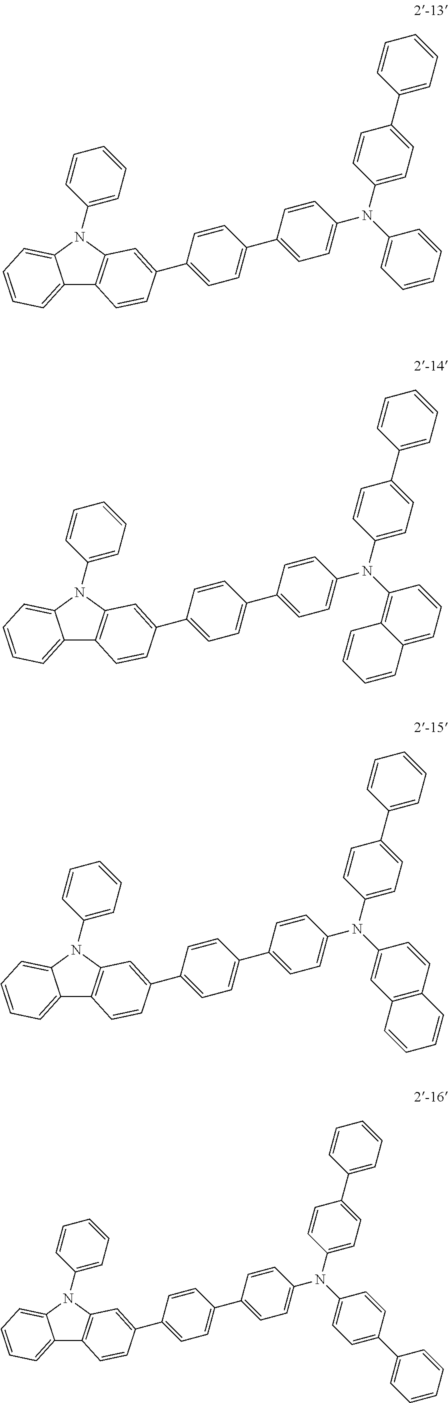 [SMILES: c1ccc(-c2ccc(N(c3ccccc3)c3ccc(-c4ccc(-c5ccc6c7ccccc7n(-c7ccccc7)c6c5)cc4)cc3)cc2)cc1 ; c1ccc(-c2ccc(N(c3ccc(-c4ccc(-c5ccc6c7ccccc7n(-c7ccccc7)c6c5)cc4)cc3)c3cccc4ccccc34)cc2)cc1 ; c1ccc(-c2ccc(N(c3ccc(-c4ccc(-c5ccc6c7ccccc7n(-c7ccccc7)c6c5)cc4)cc3)c3ccc4ccccc4c3)cc2)cc1 ; c1ccc(-c2ccc(N(c3ccc(-c4ccccc4)cc3)c3ccc(-c4ccc(-c5ccc6c7ccccc7n(-c7ccccc7)c6c5)cc4)cc3)cc2)cc1]

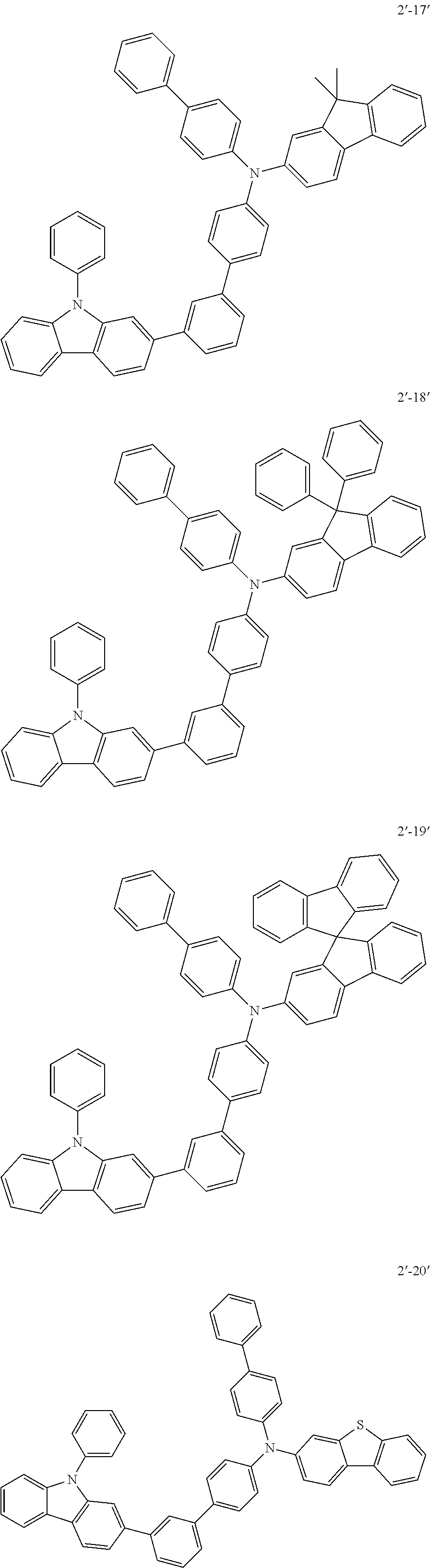 [SMILES: CC1(C)c2ccccc2-c2ccc(N(c3ccc(-c4ccccc4)cc3)c3ccc(-c4cccc(-c5ccc6c7ccccc7n(-c7ccccc7)c6c5)c4)cc3)cc21 ; c1ccc(-c2ccc(N(c3ccc(-c4cccc(-c5ccc6c7ccccc7n(-c7ccccc7)c6c5)c4)cc3)c3ccc4c(c3)C(c3ccccc3)(c3ccccc3)c3ccccc3-4)cc2)cc1 ; c1ccc(-c2ccc(N(c3ccc(-c4cccc(-c5ccc6c7ccccc7n(-c7ccccc7)c6c5)c4)cc3)c3ccc4c(c3)C3(c5ccccc5-c5ccccc53)c3ccccc3-4)cc2)cc1 ; c1ccc(-c2ccc(N(c3ccc(-c4cccc(-c5ccc6c7ccccc7n(-c7ccccc7)c6c5)c4)cc3)c3ccc4c(c3)sc3ccccc34)cc2)cc1]

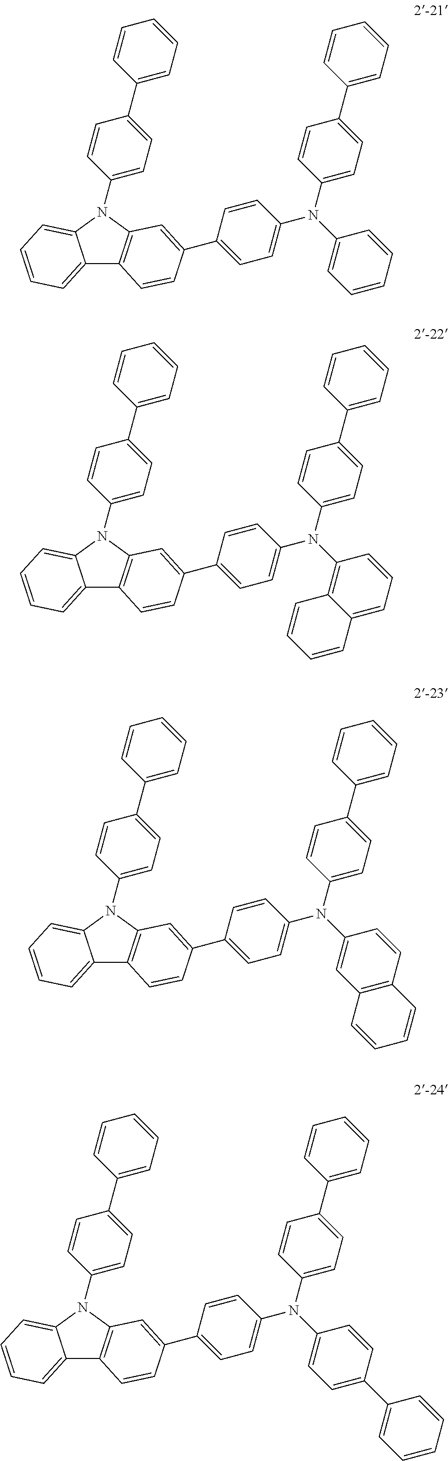 [SMILES: c1ccc(-c2ccc(N(c3ccccc3)c3ccc(-c4ccc5c6ccccc6n(-c6ccc(-c7ccccc7)cc6)c5c4)cc3)cc2)cc1 ; c1ccc(-c2ccc(N(c3ccc(-c4ccc5c6ccccc6n(-c6ccc(-c7ccccc7)cc6)c5c4)cc3)c3cccc4ccccc34)cc2)cc1 ; c1ccc(-c2ccc(N(c3ccc(-c4ccc5c6ccccc6n(-c6ccc(-c7ccccc7)cc6)c5c4)cc3)c3ccc4ccccc4c3)cc2)cc1 ; c1ccc(-c2ccc(N(c3ccc(-c4ccccc4)cc3)c3ccc(-c4ccc5c6ccccc6n(-c6ccc(-c7ccccc7)cc6)c5c4)cc3)cc2)cc1]

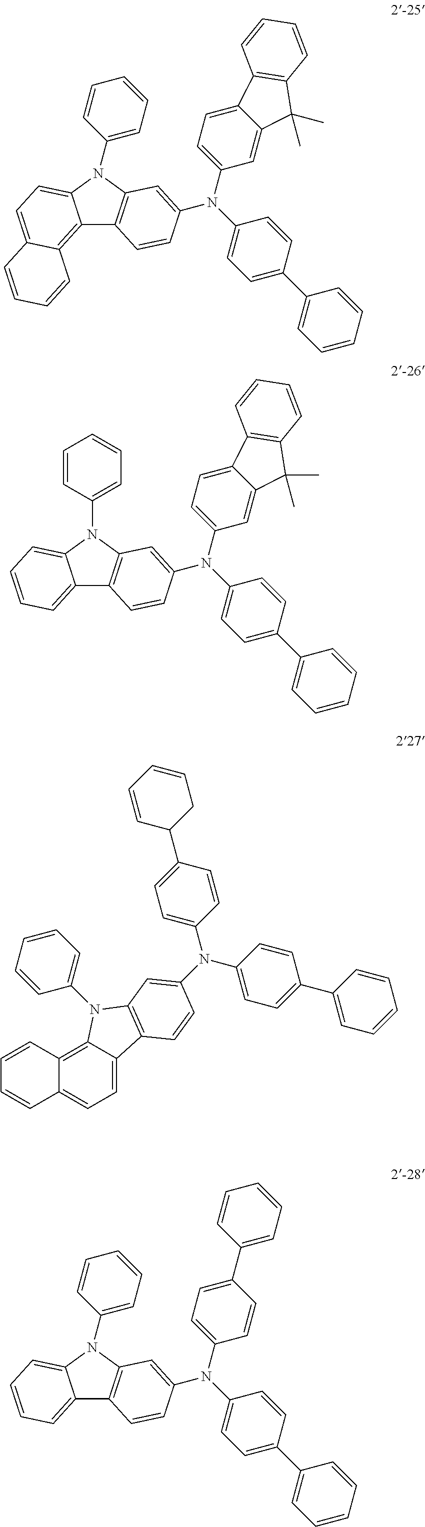

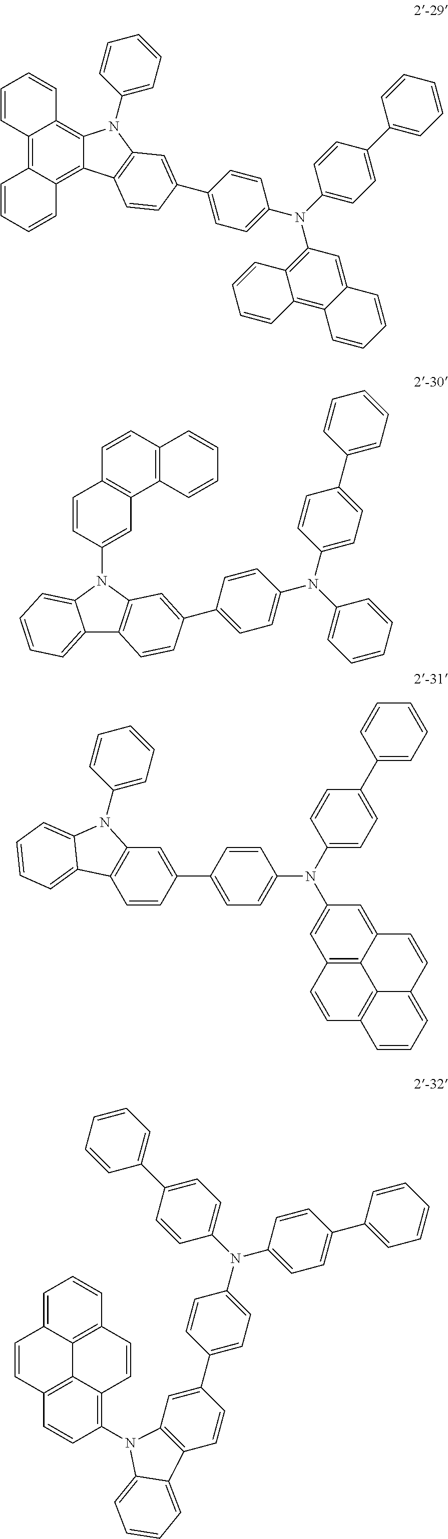

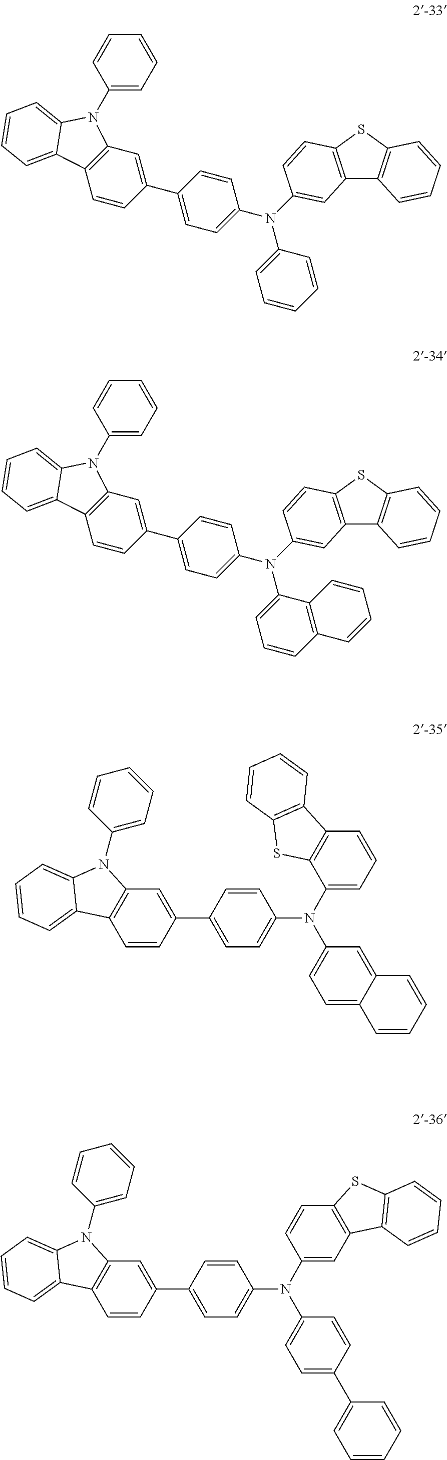 [SMILES: c1ccc(N(c2ccc(-c3ccc4c5ccccc5n(-c5ccccc5)c4c3)cc2)c2ccc3sc4ccccc4c3c2)cc1 ; c1ccc(-n2c3ccccc3c3ccc(-c4ccc(N(c5ccc6sc7ccccc7c6c5)c5cccc6ccccc56)cc4)cc32)cc1 ; c1ccc(-n2c3ccccc3c3ccc(-c4ccc(N(c5ccc6ccccc6c5)c5cccc6c5sc5ccccc56)cc4)cc32)cc1 ; c1ccc(-c2ccc(N(c3ccc(-c4ccc5c6ccccc6n(-c6ccccc6)c5c4)cc3)c3ccc4sc5ccccc5c4c3)cc2)cc1]

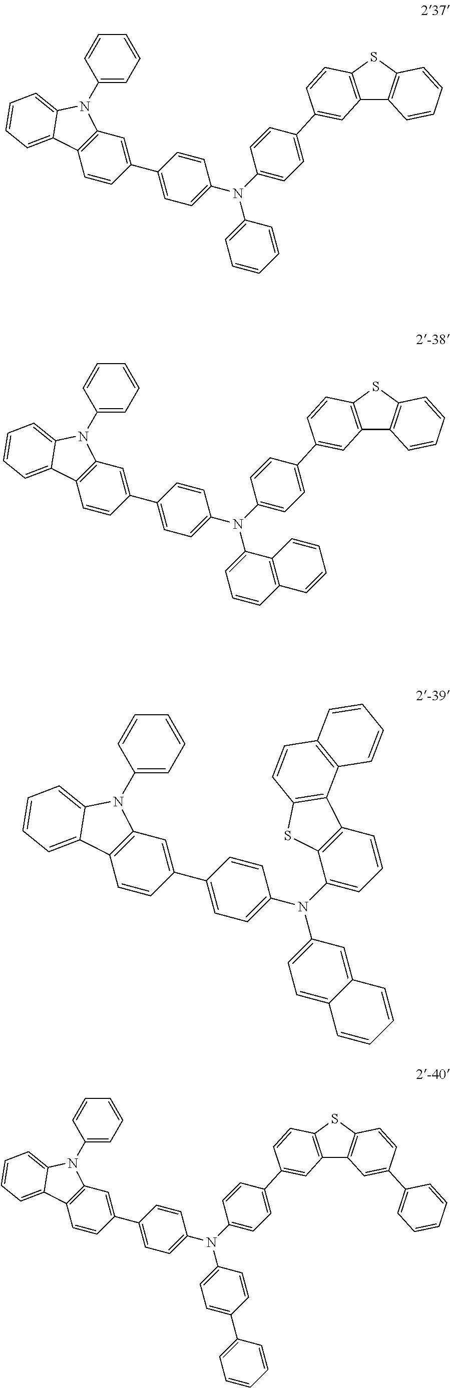 [SMILES: c1ccc(N(c2ccc(-c3ccc4sc5ccccc5c4c3)cc2)c2ccc(-c3ccc4c5ccccc5n(-c5ccccc5)c4c3)cc2)cc1 ; c1ccc(-n2c3ccccc3c3ccc(-c4ccc(N(c5ccc(-c6ccc7sc8ccccc8c7c6)cc5)c5cccc6ccccc56)cc4)cc32)cc1 ; c1ccc(-n2c3ccccc3c3ccc(-c4ccc(N(c5ccc6ccccc6c5)c5cccc6c5sc5ccc7ccccc7c56)cc4)cc32)cc1 ; c1ccc(-c2ccc(N(c3ccc(-c4ccc5sc6ccc(-c7ccccc7)cc6c5c4)cc3)c3ccc(-c4ccc5c6ccccc6n(-c6ccccc6)c5c4)cc3)cc2)cc1]

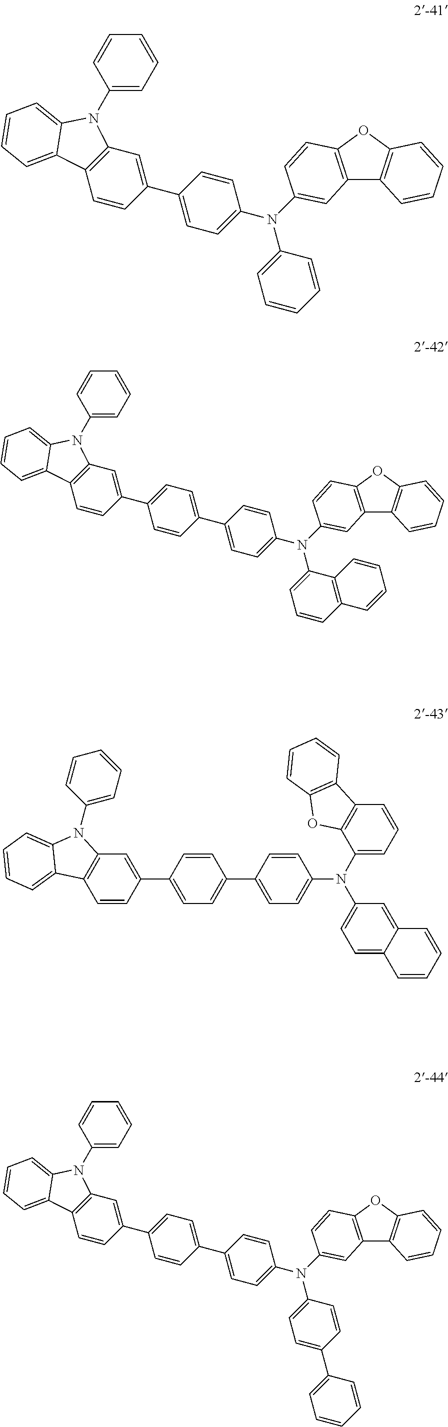 [SMILES: c1ccc(N(c2ccc(-c3ccc4c5ccccc5n(-c5ccccc5)c4c3)cc2)c2ccc3oc4ccccc4c3c2)cc1 ; c1ccc(-n2c3ccccc3c3ccc(-c4ccc(-c5ccc(N(c6ccc7oc8ccccc8c7c6)c6cccc7ccccc67)cc5)cc4)cc32)cc1 ; c1ccc(-n2c3ccccc3c3ccc(-c4ccc(-c5ccc(N(c6ccc7ccccc7c6)c6cccc7c6oc6ccccc67)cc5)cc4)cc32)cc1 ; c1ccc(-c2ccc(N(c3ccc(-c4ccc(-c5ccc6c7ccccc7n(-c7ccccc7)c6c5)cc4)cc3)c3ccc4oc5ccccc5c4c3)cc2)cc1]

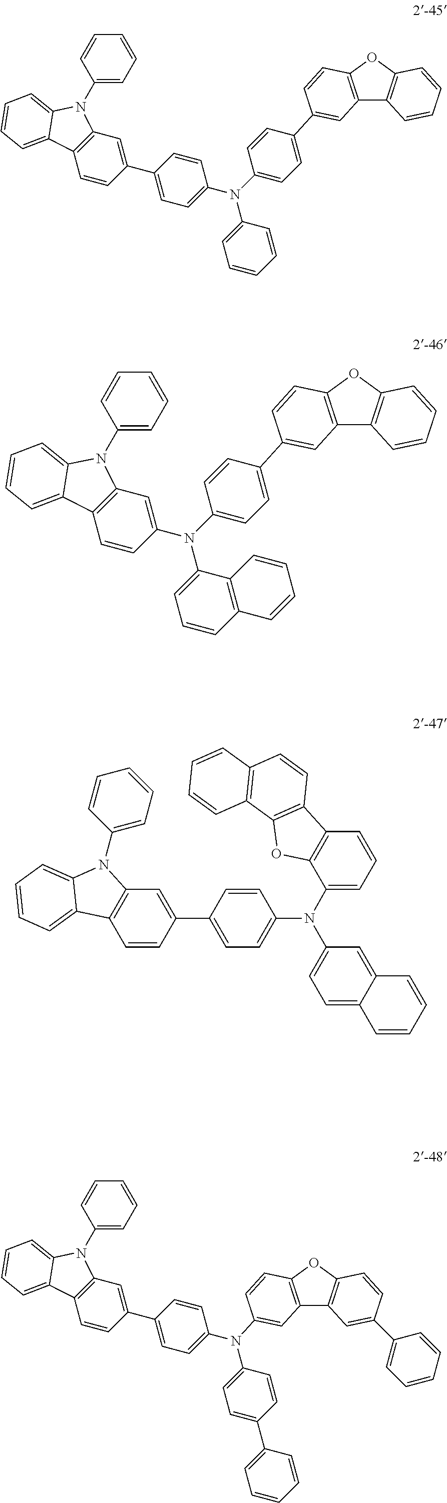 [SMILES: c1ccc(N(c2ccc(-c3ccc4oc5ccccc5c4c3)cc2)c2ccc(-c3ccc4c5ccccc5n(-c5ccccc5)c4c3)cc2)cc1 ; c1ccc(-n2c3ccccc3c3ccc(N(c4ccc(-c5ccc6oc7ccccc7c6c5)cc4)c4cccc5ccccc45)cc32)cc1 ; c1ccc(-n2c3ccccc3c3ccc(-c4ccc(N(c5ccc6ccccc6c5)c5cccc6c5oc5c7ccccc7ccc65)cc4)cc32)cc1 ; c1ccc(-c2ccc(N(c3ccc(-c4ccc5c6ccccc6n(-c6ccccc6)c5c4)cc3)c3ccc4oc5ccc(-c6ccccc6)cc5c4c3)cc2)cc1]

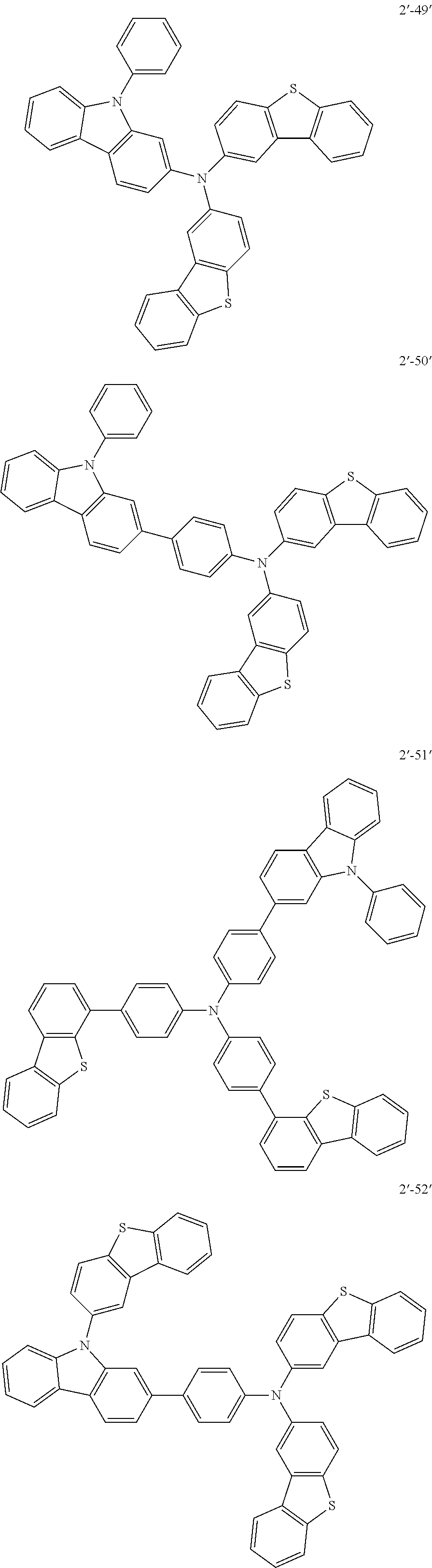

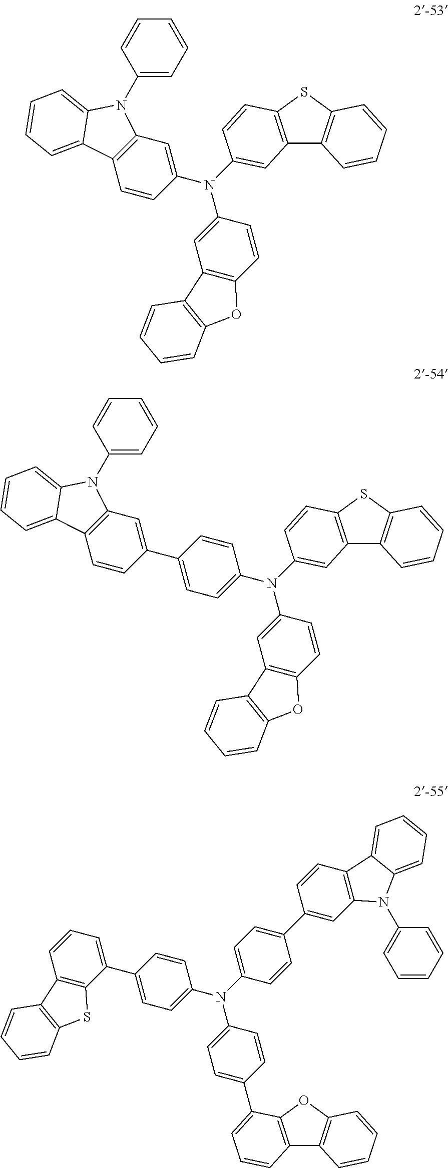

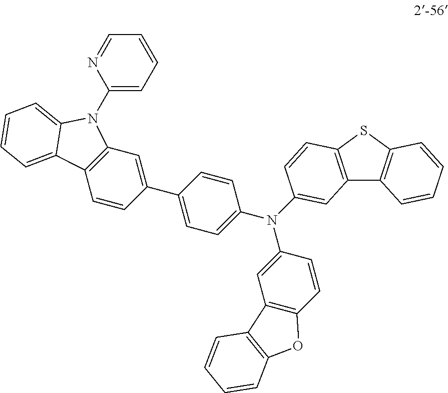 [SMILES: c1ccc(-n2c3ccccc3c3ccc(-c4ccc(N(c5ccc6oc7ccccc7c6c5)c5ccc6sc7ccccc7c6c5)cc4)cc32)nc1]

In another embodiment of the present invention, Ar¹, Ar² and Ar³ of compounds represented by the Formula 1 are all C₆-C₂₄ aryl groups, and meanwhile Ar⁴ and Ar⁵ of compounds represented by the Formula 2 or 2′ are all C₆-C₂₄ aryl groups. More preferably, Ar¹, Ar² and Ar³ of compounds represented by the Formula 1 are all C₆-C₂₄ aryl groups; and any one of Ar⁴ and Ar⁵ of the compound represented by Formula 2 or 2′ are dibenzothiophene or dibenzofuran, more preferably any one of Ar¹, Ar² and Ar³ of compounds represented by the Formula 1 is dibenzothiophene or dibenzofuran; all of Ar⁴ and Ar⁵ of the compound represented by Formula 2 or 2′ are all C₆-C₂₄ aryl groups.

In another preferred embodiment of the present invention, any one of Ar¹, Ar² and Ar³ of compounds represented by the Formula 1 is dibenzothiophene or dibenzofuran; and any one of Ar⁴ and Ar⁵ of the compound represented by Formula 2 or 2′ is dibenzothiophene or dibenzofuran.

In another embodiment of the present invention, the mixing ratio of compound represented by the Formula 1 and compound represented by the Formula 2 or 2′ is 10%˜90% of the compound represented Formula 1. More preferably, the mixing ratio of compound represented by the Formula 1 and compound represented by the Formula 2 or 2′ is at least any one of 5:5 or 6:4 or 7:3 or 8:2 or 9:1.

In another embodiment of the present invention, the mixture of compounds represented by the Formula 1 and compounds represented by the Formula 2 or 2′ further comprise one or more compounds represented by the Formula 1.

In another embodiment of the present invention, the present invention may provide a display device and the organic electric element including an emitting auxiliary layer using compounds represented by the Formula 1 between the emitting layer and the hole transport layer using the mixture of compounds represented by the Formula 1 and compounds represented by the Formula 2 or 2′.

Furthermore, the light efficiency improving layer is formed on at least one side opposite to the organic material layer among one side of the first electrode and the second electrode.

wherein the organic material layer is formed by one of a spin coating process, a nozzle printing process, an inkjet printing process, a slot coating process, a dip coating process or a roll-to-roll process.

The present invention provides an electronic device comprising the display device and the control unit including the organic electric element of various examples described above. Furthermore, the organic electric element may be applied at least one of an organic light emitting diode (OLED), an organic solar cell, an organic photo conductor, an organic transistor or a device for monochromic or white illumination.

Hereinafter, synthesis examples of the compound represented by Formula 1 and Formula 2 or 2′ comprised the organic electric element of the present invention, and preparation examples of the organic electric element of the present invention will be described in detail by way of example. However, the following examples are only for illustrative purposes and are not intended to limit the following examples of the invention.

### Synthesis Example

The final product represented by Formula 1 according to the present invention can be synthesized by reaction between Sub 1 and Sub 2 as illustrated in the following Reaction Scheme 1.

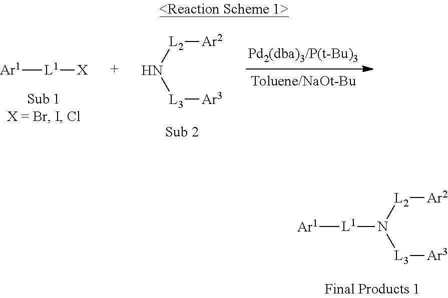

Sub 1

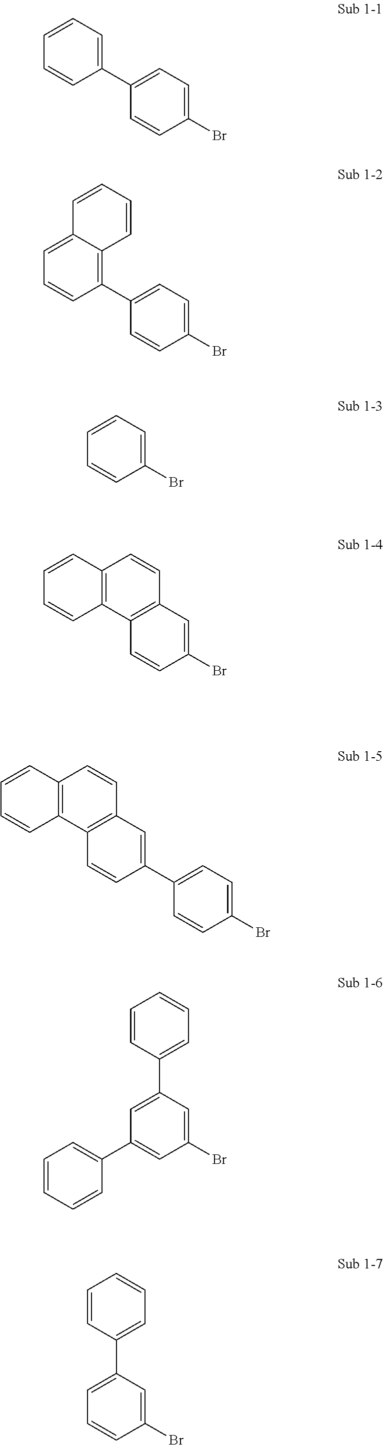

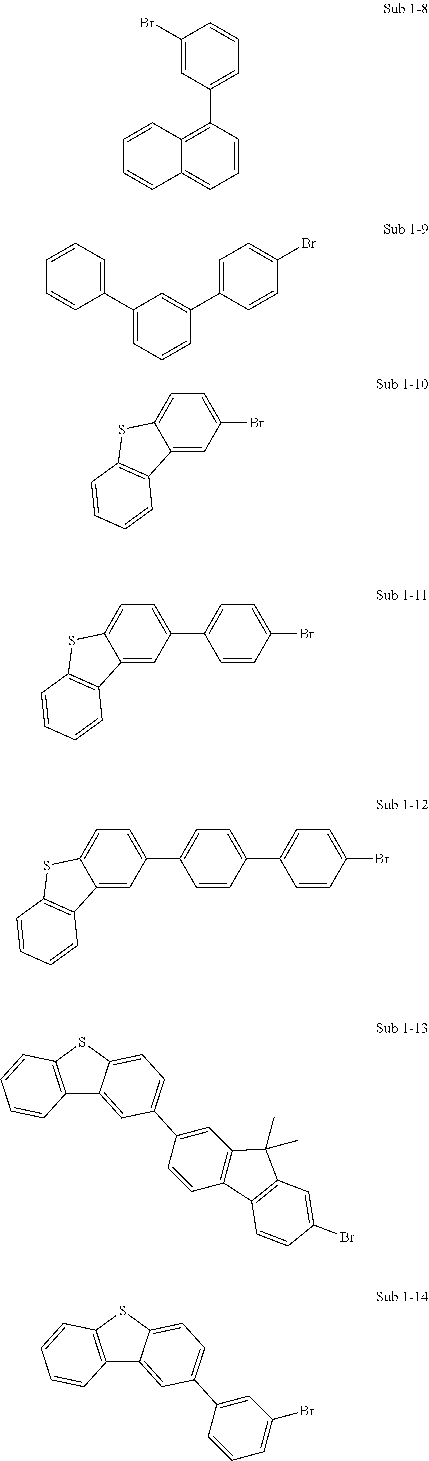

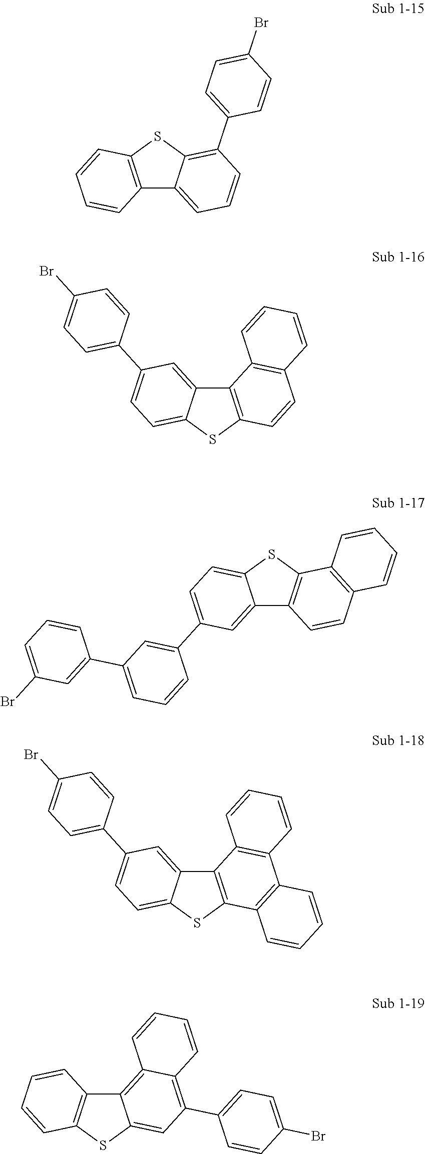 [SMILES: Brc1ccc(-c2cccc3c2sc2ccccc23)cc1 ; Brc1ccc(-c2ccc3sc4ccc5ccccc5c4c3c2)cc1 ; Brc1cccc(-c2cccc(-c3ccc4sc5c6ccccc6ccc5c4c3)c2)c1 ; Brc1ccc(-c2ccc3sc4c5ccccc5c5ccccc5c4c3c2)cc1 ; Brc1ccc(-c2cc3sc4ccccc4c3c3ccccc23)cc1]

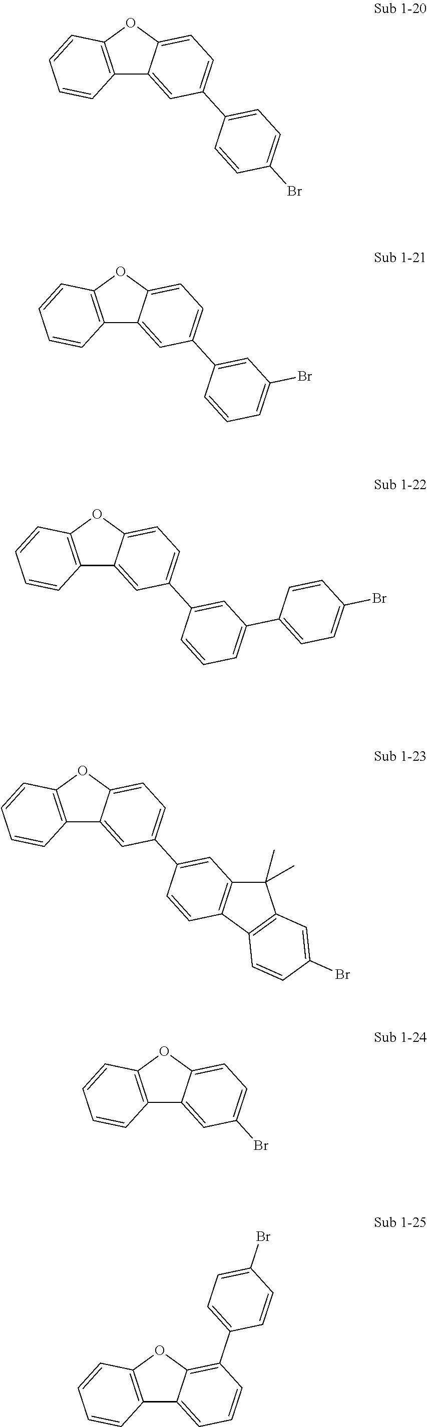 [SMILES: Brc1ccc(-c2ccc3oc4ccccc4c3c2)cc1 ; Brc1cccc(-c2ccc3oc4ccccc4c3c2)c1 ; Brc1ccc(-c2cccc(-c3ccc4oc5ccccc5c4c3)c2)cc1 ; CC1(C)c2cc(Br)ccc2-c2ccc(-c3ccc4oc5ccccc5c4c3)cc21 ; Brc1ccc2oc3ccccc3c2c1 ; Brc1ccc(-c2cccc3c2oc2ccccc23)cc1]

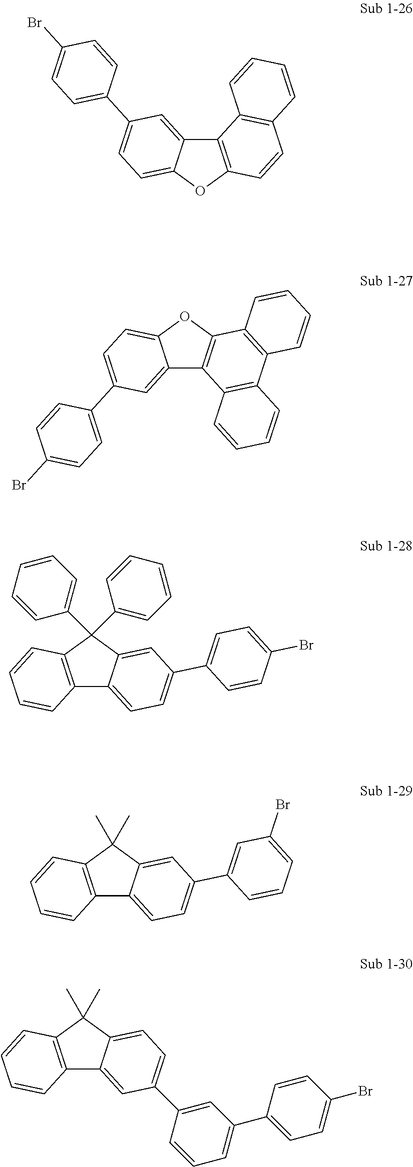 [SMILES: Brc1ccc(-c2ccc3oc4ccc5ccccc5c4c3c2)cc1 ; Brc1ccc(-c2ccc3oc4c5ccccc5c5ccccc5c4c3c2)cc1 ; Brc1ccc(-c2ccc3c(c2)C(c2ccccc2)(c2ccccc2)c2ccccc2-3)cc1 ; CC1(C)c2ccccc2-c2ccc(-c3cccc(Br)c3)cc21 ; CC1(C)c2ccccc2-c2cc(-c3cccc(-c4ccc(Br)cc4)c3)ccc21]

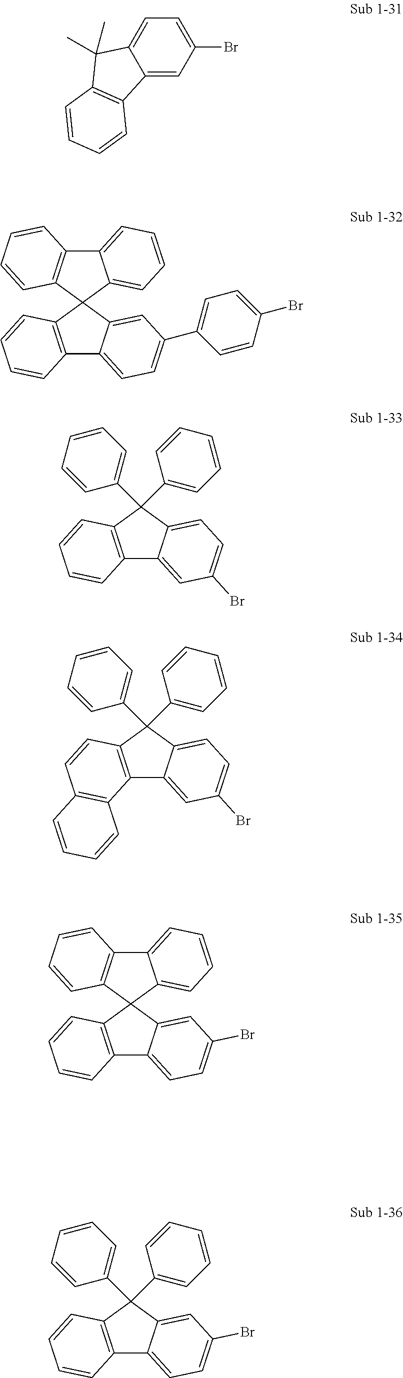 [SMILES: CC1(C)c2ccccc2-c2cc(Br)ccc21 ; Brc1ccc(-c2ccc3c(c2)C2(c4ccccc4-c4ccccc42)c2ccccc2-3)cc1 ; Brc1ccc2c(c1)-c1ccccc1C2(c1ccccc1)c1ccccc1 ; Brc1ccc2c(c1)-c1c(ccc3ccccc13)C2(c1ccccc1)c1ccccc1 ; Brc1ccc2c(c1)C1(c3ccccc3-c3ccccc31)c1ccccc1-2 ; Brc1ccc2c(c1)C(c1ccccc1)(c1ccccc1)c1ccccc1-2]

### Synthesis Example of Sub 2

Sub 2 of Reaction Scheme 1 can be synthesized according to, but not limited to, the reaction path of the following Reaction Scheme 2 or the following Reaction Scheme 3.

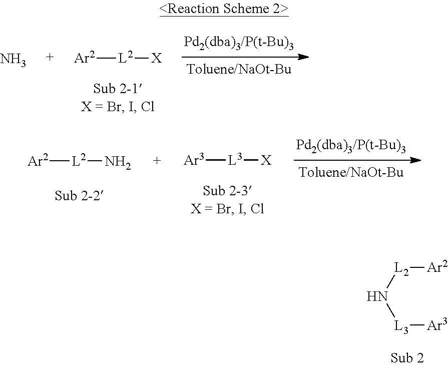

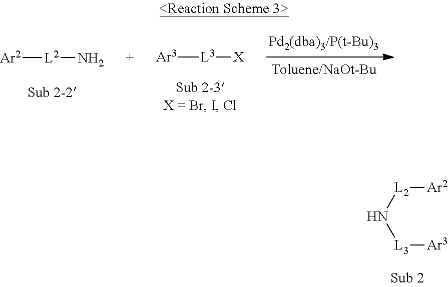

### Examples of Sub 2-1

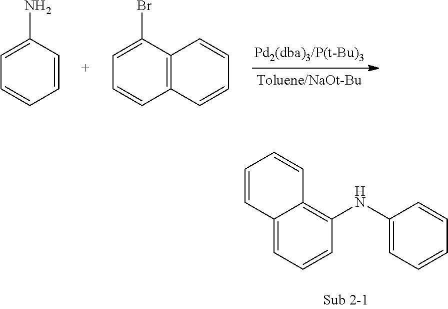 [SMILES: Nc1ccccc1 ; Brc1cccc2ccccc12 ; c1ccc(Nc2cccc3ccccc23)cc1]

After Aniline (15 g, 161.1 mmol), 1-bromonaphthalene (36.7 g, 177.2 mmol), Pd₂(dba)₃ (7.37 g, 8.05 mmol), P(t-Bu)₃ (3.26 g, 16.1 mmol), NaOt-Bu (51.08 g, 531.5 mmol), toluene (1690 mL) are added in a round bottom flask, stirring at 100° C. When the reaction is complete, the reaction product was extracted with CH₂Cl₂ and water. The organic material layer was dried over MgSO₄ and concentrated, and then the product was separated by a silicagel column chromatography and recrystallized to obtain 25.4 g of product Sub 2-1 (yield: 72%).

### Example of Sub 2-26

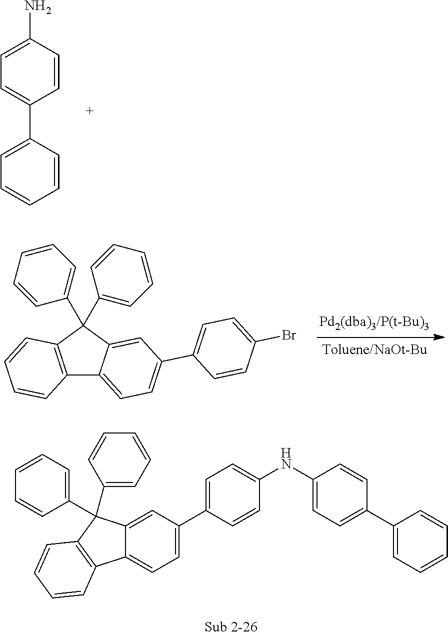

After [1,1′-biphenyl]-4-amine (15 g, 88.6 mmol), 2-(4-bromophenyl)-9,9-diphenyl-9H-fluorene (46.2 g, 97.5 mmol), Pd₂(dba)₃ (4.06 g, 4.43 mmol), P(t-Bu)₃ (1.8 g, 8.86 mmol), NaOt-Bu (28.1 g, 292.5 mmol) and toluene (931 mL) were added in a round bottom flask, and the same procedure as described in the synthesis method of Sub 2-1 above was carried out to obtain 34.9 g of Sub 2-26 (yield: 70%).

### Example of Sub 2-40

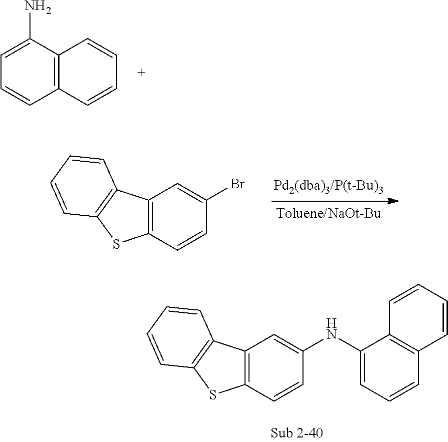 [SMILES: Nc1cccc2ccccc12 ; Brc1ccc2sc3ccccc3c2c1 ; c1ccc2c(Nc3ccc4sc5ccccc5c4c3)cccc2c1]

naphthalen-1-amine (15 g, 104.8 mmol), 2-bromodibenzo[b,d]thiophene (30.3 g, 115.2 mmol), Pd₂(dba)₃ (4.8 g, 5.24 mmol), P(t-Bu)₃ (2.12 g, 10.48 mmol), NaOt-Bu (33.22 g, 345.7 mmol), and toluene (1100 mL) were added in a round bottom flask, and the same procedure as described in the synthesis method of Sub 2-1 above was carried out to obtain 24.9 g of Sub 2-40 (yield: 73%).

### Example of Sub 2-51

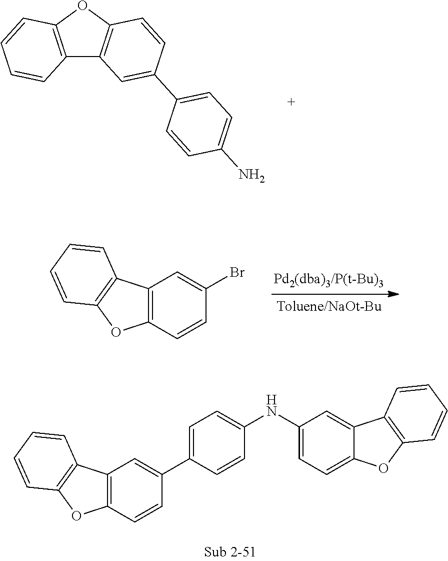 [SMILES: Nc1ccc(-c2ccc3oc4ccccc4c3c2)cc1 ; Brc1ccc2oc3ccccc3c2c1 ; c1ccc2c(c1)oc1ccc(Nc3ccc(-c4ccc5oc6ccccc6c5c4)cc3)cc12]

4-(dibenzo[b,d]furan-2-yl)aniline (15 g, 57.85 mmol), 2-bromodibenzo[b,d]furan (15.7 g, 63.63 mmol), Pd₂(dba)₃ (2.65 g, 2.89 mmol), P(t-Bu)₃ (1.17 g, 5.78 mmol), NaOt-Bu (18.35 g, 190.9 mmol), toluene (607 mL) were added in a round bottom flask, and the same procedure as described in the synthesis method of Sub 2-1 was carried out to obtain 17.2 g of Sub 2-51 (yield: 70%).

The following Sub 2-1 to Sub 2-52 were synthesized with the same procedure as described in the synthesis method, and Sub 2 cannot be limited to the followings.

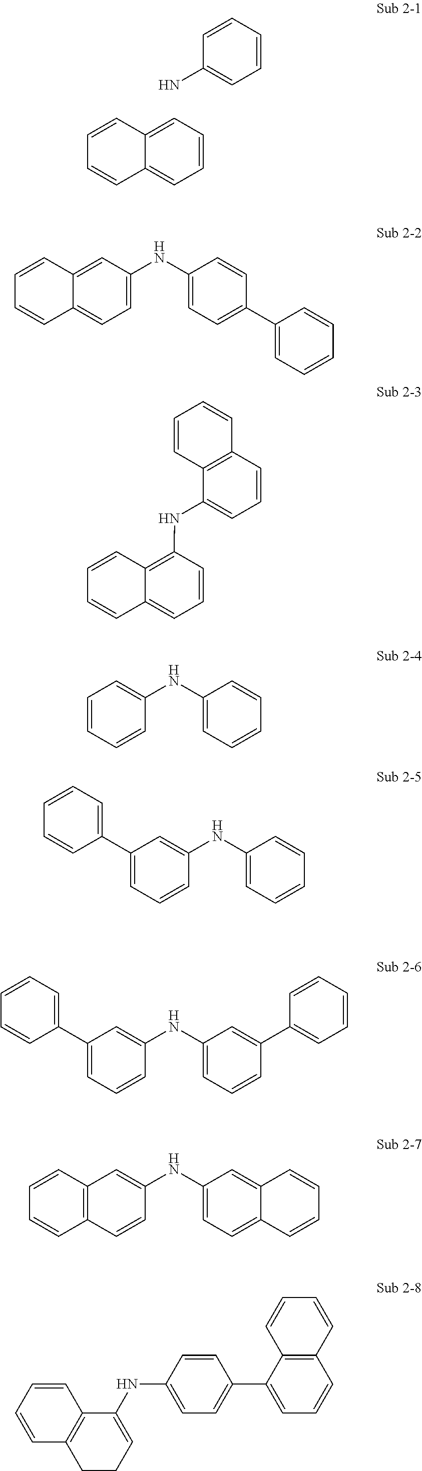

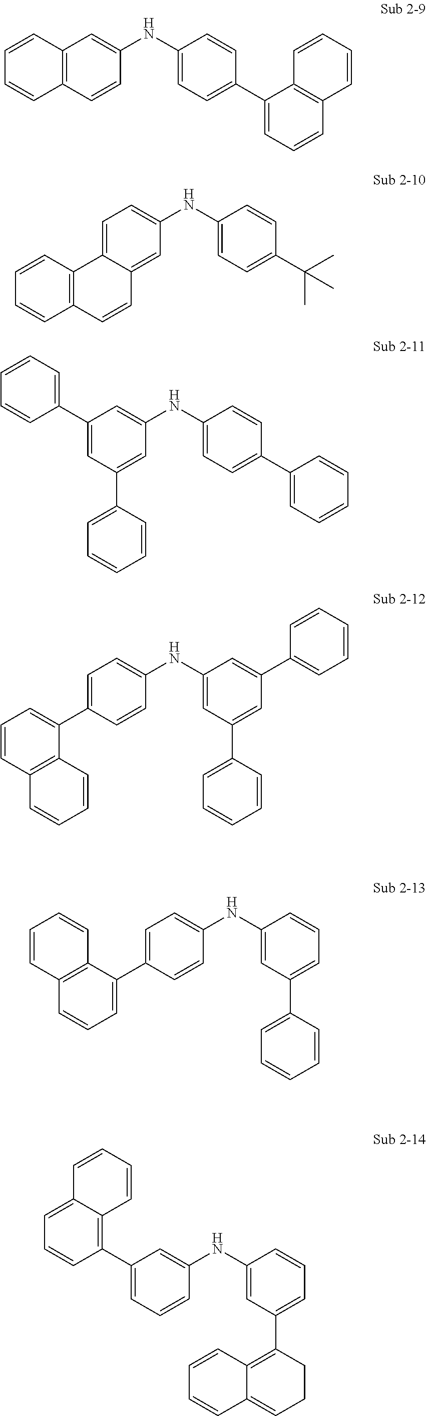

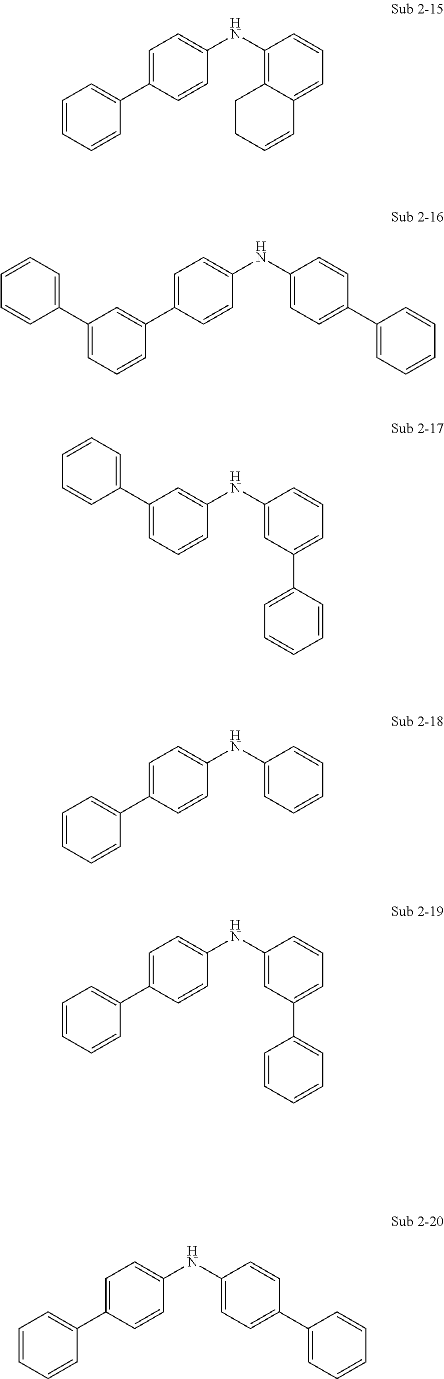 [SMILES: C1=Cc2cccc(Nc3ccc(-c4ccccc4)cc3)c2CC1 ; c1ccc(-c2ccc(Nc3ccc(-c4cccc(-c5ccccc5)c4)cc3)cc2)cc1 ; c1ccc(-c2cccc(Nc3cccc(-c4ccccc4)c3)c2)cc1 ; c1ccc(Nc2ccc(-c3ccccc3)cc2)cc1 ; c1ccc(-c2ccc(Nc3cccc(-c4ccccc4)c3)cc2)cc1 ; c1ccc(-c2ccc(Nc3ccc(-c4ccccc4)cc3)cc2)cc1]

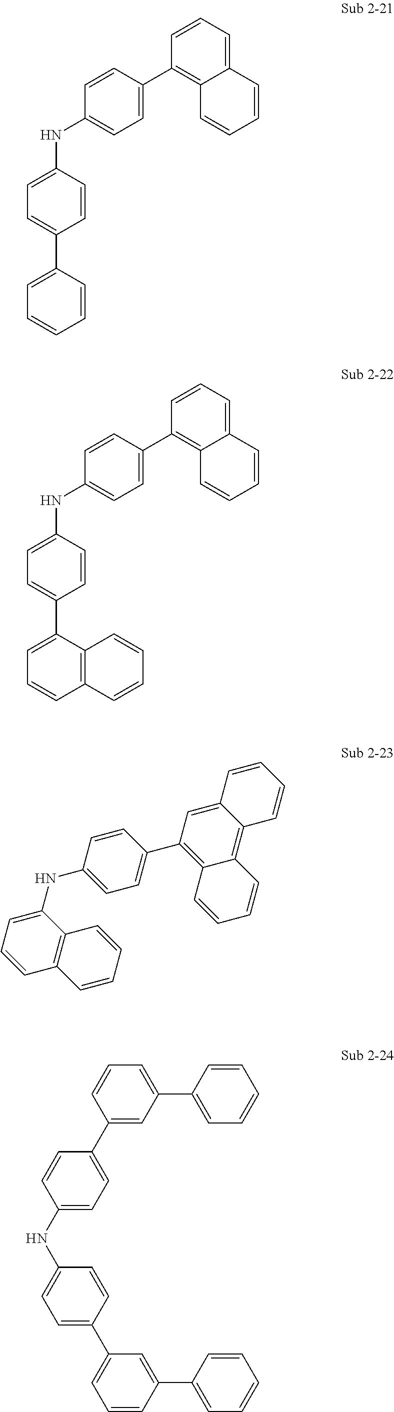 [SMILES: c1ccc(-c2ccc(Nc3ccc(-c4cccc5ccccc45)cc3)cc2)cc1 ; c1ccc2c(-c3ccc(Nc4ccc(-c5cccc6ccccc56)cc4)cc3)cccc2c1 ; c1ccc2c(Nc3ccc(-c4cc5ccccc5c5ccccc45)cc3)cccc2c1 ; c1ccc(-c2cccc(-c3ccc(Nc4ccc(-c5cccc(-c6ccccc6)c5)cc4)cc3)c2)cc1]

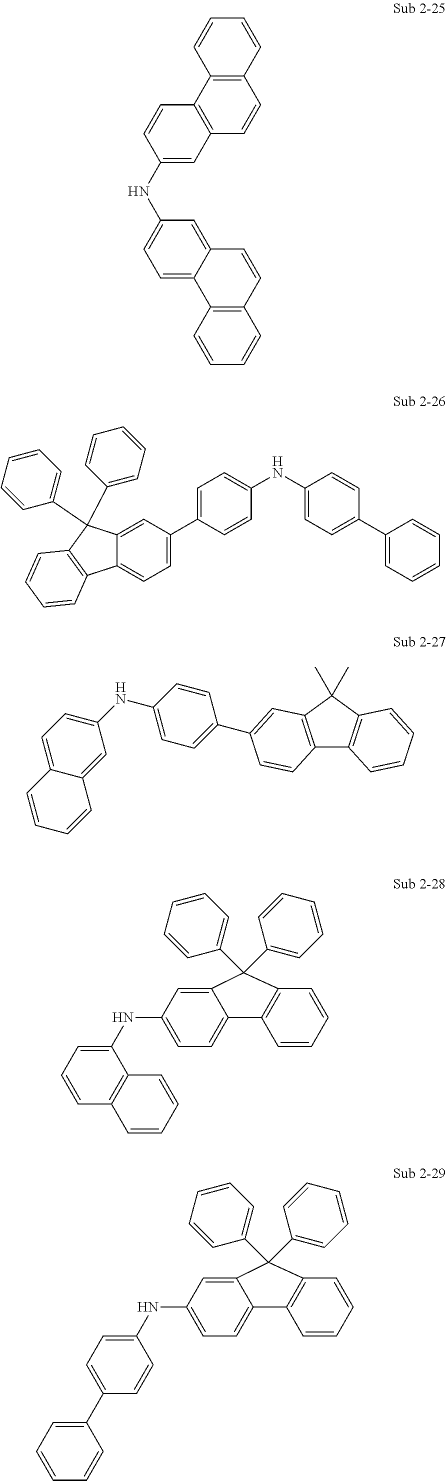 [SMILES: c1ccc2c(c1)ccc1cc(Nc3ccc4c(ccc5ccccc54)c3)ccc12 ; c1ccc(-c2ccc(Nc3ccc(-c4ccc5c(c4)C(c4ccccc4)(c4ccccc4)c4ccccc4-5)cc3)cc2)cc1 ; CC1(C)c2ccccc2-c2ccc(-c3ccc(Nc4ccc5ccccc5c4)cc3)cc21 ; c1ccc(C2(c3ccccc3)c3ccccc3-c3ccc(Nc4cccc5ccccc45)cc32)cc1 ; c1ccc(-c2ccc(Nc3ccc4c(c3)C(c3ccccc3)(c3ccccc3)c3ccccc3-4)cc2)cc1]

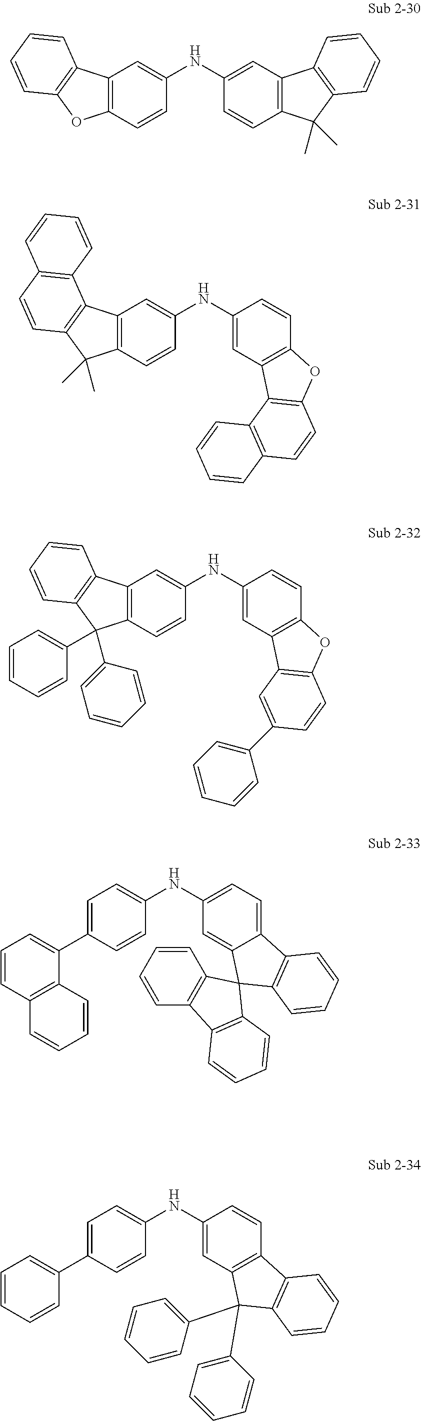 [SMILES: CC1(C)c2ccccc2-c2cc(Nc3ccc4oc5ccccc5c4c3)ccc21 ; CC1(C)c2ccc(Nc3ccc4oc5ccc6ccccc6c5c4c3)cc2-c2c1ccc1ccccc21 ; c1ccc(-c2ccc3oc4ccc(Nc5ccc6c(c5)-c5ccccc5C6(c5ccccc5)c5ccccc5)cc4c3c2)cc1 ; c1ccc2c(c1)-c1ccccc1C21c2ccccc2-c2ccc(Nc3ccc(-c4cccc5ccccc45)cc3)cc21 ; c1ccc(-c2ccc(Nc3ccc4c(c3)C(c3ccccc3)(c3ccccc3)c3ccccc3-4)cc2)cc1]

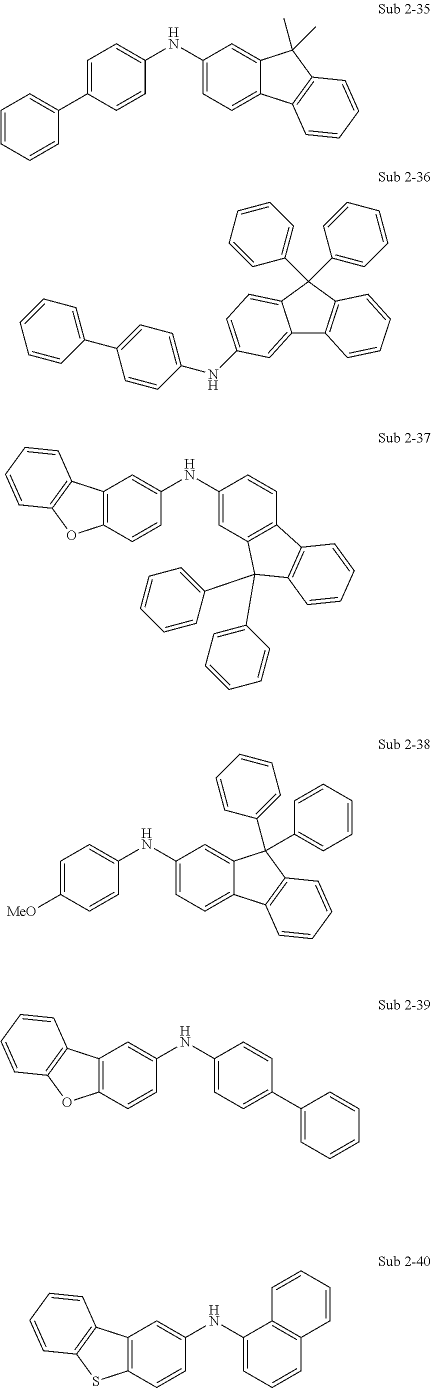 [SMILES: CC1(C)c2ccccc2-c2ccc(Nc3ccc(-c4ccccc4)cc3)cc21 ; c1ccc(-c2ccc(Nc3ccc4c(c3)-c3ccccc3C4(c3ccccc3)c3ccccc3)cc2)cc1 ; c1ccc(C2(c3ccccc3)c3ccccc3-c3ccc(Nc4ccc5oc6ccccc6c5c4)cc32)cc1 ; COc1ccc(Nc2ccc3c(c2)C(c2ccccc2)(c2ccccc2)c2ccccc2-3)cc1 ; c1ccc(-c2ccc(Nc3ccc4oc5ccccc5c4c3)cc2)cc1 ; c1ccc2c(Nc3ccc4sc5ccccc5c4c3)cccc2c1]

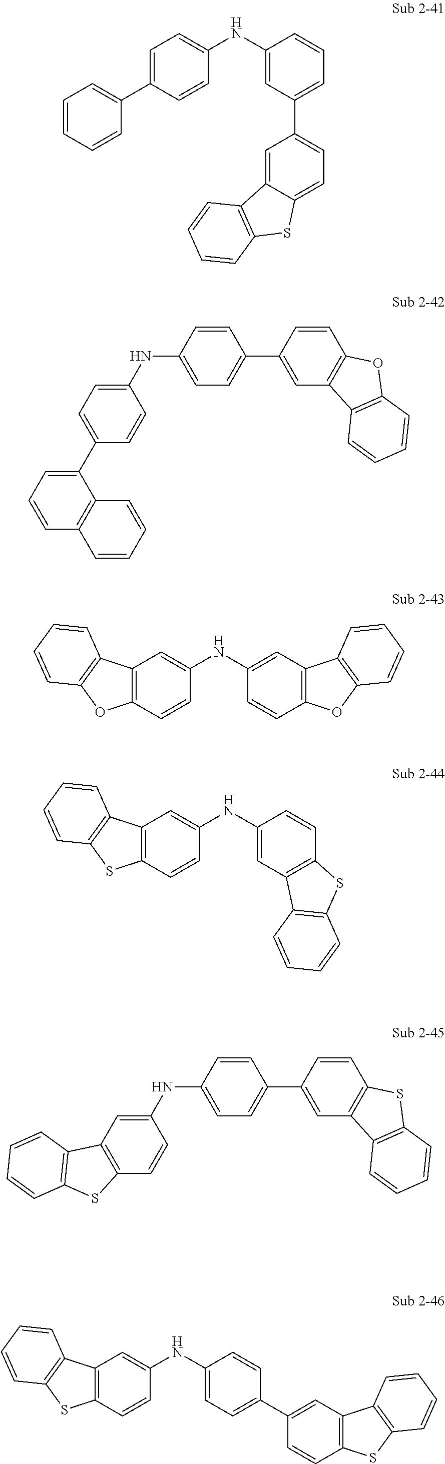 [SMILES: c1ccc(-c2ccc(Nc3cccc(-c4ccc5sc6ccccc6c5c4)c3)cc2)cc1 ; c1ccc2c(-c3ccc(Nc4ccc(-c5ccc6oc7ccccc7c6c5)cc4)cc3)cccc2c1 ; c1ccc2c(c1)oc1ccc(Nc3ccc4oc5ccccc5c4c3)cc12 ; c1ccc2c(c1)sc1ccc(Nc3ccc4sc5ccccc5c4c3)cc12 ; c1ccc2c(c1)sc1ccc(Nc3ccc(-c4ccc5sc6ccccc6c5c4)cc3)cc12 ; c1ccc2c(c1)sc1ccc(Nc3ccc(-c4ccc5sc6ccccc6c5c4)cc3)cc12]

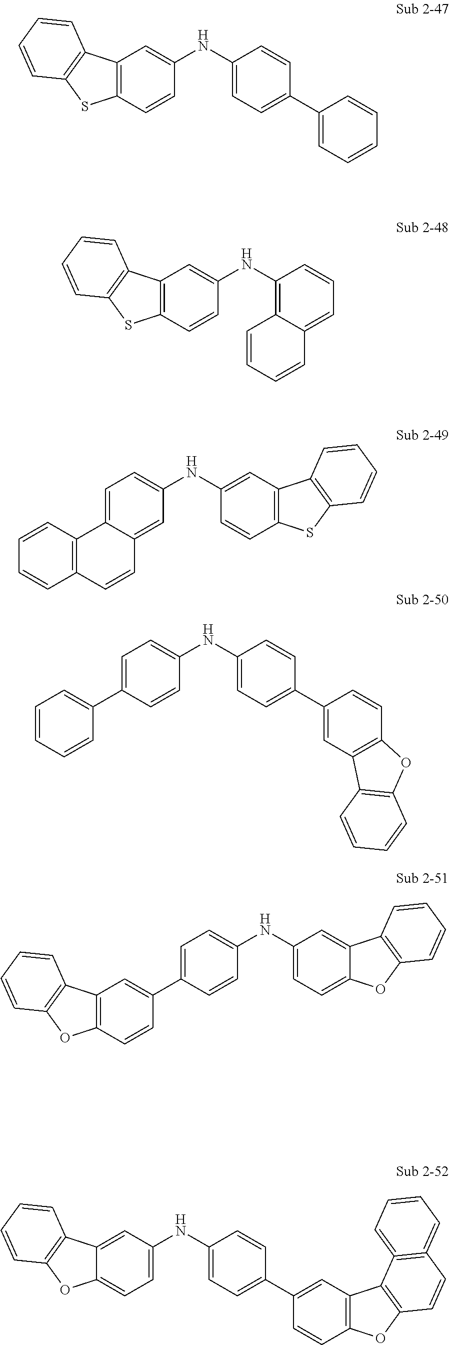

TABLE 1

| compound | FD-MS |
|---|---|
| Sub 2-1 | m/z = 219.10(C₁₆H₁₃N = 219.28) |
| Sub 2-2 | m/z = 295.14(C₂₂H₁₇N = 295.38) |
| Sub 2-3 | m/z = 269.12(C₂₀H₁₅N = 269.34) |
| Sub 2-4 | m/z = 169.09(C₁₂H₁₁N = 169.22) |
| Sub 2-5 | m/z = 245.12(C₁₈H₁₅N = 245.32) |
| Sub 2-6 | m/z = 321.15(C₂₄H₁₉N = 321.41) |
| Sub 2-7 | m/z = 269.12(C₂₀H₁₅N = 269.34) |
| Sub 2-8 | m/z = 345.15(C₂₆H₁₉N = 345.44) |
| Sub 2-9 | m/z = 345.15(C₂₆H₁₉N = 345.44) |
| Sub 2-10 | m/z = 325.18(C₂₄H₂₃N = 325.45) |
| Sub 2-11 | m/z = 397.18(C₃₀H₂₃N = 397.51) |
| Sub 2-12 | m/z = 447.20(C₃₄H₂₅N = 447.57) |
| Sub 2-13 | m/z = 371.17(C₂₈H₂₁N = 371.47) |
| Sub 2-14 | m/z = 421.18(C₃₂H₂₃N = 421.53) |
| Sub 2-15 | m/z = 295.14(C₂₂H₁₇N = 295.38) |
| Sub 2-16 | m/z = 397.18(C₃₀H₂₃N = 397.51) |
| Sub 2-17 | m/z = 321.15(C₂₄H₁₉N = 321.41) |
| Sub 2-18 | m/z = 245.12(C₁₈H₁₅N = 245.32) |
| Sub 2-19 | m/z = 321.15(C₂₄H₁₉N = 321.41) |
| Sub 2-20 | m/z = 321.15(C₂₄H₁₉N = 321.41) |
| Sub 2-21 | m/z = 371.17(C₂₈H₂₁N = 371.47) |
| Sub 2-22 | m/z = 421.18(C₃₂H₂₃N = 421.53) |
| Sub 2-23 | m/z = 395.17(C₃₀H₂₁N = 395.49) |
| Sub 2-24 | m/z = 473.21(C₃₆H₂₇N = 473.61) |
| Sub 2-25 | m/z = 369.15(C₂₈H₁₉N = 369.46) |
| Sub 2-26 | m/z = 561.25(C₄₃H₃₁N = 561.71) |
| Sub 2-27 | m/z = 411.20(C₃₁H₂₅N = 411.54) |
| Sub 2-28 | m/z = 459.20(C₃₅H₂₅N = 459.58) |
| Sub 2-29 | m/z = 483.20(C₃₇H₂₅N = 483.60) |
| Sub 2-30 | m/z = 375.16(C₂₇H₂₁NO = 375.46) |
| Sub 2-31 | m/z = 475.19(C₃₅H₂₅NO = 475.58) |
| Sub 2-32 | m/z = 575.22(C₄₃H₂₉NO = 575.70) |
| Sub 2-33 | m/z = 533.21(C₄₁H₂₇N = 533.66) |
| Sub 2-34 | m/z = 485.21(C₃₇H₂₇N = 485.62) |
| Sub 2-35 | m/z = 361.18(C₂₇H₂₃N = 361.48) |
| Sub 2-36 | m/z = 485.21(C₃₇H₂₇N = 485.62) |
| Sub 2-37 | m/z = 499.19(C₃₇H₂₅NO = 499.60) |
| Sub 2-38 | m/z = 439.19(C₃₂H₂₅NO = 439.55) |
| Sub 2-39 | m/z = 335.13(C₂₄H₁₇NO = 335.40) |
| Sub 2-40 | m/z = 325.09(C₂₂H₁₅NS = 325.43) |
| Sub 2-41 | m/z = 427.14(C₃₀H₂₁NS = 427.56) |
| Sub 2-42 | m/z = 461.18(C₃₄H₂₃NO = 461.55) |
| Sub 2-43 | m/z = 349.11(C₂₄H₁₅NO₂ = 349.38) |
| Sub 2-44 | m/z = 381.06(C₂₄H₁₅NS₂ = 381.51) |
| Sub 2-45 | m/z = 457.10(C₃₀H₁₉NS₂ = 457.61) |
| Sub 2-46 | m/z = 533.13(C₃₆H₂₃NS₂ = 533.70) |
| Sub 2-47 | m/z = 353.10(C₂₂H₁₅N₃S = 353.44) |
| Sub 2-48 | m/z = 327.0(C₂₀H₁₃N₃S = 327.40) |
| Sub 2-49 | m/z = 375.11(C₂₆H₁₇NS = 375.48) |
| Sub 2-50 | m/z = 411.16(C₃₀H₂₁NO = 411.49) |
| Sub 2-51 | m/z = 425.14(C₃₀H₁₉NO₂ = 425.48) |
| Sub 2-52 | m/z = 475.16(C₃₄H₂₁NO₂ = 475.54) |

### Synthesis of Final Product of the Formula 1 (the Same Procedure of Sub 2)

In a round bottom flask, Sub 2 (1 eq.) and Sub 1 (1.1 eq.) was dissolved in Toluene, Pd₂(dba)₃ (0.05 eq.), PPh₃ (0.1 eq.), NaOt-Bu (3 eq.) were added and followed by stirring and reflux at 100° C. When the reaction is complete, the product was extracted with ether and water. The organic layer was dried over MgSO₄ and concentrated, and then the product was separated by a silicagel column chromatography and recrystallized to obtain the Final Product.

### Synthesis Example of Final Product

### Synthesis 1-1′

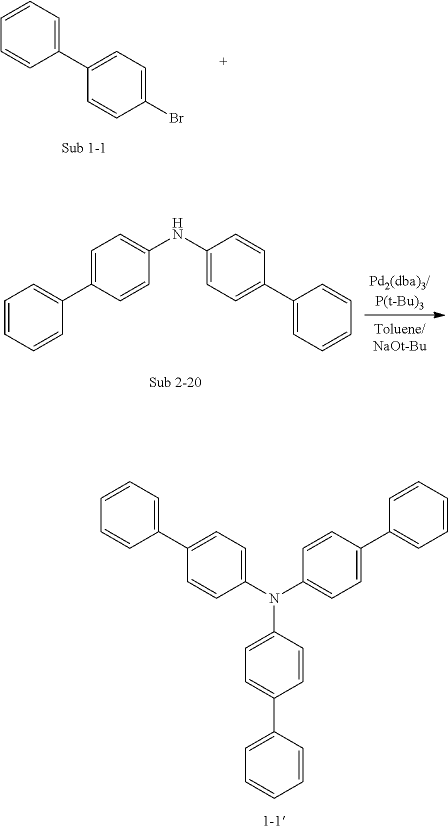 [SMILES: Brc1ccc(-c2ccccc2)cc1 ; c1ccc(-c2ccc(Nc3ccc(-c4ccccc4)cc3)cc2)cc1 ; c1ccc(-c2ccc(N(c3ccc(-c4ccccc4)cc3)c3ccc(-c4ccccc4)cc3)cc2)cc1]

After di([1,1′-biphenyl]-4-yl)amine (10 g, 31.1 mmol), 4-bromo-1,1′-biphenyl(8 g, 34.2 mmol), Pd₂(dba)₃ (1.42 g, 1.56 mmol), P(t-Bu)₃ (0.63 g, 3.11 mmol), NaOt-Bu (9.87 g, 102.7 mmol), toluene (330 mL) are added in a round bottom flask, stirring at 100° C. When the reaction is complete, the product was extracted with CH₂Cl₂ and water. The organic layer was dried over MgSO₄ and concentrated, and then the product was separated by a silicagel column chromatography and recrystallized to obtain 11.3 g of Product 1-1′(yield: 77%).

### Synthesis 1-4′

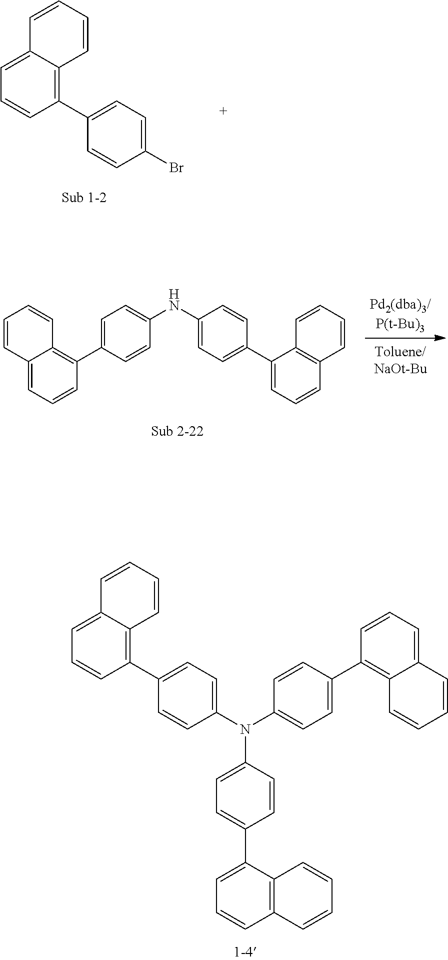 [SMILES: Brc1ccc(-c2cccc3ccccc23)cc1 ; c1ccc2c(-c3ccc(Nc4ccc(-c5cccc6ccccc56)cc4)cc3)cccc2c1 ; c1ccc2c(-c3ccc(N(c4ccc(-c5cccc6ccccc56)cc4)c4ccc(-c5cccc6ccccc56)cc4)cc3)cccc2c1]

Bis(4-(naphthalen-1-yl)phenyl)amine (10 g, 23.7 mmol), 1-(4-bromophenyl)naphthalene (7.4 g, 26.1 mmol), Pd₂(dba)₃ (1.09 g, 1.19 mmol), P(t-Bu)₃ (0.5 g, 2.4 mmol), NaOt-Bu (7.52 g, 78.3 mmol), toluene (250 mL) were added in a round bottom flask, and the same procedure as described in the synthesis method of Product 1-1′ was carried out to obtain 11.5 g of Product 1-4′ (yield:78%).

### Synthesis 1-10′

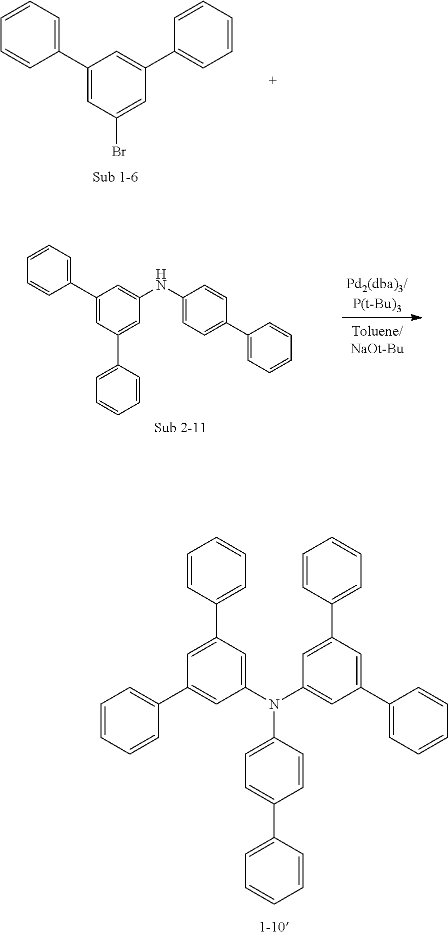 [SMILES: Brc1cc(-c2ccccc2)cc(-c2ccccc2)c1 ; c1ccc(-c2ccc(Nc3cc(-c4ccccc4)cc(-c4ccccc4)c3)cc2)cc1 ; c1ccc(-c2ccc(N(c3cc(-c4ccccc4)cc(-c4ccccc4)c3)c3cc(-c4ccccc4)cc(-c4ccccc4)c3)cc2)cc1]

N-([1,1′-biphenyl]-4-yl)[1,1′:3′,1″-terphenyl]-5′-amine (10 g, 25.2 mmol), 5′-bromo-1,1′:3′,1″-terphenyl(8.56 g, 27.7 mmol), Pd₂(dba)₃ (1.15 g, 1.26 mmol), P(t-Bu)₃ (0.51 g, 2.52 mmol), NaOt-Bu (7.98 g, 83.02 mmol), toluene (264 mL) were added in a round bottom flask, and the same procedure as described in the synthesis method of Product 1-1′ was carried out to obtain 11.8 g of Product 1-10′(yield:75%).

### Synthesis 1-19′

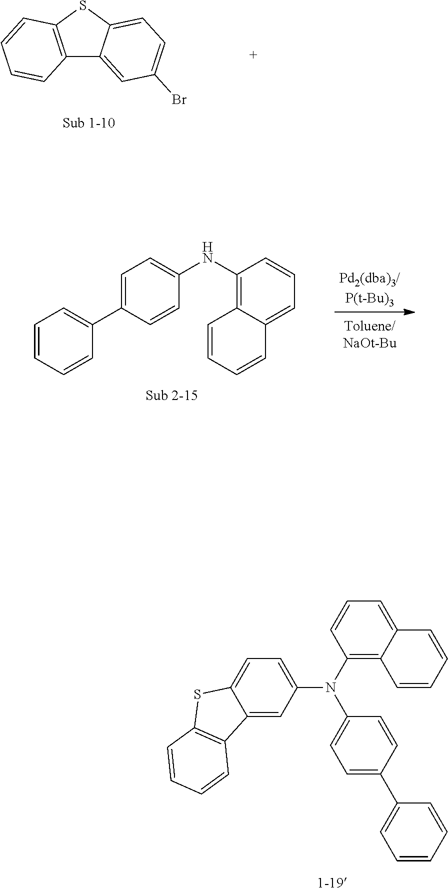 [SMILES: Brc1ccc2sc3ccccc3c2c1 ; c1ccc(-c2ccc(Nc3cccc4ccccc34)cc2)cc1 ; c1ccc(-c2ccc(N(c3ccc4sc5ccccc5c4c3)c3cccc4ccccc34)cc2)cc1]

N-([1,1′-biphenyl]-4-yl)naphthalen-1-amine (10 g, 33.6 mmol), 2-bromodibenzo[b,d]thiophene (9.8 g, 37.2 mmol), Pd₂(dba)₃ (1.55 g, 1.7 mmol), P(t-Bu)₃ (0.68 g, 3.38 mmol), NaOt-Bu (10.76 g, 112 mmol), toluene (355 mL) were added in a round bottom flask, and the same procedure as described in the synthesis method of Product 1-1′ was carried out to obtain 12.3 g of Product 1-19′(yield:76%).

### Synthesis 1-20′

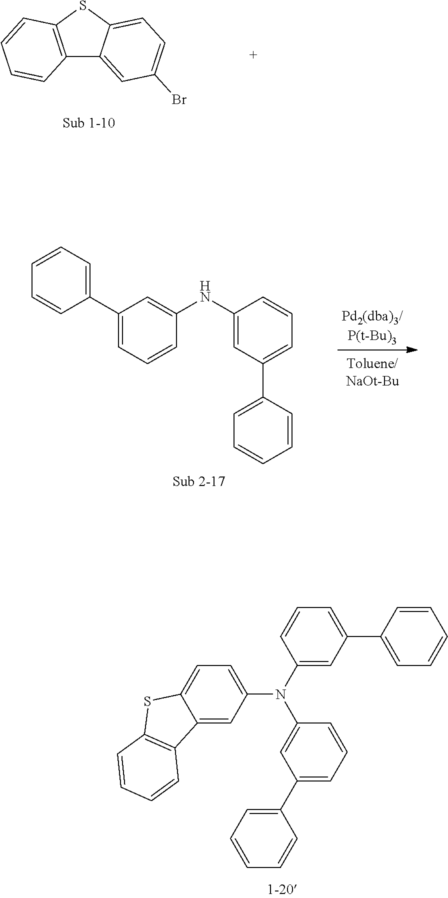 [SMILES: Brc1ccc2sc3ccccc3c2c1 ; c1ccc(-c2cccc(Nc3cccc(-c4ccccc4)c3)c2)cc1 ; c1ccc(-c2cccc(N(c3cccc(-c4ccccc4)c3)c3ccc4sc5ccccc5c4c3)c2)cc1]

Di([1,1′-biphenyl]-3-yl)amine (10 g, 31.1 mmol), 2-54-bromodibenzo[b,d]thiophene (9 g, 34.2 mmol), Pd₂(dba)₃ (1.42 g, 1.56 mmol), P(t-Bu)₃ (0.63 g, 3.11 mmol), NaOt-Bu (9.87 g, 102.7 mmol), toluene (327 mL) were added in a round bottom flask, and the same procedure as described in the synthesis method of Product 1-1′ was carried out to obtain 12.2 g of Product 1-20′(yield:78%).

### Synthesis 1-23′

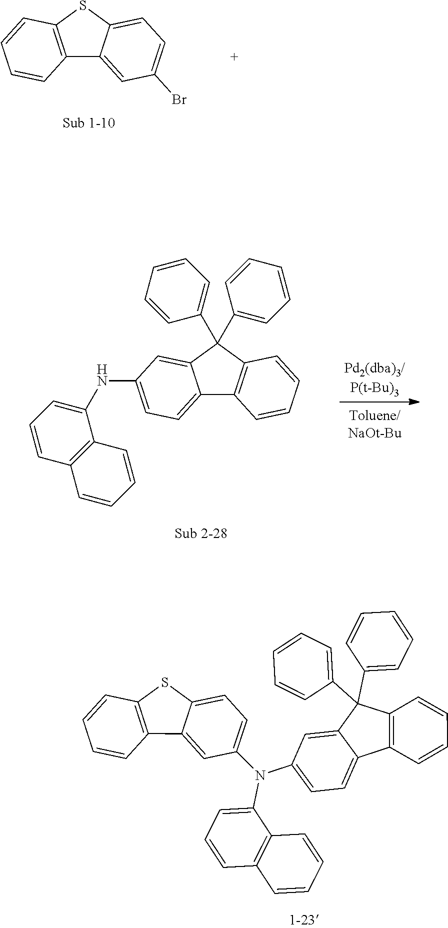 [SMILES: Brc1ccc2sc3ccccc3c2c1 ; c1ccc(C2(c3ccccc3)c3ccccc3-c3ccc(Nc4cccc5ccccc45)cc32)cc1 ; c1ccc(C2(c3ccccc3)c3ccccc3-c3ccc(N(c4ccc5sc6ccccc6c5c4)c4cccc5ccccc45)cc32)cc1]

N-(naphthalen-1-yl)-9,9-diphenyl-9H-fluoren-2-amine (10 g, 21.8 mmol), 2-bromodibenzo[b,d]thiophene (6.3 g, 23.9 mmol), Pd₂(dba)₃ (1 g, 1.09 mmol), P(t-Bu)₃ (0.44 g, 2.2 mmol), NaOt-Bu (6.9 g, 71.8 mmol), toluene (230 mL) were added in a round bottom flask, and the same procedure as described in the synthesis method of Product 1-1′ was carried out to obtain 10.2 g of Product 1-23′(yield:73%).

### Synthesis 1-24′

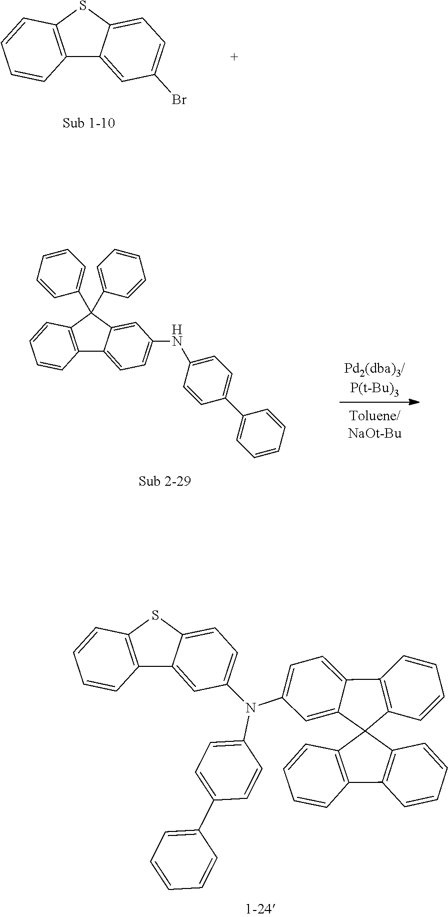 [SMILES: Brc1ccc2sc3ccccc3c2c1 ; c1ccc(-c2ccc(Nc3ccc4c(c3)C(c3ccccc3)(c3ccccc3)c3ccccc3-4)cc2)cc1 ; c1ccc(-c2ccc(N(c3ccc4c(c3)C3(c5ccccc5-c5ccccc53)c3ccccc3-4)c3ccc4sc5ccccc5c4c3)cc2)cc1]

N-([1,1′-biphenyl]-4-yl)-9,9′-spirobi[fluoren]-2-amine (10 g, 20.7 mmol), 2-bromodibenzo[b,d]thiophene (6 g, 22.7 mmol), Pd₂(dba)₃ (0.95 g, 1.03 mmol), P(t-Bu)₃ (0.42 g, 2.07 mmol), NaOt-Bu (6.55 g, 68.2 mmol), and toluene (220 mL) were added in a round bottom flask, and the same procedure as described in the synthesis method of Product 1-1′ was carried out to obtain 10.2 g of Product 1-24′ (yield:74%).

### Synthesis 1-29′

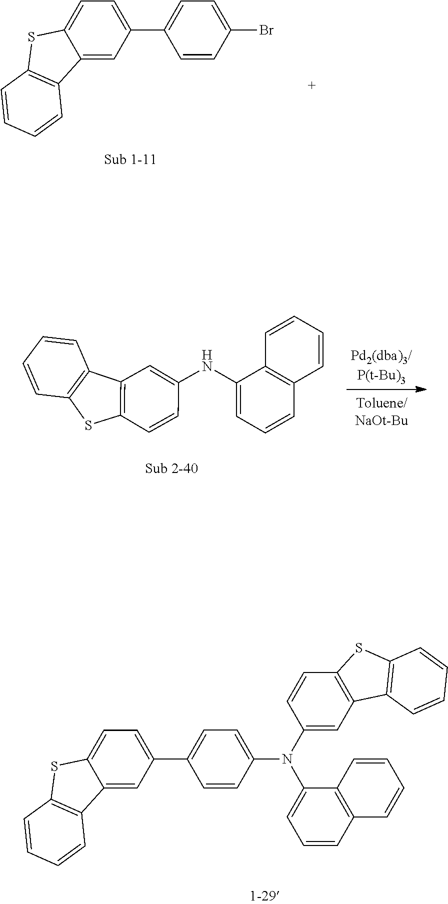 [SMILES: Brc1ccc(-c2ccc3sc4ccccc4c3c2)cc1 ; c1ccc2c(Nc3ccc4sc5ccccc5c4c3)cccc2c1 ; c1ccc2c(N(c3ccc(-c4ccc5sc6ccccc6c5c4)cc3)c3ccc4sc5ccccc5c4c3)cccc2c1]

N-(naphthalen-1-yl)dibenzo[b,d]thiophen-2-amine (10 g, 30.7 mmol), 2-(4-bromophenyl)dibenzo[b,d]thiophene (11.5 g, 33.8 mmol), Pd₂(dba)₃ (1.41 g, 1.54 mmol), P(t-Bu)₃ (0.62 g, 3.07 mmol), NaOt-Bu (9.75 g, 101.4 mmol) and toluene (325 mL) were added in a round bottom flask, and the same procedure as described in the synthesis method of Product 1-1′ was carried out to obtain 12.9 g of Product 1-29′ (yield:72%).

### Synthesis 1-30′

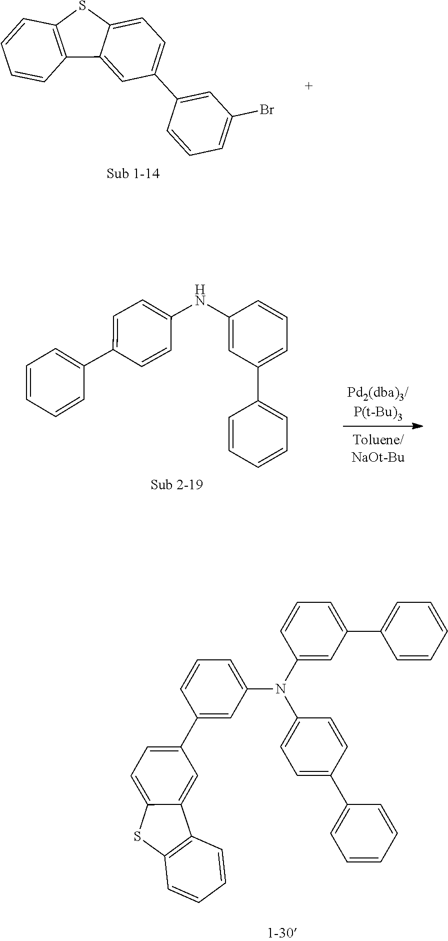 [SMILES: Brc1cccc(-c2ccc3sc4ccccc4c3c2)c1 ; c1ccc(-c2ccc(Nc3cccc(-c4ccccc4)c3)cc2)cc1 ; c1ccc(-c2ccc(N(c3cccc(-c4ccccc4)c3)c3cccc(-c4ccc5sc6ccccc6c5c4)c3)cc2)cc1]

N-([1,1′-biphenyl]-4-yl)[1,1′-biphenyl]-3-amine (10 g, 31.1 mmol), 2-(3-bromophenyl)dibenzo[b,d]thiophene (11.6 g, 34.2 mmol), Pd₂(dba)₃ (1.42 g, 1.55 mmol), P(t-Bu)₃ (0.63 g, 3.11 mmol), NaOt-Bu (9.9 g, 103 mmol) and toluene (330 mL) were added in a round bottom flask, and the same procedure as described in the synthesis method of Product 1-1′ was carried out to obtain 12.8 g of Product 1-30′ (yield:71%).

### Synthesis 1-36′

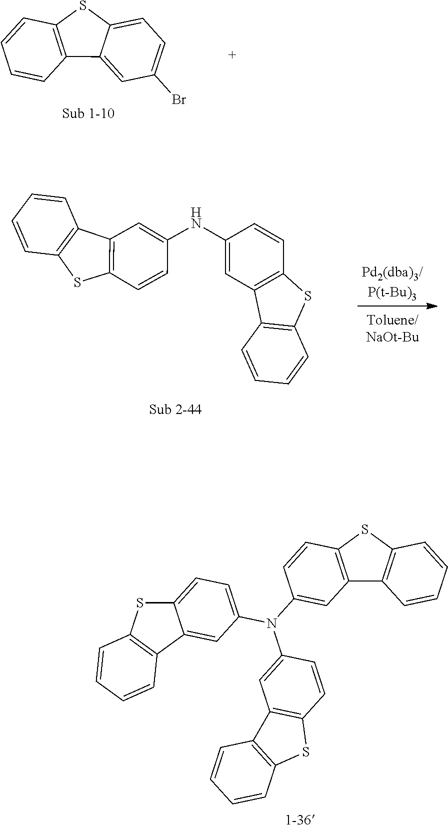 [SMILES: Brc1ccc2sc3ccccc3c2c1 ; c1ccc2c(c1)sc1ccc(Nc3ccc4sc5ccccc5c4c3)cc12 ; c1ccc2c(c1)sc1ccc(N(c3ccc4sc5ccccc5c4c3)c3ccc4sc5ccccc5c4c3)cc12]

Bis(dibenzo[b,d]thiophen-2-yl)amine (10 g, 26.2 mmol), 2-bromodibenzo[b,d]thiophene (7.59 g, 28.8 mmol), Pd₂(dba)₃ (1.2 g, 1.31 mmol), P(t-Bu)₃ (0.53 g, 2.62 mmol), NaOt-Bu (8.31 g, 86.5 mmol) and toluene (275 mL) were added in a round bottom flask, and the same procedure as described in the synthesis method of Product 1-1′ was carried out to obtain 11.4 g of Product 1-36′ (yield:77%).

### Synthesis 1-49′

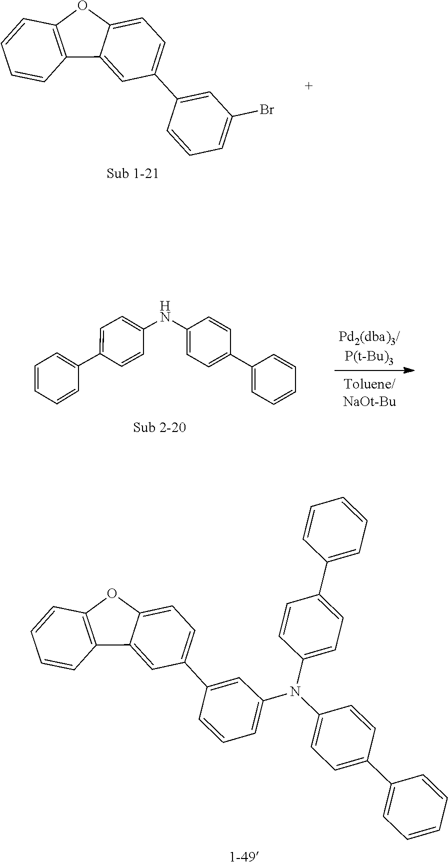 [SMILES: Brc1cccc(-c2ccc3oc4ccccc4c3c2)c1 ; c1ccc(-c2ccc(Nc3ccc(-c4ccccc4)cc3)cc2)cc1 ; c1ccc(-c2ccc(N(c3ccc(-c4ccccc4)cc3)c3cccc(-c4ccc5oc6ccccc6c5c4)c3)cc2)cc1]

Di([1,1′-biphenyl]-4-yl)amine (10 g, 31.1 mmol), 2-(3-bromophenyl)dibenzo[b,d]furan (11.1 g, 34.2 mmol), Pd₂(dba)₃ (1.42 g, 1.56 mmol), P(t-Bu)₃ (0.63 g, 3.11 mmol), NaOt-Bu (9.9 g, 103 mmol), toluene (330 mL) were added in a round bottom flask, and the same procedure as described in the synthesis method of Product 1-1′ was carried out to obtain 13.3 g of Product 1-49′ (yield:76%).

### Synthesis 1-51′

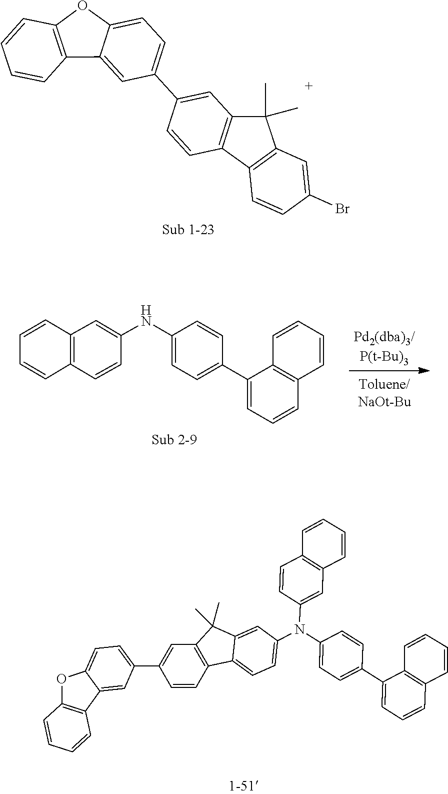 [SMILES: CC1(C)c2cc(Br)ccc2-c2ccc(-c3ccc4oc5ccccc5c4c3)cc21 ; c1ccc2cc(Nc3ccc(-c4cccc5ccccc45)cc3)ccc2c1 ; CC1(C)c2cc(-c3ccc4oc5ccccc5c4c3)ccc2-c2ccc(N(c3ccc(-c4cccc5ccccc45)cc3)c3ccc4ccccc4c3)cc21]

N-(4-(naphthalen-1-yl)phenyl)naphthalen-2-amine (10 g, 28.9 mmol), 2-(7-bromo-9,9-dimethyl-9H-fluoren-2-yl)dibenzo[b,d]furan (14 g, 32 mmol), Pd₂(dba)₃ (1.33 g, 1.45 mmol), P(t-Bu)₃ (0.59 g, 2.9 mmol), NaOt-Bu (9.2 g, 95.5 mmol) and toluene (310 mL) were added in a round bottom flask, and the same procedure as described in the synthesis method of Product 1-1′ was carried out to obtain 14.5 g of Product 1-51′ (yield:71%).

### Synthesis 1-59′

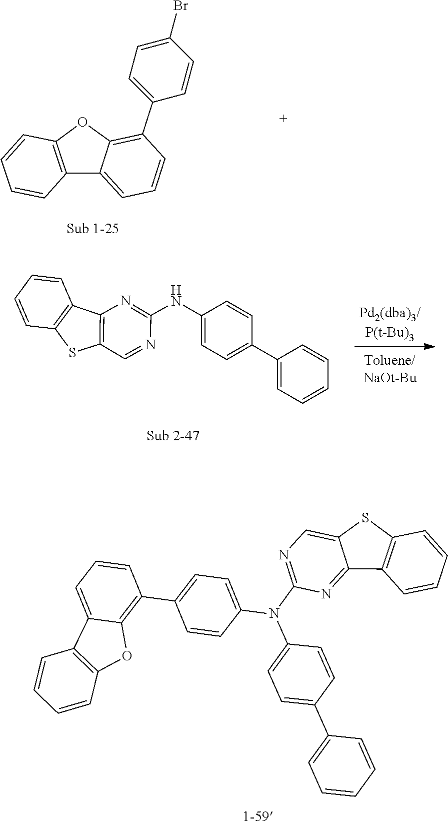 [SMILES: Brc1ccc(-c2cccc3c2oc2ccccc23)cc1 ; c1ccc(-c2ccc(Nc3ncc4sc5ccccc5c4n3)cc2)cc1 ; c1ccc(-c2ccc(N(c3ccc(-c4cccc5c4oc4ccccc45)cc3)c3ncc4sc5ccccc5c4n3)cc2)cc1]

N-([1,1′-biphenyl]-4-yl)benzo[4,5]thieno[3,2-d]pyrimidin-2-amine (10 g, 28.3 mmol), 4-(4-bromophenyl)dibenzo[b,d]furan (10.1 g, 31.1 mmol), Pd₂(dba)₃ (1.3 g, 1.41 mmol), P(t-Bu)₃ (0.57 g, 2.83 mmol), NaOt-Bu (8.98 g, 93.4 mmol) and toluene (300 mL) were added in a round bottom flask, and the same procedure as described in the synthesis method of Product 1-1′ above was carried out to obtain 12.3 g of Product 1-59′ (yield:73%).

### Synthesis 1-71′

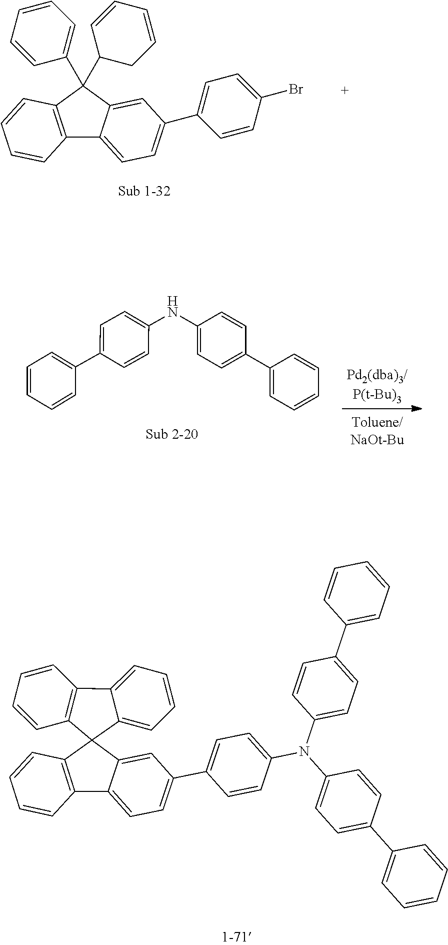 [SMILES: Brc1ccc(-c2ccc3c(c2)C(c2ccccc2)(C2C=CC=CC2)c2ccccc2-3)cc1 ; c1ccc(-c2ccc(Nc3ccc(-c4ccccc4)cc3)cc2)cc1 ; c1ccc(-c2ccc(N(c3ccc(-c4ccccc4)cc3)c3ccc(-c4ccc5c(c4)C4(c6ccccc6-c6ccccc64)c4ccccc4-5)cc3)cc2)cc1]

Di([1,1′-biphenyl]-4-yl)amine (10 g, 31.1 mmol), 2-(4-bromophenyl)-9,9′-spirobi[fluorene] (16.1 g, 34.2 mmol), Pd₂(dba)₃ (1.42 g, 1.56 mmol), P(t-Bu)₃ (0.63 g, 3.11 mmol), NaOt-Bu (9.87 g, 102.7 mmol), and toluene (330 mL) were added in a round bottom flask, and the same procedure as described in the synthesis method of Product 1-1′ above was carried out to obtain 15.5 g of Product 1-71′(yield:70%).

### Synthesis 1-75′

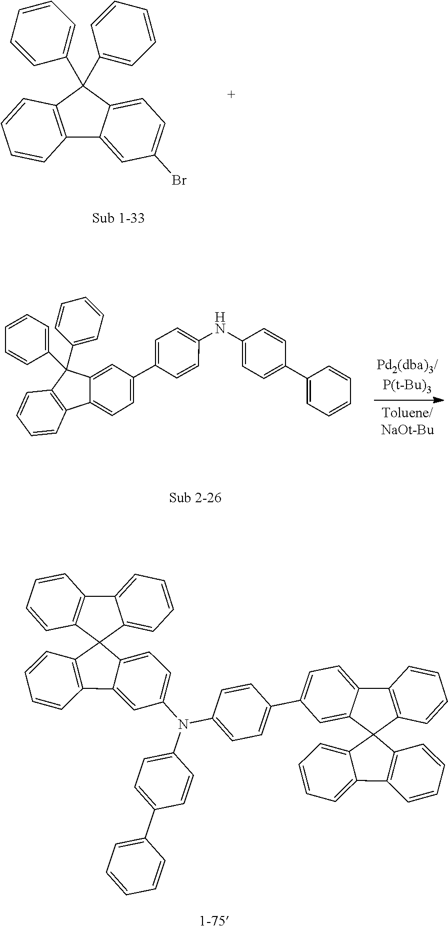 [SMILES: Brc1ccc2c(c1)-c1ccccc1C2(c1ccccc1)c1ccccc1 ; c1ccc(-c2ccc(Nc3ccc(-c4ccc5c(c4)C(c4ccccc4)(c4ccccc4)c4ccccc4-5)cc3)cc2)cc1 ; c1ccc(-c2ccc(N(c3ccc(-c4ccc5c(c4)C4(c6ccccc6-c6ccccc64)c4ccccc4-5)cc3)c3ccc4c(c3)-c3ccccc3C43c4ccccc4-c4ccccc43)cc2)cc1]

N-(4-(9,9-diphenyl-9H-fluoren-2-yl)phenyl)-[1,1′-biphenyl]-4-amine (10 g, 17.8 mmol), 3-bromo-9,9-diphenyl-9H-fluorene (7.78 g, 19.6 mmol), Pd₂(dba)₃ (0.82 g, 0.89 mmol), P(t-Bu)₃ (0.36 g, 1.78 mmol), NaOt-Bu (5.65 g, 58.75 mmol), and toluene (190 mL) were added in a round bottom flask, and the same procedure as described in the synthesis method of Product 1-1′ above was carried out to obtain 11.3 g of Product 1-75′ (yield:72%).

TABLE 2

| compound | FD-MS |
|---|---|
| 1-1′ | m/z = 473.21(C₃₆H₂₇N = 473.61) |
| 1-2′ | m/z = 523.23(C₄₀H₂₉N = 523.66) |
| 1-3′ | m/z = 573.25(C₄₄H₃₁N = 573.72) |
| 1-4′ | m/z = 623.26(C₄₈H₃₃N = 623.78) |
| 1-5′ | m/z = 447.20(C₃₄H₂₅N = 447.57) |
| 1-6′ | m/z = 371.17(C₂₈H₂₁N = 371.47) |
| 1-7′ | m/z = 471.20(C₃₆H₂₅N = 471.59) |
| 1-8′ | m/z = 521.21(C₄₀H₂₇N = 521.65) |
| 1-9′ | m/z = 549.25(C₄₂H₃₁N = 549.70) |
| 1-10′ | m/z = 625.28(C₄₈H₃₅N = 625.80) |
| 1-11′ | m/z = 675.29(C₅₂H₃₇N = 675.86) |
| 1-12′ | m/z = 473.21(C₃₆H₂₇N = 473.61) |
| 1-13′ | m/z = 523.23(C₄₀H₂₉N = 523.66) |
| 1-14′ | m/z = 623.26(C₄₈H₃₃N = 623.78) |
| 1-15′ | m/z = 549.25(C₄₂H₃₁N = 549.70) |
| 1-16′ | m/z = 625.28(C₄₈H₃₅N = 625.80) |
| 1-17′ | m/z = 503.17(C₃₆H₂₅NS = 503.66) |
| 1-18′ | m/z = 603.20(C₄₄H₂₉NS = 603.77) |
| 1-19′ | m/z = 477.16(C₃₄H₂₃NS = 477.62) |
| 1-20′ | m/z = 503.17(C₃₆H₂₅NS = 503.66) |
| 1-21′ | m/z = 451.14(C₃₂H₂₁NS = 451.58) |
| 1-22′ | m/z = 593.22(C₄₃H₃₁NS = 593.78) |
| 1-23′ | m/z = 641.22(C₄₇H₃₁NS = 641.82) |
| 1-24′ | m/z = 665.22(C₄₉H₃₁NS = 665.84) |
| 1-25′ | m/z = 503.17(C₃₆H₂₅NS = 503.66) |
| 1-26′ | m/z = 655.23(C₄₈H₃₃NS = 655.85) |
| 1-27′ | m/z = 695.26(C₅₁H₃₇NS = 695.91) |
| 1-28′ | m/z = 593.18(C₄₂H₂₇NOS = 593.73) |
| 1-29′ | m/z = 583.14(C₄₀H₂₅NS₂ = 583.76) |
| 1-30′ | m/z = 579.20(C₄₂H₂₉NS = 579.75) |
| 1-31′ | m/z = 685.19(C₄₈H₃₁NS₂ = 685.90) |
| 1-32′ | m/z = 719.23(C₅₂H₃₃NOS = 719.89) |
| 1-33′ | m/z = 629.22(C₄₆H₃₁NS = 629.81) |
| 1-34′ | m/z = 629.22(C₄₆H₃₁NS = 629.81) |
| 1-35′ | m/z = 603.20(C₄₄H₂₉NS = 603.77) |
| 1-36′ | m/z = 563.08(C₃₆H₂₁NS₃ = 563.75) |
| 1-37′ | m/z = 639.11(C₄₂H₂₅NS₃ = 639.85) |
| 1-38′ | m/z = 715.15(C₄₈H₂₉NS₃ = 715.95) |
| 1-39′ | m/z = 791.18(C₅₄H₃₃NS₃ = 792.04) |
| 1-40′ | m/z = 607.16(C₄₂H₂₅NO₂S = 607.72) |
| 1-41′ | m/z = 633.21(C₄₅H₃₁NOS = 633.80) |
| 1-42′ | m/z = 733.24(C₅₃H₃₅NOS = 733.92) |
| 1-43′ | m/z = 883.29(C₆₅H₄₁NOS = 884.09) |
| 1-44′ | m/z = 585.13(C₃₈H₂₃N₃S₂ = 585.74) |
| 1-45′ | m/z = 553.19(C₄₀H₂₇NS = 553.71) |
| 1-46′ | m/z = 603.20(C₄₄H₂₉NS = 603.77) |
| 1-47′ | m/z = 841.28(C₆₃H₃₉NS = 842.06) |
| 1-48′ | m/z = 563.22(C₄₂H₂₉NO = 563.69) |
| 1-49′ | m/z = 563.22(C₄₂H₂₉NO = 563.69) |
| 1-50′ | m/z = 613.24(C₄₆H₃₁NO = 613.74) |
| 1-51′ | m/z = 703.29(C₅₃H₃₇NO = 703.87) |
| 1-52′ | m/z = 587.22(C₄₄H₂₉NO = 587.71) |
| 1-53′ | m/z = 639.26(C₄₈H₃₃NO = 639.78) |
| 1-54′ | m/z = 639.26(C₄₈H₃₃NO = 639.78) |
| 1-55′ | m/z = 653.24(C₄₈H₃₁NO₂ = 653.77) |
| 1-56′ | m/z = 603.26(C₄₅H₃₃NO = 603.75) |
| 1-57′ | m/z = 727.29(C₅₅H₃₇NO = 727.89) |
| 1-58′ | m/z = 725.27(C₅₅H₃₅NO = 725.87) |
| 1-59′ | m/z = 595.17(C₄₀H₂₅N₃OS = 595.71) |
| 1-60′ | m/z = 567.26(C₄₂H₃₃NO = 567.72) |
| 1-61′ | m/z = 611.22(C₄₆H₂₉NO = 611.73) |
| 1-62′ | m/z = 617.18(C₄₄H₂₇NOS = 617.76) |
| 1-63′ | m/z = 637.24(C₄₈H₃₁NO = 637.7) |
| 1-64′ | m/z = 667.21(C₄₈H₂₉NO₃ = 667.75) |
| 1-65′ | m/z = 767.25(C₅₆H₃₃NO₃ = 767.87) |
| 1-66′ | m/z = 681.27(C₅₀H₃₅NO₂ = 681.82) |
| 1-67′ | m/z = 713.31(C₅₅H₃₉N = 713.90) |
| 1-68′ | m/z = 589.28(C₄₅H₃₅N = 589.77) |
| 1-69′ | m/z = 639.29(C₄₉H₃₇N = 639.82) |
| 1-70′ | m/z = 613.28(C₄₇H₃₅N = 613.79) |
| 1-71′ | m/z = 711.29(C₅₆H₃₇N = 711.89) |
| 1-72′ | m/z = 637.28(C₄₉H₃₅N = 637.81) |
| 1-73′ | m/z = 761.31(C₅₉H₃₉N = 761.95) |
| 1-74′ | m/z = 637.28(C₄₉H₃₅N = 637.81) |
| 1-75′ | m/z = 877.37(C₆₈H₄₇N = 878.11) |
| 1-76′ | m/z = 875.36(C₆₈H₄₅N = 876.09) |
| 1-77′ | m/z = 813.30(C₆₂H₃₉NO = 813.98) |

The final product represented by Formula 2 or 2′ according to the present invention can be synthesized by reaction between Sub 3 and Sub 4 as illustrated in the following Reaction Scheme 4.

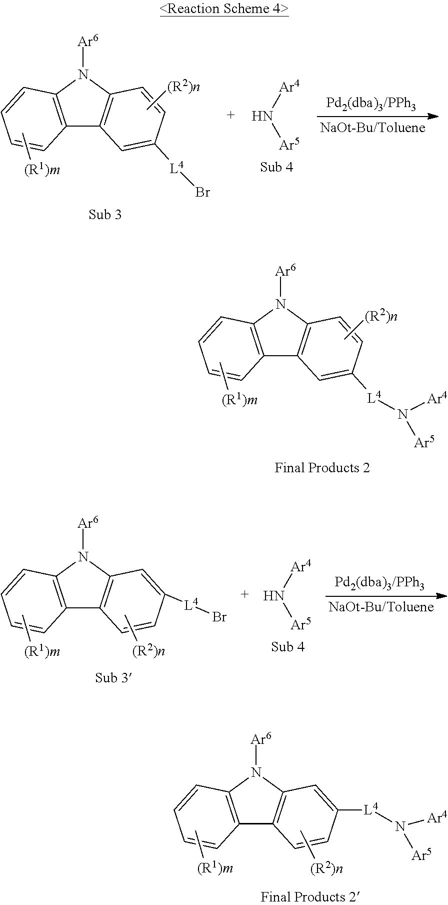

### Synthesis Example of Sub 3 (L4 is not a Single Bond)

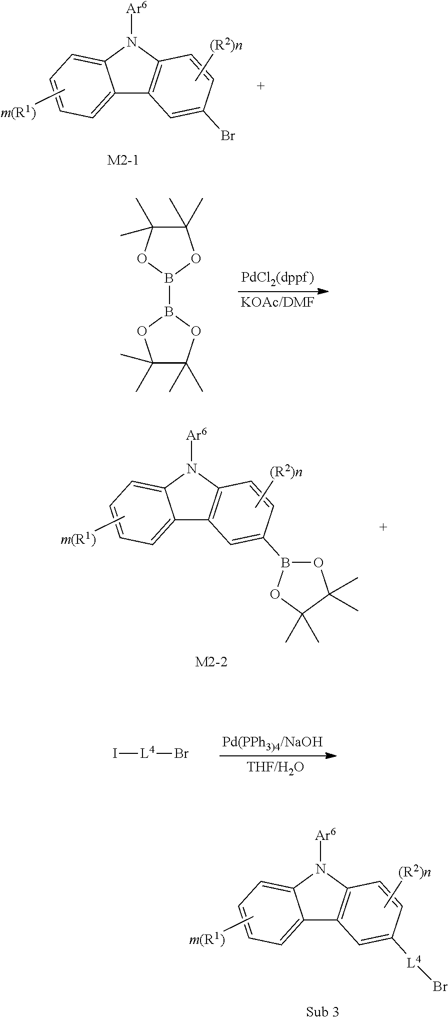

### Synthesis Example of Sub 3′(L4 is not a Single Bond)

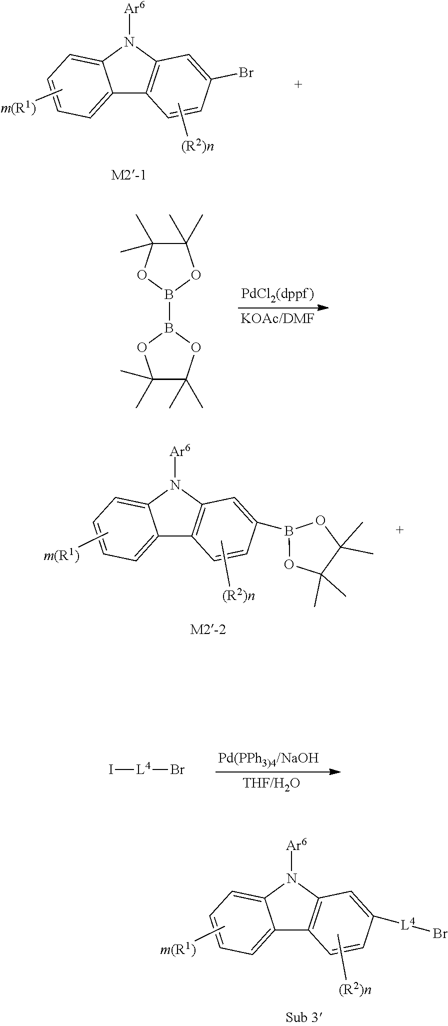

### 1) Synthesis Example of M3-2-1

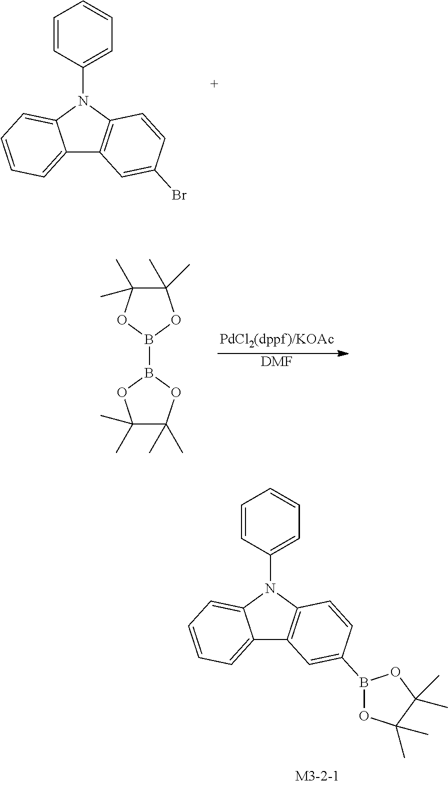

After 3-bromo-9-phenyl-9H-carbazole (45.1 g, 140 mmol) was dissolved in DMF 980 mL, Bispinacolborate (39.1 g, 154 mmol), PdCl₂(dppf) catalyst (3.43 g, 4.2 mmol), KOAc (41.3 g, 420 mmol) was added in order and stirred for 24 hours to synthesize a borate compound, and then the resulting compound was separated over a silicagel column chromatography and recrystallization to give the borate compound 35.2 g (68%).

### 2) Synthesis Example of M3-2-2

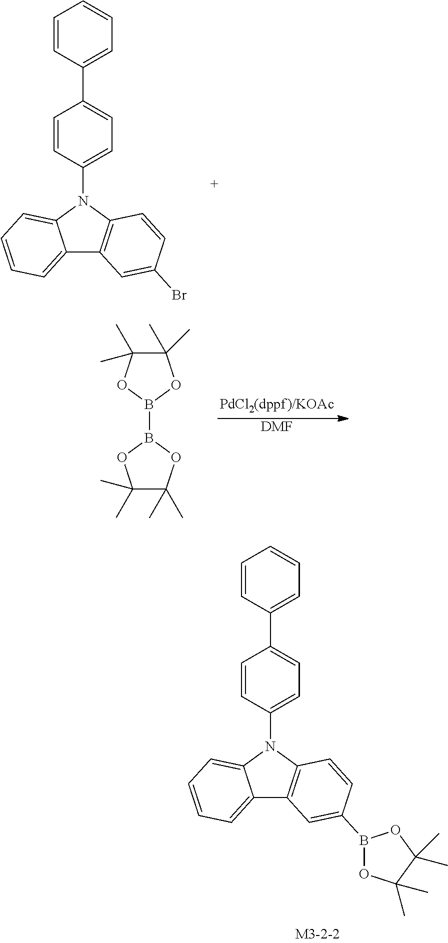

Through the same experimental procedure as M3-2-1, 40 g (64%) was obtained.

### 3) Synthesis Example of Sub 3-1-1

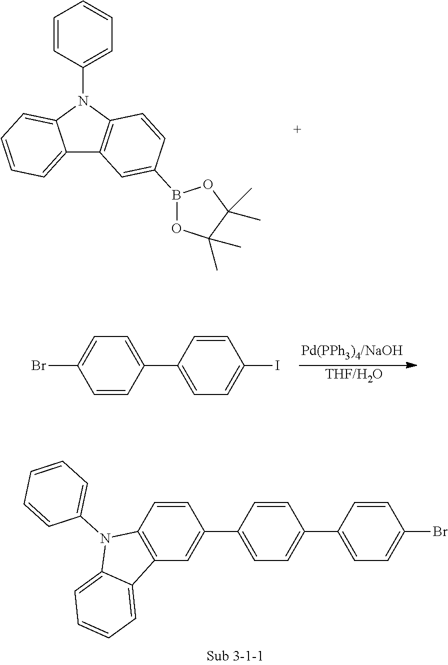 [SMILES: CC1(C)OB(c2ccc3c(c2)c2ccccc2n3-c2ccccc2)OC1(C)C ; Brc1ccc(-c2ccc(I)cc2)cc1 ; "THF/H2O" ; Brc1ccc(-c2ccc(-c3ccc4c(c3)c3ccccc3n4-c3ccccc3)cc2)cc1]

After M3-2-1 (29.5 g, 80 mmol) was dissolved in THF 360 mL, 4-bromo-4′-iodo-1,1′-biphenyl (30.16 g, 84 mmol), Pd(PPh₃)₄ (2.8 g, 2.4 mmol), NaOH (9.6 g, 240 mmol), 180 mL of water were added, and followed by stirring under reflux. When the reaction is complete, the mixture is extracted with ether and water, and the organic layer was dried over MgSO₄ and concentrated, and then the product is separated by silicagel column chromatography and recrystallized to give 26.56 g (70%) of the product.

### Synthesis Example of Sub 3-1-2

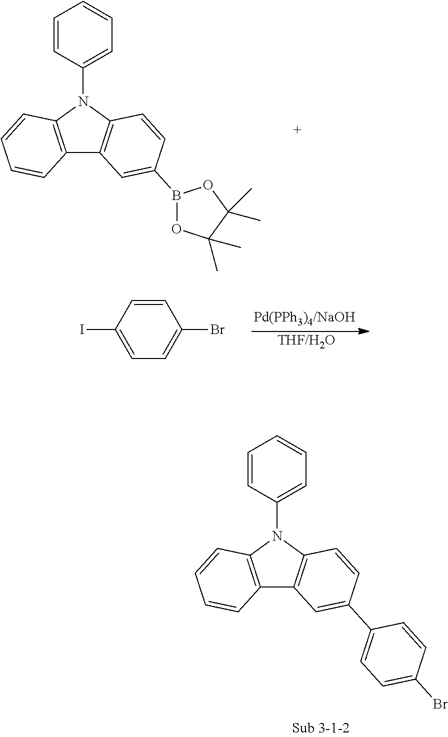 [SMILES: CC1(C)OB(c2ccc3c(c2)c2ccccc2n3-c2ccccc2)OC1(C)C ; Brc1ccc(I)cc1 ; "THF/H2O" ; Brc1ccc(-c2ccc3c(c2)c2ccccc2n3-c2ccccc2)cc1]

After M3-2-1 (29.5 g, 80 mmol) was dissolved in THF 360 mL, 1-bromo-4-iodobenzene (23.8 g, 84 mmol), Pd(PPh₃)₄ (2.8 g, 2.4 mmol), NaOH (9.6 g, 240 mmol), 180 mL of water were added, and followed by stirring under reflux. When the reaction is complete, the mixture is extracted with ether and water, and the organic layer was dried over MgSO₄ and concentrated, and then the product is separated by silicagel column chromatography and recrystallized to give 22.9 g (72%) of the product.

### 5) Synthesis Example of Sub 3-1-3

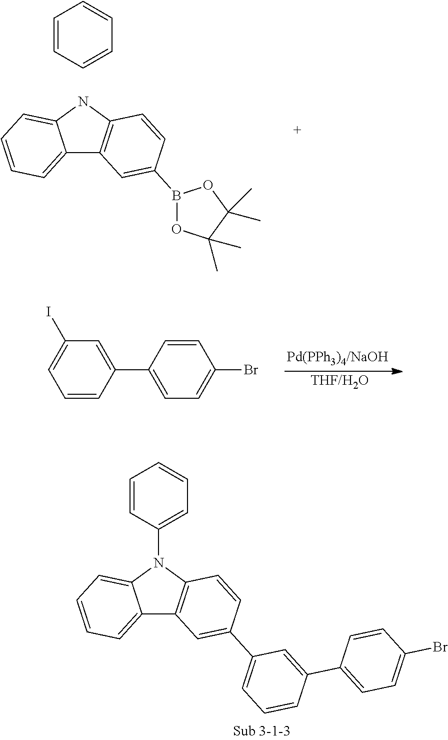

After M3-2-1 (29.5 g, 80 mmol) was dissolved in THF 360 mL, 4′-bromo-3-iodo-1,1′-biphenyl (30.16 g, 84 mmol), Pd(PPh₃)₄ (2.8 g, 2.4 mmol), NaOH (9.6 g, 240 mmol), 180 mL of water were added, followed by stirring under reflux. When the reaction is complete, the mixture is extracted with ether and water, and the organic layer was dried over MgSO₄ and concentrated, and then the product is separated by silicagel column chromatography and recrystallized to give 24.7 g (65%) of the product.

### 6) Synthesis Example of Sub 3-1-4

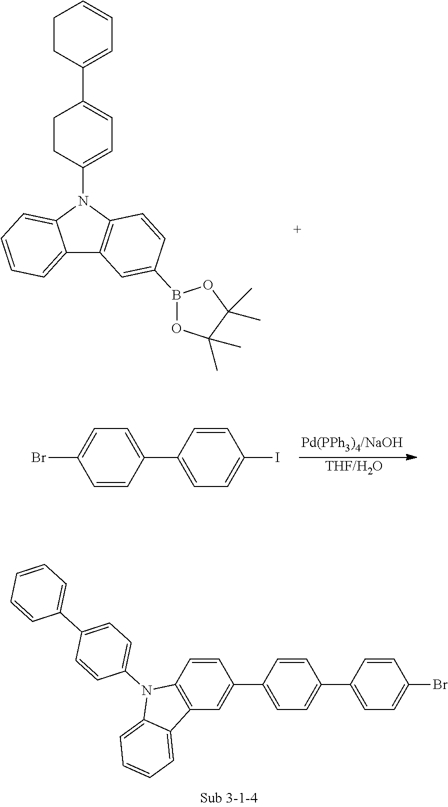 [SMILES: CC1(C)OB(c2ccc3c(c2)c2ccccc2n3C2=CC=C(C3=CC=CCC3)CC2)OC1(C)C ; Brc1ccc(-c2ccc(I)cc2)cc1 ; "THF/H2O" ; Brc1ccc(-c2ccc(-c3ccc4c(c3)c3ccccc3n4-c3ccc(-c4ccccc4)cc3)cc2)cc1]

After the obtained M3-2-2 (35.63 g, 80 mmol) was dissolved in THF 360 mL, 4-bromo-4′-iodo-1,1′-biphenyl (30.16 g, 84 mmol), Pd(PPh₃)₄ (2.8 g, 2.4 mmol), NaOH (9.6 g, 240 mmol), 180 mL of water were added, and followed by stirring under reflux. When the reaction is complete, the mixture is extracted with ether and water, and the organic layer was dried over MgSO₄ and concentrated, and then the product is separated by silicagel column chromatography and recrystallized to give 29.51 g (67%) of the product.

### 7) Synthesis Example of M3′-2-1

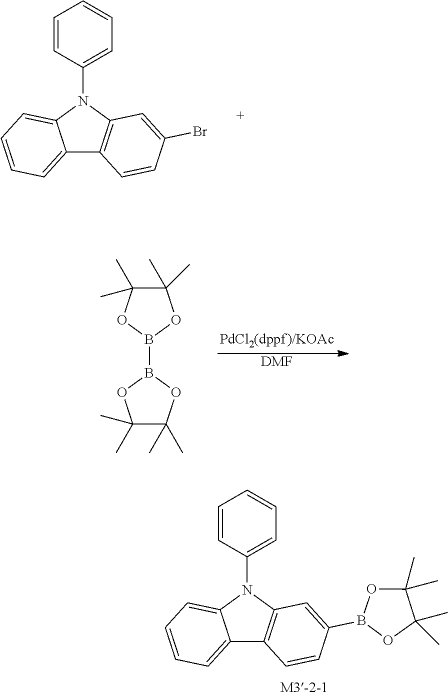

After 2-bromo-9-phenyl-9H-carbazole (45.1 g, 140 mmol) was dissolved in DMF 980 mL, Bispinacolborate (39.1 g, 154 mmol), PdCl₂(dppf) catalyst (3.43 g, 4.2 mmol), KOAc (41.3 g, 420 mmol) was added in order and stirred for 24 hours to synthesize a borate compound, and then the product was separated by a silicagel column chromatography and recrystallized to give the borate compound 36.2 g (70%).

### 8) Synthesis Example of M3′-2-2

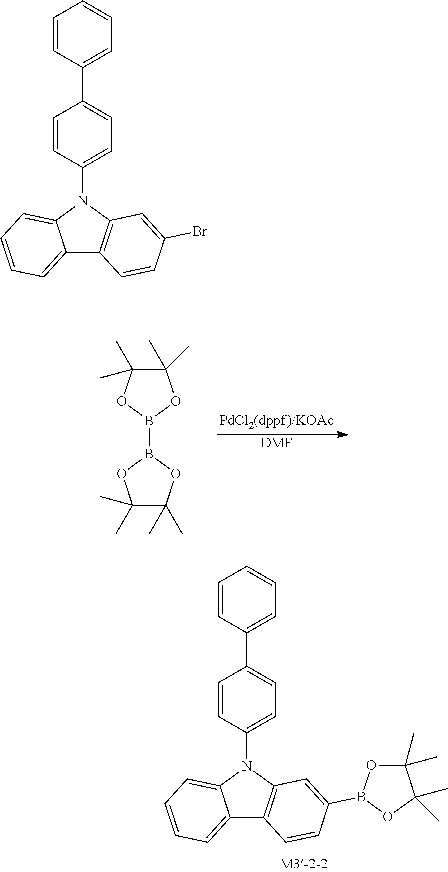

Through the same experimental procedure as M3′-2-1, 43.6 g (67%) was obtained.

### 9) Synthesis Example of Sub3′-1-1

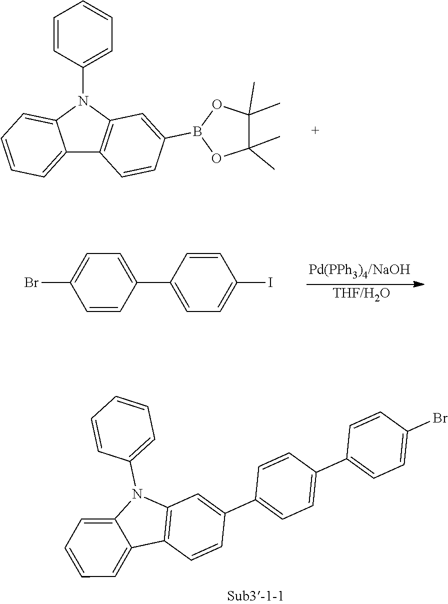 [SMILES: CC1(C)OB(c2ccc3c4ccccc4n(-c4ccccc4)c3c2)OC1(C)C ; Brc1ccc(-c2ccc(I)cc2)cc1 ; "THF/H2O" ; Brc1ccc(-c2ccc(-c3ccc4c5ccccc5n(-c5ccccc5)c4c3)cc2)cc1]

After M3′-2-1 (29.5 g, 80 mmol) was dissolved in THF 360 mL, 4-bromo-4′-iodo-1,1′-biphenyl (30.16 g, 84 mmol), Pd(PPh₃)₄ (2.8 g, 2.4 mmol), NaOH (9.6 g, 240 mmol), 180 mL of water were added, and followed by stirring under reflux. When the reaction is complete, the mixture is extracted with ether and water, and the organic layer was dried over MgSO₄ and concentrated, and then the product is separated by silicagel column chromatography and recrystallized to give 26.95 g (70%) of the product.

### 10) Synthesis Example of Sub 3′-1-2

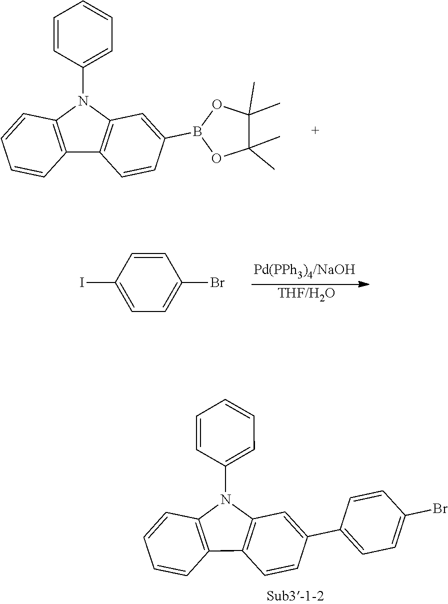 [SMILES: CC1(C)OB(c2ccc3c4ccccc4n(-c4ccccc4)c3c2)OC1(C)C ; Brc1ccc(I)cc1 ; "THF/H2O" ; Brc1ccc(-c2ccc3c4ccccc4n(-c4ccccc4)c3c2)cc1]

After M3′-2-1 (29.5 g, 80 mmol) was dissolved in THF 360 mL, 1-bromo-4′-iodo-1,1′-biphenyl(23.8 g, 84 mmol), Pd(PPh₃)₄ (2.8 g, 2.4 mmol), NaOH (9.6 g, 240 mmol), 180 mL of water were added, and the same procedure as described in the synthesis method of Sub 3-1-1 was carried out to obtain 23.26 g of Product (yield:73%).

### 11) Synthesis Example of Sub 3′-1-3

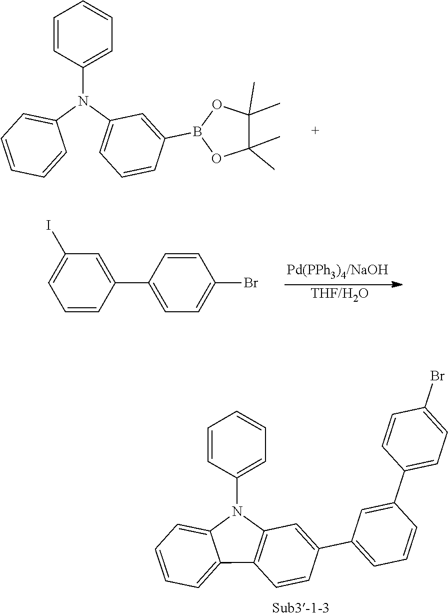 [SMILES: CC1(C)OB(c2cccc(N(c3ccccc3)c3ccccc3)c2)OC1(C)C ; Brc1ccc(-c2cccc(I)c2)cc1 ; "THF/H2O" ; Brc1ccc(-c2cccc(-c3ccc4c5ccccc5n(-c5ccccc5)c4c3)c2)cc1]

After M3′-2-1 (29.5 g, 80 mmol) was dissolved in THF 360 mL, 4′-bromo-4′-iodo-1,1′-biphenyl(30.16 g, 84 mmol), Pd(PPh₃)₄ (2.8 g, 2.4 mmol), NaOH (9.6 g, 240 mmol), 180 mL of water were added, and the same procedure as described in the synthesis method of Sub 3-1-1 was carried out to obtain 25.8 g of Product (yield:68%).

### 12) Synthesis Example of Sub 3′-1-4

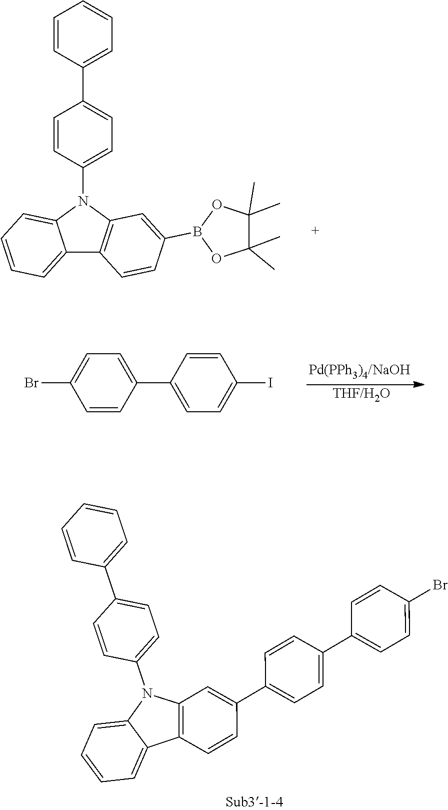 [SMILES: CC1(C)OB(c2ccc3c4ccccc4n(-c4ccc(-c5ccccc5)cc4)c3c2)OC1(C)C ; Brc1ccc(-c2ccc(I)cc2)cc1 ; "THF/H2O" ; Brc1ccc(-c2ccc(-c3ccc4c5ccccc5n(-c5ccc(-c6ccccc6)cc5)c4c3)cc2)cc1]

After M3′-2-2 (35.63 g, 80 mmol) was dissolved in THF 360 mL, 4-bromo-4′-iodo-1,1′-biphenyl (30.16 g, 84 mmol), Pd(PPh₃)₄ (2.8 g, 2.4 mmol), NaOH (9.6 g, 240 mmol), 180 mL of water were added, and followed by stirring under reflux. When the reaction is complete, the mixture is extracted with ether and water, and the organic layer was dried over MgSO₄ and concentrated, and then the resultant organic material is separated by silicagel column chromatography and recrystallized to give 30.4 g (69%) of the product.

### Synthesis Example of Sub 4

Sub 4 of the reaction scheme 1 can be synthesized by reaction path of the following Reaction Scheme 5.

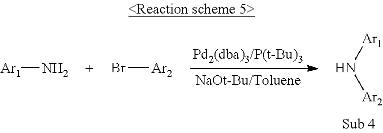

### Synthesis Example of Sub 4-28

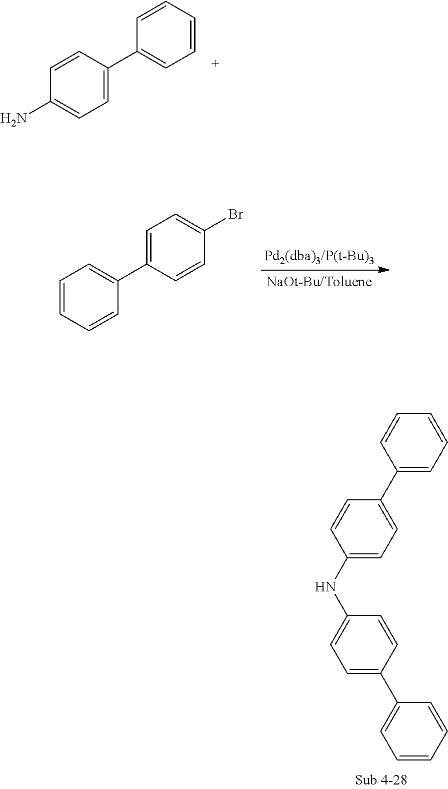 [SMILES: Nc1ccc(-c2ccccc2)cc1 ; Brc1ccc(-c2ccccc2)cc1 ; "Pd2(dba)3/P(t-Bu)3" ; "NaOt-Bu/Toluene" ; c1ccc(-c2ccc(Nc3ccc(-c4ccccc4)cc3)cc2)cc1]

After 4-bromo-1,1′-biphenyl(5.6 g, 24 mmol) was dissolved in toluene, [1,1′-biphenyl]-4-amine (3.4 g, 20 mmol), Pd₂(dba)₃ (0.5 g, 0.6 mmol), P(t-Bu)₃ (0.2 g, 2 mmol), NaOt-Bu (5.8 g, 60 mmol), toluene (300 mL) were added, and followed by stirring under reflux at 100° C. for 24 hours. When the reaction is complete, the mixture is extracted with ether and water, and the organic layer was dried over MgSO₄ and concentrated, and then the product is separated by silicagel column chromatography and recrystallized to give 6.2 g (80%) of the product.

Examples of Sub 4 are followings, but not limit to.

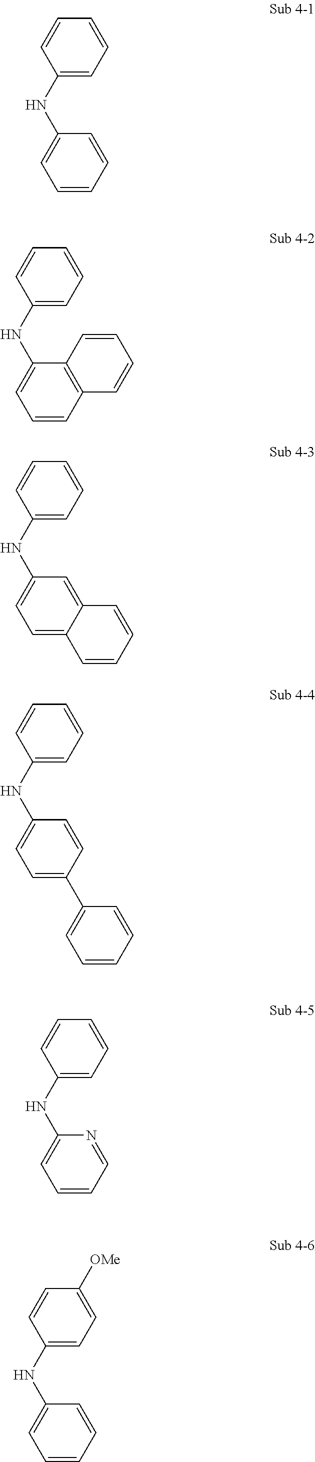

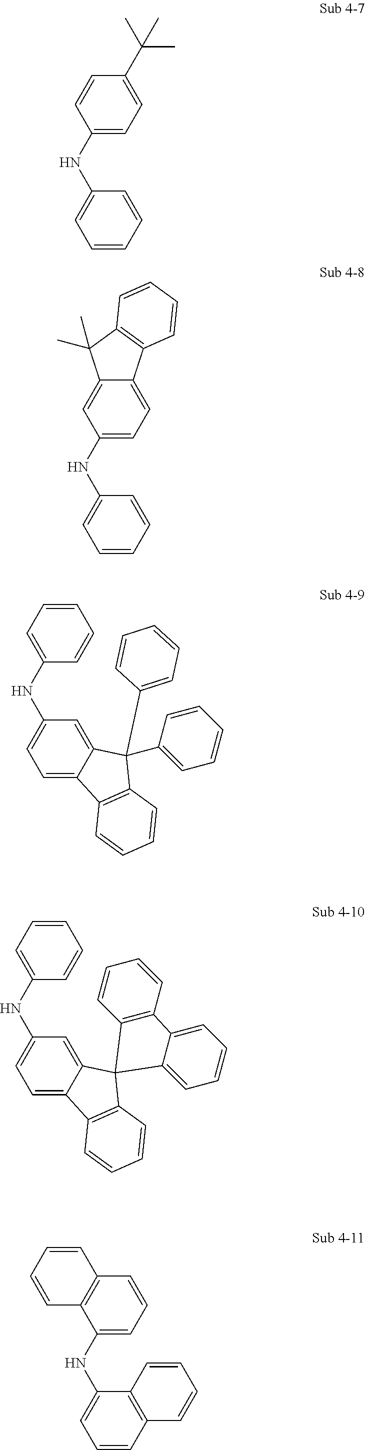

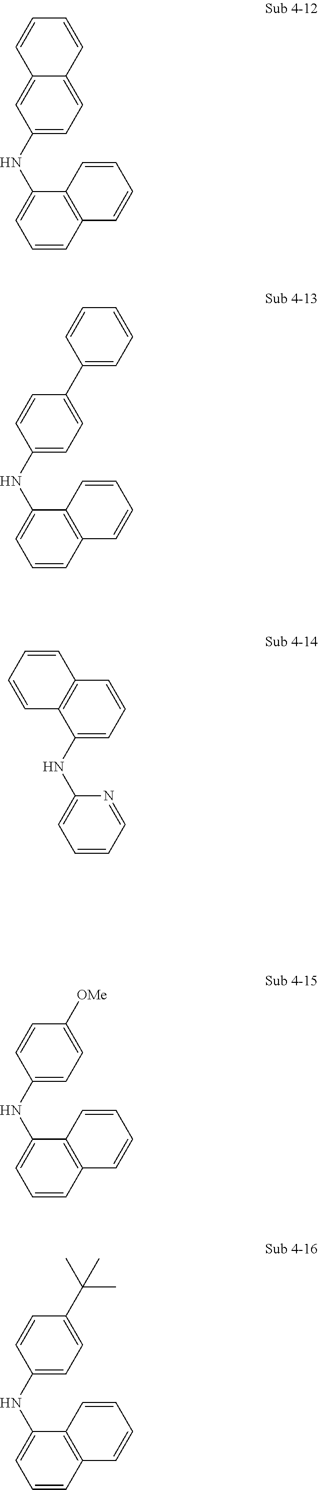 [SMILES: c1ccc2cc(Nc3cccc4ccccc34)ccc2c1 ; c1ccc(-c2ccc(Nc3cccc4ccccc34)cc2)cc1 ; c1ccc(Nc2cccc3ccccc23)nc1 ; COc1ccc(Nc2cccc3ccccc23)cc1 ; CC(C)(C)c1ccc(Nc2cccc3ccccc23)cc1]

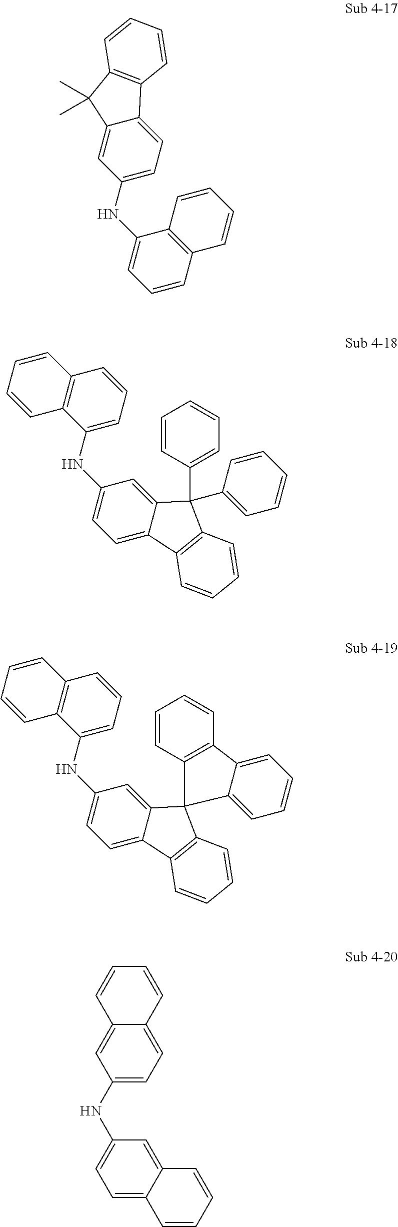 [SMILES: CC1(C)c2ccccc2-c2ccc(Nc3cccc4ccccc34)cc21 ; c1ccc(C2(c3ccccc3)c3ccccc3-c3ccc(Nc4cccc5ccccc45)cc32)cc1 ; c1ccc2c(c1)-c1ccccc1C21c2ccccc2-c2ccc(Nc3cccc4ccccc34)cc21 ; c1ccc2cc(Nc3ccc4ccccc4c3)ccc2c1]

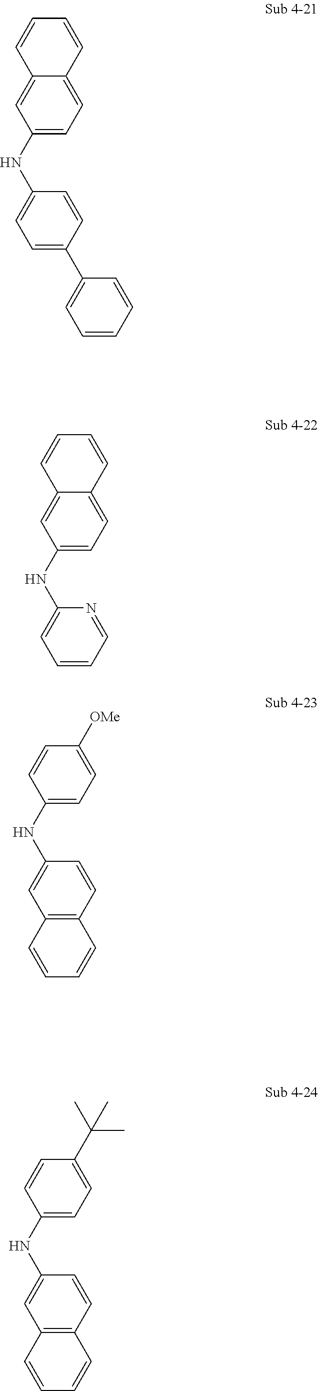 [SMILES: c1ccc(-c2ccc(Nc3ccc4ccccc4c3)cc2)cc1 ; c1ccc(Nc2ccc3ccccc3c2)nc1 ; COc1ccc(Nc2ccc3ccccc3c2)cc1 ; CC(C)(C)c1ccc(Nc2ccc3ccccc3c2)cc1]

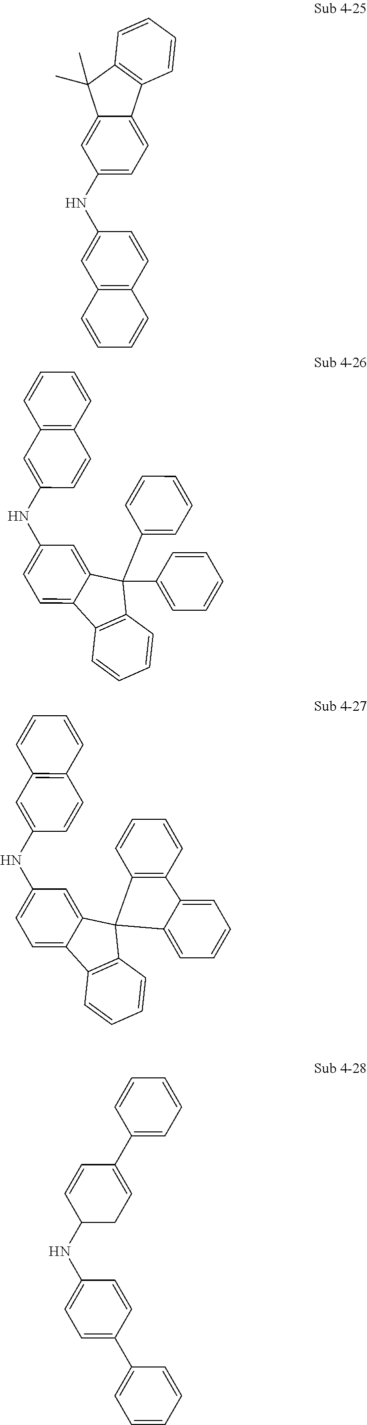

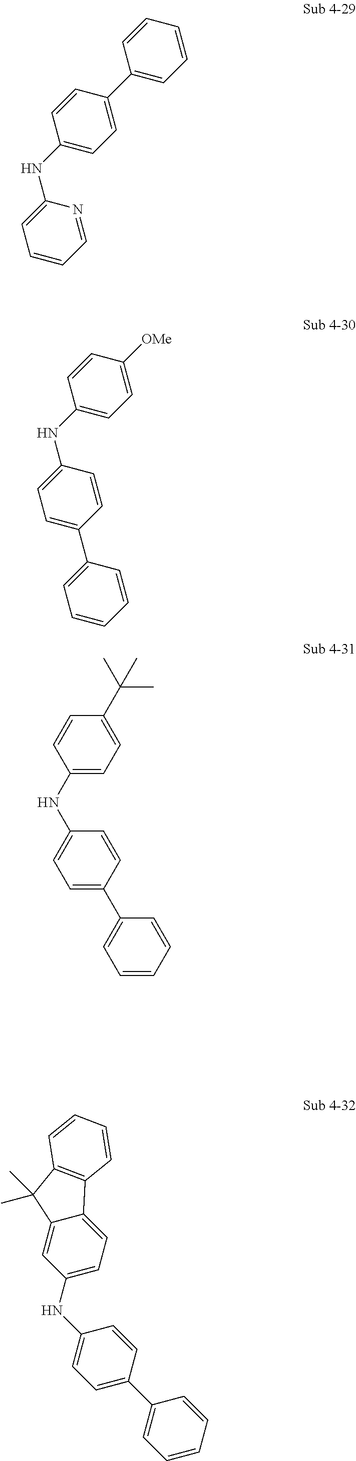

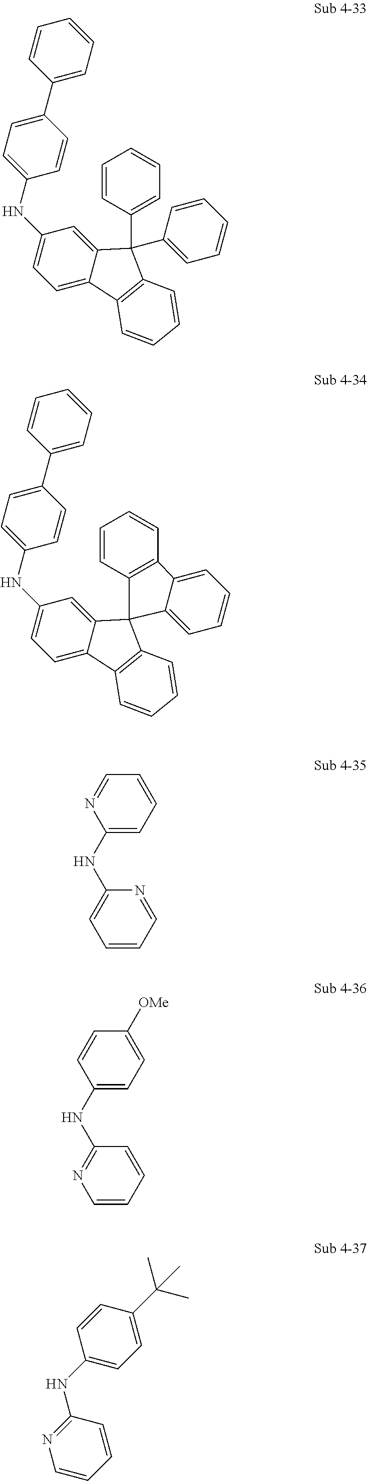 [SMILES: c1ccc(-c2ccc(Nc3ccc4c(c3)C(c3ccccc3)(c3ccccc3)c3ccccc3-4)cc2)cc1 ; c1ccc(-c2ccc(Nc3ccc4c(c3)C3(c5ccccc5-c5ccccc53)c3ccccc3-4)cc2)cc1 ; c1ccc(Nc2ccccn2)nc1 ; COc1ccc(Nc2ccccn2)cc1 ; CC(C)(C)c1ccc(Nc2ccccn2)cc1]

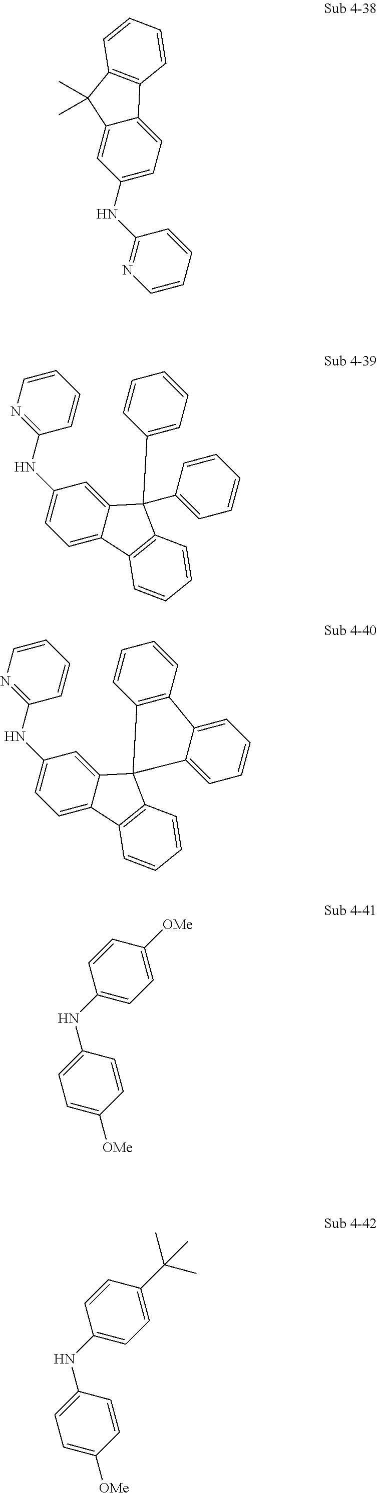

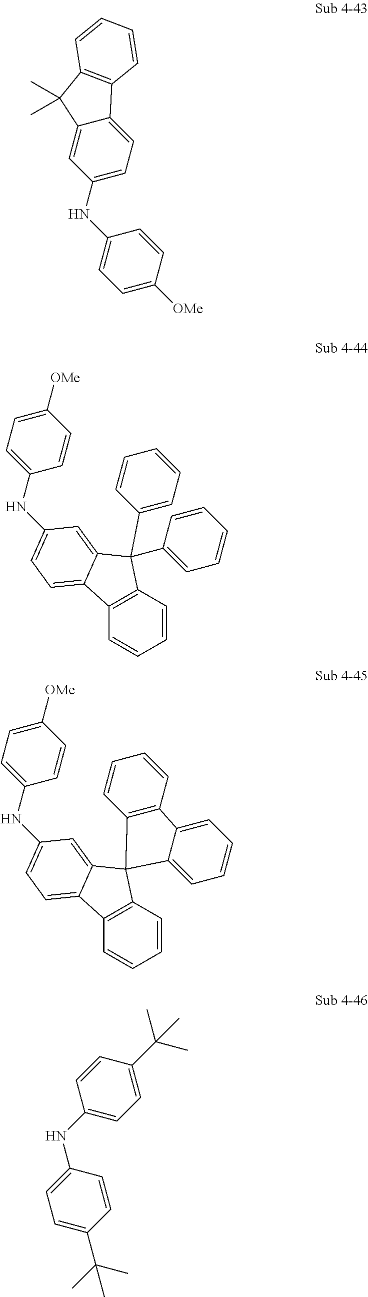

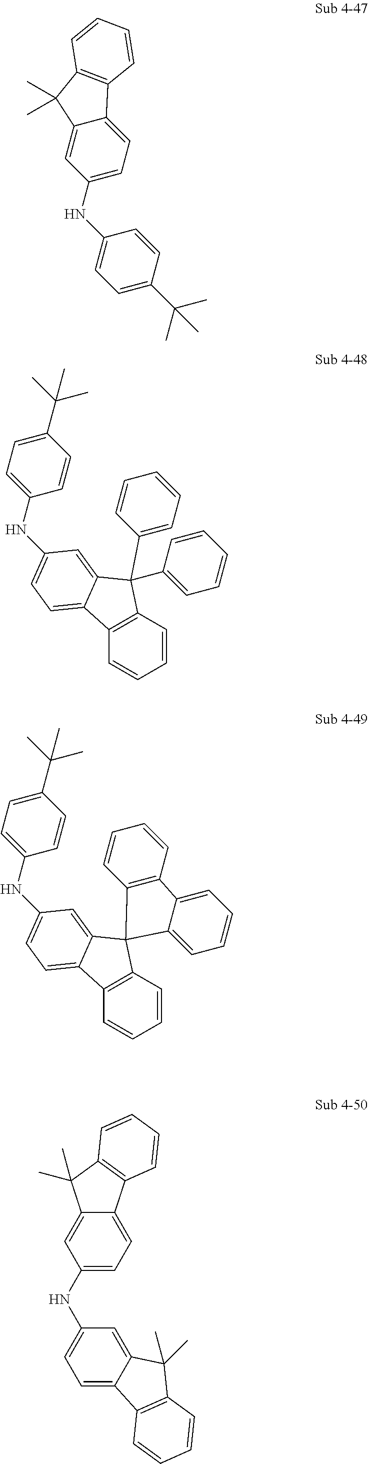

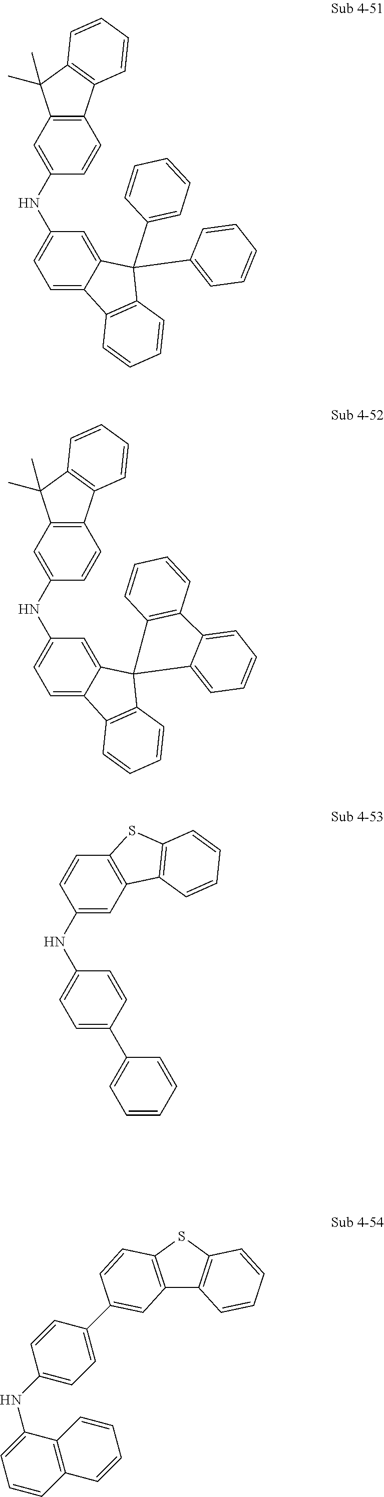 [SMILES: CC1(C)c2ccccc2-c2ccc(Nc3ccc4c(c3)C(c3ccccc3)(c3ccccc3)c3ccccc3-4)cc21 ; CC1(C)c2ccccc2-c2ccc(Nc3ccc4c(c3)C3(c5ccccc5-c5ccccc53)c3ccccc3-4)cc21 ; c1ccc(-c2ccc(Nc3ccc4sc5ccccc5c4c3)cc2)cc1 ; c1ccc2c(Nc3ccc(-c4ccc5sc6ccccc6c5c4)cc3)cccc2c1]

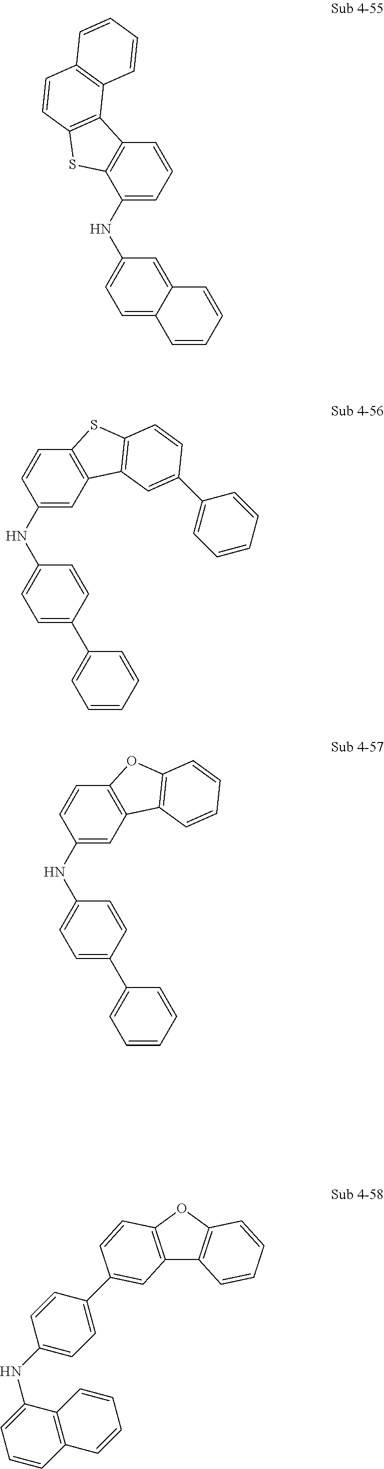 [SMILES: c1ccc2cc(Nc3cccc4c3sc3ccc5ccccc5c34)ccc2c1 ; c1ccc(-c2ccc(Nc3ccc4sc5ccc(-c6ccccc6)cc5c4c3)cc2)cc1 ; c1ccc(-c2ccc(Nc3ccc4oc5ccccc5c4c3)cc2)cc1 ; c1ccc2c(Nc3ccc(-c4ccc5oc6ccccc6c5c4)cc3)cccc2c1]

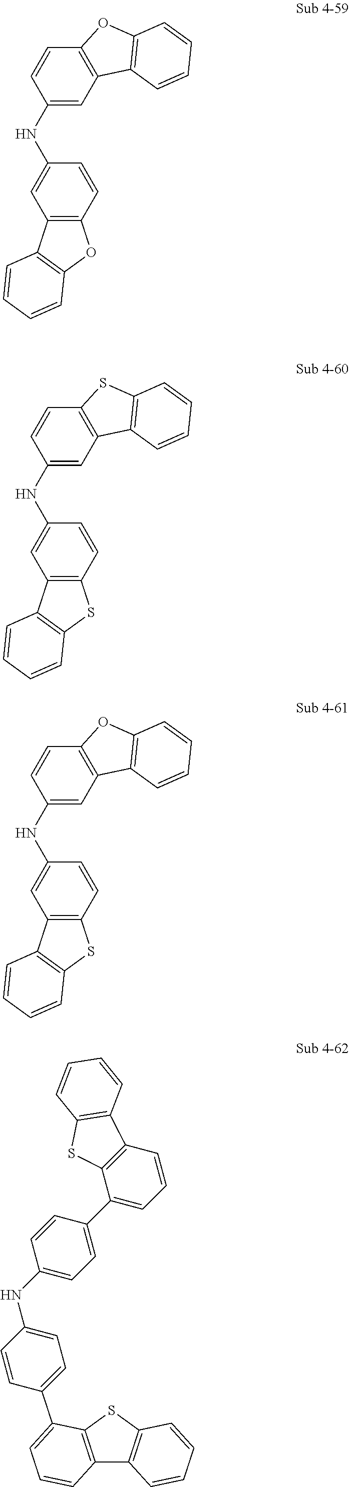 [SMILES: c1ccc2c(c1)oc1ccc(Nc3ccc4oc5ccccc5c4c3)cc12 ; c1ccc2c(c1)sc1ccc(Nc3ccc4sc5ccccc5c4c3)cc12 ; c1ccc2c(c1)oc1ccc(Nc3ccc4sc5ccccc5c4c3)cc12 ; c1ccc2c(c1)sc1c(-c3ccc(Nc4ccc(-c5cccc6c5sc5ccccc56)cc4)cc3)cccc12]

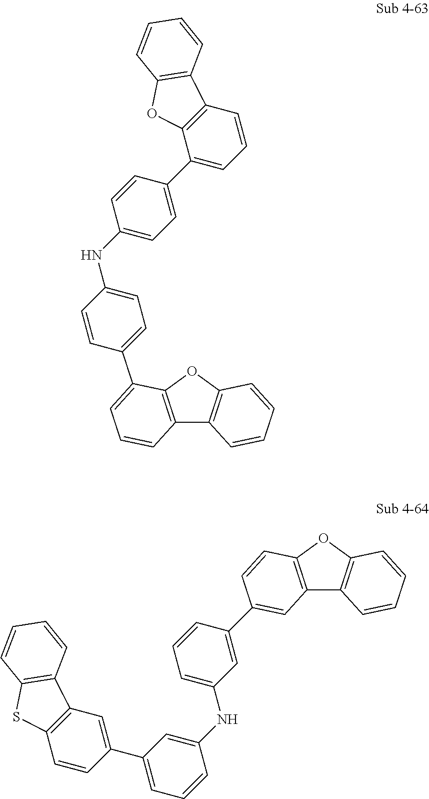 [SMILES: c1ccc2c(c1)oc1c(-c3ccc(Nc4ccc(-c5cccc6c5oc5ccccc56)cc4)cc3)cccc12 ; c1cc(Nc2cccc(-c3ccc4sc5ccccc5c4c3)c2)cc(-c2ccc3oc4ccccc4c3c2)c1]

TABLE 3

| compound | FD-MS |
|---|---|
| Sub 4-1 | m/z = 169.09(C₁₂H₁₁N = 169.22) |
| Sub 4-2 | m/z = 219.10(C₁₆H₁₃N = 219.28) |
| Sub 4-3 | m/z = 219.10(C₁₆H₁₃N = 219.28) |
| Sub 4-4 | m/z = 245.12(C₁₈H₁₅N = 245.32) |
| Sub 4-5 | m/z = 170.08(C₁₁H₁₀N₂ = 170.21) |
| Sub 4-6 | m/z = 199.10(C₁₀H₁₃NO = 199.25) |
| Sub 4-7 | m/z = 225.15(C₁₆H₁₉N = 225.33) |
| Sub 4-8 | m/z = 285.15(C₂₁H₁₉N = 285.38) |
| Sub 4-9 | m/z = 409.18(C₃₁H₂₃N = 409.52) |
| Sub 4-10 | m/z = 407.17(C₃₁H₂₁N = 407.51) |
| Sub 4-11 | m/z = 269.12(C₂₀H₁₅N = 269.34) |
| Sub 4-12 | m/z = 269.12(C₂₀H₁₅N = 269.34) |
| Sub 4-13 | m/z = 295.14(C₂₂H₁₇N = 295.38) |
| Sub 4-14 | m/z = 220.10(C₁₅H₁₂N₂ = 220.27) |
| Sub 4-15 | m/z = 249.12(C₁₇H₁₂NO = 249.31) |
| Sub 4-16 | m/z = 275.17(C₂₀H₂₁N = 275.39) |
| Sub 4-17 | m/z = 335.17(C₂₅H₂₁N = 335.44) |
| Sub 4-18 | m/z = 459.20(C₃₅H₂₅N = 459.58) |
| Sub 4-19 | m/z = 457.18(C₃₅H₂₃N = 457.56) |
| Sub 4-20 | m/z = 269.12(C₂₀H₁₅N = 269.34) |
| Sub 4-21 | m/z = 295.14(C₂₂H₁₇N = 295.38) |
| Sub 4-22 | m/z = 220.10(C₁₅H₂N₂ = 220.27) |
| Sub 4-23 | m/z = 249.12(C₁₇H₁₅NO = 249.31) |
| Sub 4-24 | m/z = 275.17(C₂₀H₂₁N = 275.39) |
| Sub 4-25 | m/z = 335.17(C₂₅H₂₁N = 335.44) |
| Sub 4-26 | m/z = 459.20(C₃₅H₂₅N = 459.58) |
| Sub 4-27 | m/z = 457.18(C₃₅H₂₃N = 457.56) |
| Sub 4-28 | m/z = 321.15(C₂₄H₁₉N = 321.41) |
| Sub 4-29 | m/z = 246.12(C₁₇H₁₄N₂ = 246.31) |
| Sub 4-30 | m/z = 275.13(C₁₉H₁₇NO = 275.34) |
| Sub 4-31 | m/z = 301.18(C₂₂H₂₃N = 301.42) |
| Sub 4-32 | m/z = 361.18(C₂₇H₂₃N = 361.48) |
| Sub 4-33 | m/z = 485.21(C₃₇H₂₇N = 485.62) |
| Sub 4-34 | m/z = 483.20(C₃₇H₂₅N = 483.60) |
| Sub 4-35 | m/z = 171.08(C₁₀H₀₉N₃ = 171.20) |
| Sub 4-36 | m/z = 200.09(C₁₂H₁₂N₂O = 200.24) |
| Sub 4-37 | m/z = 226.15(C₁₅H₁₈N₂ = 226.32) |
| Sub 4-38 | m/z = 286.15(C₂₀H₁₈N₂ = 286.37) |
| Sub 4-39 | m/z = 410.18(C₃₀H₂₂N₂ = 410.51) |
| Sub 4-40 | m/z = 408.16(C₃₀H₂₀N₂ = 408.49) |
| Sub 4-41 | m/z = 229.11(C₁₄H₁₅NO₂ = 229.27) |
| Sub 4-42 | m/z = 255.16(C₁₇H₂₁NO = 255.35) |
| Sub 4-43 | m/z = 315.16(C₂₂H₂₁NO = 315.41) |
| Sub 4-44 | m/z = 439.19(C₃₂H₂₅NO = 439.55) |
| Sub 4-45 | m/z = 437.18(C₃₂H₂₃NO = 437.53) |
| Sub 4-46 | m/z = 281.21(C₂₀H₂₇N = 281.44) |
| Sub 4-47 | m/z = 341.21(C₂₅H₂₇N = 341.49) |
| Sub 4-48 | m/z = 465.25(C₃₅H₃₁N = 465.63) |
| Sub 4-49 | m/z = 463.23(C₃₅H₂₉N = 463.61) |
| Sub 4-50 | m/z = 401.21(C₃₀H₂₇N = 401.54) |
| Sub 4-51 | m/z = 525.25(C₄₀H₃₁N = 525.68) |
| Sub 4-52 | m/z = 523.23(C₄₀H₂₉N = 523.66) |
| Sub 4-53 | m/z = 351.11(C₂₄H₁₇NS = 351.46) |
| Sub 4-54 | m/z = 401.12(C₂₈H₁₉NS = 401.52) |
| Sub 4-55 | m/z = 357.11(C₂₆H₁₇NS = 375.48) |
| Sub 4-56 | m/z = 427.14(C₃₀H₂₁NS = 427.56) |
| Sub 4-57 | m/z = 335.13(C₂₄H₁₇NO = 335.40) |
| Sub 4-58 | m/z = 385.15(C₂₈H₁₉NO = 385.46) |
| Sub 4-59 | m/z = 349.11(C₂₄H₁₅NO₂ = 349.38) |
| Sub 4-60 | m/z = 381.06(C₂₄H₁₅NS₂ = 381.51) |
| Sub 4-61 | m/z = 365.09(C₂₄H₁₅NOS = 365.45) |
| Sub 4-62 | m/z = 533.13(C₃₆H₂₃NS₂ = 533.70) |
| Sub 4-63 | m/z = 501.17(C₃₆H₂₃NO₂ = 501.57) |
| Sub 4-64 | m/z = 517.15(C₃₆H₂₃NOS = 349.38) |

### Synthesis of Final Product 2 of Formula (2)

### Synthesis Example of 2-5′

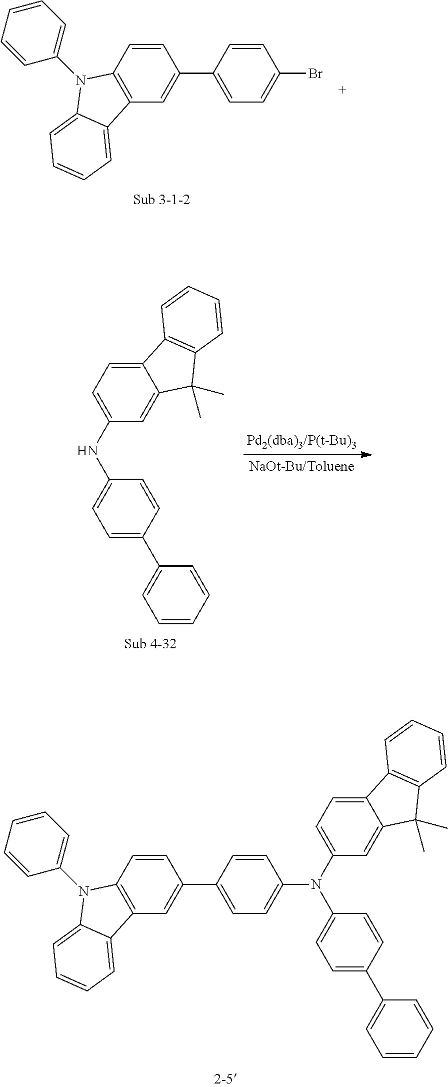 [SMILES: Brc1ccc(-c2ccc3c(c2)c2ccccc2n3-c2ccccc2)cc1 ; CC1(C)c2ccccc2-c2ccc(Nc3ccc(-c4ccccc4)cc3)cc21 ; "Pd2(dba)3/P(t-Bu)3" ; "NaOt-Bu/Toluene" ; CC1(C)c2ccccc2-c2ccc(N(c3ccc(-c4ccccc4)cc3)c3ccc(-c4ccc5c(c4)c4ccccc4n5-c4ccccc4)cc3)cc21]

After Sub 3-1-2 (9.6 g, 24 mmol) was dissolved in toluene, Sub 4-32 (7.2 g, 20 mmol), Pd₂(dba)₃ (0.5 g, 0.6 mmol), P(t-Bu)₃ (0.2 g, 2 mmol), NaOt-Bu (5.8 g, 60 mmol), toluene (300 mL) were added, and the mixture is refluxed with stirring at 100° C. for 24 hours. When the reaction is complete, the mixture is extracted with ether and water, and the organic layer was dried over MgSO₄ and concentrated, and then the product is separated by silicagel column chromatography and recrystallized to give 13.8 g (85%) of the product.

### Synthesis Example of 2-10′

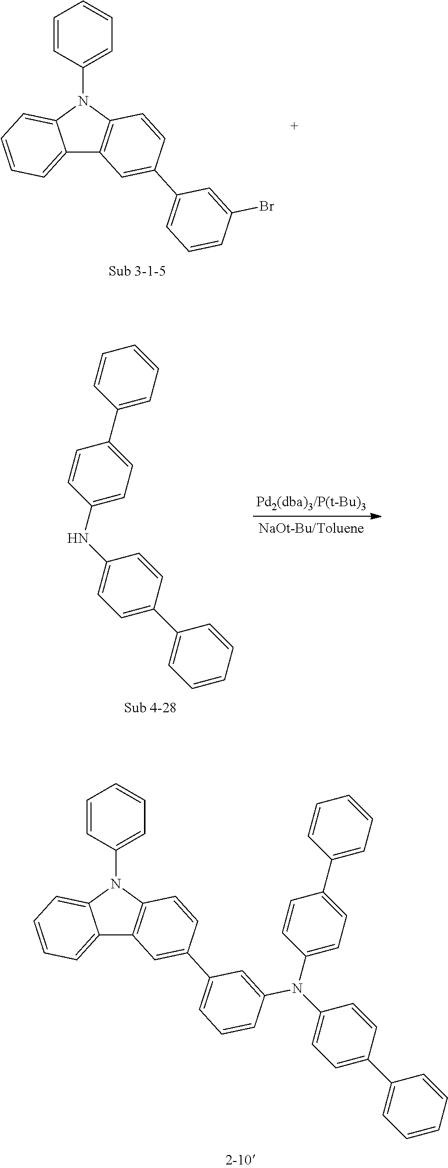 [SMILES: Brc1cccc(-c2ccc3c(c2)c2ccccc2n3-c2ccccc2)c1 ; c1ccc(-c2ccc(Nc3ccc(-c4ccccc4)cc3)cc2)cc1 ; "Pd2(dba)3/P(t-Bu)3" ; "NaOt-Bu/Toluene" ; c1ccc(-c2ccc(N(c3ccc(-c4ccccc4)cc3)c3cccc(-c4ccc5c(c4)c4ccccc4n5-c4ccccc4)c3)cc2)cc1]

After Sub 3-1-5 (9.6 g, 24 mmol) was dissolved in toluene, Sub 4-28 (6.4 g, 20 mmol), Pd₂(dba)₃ (0.5 g, 0.6 mmol), P(t-Bu)₃ (0.2 g, 2 mmol), NaOt-Bu (5.8 g, 60 mmol), toluene (300 mL) were added, and the same procedure as described in the synthesis method of 2-5′ was carried out to obtain 12.0 g of Product (yield:78%).

### Synthesis Example of 2-14′

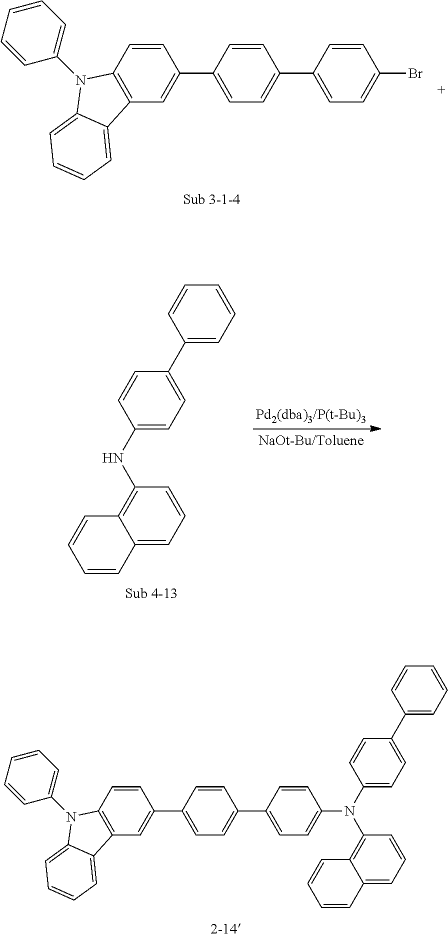 [SMILES: Brc1ccc(-c2ccc(-c3ccc4c(c3)c3ccccc3n4-c3ccccc3)cc2)cc1 ; c1ccc(-c2ccc(Nc3cccc4ccccc34)cc2)cc1 ; "Pd2(dba)3/P(t-Bu)3" ; "NaOt-Bu/Toluene" ; c1ccc(-c2ccc(N(c3ccc(-c4ccc(-c5ccc6c(c5)c5ccccc5n6-c5ccccc5)cc4)cc3)c3cccc4ccccc34)cc2)cc1]

After Sub 3-1-4 (11.4 g, 24 mmol) was dissolved in toluene, Sub 4-13 (5.9 g, 20 mmol), Pd₂(dba)₃ (0.5 g, 0.6 mmol), P(t-Bu)₃ (0.2 g, 2 mmol), NaOt-Bu (5.8 g, 60 mmol), toluene (300 mL) were added, and the same procedure as described in the synthesis method of 2-5′ was carried out to obtain 13.4 g of Product (yield:81%).

### Synthesis Example of 2-36′

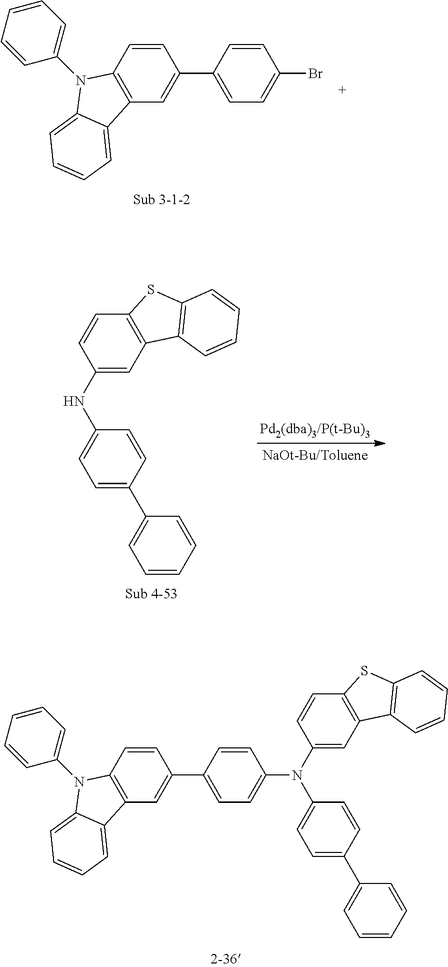 [SMILES: Brc1ccc(-c2ccc3c(c2)c2ccccc2n3-c2ccccc2)cc1 ; c1ccc(-c2ccc(Nc3ccc4sc5ccccc5c4c3)cc2)cc1 ; "Pd2(dba)3/P(t-Bu)3" ; "NaOt-Bu/Toluene" ; c1ccc(-c2ccc(N(c3ccc(-c4ccc5c(c4)c4ccccc4n5-c4ccccc4)cc3)c3ccc4sc5ccccc5c4c3)cc2)cc1]

After Sub 3-1-2 (9.6 g, 24 mmol) was dissolved in toluene, Sub 4-53 (7.0 g, 20 mmol), Pd₂(dba)₃ (0.5 g, 0.6 mmol), P(t-Bu)₃ (0.2 g, 2 mmol), NaOt-Bu (5.8 g, 60 mmol), toluene (300 mL) were added, and the same procedure as described in the synthesis method of 2-5′ was carried out to obtain 13.3 g of Product (yield:83%).

### Synthesis Example of 2-46′

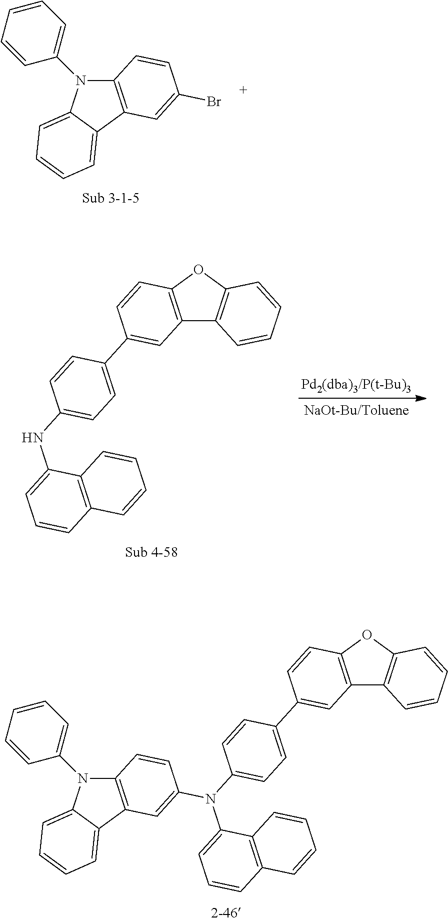 [SMILES: Brc1ccc2c(c1)c1ccccc1n2-c1ccccc1 ; c1ccc2c(Nc3ccc(-c4ccc5oc6ccccc6c5c4)cc3)cccc2c1 ; "Pd2(dba)3/P(t-Bu)3" ; "NaOt-Bu/Toluene" ; c1ccc(-n2c3ccccc3c3cc(N(c4ccc(-c5ccc6oc7ccccc7c6c5)cc4)c4cccc5ccccc45)ccc32)cc1]

After Sub 3-1-5 (7.7 g, 24 mmol) was dissolved in toluene, Sub 4-58 (7.7 g, 20 mmol), Pd₂(dba)₃ (0.5 g, 0.6 mmol), P(t-Bu)₃ (0.2 g, 2 mmol), NaOt-Bu (5.8 g, 60 mmol), toluene (300 mL) were added, and the same procedure as described in the synthesis method of 2-5′ was carried out to obtain 12.0 g of Product (yield: 80%).

### Synthesis Example of 2-56′

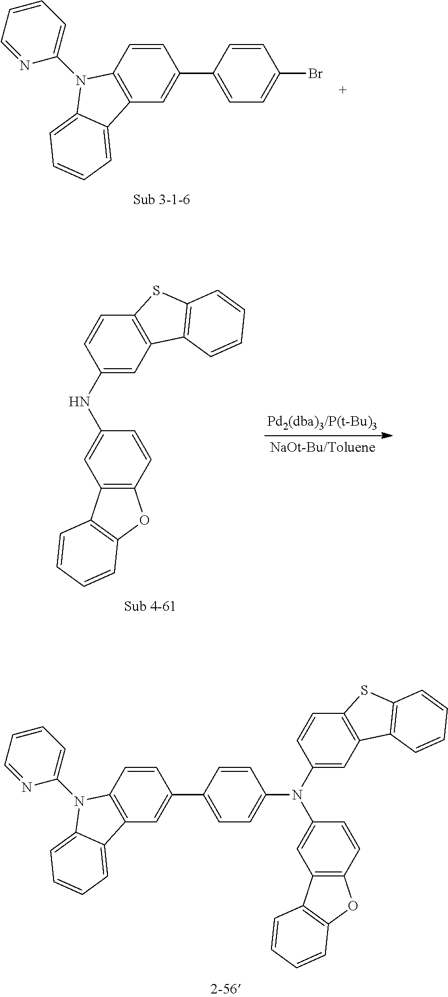 [SMILES: Brc1ccc(-c2ccc3c(c2)c2ccccc2n3-c2ccccn2)cc1 ; c1ccc2c(c1)oc1ccc(Nc3ccc4sc5ccccc5c4c3)cc12 ; "Pd2(dba)3/P(t-Bu)3" ; "NaOt-Bu/Toluene" ; c1ccc(-n2c3ccccc3c3cc(-c4ccc(N(c5ccc6oc7ccccc7c6c5)c5ccc6sc7ccccc7c6c5)cc4)ccc32)nc1]

After Sub 3-1-6 (9.6 g, 24 mmol) was dissolved in toluene, Sub 4-61 (7.3 g, 20 mmol), Pd₂(dba)₃ (0.5 g, 0.6 mmol), P(t-Bu)₃ (0.2 g, 2 mmol), NaOt-Bu (5.8 g, 60 mmol), toluene (300 mL) were added, and the same procedure as described in the synthesis method of 2-5′ was carried out to obtain 12.6 g of Product (yield:77%).

The obtained product was confirmed by the following Mass Data.

TABLE 4

| compound | FD-MS |
|---|---|
| 2-1′ | m/z = 562.24(C₄₂H₃₀N₂ = 562.70) |
| 2-2′ | m/z = 612.26(C₄₆H₃₂N₂ = 612.76) |
| 2-3′ | m/z = 562.24(C₄₂H₃₀N₂ = 562.70) |
| 2-4′ | m/z = 638.27(C₄₈H₃₄N₂ = 638.80) |
| 2-5′ | m/z = 678.30(C₄₂H₃₈N₂ = 678.86) |
| 2-6′ | m/z = 802.33(C₆₁H₄₂N₂ = 803.00) |
| 2-7′ | m/z = 800.32(C₆₁H₄₀N₂ = 800.98) |
| 2-8′ | m/z = 602.27(C₄₅H₃₄N₂ = 602.76) |
| 2-9′ | m/z = 774.30(C₅₉H₃₈N₂ = 774.95) |
| 2-10′ | m/z = 638.27(C₄₈H₃₄N₂ = 638.80) |
| 2-11′ | m/z = 678.30(C₅₁H₃₈N₂ = 678.86) |
| 2-12′ | m/z = 802.33(C₆₁H₄₂N₂ = 803.00) |
| 2-13′ | m/z = 638.27(C₄₈H₃₄N₂ = 638.80) |
| 2-14′ | m/z = 688.29(C₅₂H₃₆N₂ = 688.86) |
| 2-15′ | m/z = 688.29(C₅₂H₃₆N₂ = 688.86)) |
| 2-16′ | m/z = 714.30(C₅₄H₃₈N₂ = 714.89) |
| 2-17′ | m/z = 754.33(C₅₇H₄₂N₂ = 754.96) |
| 2-18′ | m/z = 878.37(C₆₇H₄₆N₂ = 879.10) |
| 2-19′ | m/z = 876.35(C₆₇H₄₄N₂ = 877.08) |
| 2-20′ | m/z = 744.26(C₅₄H₃₆N₂S = 744.94) |
| 2-21′ | m/z = 638.27(C₄₈H₃₄N₂ = 638.80) |
| 2-22′ | m/z = 688.29(C₅₂H₃₆N₂ = 688.86) |
| 2-23′ | m/z = 688.29(C₅₂H₃₆N₂ = 688.86) |
| 2-24′ | m/z = 714.30(C₅₄H₃₈N₂ = 714.89) |
| 2-25′ | m/z = 652.29(C₄₉H₃₆N₂ = 652.82) |
| 2-26′ | m/z = 602.27(C₄₅H₃₄N₂ = 602.76) |
| 2-27′ | m/z = 612.26(C₄₆H₃₂N₂ = 612.76) |
| 2-28′ | m/z = 562.24(C₄₂H₃₀N₂ = 562.70) |
| 2-29′ | m/z = 762.30(C₅₈H₃₈N₂ = 762.94) |
| 2-30′ | m/z = 662.27(C₅₀H₃₄N₂ = 662.82) |
| 2-31′ | m/z = 686.27(C₅₂H₃₄N₂ = 686.84) |
| 2-32′ | m/z = 762.30(C₅₈H₃₈N₂ = 762.94) |
| 2-33′ | m/z = 592.20(C₄₂H₂₈N₂S = 592.75) |
| 2-34′ | m/z = 642.21(C₄₆H₃₀N₂S = 642.81) |
| 2-35′ | m/z = 642.21(C₄₆H₃₀N₂S = 642.81) |
| 2-36′ | m/z = 668.23(C₄₈H₃₂N₂S = 668.85) |
| 2-37′ | m/z = 668.23(C₄₈H₃₂N₂S = 668.85) |
| 2-38′ | m/z = 642.21(C₄₆H₃₀N₂S = 642.81) |
| 2-39′ | m/z = 692.23(C₅₀H₃₂N₂S = 692.87) |
| 2-40′ | m/z = 744.26(C₅₄H₃₆N₂S = 744.94) |
| 2-41′ | m/z = 576.22(C₄₂H₂₈N₂O = 576.22) |
| 2-42′ | m/z = 702.27(C₅₂H₃₄N₂O = 702.84) |
| 2-43′ | m/z = 702.27(C₅₂H₃₄N₂O = 702.84) |
| 2-44′ | m/z = 652.25(C₄₈H₃₂N₂O = 652.78) |
| 2-45′ | m/z = 652.25(C₄₈H₃₂N₂O = 652.78) |
| 2-46′ | m/z = 626.24(C₄₆H₃₀N₂O = 626.74) |
| 2-47′ | m/z = 676.25(C₅₀H₃₂N₂O = 676.80) |
| 2-48′ | m/z = 728.28(C₅₄H₃₆N₂O = 728.88) |
| 2-49′ | m/z = 622.15(C₄₂H₂₆N₂S₂ = 622.80) |
| 2-50′ | m/z = 698.19(C₄₈H₃₀N₂S₂ = 698.90) |
| 2-51′ | m/z = 850.25(C₆₀H₃₈N₂S₂ = 851.09) |
| 2-52′ | m/z = 804.17(C₅₄H₃₂N₂S₃ = 805.04) |
| 2-53′ | m/z = 606.18(C₄₂H₂₆N₂OS = 606.73) |
| 2-54′ | m/z = 682.21(C₄₈H₃₀N₂OS = 682.83) |
| 2-55′ | m/z = 834.27(C₆₀H₃₈N₂OS = 835.02) |
| 2-56′ | m/z = 683.20(C₄₇H₂₉N₃OS = 683.82) |

### Synthesis of Final Product 2′ of Formula (2′)

### Synthesis Example of 2′-5′

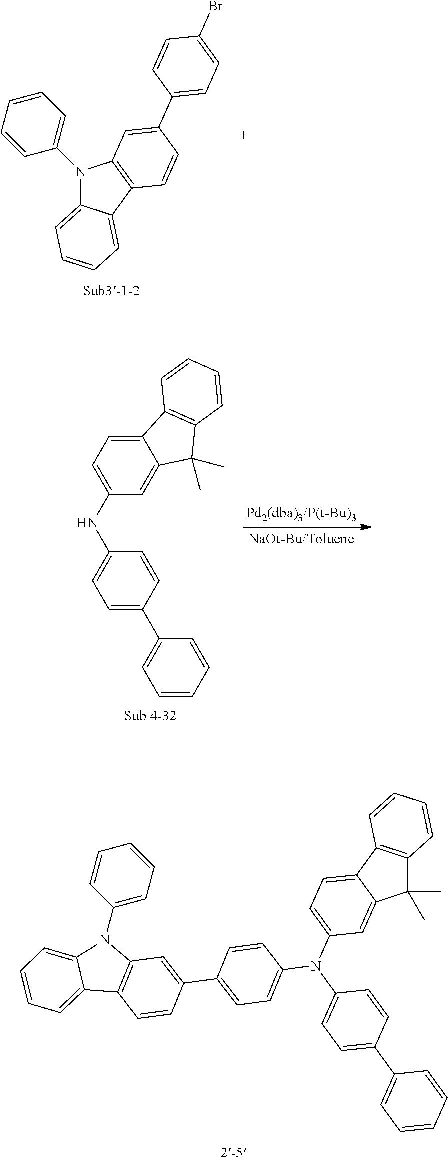 [SMILES: Brc1ccc(-c2ccc3c4ccccc4n(-c4ccccc4)c3c2)cc1 ; CC1(C)c2ccccc2-c2ccc(Nc3ccc(-c4ccccc4)cc3)cc21 ; "Pd2(dba)3/P(t-Bu)3" ; "NaOt-Bu/Toluene" ; CC1(C)c2ccccc2-c2ccc(N(c3ccc(-c4ccccc4)cc3)c3ccc(-c4ccc5c6ccccc6n(-c6ccccc6)c5c4)cc3)cc21]

After Sub 3′-1-2 (9.6 g, 24 mmol) was dissolved in toluene, Sub 4-32 (7.2 g, 20 mmol), Pd₂(dba)₃ (0.5 g, 0.6 mmol), P(t-Bu)₃ (0.2 g, 2 mmol), NaOt-Bu (5.8 g, 60 mmol), toluene (300 mL) were added, and the mixture is refluxed with stirring at 100° C. for 24 hours. When the reaction is complete, the mixture is extracted with ether and water, and the organic layer was dried over MgSO₄ and concentrated, and then the product is separated by silicagel column chromatography and recrystallized to give 13.2 g (81%) of the product.

### Synthesis Example of 2′-10′

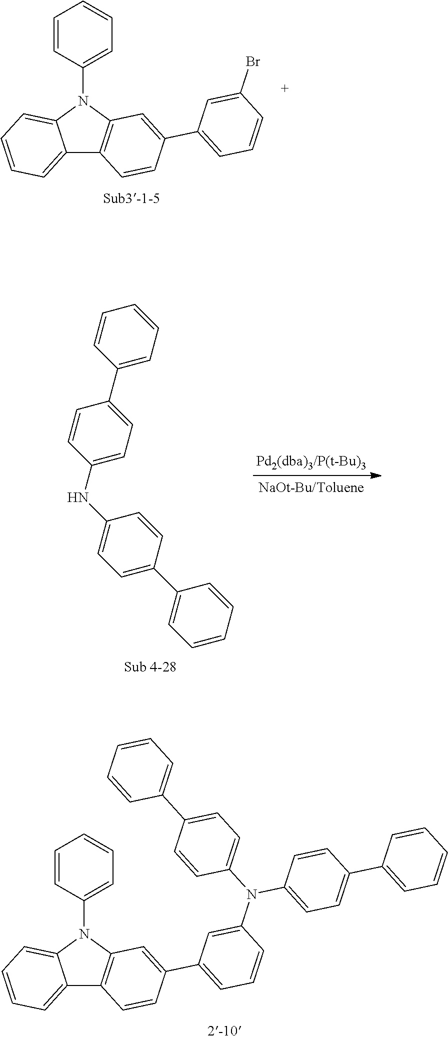 [SMILES: Brc1cccc(-c2ccc3c4ccccc4n(-c4ccccc4)c3c2)c1 ; c1ccc(-c2ccc(Nc3ccc(-c4ccccc4)cc3)cc2)cc1 ; "Pd2(dba)3/P(t-Bu)3" ; "NaOt-Bu/Toluene" ; c1ccc(-c2ccc(N(c3ccc(-c4ccccc4)cc3)c3cccc(-c4ccc5c6ccccc6n(-c6ccccc6)c5c4)c3)cc2)cc1]

After Sub 3′-1-5 (9.6 g, 24 mmol) was dissolved in toluene, Sub 4-28 (6.4 g, 20 mmol), Pd₂(dba)₃ (0.5 g, 0.6 mmol), P(t-Bu)₃ (0.2 g, 2 mmol), NaOt-Bu (5.8 g, 60 mmol), toluene (300 mL) were added, and the same procedure as described in the synthesis method of 2′-5′ was carried out to obtain 12.0 g of Product (yield:78%).

### Synthesis Example of 2′-14′

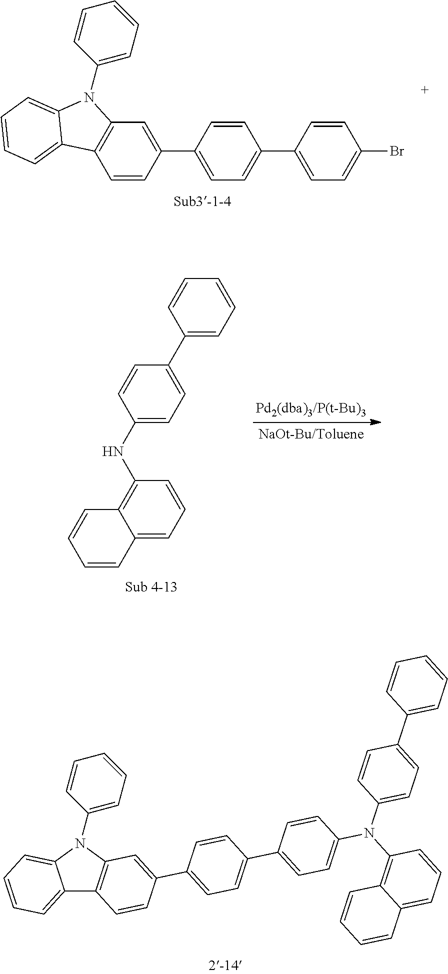 [SMILES: Brc1ccc(-c2ccc(-c3ccc4c5ccccc5n(-c5ccccc5)c4c3)cc2)cc1 ; c1ccc(-c2ccc(Nc3cccc4ccccc34)cc2)cc1 ; "Pd2(dba)3/P(t-Bu)3" ; "NaOt-Bu/Toluene" ; c1ccc(-c2ccc(N(c3ccc(-c4ccc(-c5ccc6c7ccccc7n(-c7ccccc7)c6c5)cc4)cc3)c3cccc4ccccc34)cc2)cc1]

After Sub 3′-1-4 (11.4 g, 24 mmol) was dissolved in toluene, Sub 4-13 (5.9 g, 20 mmol), Pd₂(dba)₃ (0.5 g, 0.6 mmol), P(t-Bu)₃ (0.2 g, 2 mmol), NaOt-Bu (5.8 g, 60 mmol), toluene (300 mL) were added, and the same procedure as described in the synthesis method of 2′-5′ was carried out to obtain 13.7 g of Product (yield:83%).

### Synthesis Example of 2′-36′

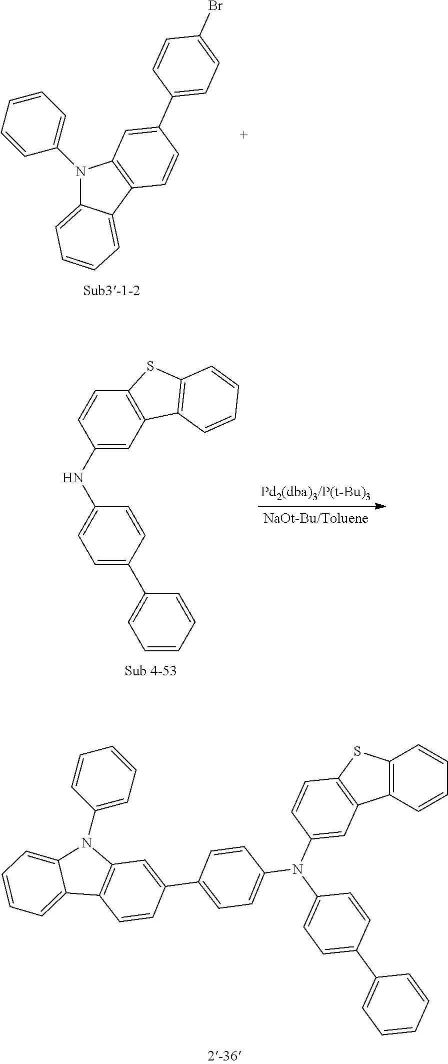 [SMILES: Brc1ccc(-c2ccc3c4ccccc4n(-c4ccccc4)c3c2)cc1 ; c1ccc(-c2ccc(Nc3ccc4sc5ccccc5c4c3)cc2)cc1 ; "Pd2(dba)3/P(t-Bu)3" ; "NaOt-Bu/Toluene" ; c1ccc(-c2ccc(N(c3ccc(-c4ccc5c6ccccc6n(-c6ccccc6)c5c4)cc3)c3ccc4sc5ccccc5c4c3)cc2)cc1]

After Sub 3′-1-2 (9.6 g, 24 mmol) was dissolved in toluene, Sub 4-53 (7.0 g, 20 mmol), Pd₂(dba)₃ (0.5 g, 0.6 mmol), P(t-Bu)₃ (0.2 g, 2 mmol), NaOt-Bu (5.8 g, 60 mmol), toluene (300 mL) were added, and the same procedure as described in the synthesis method of 2′-5′ was carried out to obtain 12.5 g of Product (yield:78%).

### Synthesis Example of 2′-46′

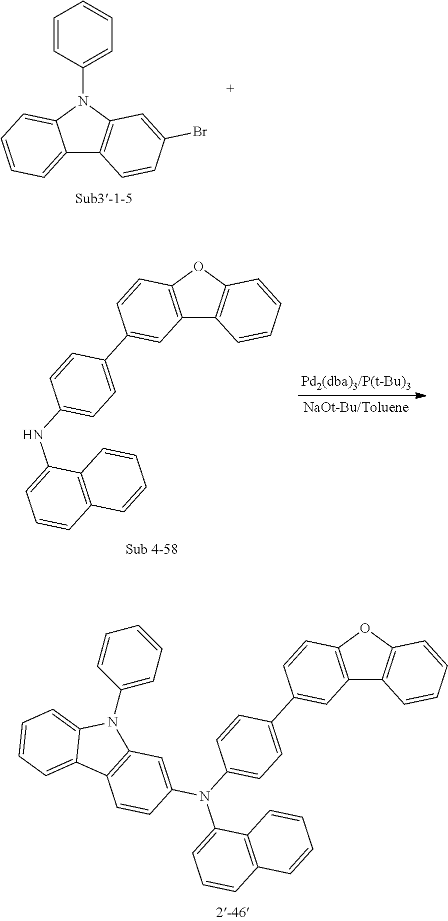 [SMILES: Brc1ccc2c3ccccc3n(-c3ccccc3)c2c1 ; c1ccc2c(Nc3ccc(-c4ccc5oc6ccccc6c5c4)cc3)cccc2c1 ; "Pd2(dba)3/P(t-Bu)3" ; "NaOt-Bu/Toluene" ; c1ccc(-n2c3ccccc3c3ccc(N(c4ccc(-c5ccc6oc7ccccc7c6c5)cc4)c4cccc5ccccc45)cc32)cc1]

After Sub 3′-1-5 (7.7 g, 24 mmol) was dissolved in toluene, Sub 4-58 (7.7 g, 20 mmol), Pd₂(dba)₃ (0.5 g, 0.6 mmol), P(t-Bu)₃ (0.2 g, 2 mmol), NaOt-Bu (5.8 g, 60 mmol), toluene (300 mL) were added, and the same procedure as described in the synthesis method of 2′-5′ was carried out to obtain 12.2 g of Product (yield:81%).

### Synthesis Example of 2′-56′

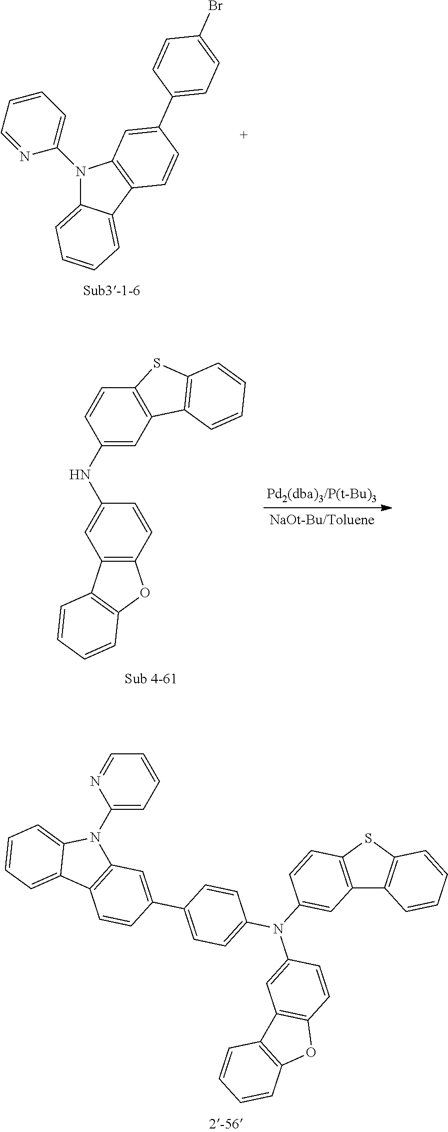 [SMILES: Brc1ccc(-c2ccc3c4ccccc4n(-c4ccccn4)c3c2)cc1 ; c1ccc2c(c1)oc1ccc(Nc3ccc4sc5ccccc5c4c3)cc12 ; "Pd2(dba)3/P(t-Bu)3" ; "NaOt-Bu/Toluene" ; c1ccc(-n2c3ccccc3c3ccc(-c4ccc(N(c5ccc6oc7ccccc7c6c5)c5ccc6sc7ccccc7c6c5)cc4)cc32)nc1]

After Sub 3′-1-6 (9.6 g, 24 mmol) was dissolved in toluene, Sub 4-61 (7.3 g, 20 mmol), Pd₂(dba)₃ (0.5 g, 0.6 mmol), P(t-Bu)₃ (0.2 g, 2 mmol), NaOt-Bu (5.8 g, 60 mmol), toluene (300 mL) were added, and the same procedure as described in the synthesis method of 2′-5′ was carried out to obtain 12.3 g of Product (yield:75%).

The obtained product was confirmed by the following Mass Data.

TABLE 5

| compound | FD-MS |
|---|---|
| 2′-1′ | m/z = 562.24(C₄₂H₃₀N₂ = 562.70) |
| 2′-2′ | m/z = 612.26(C₄₆H₃₂N₂ = 612.76) |
| 2′-3′ | m/z = 562.24(C₄₂H₃₀N₂ = 562.70) |
| 2′-4′ | m/z = 638.27(C₄₈H₃₄N₂ = 638.80) |
| 2′-5′ | m/z = 678.30(C₄₂H₃₈N₂ = 678.86) |
| 2′-6′ | m/z = 802.33(C₆₁H₄₂N₂ = 803.00) |
| 2′-7′ | m/z = 800.32(C₆₁H₄₀N₂ = 800.98) |
| 2′-8′ | m/z = 602.27(C₄₅H₃₄N₂ = 602.76) |
| 2′-9′ | m/z = 774.30(C₅₉H₃₈N₂ = 774.95) |
| 2′-10′ | m/z = 638.27(C₄₈H₃₄N₂ = 638.80) |
| 2′-11′ | m/z = 678.30(C₅₁H₃₈N₂ = 678.86) |
| 2′-12′ | m/z = 802.33(C₆₁H₄₂N₂ = 803.00) |
| 2′-13′ | m/z = 638.27(C₄₈H₃₄N₂ = 638.80) |
| 2′-14′ | m/z = 688.29(C₅₂H₃₆N₂ = 688.86) |
| 2′-15′ | m/z = 688.29(C₅₂H₃₆N₂ = 688.86)) |
| 2′-16′ | m/z = 714.30(C₅₄H₃₈N₂ = 714.89) |
| 2′-17′ | m/z = 754.33(C₅₇H₄₂N₂ = 754.96) |
| 2′-18′ | m/z = 878.37(C₆₇H₄₆N₂ = 879.10) |
| 2′-19′ | m/z = 876.35(C₆₇H₄₄N₂ = 877.08) |
| 2′-20′ | m/z = 744.26(C₅₄H₃₆N₂S = 744.94) |
| 2′-21′ | m/z = 638.27(C₄₈H₃₄N₂ = 638.80) |
| 2′-22′ | m/z = 688.29(C₅₂H₃₆N₂ = 688.86) |
| 2′-23′ | m/z = 688.29(C₅₂H₃₆N₂ = 688.86) |
| 2′-24′ | m/z = 714.30(C₅₄H₃₈N₂ = 714.89) |
| 2′-25′ | m/z = 652.29(C₄₉H₃₆N₂ = 652.82) |
| 2′-26′ | m/z = 602.27(C₄₅H₃₄N₂ = 602.76) |
| 2′-27′ | m/z = 612.26(C₄₆H₃₂N₂ = 612.76) |
| 2′-28′ | m/z = 562.24(C₄₂H₃₀N₂ = 562.70) |
| 2′-29′ | m/z = 762.30(C₅₈H₃₈N₂ = 762.94) |
| 2′-30′ | m/z = 662.27(C₅₀H₃₄N₂ = 662.82) |
| 2′-31′ | m/z = 686.27(C₅₂H₃₄N₂ = 686.84) |
| 2′-32′ | m/z = 762.30(C₅₈H₃₈N₂ = 762.94) |
| 2′-33′ | m/z = 592.20(C₄₂H₂₈N₂S = 592.75) |
| 2′-34′ | m/z = 642.21(C₄₆H₃₀N₂S = 642.81) |
| 2′-35′ | m/z = 642.21(C₄₆H₃₀N₂S = 642.81) |
| 2′-36′ | m/z = 668.23(C₄₈H₃₂N₂S = 668.85) |
| 2′-37′ | m/z = 668.23(C₄₈H₃₂N₂S = 668.85) |
| 2′-38′ | m/z = 642.21(C₄₆H₃₀N₂S = 642.81) |
| 2′-39′ | m/z = 692.23(C₅₀H₃₂N₂S = 692.87) |
| 2′-40′ | m/z = 744.26(C₅₄H₃₆N₂S = 744.94) |
| 2′-41′ | m/z = 576.22(C₄₂H₂₈N₂O = 576.22) |
| 2′-42′ | m/z = 702.27(C₅₂H₃₄N₂O = 702.84) |
| 2′-43′ | m/z = 702.27(C₅₂H₃₄N₂O = 702.84) |
| 2′-44′ | m/z = 652.25(C₄₈H₃₂N₂O = 652.78) |
| 2′-45′ | m/z = 652.25(C₄₈H₃₂N₂O = 652.78) |
| 2′-46′ | m/z = 626.24(C₄₆H₃₀N₂O = 626.74) |
| 2′-47′ | m/z = 676.25(C₅₀H₃₂N₂O = 676.80) |
| 2′-48′ | m/z = 728.28(C₅₄H₃₆N₂O = 728.88) |
| 2′-49′ | m/z = 622.15(C₄₂H₂₆N₂S₂ = 622.80) |
| 2′-50′ | m/z = 698.19(C₄₈H₃₀N₂S₂ = 698.90) |
| 2′-51′ | m/z = 850.25(C₆₀H₃₈N₂S₂ = 851.09) |
| 2′-52′ | m/z = 804.17(C₅₄H₃₂N₂S₃ = 805.04) |
| 2′-53′ | m/z = 606.18(C₄₂H₂₆N₂OS = 606.73) |
| 2′-54′ | m/z = 682.21(C₄₈H₃₀N₂OS = 682.83) |
| 2′-55′ | m/z = 834.27(C₆₀H₃₈N₂OS = 835.02) |
| 2′-56′ | m/z = 683.20(C₄₇H₂₉N₃OS = 683.82) |

Manufacture and Evaluation of Organic Electric Element

### [Example I-1] Blue Organic Light Emitting Diode(Hole Transport Layer)

Using the compound of the present invention as a hole transport layer material, an organic electric element was manufactured according to a conventional method. First, on an ITO layer(anode) formed on a glass substrate, 4,4′,4″-Tris[2-naphthyl(phenyl)amino]triphenylamine (hereinafter will be abbreviated as 2-TNATA) was vacuum deposited to form a hole injection layer with a thickness of 60 nm. And the mixture of the present invention was vacuum deposited on the hole transport layer to form a hole transport layer with a thickness of 60 nm. Then, on the hole transport layer, an emitting layer with a thickness of 30 nm was deposited using 9,10-di(naphthalen-2-yl)anthracene as a host doped with BD-052X(Idemitsukosan) as a dopant in a weight ratio of 95:5. (1,1′-bisphenyl)-4-olato)bis(2-methyl-8-quinolinolato)aluminum (hereinafter will be abbreviated as BAlq) was vacuum deposited to form a hole blocking layer with a thickness of 10 nm, and an electron transport layer was formed by vacuum-depositing tris(8-quinolinol)aluminum (hereinafter will be abbreviated as Alq3) to a thickness of 40 nm. After that, an alkali metal halide, LiF was deposited as an electron injection layer to a thickness of 0.2 nm, and Al was deposited to a thickness of 150 nm to form a cathod to manufacture an OLED.

### Comparative Example 2

Except for using the following comparative compound 1 for the hole transport layer material instead of using the mixture of the present invention, an OLED was manufactured in the same manner as described in the example I-1.

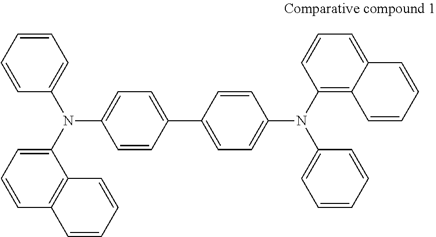 [SMILES: c1ccc(N(c2ccc(-c3ccc(N(c4ccccc4)c4cccc5ccccc45)cc3)cc2)c2cccc3ccccc23)cc1]

### Comparative Example 3

Except for using the inventive compound 1-17′ alone instead of the mixture of the present invention as the hole transporting layer material, an OLED was manufactured in the same manner as described in Example I-1.

### Comparative Example 4

Except for using the inventive compound 2-5′ alone instead of the mixture of the present invention as the hole transporting layer material, an OLED was manufactured in the same manner as described in Example I-1.

### Comparative Example 4′

Except for using the inventive compound 2′-5′ alone instead of the mixture of the present invention as the hole transporting layer material, an OLED was manufactured in the same manner as described in Example I-1.

To the OLEDs which were manufactured by examples and comparative example 1 to comparative example 4, a forward bias direct current voltage was applied, and electroluminescent (EL) properties were measured using PR-650 of Photoresearch Co., and T95 life was measured using a life measuring apparatus manufactured by McScience Inc. with a reference luminance of 500 cd/m². In the following table, the results on the manufacture of a device and evaluation are shown.

TABLE 6

| | | | | | Electric | | | | |
|---|---|---|---|---|---|---|---|---|---|
| | Mixing | | | Driving | current | luminance | efficiency | Luminous | |
| | ratio | Compound A | Compound B | voltage | (mA/cm²) | (cd/m²) | (cd/A) | color | T(95) |
| Comparative | Single | compound (1) | None | 4.5 | 15.2 | 500.0 | 3.3 | Blue | 83 |
| example (1) | compound | | | | | | | | |
| Comparative | Single | compound 1-1 | None | 4.6 | 11.4 | 500.0 | 4.4 | Blue | 90 |
| example (2) | compound | | | | | | | | |
| Comparative | Single | compound 1- | None | 4.7 | 11.9 | 500.0 | 4.2 | Blue | 93 |
| example (3) | compound | 17 | | | | | | | |
| Comparative | Single | compound 2-5 | None | 4.1 | 9.8 | 500.0 | 4.9 | Blue | 97 |
| example (4) | compound | | | | | | | | |
| Example (1) | A(2):B(8) | compound 1- | compound | 4.0 | 9.8 | 500.0 | 5.1 | Blue | 105.6 |
| | | 1′ | 2-4′ | | | | | | |
| Example (2) | A(2):B(8) | compound 1- | compound | 4.1 | 9.5 | 500.0 | 5.3 | Blue | 107.2 |
| | | 1′ | 2-5′ | | | | | | |
| Example (3) | A(2):B(8) | compound 1- | compound | 4.0 | 9.9 | 500.0 | 5.0 | Blue | 102.6 |
| | | 1′ | 2-6′ | | | | | | |
| Example (4) | A(2):B(8) | compound 1- | compound | 4.1 | 9.8 | 500.0 | 5.1 | Blue | 104.7 |
| | | 1′ | 2-7′ | | | | | | |
| Example (5) | A(2):B(8) | compound 1- | compound | 4.0 | 9.7 | 500.0 | 5.1 | Blue | 110.0 |
| | | 1′ | 2-11′ | | | | | | |
| Example (6) | A(2):B(8) | compound 1- | compound | 4.1 | 9.9 | 500.0 | 5.1 | Blue | 107.6 |
| | | 1′ | 2-28′ | | | | | | |
| Example (7) | A(2):B(8) | compound 1- | compound | 4.1 | 9.7 | 500.0 | 5.2 | Blue | 106.6 |
| | | 1′ | 2-36′ | | | | | | |
| Example (8) | A(2):B(8) | compound 1- | compound | 4.1 | 9.6 | 500.0 | 5.2 | Blue | 102.6 |
| | | 1′ | 2-48′ | | | | | | |
| Example (9) | A(2):B(8) | compound 1- | compound | 4.0 | 9.9 | 500.0 | 5.0 | Blue | 109.6 |
| | | 1′ | 2-50′ | | | | | | |
| Example (10) | A(2):B(8) | compound 1- | compound | 4.0 | 9.8 | 500.0 | 5.1 | Blue | 105.1 |
| | | 1′ | 2-54′ | | | | | | |
| Example (11) | A(3):B(7) | compound 1- | compound | 4.1 | 9.0 | 500.0 | 5.6 | Blue | 118.6 |
| | | 1′ | 2-4′ | | | | | | |
| Example (12) | A(3):B(7) | compound 1- | compound | 4.1 | 8.8 | 500.0 | 5.7 | Blue | 117.4 |
| | | 1′ | 2-5′ | | | | | | |
| Example (13) | A(3):B(7) | compound 1- | compound | 4.1 | 9.2 | 500.0 | 5.4 | Blue | 115.1 |
| | | 1′ | 2-6′ | | | | | | |
| Example (14) | A(3):B(7) | compound 1- | compound | 4.1 | 9.2 | 500.0 | 5.4 | Blue | 114.5 |
| | | 1′ | 2-7′ | | | | | | |
| Example (15) | A(3):B(7) | compound 1- | compound | 4.0 | 8.9 | 500.0 | 5.6 | Blue | 113.6 |
| | | 1′ | 2-11′ | | | | | | |
| Example (16) | A(3):B(7) | compound 1- | compound | 4.1 | 8.8 | 500.0 | 5.7 | Blue | 113.5 |
| | | 1′ | 2-28′ | | | | | | |
| Example (17) | A(3):B(7) | compound 1- | compound | 4.1 | 8.9 | 500.0 | 5.6 | Blue | 112.6 |
| | | 1′ | 2-36′ | | | | | | |
| Example (18) | A(3):B(7) | compound 1- | compound | 4.0 | 9.2 | 500.0 | 5.4 | Blue | 111.2 |
| | | 1′ | 2-48′ | | | | | | |
| Example (19) | A(3):B(7) | compound 1- | compound | 4.1 | 9.1 | 500.0 | 5.5 | Blue | 115.6 |
| | | 1′ | 2-50′ | | | | | | |
| Example (20) | A(3):B(7) | compound 1- | compound | 4.0 | 9.0 | 500.0 | 5.6 | Blue | 113.2 |
| | | 1′ | 2-54′ | | | | | | |
| Example (21) | A(4):B(6) | compound 1- | compound | 4.1 | 8.3 | 500.0 | 6.0 | Blue | 124.7 |
| | | 1′ | 2-4′ | | | | | | |
| Example (22) | A(4):B(6) | compound 1- | compound | 4.1 | 8.6 | 500.0 | 5.8 | Blue | 127.7 |
| | | 1′ | 2-5′ | | | | | | |
| Example (23) | A(4):B(6) | compound 1- | compound | 4.0 | 8.6 | 500.0 | 5.8 | Blue | 129.5 |
| | | 1′ | 2-6′ | | | | | | |
| Example (24) | A(4):B(6) | compound 1- | compound | 4.0 | 8.4 | 500.0 | 5.9 | Blue | 123.8 |
| | | 1′ | 2-7′ | | | | | | |
| Example (25) | A(4):B(6) | compound 1- | compound | 4.0 | 8.3 | 500.0 | 6.0 | Blue | 124.4 |
| | | 1′ | 2-11′ | | | | | | |
| Example (26) | A(4):B(6) | compound 1- | compound | 4.0 | 8.4 | 500.0 | 5.9 | Blue | 128.4 |
| | | 1′ | 2-28′ | | | | | | |
| Example (27) | A(4):B(6) | compound 1- | compound | 4.1 | 8.6 | 500.0 | 5.8 | Blue | 127.4 |
| | | 1′ | 2-36′ | | | | | | |
| Example (28) | A(4):B(6) | compound 1- | compound | 4.0 | 8.5 | 500.0 | 5.9 | Blue | 126.6 |
| | | 1′ | 2-48′ | | | | | | |
| Example (29) | A(4):B(6) | compound 1- | compound | 4.1 | 8.6 | 500.0 | 5.8 | Blue | 126.9 |
| | | 1′ | 2-50′ | | | | | | |
| Example (30) | A(4):B(6) | compound 1- | compound | 4.0 | 8.6 | 500.0 | 5.8 | Blue | 125.6 |
| | | 1′ | 2-54′ | | | | | | |
| Example (31) | A(5):B(5) | compound 1- | compound | 4.6 | 7.4 | 500.0 | 6.8 | Blue | 134.6 |
| | | 1′ | 2-4′ | | | | | | |
| Example (32) | A(5):B(5) | compound 1- | compound | 4.5 | 7.9 | 500.0 | 6.3 | Blue | 131.6 |
| | | 1′ | 2-5′ | | | | | | |
| Example (33) | A(5):B(5) | compound 1- | compound | 4.6 | 7.8 | 500.0 | 6.4 | Blue | 131.4 |
| | | 1′ | 2-6′ | | | | | | |
| Example (34) | A(5):B(5) | compound 1- | compound | 4.5 | 7.5 | 500.0 | 6.6 | Blue | 130.7 |
| | | 1′ | 2-7′ | | | | | | |
| Example (35) | A(5):B(5) | compound 1- | compound | 4.4 | 7.4 | 500.0 | 6.8 | Blue | 130.2 |
| | | 1′ | 2-11′ | | | | | | |
| Example (36) | A(5):B(5) | compound 1- | compound | 4.6 | 7.5 | 500.0 | 6.7 | Blue | 134.1 |
| | | 1′ | 2-28′ | | | | | | |
| Example (37) | A(5):B(5) | compound 1- | compound | 4.5 | 8.3 | 500.0 | 6.1 | Blue | 134.1 |
| | | 1′ | 2-36′ | | | | | | |
| Example (38) | A(5):B(5) | compound 1- | compound | 4.6 | 7.8 | 500.0 | 6.4 | Blue | 132.0 |
| | | 1′ | 2-48′ | | | | | | |
| Example (39) | A(5):B(5) | compound 1- | compound | 4.6 | 7.2 | 500.0 | 6.9 | Blue | 132.4 |
| | | 1′ | 2-50′ | | | | | | |
| Example (40) | A(5):B(5) | compound 1- | compound | 4.6 | 7.8 | 500.0 | 6.4 | Blue | 134.0 |
| | | 1′ | 2-54′ | | | | | | |
| Example (41) | A(7):B(3) | compound 1- | compound | 4.6 | 8.6 | 500.0 | 5.8 | Blue | 126.0 |
| | | 1′ | 2-4′ | | | | | | |
| Example (42) | A(7):B(3) | compound 1- | compound | 4.5 | 8.4 | 500.0 | 5.9 | Blue | 125.0 |
| | | 1′ | 2-5′ | | | | | | |
| Example (43) | A(7):B(3) | compound 1- | compound | 4.5 | 8.6 | 500.0 | 5.8 | Blue | 122.9 |
| | | 1′ | 2-6′ | | | | | | |
| Example (44) | A(7):B(3) | compound 1- | compound | 4.5 | 8.4 | 500.0 | 6.0 | Blue | 128.2 |
| | | 1′ | 2-7′ | | | | | | |
| Example (45) | A(7):B(3) | compound 1- | compound | 4.5 | 8.5 | 500.0 | 5.9 | Blue | 120.9 |
| | | 1′ | 2-11′ | | | | | | |
| Example (46) | A(7):B(3) | compound 1- | compound | 4.4 | 8.5 | 500.0 | 5.9 | Blue | 128.2 |
| | | 1′ | 2-28′ | | | | | | |
| Example (47) | A(7):B(3) | compound 1- | compound | 4.6 | 8.4 | 500.0 | 6.0 | Blue | 123.7 |
| | | 1′ | 2-36′ | | | | | | |
| Example (48) | A(7):B(3) | compound 1- | compound | 4.5 | 8.5 | 500.0 | 5.9 | Blue | 128.6 |
| | | 1′ | 2-48′ | | | | | | |
| Example (49) | A(7):B(3) | compound 1- | compound | 4.5 | 8.4 | 500.0 | 5.9 | Blue | 126.4 |
| | | 1′ | 2-50′ | | | | | | |
| Example (50) | A(7):B(3) | compound 1- | compound | 4.6 | 8.6 | 500.0 | 5.8 | Blue | 124.9 |
| | | 1′ | 2-54′ | | | | | | |
| Example (51) | A(5):B(5) | compound 1- | compound | 4.6 | 6.8 | 500.0 | 7.4 | Blue | 117.4 |
| | | 17′ | 2-4′ | | | | | | |
| Example (52) | A(5):B(5) | compound 1- | compound | 4.6 | 7.0 | 500.0 | 7.1 | Blue | 116.2 |
| | | 17′ | 2-5′ | | | | | | |
| Example (53) | A(5):B(5) | compound 1- | compound | 4.6 | 6.8 | 500.0 | 7.3 | Blue | 113.5 |
| | | 17′ | 2-6′ | | | | | | |
| Example (54) | A(5):B(5) | compound 1- | compound | 4.5 | 6.8 | 500.0 | 7.4 | Blue | 112.5 |
| | | 17′ | 2-7′ | | | | | | |
| Example (55) | A(5):B(5) | compound 1- | compound | 4.6 | 6.8 | 500.0 | 7.3 | Blue | 131.8 |
| | | 17′ | 2-11′ | | | | | | |
| Example (56) | A(5):B(5) | compound 1- | compound | 4.5 | 6.7 | 500.0 | 7.5 | Blue | 134.6 |
| | | 17′ | 2-28′ | | | | | | |
| Example (57) | A(5):B(5) | compound 1- | compound | 4.5 | 7.0 | 500.0 | 7.2 | Blue | 131.5 |
| | | 17′ | 2-36′ | | | | | | |
| Example (58) | A(5):B(5) | compound 1- | compound | 4.7 | 6.8 | 500.0 | 7.3 | Blue | 131.0 |
| | | 17′ | 2-48′ | | | | | | |
| Example (59) | A(5):B(5) | compound 1- | compound | 4.7 | 7.0 | 500.0 | 7.1 | Blue | 132.2 |
| | | 17′ | 2-50′ | | | | | | |
| Example (60) | A(5):B(5) | compound 1- | compound | 4.7 | 6.7 | 500.0 | 7.5 | Blue | 134.5 |
| | | 17′ | 2-54′ | | | | | | |
| Example (61) | A(5):B(5) | compound 1- | compound | 4.6 | 6.9 | 500.0 | 7.2 | Blue | 134.7 |
| | | 52′ | 2-4′ | | | | | | |
| Example (62) | A(5):B(5) | compound 1- | compound | 4.6 | 6.9 | 500.0 | 7.2 | Blue | 130.7 |
| | | 52′ | 2-5′ | | | | | | |
| Example (63) | A(5):B(5) | compound 1- | compound | 4.5 | 6.8 | 500.0 | 7.4 | Blue | 133.0 |
| | | 52′ | 2-6′ | | | | | | |
| Example (64) | A(5):B(5) | compound 1- | compound | 4.6 | 6.8 | 500.0 | 7.4 | Blue | 133.8 |
| | | 52′ | 2-7′ | | | | | | |
| Example (65) | A(5):B(5) | compound 1- | compound | 4.5 | 6.9 | 500.0 | 7.2 | Blue | 132.3 |
| | | 52′ | 2-11′ | | | | | | |
| Example (66) | A(5):B(5) | compound 1- | compound | 4.7 | 7.0 | 500.0 | 7.2 | Blue | 130.8 |
| | | 52′ | 2-28′ | | | | | | |
| Example (67) | A(5):B(5) | compound 1- | compound | 4.6 | 6.8 | 500.0 | 7.3 | Blue | 130.9 |
| | | 52′ | 2-36′ | | | | | | |
| Example (68) | A(5):B(5) | compound 1- | compound | 4.6 | 6.8 | 500.0 | 7.4 | Blue | 133.7 |
| | | 52′ | 2-48′ | | | | | | |
| Example (69) | A(5):B(5) | compound 1- | compound | 4.5 | 6.9 | 500.0 | 7.2 | Blue | 130.4 |
| | | 52′ | 2-50′ | | | | | | |
| Example (70) | A(5):B(5) | compound 1- | compound | 4.5 | 6.9 | 500.0 | 7.3 | Blue | 134.7 |
| | | 52′ | 2-54′ | | | | | | |

TABLE 7

| | | | | | Electric | | | | |
|---|---|---|---|---|---|---|---|---|---|
| | Mixing | | | Driving | current | luminance | efficiency | Luminous | |
| | ratio | Compound A | compound B | voltage | (mA/cm²) | (cd/m²) | (cd/A) | color | T(95) |
| Comparative | Single | Comparative | None | 4.5 | 15.2 | 500.0 | 3.3 | Blue | 83 |
| Example | compound | compound (1) | | | | | | | |
| (1) | | | | | | | | | |
| Comparative | Single | Compound | None | 4.6 | 11.4 | 500.0 | 4.4 | Blue | 90 |
| Example | compound | 1-1 | | | | | | | |
| (2) | | | | | | | | | |
| Comparative | Single | Compound | None | 4.7 | 11.9 | 500.0 | 4.2 | Blue | 93 |
| Example | compound | 1-17 | | | | | | | |
| (3) | | | | | | | | | |
| Comparative | Single | compound | None | 4.1 | 9.8 | 500.0 | 4.9 | Blue | 97 |
| Example | compound | 2′-5 | | | | | | | |
| (4′) | | | | | | | | | |
| Example (1′) | A(2):B(8) | Compound | Compound | 4.0 | 84.7 | 5000.0 | 5.9 | Blue | 126.0 |
| | | 1-1′ | 2′-4′ | | | | | | |
| Example (2′) | A(2):B(8) | Compound | Compound | 4.1 | 86.4 | 5000.0 | 5.8 | Blue | 120.9 |
| | | 1-1′ | 2′-5′ | | | | | | |
| Example (3′) | A(2):B(8) | compound | Compound | 4.0 | 83.9 | 5000.0 | 6.0 | Blue | 127.4 |
| | | 1-1′ | 2′-6′ | | | | | | |
| Example (4′) | A(2):B(8) | Compound | Compound | 4.0 | 87.0 | 5000.0 | 5.7 | Blue | 119.6 |
| | | 1-1′ | 2′-7′ | | | | | | |
| Example (5′) | A(2):B(8) | Compound | Compound | 4.1 | 92.3 | 5000.0 | 5.4 | Blue | 128.9 |
| | | 1-1′ | 2′-11′ | | | | | | |
| Example (6′) | A(2):B(8) | compound | Compound | 4.0 | 90.0 | 5000.0 | 5.6 | Blue | 120.3 |
| | | 1-1′ | 2′-28′ | | | | | | |
| Example (7′) | A(2):B(8) | compound | Compound | 4.1 | 86.3 | 5000.0 | 5.8 | Blue | 113.7 |
| | | 1-1′ | 2′-36′ | | | | | | |
| Example (8′) | A(2):B(8) | Compound | Compound | 4.1 | 94.0 | 5000.0 | 5.3 | Blue | 106.4 |
| | | 1-1′ | 2′-48′ | | | | | | |
| Example (9′) | A(2):B(8) | Compound | Compound | 4.1 | 85.5 | 5000.0 | 5.8 | Blue | 100.3 |
| | | 1-1′ | 2′-50′ | | | | | | |
| Example (10′) | A(2):B(8) | Compound | Compound | 4.1 | 84.6 | 5000.0 | 5.9 | Blue | 115.5 |
| | | 1-1′ | 2′-54′ | | | | | | |
| Example (11′) | A(3):B(7) | compound | Compound | 4.0 | 89.4 | 5000.0 | 5.6 | Blue | 109.5 |
| | | 1-1′ | 2′-4′ | | | | | | |
| Example (12′) | A(3):B(7) | Compound | Compound | 4.0 | 88.9 | 5000.0 | 5.6 | Blue | 122.6 |
| | | 1-1′ | 2′-5′ | | | | | | |
| Example (13′) | A(3):B(7) | Compound | Compound | 4.1 | 87.5 | 5000.0 | 5.7 | Blue | 109.9 |
| | | 1-1′ | 2′-6′ | | | | | | |
| Example (14′) | A(3):B(7) | compound | Compound | 4.0 | 84.5 | 5000.0 | 5.9 | Blue | 101.1 |
| | | 1-1′ | 2′-7′ | | | | | | |
| Example (15′) | A(3):B(7) | Compound | Compound | 4.0 | 91.8 | 5000.0 | 5.4 | Blue | 119.5 |
| | | 1-1′ | 2′-11′ | | | | | | |
| Example (16′) | A(3):B(7) | compound | Compound | 4.1 | 85.2 | 5000.0 | 5.9 | Blue | 108.6 |
| | | 1-1′ | 2′-28′ | | | | | | |
| Example (17′) | A(3):B(7) | Compound | Compound | 4.0 | 85.5 | 5000.0 | 5.8 | Blue | 118.5 |
| | | 1-1′ | 2′-36′ | | | | | | |
| Example (18′) | A(3):B(7) | Compound | Compound | 4.0 | 88.5 | 5000.0 | 5.6 | Blue | 117.4 |
| | | 1-1′ | 2′-48′ | | | | | | |
| Example (19′) | A(3):B(7) | Compound | Compound | 4.1 | 83.4 | 5000.0 | 6.0 | Blue | 109.5 |
| | | 1-1′ | 2′-50′ | | | | | | |
| Example (20′) | A(3):B(7) | Compound | Compound | 4.1 | 91.3 | 5000.0 | 5.5 | Blue | 101.0 |
| | | 1-1′ | 2′-54′ | | | | | | |
| Example (21′) | A(4):B(6) | compound | Compound | 4.1 | 83.7 | 5000.0 | 6.0 | Blue | 124.4 |
| | | 1-1′ | 2′-4′ | | | | | | |
| Example (22′) | A(4):B(6) | Compound | Compound | 4.0 | 85.4 | 5000.0 | 5.9 | Blue | 111.6 |
| | | 1-1′ | 2′-5′ | | | | | | |
| Example (23′) | A(4):B(6) | Compound | Compound | 4.0 | 90.0 | 5000.0 | 5.6 | Blue | 128.2 |
| | | 1-1′ | 2′-6′ | | | | | | |
| Example (24′) | A(4):B(6) | Compound | compound | 4.1 | 83.6 | 5000.0 | 6.0 | Blue | 127.6 |
| | | 1-1′ | 2′-7′ | | | | | | |
| Example (25′) | A(4):B(6) | Compound | Compound | 4.1 | 84.7 | 5000.0 | 5.9 | Blue | 109.8 |
| | | 1-1′ | 2′-11′ | | | | | | |
| Example (26′) | A(4):B(6) | compound | Compound | 4.1 | 88.9 | 5000.0 | 5.6 | Blue | 116.9 |
| | | 1-1′ | 2′-28′ | | | | | | |
| Example (27′) | A(4):B(6) | Compound | Compound | 4.1 | 86.4 | 5000.0 | 5.8 | Blue | 129.8 |
| | | 1-1′ | 2′-36′ | | | | | | |
| Example (28′) | A(4):B(6) | compound | Compound | 4.1 | 87.8 | 5000.0 | 5.7 | Blue | 104.7 |
| | | 1-1′ | 2′-48′ | | | | | | |
| Example (29′) | A(4):B(6) | Compound | Compound | 4.1 | 86.1 | 5000.0 | 5.8 | Blue | 128.8 |
| | | 1-1′ | 2′-50′ | | | | | | |
| Example (30′) | A(4):B(6) | Compound | Compound | 4.1 | 87.5 | 5000.0 | 5.7 | Blue | 115.4 |
| | | 1-1′ | 2′-54′ | | | | | | |
| Example (31′) | A(5):B(5) | compound | Compound | 4.2 | 82.1 | 5000.0 | 6.1 | Blue | 130.3 |
| | | 1-1′ | 2′-4′ | | | | | | |
| Example (32′) | A(5):B(5) | compound | Compound | 4.3 | 73.0 | 5000.0 | 6.8 | Blue | 133.2 |
| | | 1-1′ | 2′-5′ | | | | | | |
| Example (33′) | A(5):B(5) | Compound | Compound | 4.3 | 75.4 | 5000.0 | 6.6 | Blue | 130.0 |
| | | 1-1′ | 2′-6′ | | | | | | |
| Example (34′) | A(5):B(5) | Compound | Compound | 4.2 | 79.1 | 5000.0 | 6.3 | Blue | 132.7 |
| | | 1-1′ | 2′-7′ | | | | | | |
| Example (35′) | A(5):B(5) | Compound | Compound | 4.2 | 81.4 | 5000.0 | 6.1 | Blue | 131.1 |
| | | 1-1′ | 2′-11′ | | | | | | |
| Example (36′) | A(5):B(5) | Compound | Compound | 4.3 | 72.8 | 5000.0 | 6.9 | Blue | 130.8 |
| | | 1-1′ | 2′-28′ | | | | | | |
| Example (37′) | A(5):B(5) | compound | Compound | 4.3 | 82.1 | 5000.0 | 6.1 | Blue | 133.1 |
| | | 1-1′ | 2′-36′ | | | | | | |
| Example (38′) | A(5):B(5) | Compound | Compound | 4.3 | 71.8 | 5000.0 | 7.0 | Blue | 131.7 |
| | | 1-1′ | 2′-48′ | | | | | | |
| Example (39′) | A(5):B(5) | compound | Compound | 4.2 | 75.7 | 5000.0 | 6.6 | Blue | 132.7 |
| | | 1-1′ | 2′-50′ | | | | | | |
| Example (40′) | A(5):B(5) | compound | Compound | 4.3 | 82.0 | 5000.0 | 6.1 | Blue | 130.1 |
| | | 1-1′ | 2′-54′ | | | | | | |
| Example (41′) | A(7):B(3) | Compound | Compound | 4.6 | 5.4 | 300.0 | 5.6 | Blue | 120.9 |
| | | 1-1′ | 2′-4′ | | | | | | |
| Example (42′) | A(7):B(3) | Compound | compound | 4.4 | 5.1 | 300.0 | 5.8 | Blue | 121.2 |
| | | 1-1′ | 2′-5′ | | | | | | |
| Example (43′) | A(7):B(3) | Compound | Compound | 4.5 | 5.3 | 300.0 | 5.7 | Blue | 111.7 |
| | | 1-1′ | 2′-6′ | | | | | | |
| Example (44′) | A(7):B(3) | Compound | Compound | 4.5 | 5.0 | 300.0 | 6.0 | Blue | 100.8 |
| | | 1-1′ | 2′-7′ | | | | | | |
| Example (45′) | A(7):B(3) | Compound | Compound | 4.4 | 5.2 | 300.0 | 5.7 | Blue | 101.5 |
| | | 1-1′ | 2′-11′ | | | | | | |
| Example (46′) | A(7):B(3) | Compound | Compound | 4.6 | 5.1 | 300.0 | 5.9 | Blue | 113.2 |
| | | 1-1′ | 2′-28′ | | | | | | |
| Example (47′) | A(7):B(3) | Compound | Compound | 4.4 | 5.4 | 300.0 | 5.6 | Blue | 107.9 |
| | | 1-1′ | 2′-36′ | | | | | | |
| Example (48′) | A(7):B(3) | Compound | Compound | 4.5 | 5.5 | 300.0 | 5.5 | Blue | 109.4 |
| | | 1-1′ | 2′-48′ | | | | | | |
| Example (49′) | A(7):B(3) | Compound | Compound | 4.5 | 5.5 | 300.0 | 5.5 | Blue | 123.6 |
| | | 1-1′ | 2′-50′ | | | | | | |
| Example (50′) | A(7):B(3) | Compound | Compound | 4.5 | 5.1 | 300.0 | 5.9 | Blue | 101.0 |
| | | 1-1′ | 2′-54′ | | | | | | |
| Example (51′) | A(5):B(5) | Compound | Compound | 4.2 | 4.3 | 300.0 | 7.1 | Blue | 147.0 |
| | | 1-17′ | 2′-4′ | | | | | | |
| Example (52′) | A(5):B(5) | compound | Compound | 4.0 | 4.2 | 300.0 | 7.1 | Blue | 143.5 |
| | | 1-17′ | 2′-5′ | | | | | | |
| Example (53′) | A(5):B(5) | Compound | Compound | 4.0 | 4.2 | 300.0 | 7.2 | Blue | 137.2 |
| | | 1-17′ | 2′-6′ | | | | | | |
| Example (54′) | A(5):B(5) | Compound | Compound | 4.0 | 4.1 | 300.0 | 7.3 | Blue | 139.6 |
| | | 1-17′ | 2′-7′ | | | | | | |
| Example (55′) | A(5):B(5) | Compound | Compound | 4.1 | 4.1 | 300.0 | 7.3 | Blue | 146.8 |
| | | 1-17′ | 2′-11′ | | | | | | |
| Example (56′) | A(5):B(5) | compound | Compound | 4.2 | 4.2 | 300.0 | 7.2 | Blue | 147.8 |
| | | 1-17′ | 2′-28′ | | | | | | |
| Example (57′) | A(5):B(5) | Compound | Compound | 4.2 | 4.1 | 300.0 | 7.4 | Blue | 135.5 |
| | | 1-17′ | 2′-36′ | | | | | | |
| Example (58′) | A(5):B(5) | Compound | Compound | 4.1 | 4.3 | 300.0 | 7.0 | Blue | 145.9 |
| | | 1-17′ | 2′-48′ | | | | | | |
| Example (59′) | A(5):B(5) | Compound | Compound | 4.0 | 4.2 | 300.0 | 7.2 | Blue | 142.1 |
| | | 1-17′ | 2′-50′ | | | | | | |
| Example (60′) | A(5):B(5) | Compound | Compound | 4.1 | 4.1 | 300.0 | 7.2 | Blue | 140.4 |
| | | 1-17′ | 2′-54′ | | | | | | |
| Example (61′) | A(5):B(5) | compound | Compound | 4.0 | 4.1 | 300.0 | 7.4 | Blue | 146.9 |
| | | 1-52′ | 2′-4′ | | | | | | |
| Example (62′) | A(5):B(5) | Compound | Compound | 4.2 | 4.1 | 300.0 | 7.3 | Blue | 137.6 |
| | | 1-52′ | 2′-5′ | | | | | | |
| Example (63′) | A(5):B(5) | Compound | Compound | 4.1 | 4.0 | 300.0 | 7.4 | Blue | 143.8 |
| | | 1-52′ | 2′-6′ | | | | | | |
| Example (64′) | A(5):B(5) | compound | Compound | 4.2 | 4.2 | 300.0 | 7.2 | Blue | 141.7 |
| | | 1-52′ | 2′-7′ | | | | | | |
| Example (65′) | A(5):B(5) | Compound | Compound | 4.1 | 4.0 | 300.0 | 7.5 | Blue | 135.6 |
| | | 1-52′ | 2′-11′ | | | | | | |
| Example (66′) | A(5):B(5) | Compound | Compound | 4.1 | 4.1 | 300.0 | 7.4 | Blue | 143.0 |
| | | 1-52′ | 2-28′ | | | | | | |
| Example (67′) | A(5):B(5) | compound | Compound | 4.1 | 4.0 | 300.0 | 7.5 | Blue | 144.7 |
| | | 1-52′ | 2-36′ | | | | | | |
| Example (68′) | A(5):B(5) | Compound | Compound | 4.1 | 4.1 | 300.0 | 7.4 | Blue | 139.4 |
| | | 1-52′ | 2-48′ | | | | | | |
| Example (69′) | A(5):B(5) | Compound | Compound | 4.1 | 4.0 | 300.0 | 7.5 | Blue | 142.9 |
| | | 1-52′ | 2-50′ | | | | | | |
| Example (70′) | A(5):B(5) | Compound | Compound | 4.1 | 4.3 | 300.0 | 7.0 | Blue | 150.0 |
| | | 1-52′ | 2-54′ | | | | | | |

As it is apparent from the Table 7, when the mixture of the present invention is used as hole transport layer, the luminous efficiency and life span can be remarkably improved as compared with the comparative example 1˜4 which are single compound.

The results of Table 7 will be described in more detail. First, the result of example 1˜10 or example 1˜10′ using the hole transport layer mixing a tertiary amine compound 1-1′ substituted with an aryl group (biphenyl) and a compound represented by Formula 2 (2-4′, 2-5′, 2-6′, 2-7′, 2-11′, 2-28′, 2-36′, 2-48′, 2-50′, 2-54′) or a compound represented by Formula 2′(2′-4′, 2′-5′, 2′-6′, 2′-7′, 2′-11′, 2′-28′, 2′-36′, 2′-48′, 2′-50′, 2′-54′) in the ratio of 2:8 (mixing ratio) increase efficiency and life span, and reduces the driving voltage when compared to those for the Comparative example 1˜4 using the hole transport layer having single compound.

In particular, when comparing the results with Comparative Example 2 using 1-1′ as a single compound, it may be confirmed that a carbazole-containing compound represented by the Formula 2 or 2′ is mixed and used as a hole transport layer, therefore the efficiency is increased by 120%, and the life span also increased by 120%.

As a result of proceeding Examples 1-70 to 1-70′ in order to investigate the differences in the characteristics of the mixing ratios, when the mixing ratio was 5:5, the highest efficiency was obtained and the life span was increased. Consequently, when the mixing ratio is 5:5, the result of the mixture of compound 1-17′ and 1-52′ substituted with Dibenzofuran of Dibenzothiophen with the compound represented by the Formula 2 or 2′ was better than the result of the mixture of compound 1-1′ substituted with aryl group.

That is, when the compound represented by the Formula 1 and the compound represented by the Formula 2 or 2′ were mixed and used in the hole transport layer, it was confirmed that the life span and the efficiency were superior to those using the single compound, and that the result was slightly different depending on the mixing ratio, and the result of the element with the mixing ratio of 5:5 was the best. This proves that the present invention is more advanced than an example using a conventional single compound as a hole transport layer.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment. The scope of the present invention shall be construed on the basis of the accompanying claims, and it shall be construed that all of the technical ideas included within the scope equivalent to the claims belong to the present invention.

1. An organic electric element comprising:
a first electrode;
a second electrode; and
an organic material layer, disposed between the first electrode and the second electrode, and comprising at least one hole transport layer and emitting layer comprising emitting compounds, wherein the hole transport layer is composed of a composition comprising a mixture of a compound represented by Formula 1 and a compound represented by Formula 2, or a composition comprising a mixture of a compound represented by Formula 1 and a compound represented by Formula 2′: 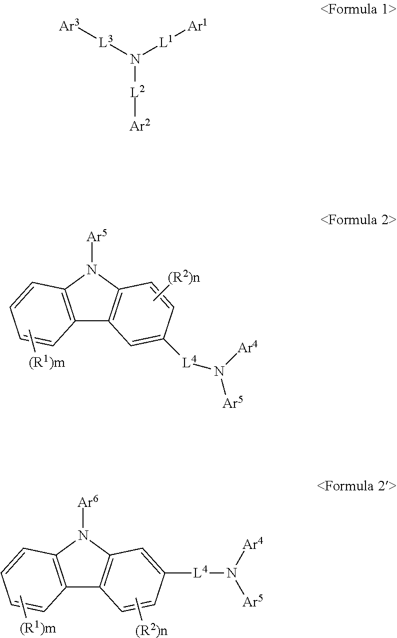
In Formula 1, Formula 2 or Formula 2′,
1) Ar¹, Ar², Ar³ are each independently selected from the group consisting of a C₆-C₆₀ aryl group; a C₂-C₆₀ heteroaryl group; and a fluorenyl group,
2) L¹, L², L³ are each independently selected from the group consisting of a single bond; a C₆-C₆₀ arylene group; a divalent of C₂-C₆₀ heterocyclic group; a fluorenylene group; and a divalent fused ring group of a C₃-C₆₀ aliphatic ring and a C₆-C₆₀ aromatic ring,
3) Ar⁴, Ar⁵, Ar⁶ are each independently selected from the group consisting of a C₆-C₆₀ aryl group; a C₂-C₆₀ heterocyclic group; and a fluorenyl group,
4) L⁴ may be selected from the group consisting of a single bond; a C₆-C₆₀ arylene group; a divalent of C₂-C₆₀ heterocyclic group; a fluorenylene group; and a fused ring group of a C₃-C₆₀ aliphatic ring and a C₆-C₆₀ aromatic ring,
5) m is an integer of 0 to 4; n is an integer of 0 to 3, and
6) R¹ and R² are the same or different from each other, and are each independently selected from the group consisting of deuterium; halogen; a C₆-C₆₀ aryl group; a fluorenyl group; a C₂-C₆₀ heterocyclic group including at least one heteroatom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; a C₆-C₃₀ aryloxy group; and L′-N(Rₐ)(R_{b}),
wherein L′ may be selected from the group consisting of a single bond; a C₆-C₆₀ arylene group; a fluorenylene group; a fused ring group of a C₃-C₆₀ aliphatic ring and a C₆-C₆₀ aromatic ring; and a C₂-C₆₀ heterocyclic, and Rₐ and R_{b} may be independently selected from the group consisting of a C₆-C₆₀ aryl group; a fluorenyl group; a fused ring group of a C₃-C₆₀ aliphatic ring and a C₆-C₆₀ aromatic ring; and a C₂-C₆₀ heterocyclic group containing at least one heteroatom of O, N, S, Si, or P, or
in case m, n are 2 or more, R¹ and R² are each in plural and are the same or different from each other, and a plurality of R¹ or a plurality of R² may combine to each other to form a ring,
7) wherein, the aryl group, heteroaryl group, fluorenyl group, arylene group, heterocyclic group and fused ring group may be substituted with one or more substituents selected from the group consisting of deuterium; halogen; a silane group; a siloxane group; boron group; a germanium group; a cyano group; a nitro group; -L′-N(Rₐ)(R_{b}); a C₁-C₂₀ alkylthio group; a C₁-C₂₀ alkoxyl group; a C₁-C₂₀ alkyl group; a C₂-C₂₀ alkenyl group; a C₂-C₂₀ alkynyl group; a C₆-C₆₀ aryl group; a C₆-C₆₀ aryl group substituted with deuterium; a fluorenyl group; a C₂-C₂₀ heterocyclic group; a C₃-C₂₀ cycloalkyl group; a C₇-C₂₀ arylalkyl group; and a C₈-C₂₀ arylalkenyl group,
wherein the substituents may combine each other and form a saturated or unsaturated ring selected from the group consisting of a C₃-C₆₀ aliphatic ring, a C₆-C₆₀ aromatic ring, a C₂-C₆₀ heterocyclic ring, and a fused ring formed by the combination there.

2. The organic electric element according to claim 1, wherein the compound of Formula 1 is one of the following Formulae 1-2, 1-3, and 1-4: 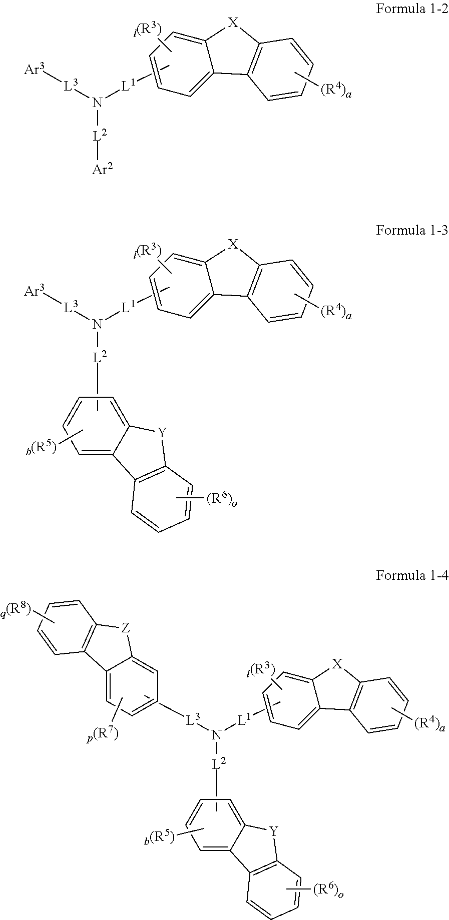
In Formula 1-2, 1-3 and 1-4,
1) Ar², Ar³, L¹, L² and L³ are the same as defined in claim 1,
2) X, Y and Z are each independently S, O or CR′R″,
3) R′ and R″ are each independently selected from the group consisting of a C₆-C₂₄ aryl group; C₁-C₂₀ alkyl group; C₂-C₂₀ alkenyl group; and C₁-C₂₀ alkoxy group, and R′ and R″ may combine each other and form a spiro,
4) R³, R⁴, R⁵, R⁶, R⁷ and R⁸ are each independently selected from the group consisting of deuterium; tritium; a cyano group; nitro group; halogen; aryl group; alkenyl group; alkylene group; alkoxy group; and hetrocyclic group, and when each of R³, R⁴, R⁵, R⁶, R⁷ and/or R⁸ are in plural a plurality of R³ or plurality of R⁴ or plurality of R⁵ or plurality of R⁶ or plurality of R⁷ or plurality of R⁸ may combine to each other to form a ring,
5) l, b, p are an integer of 0 to 3, and a, o, q are an integer of 0 to 4.

3. The organic electric element according to claim 1, wherein the compound represented by Formula 2 or Formula 2′ is represented by a compound of Formula 2-2, 2-3, 2′-2, and 2′-3 below: 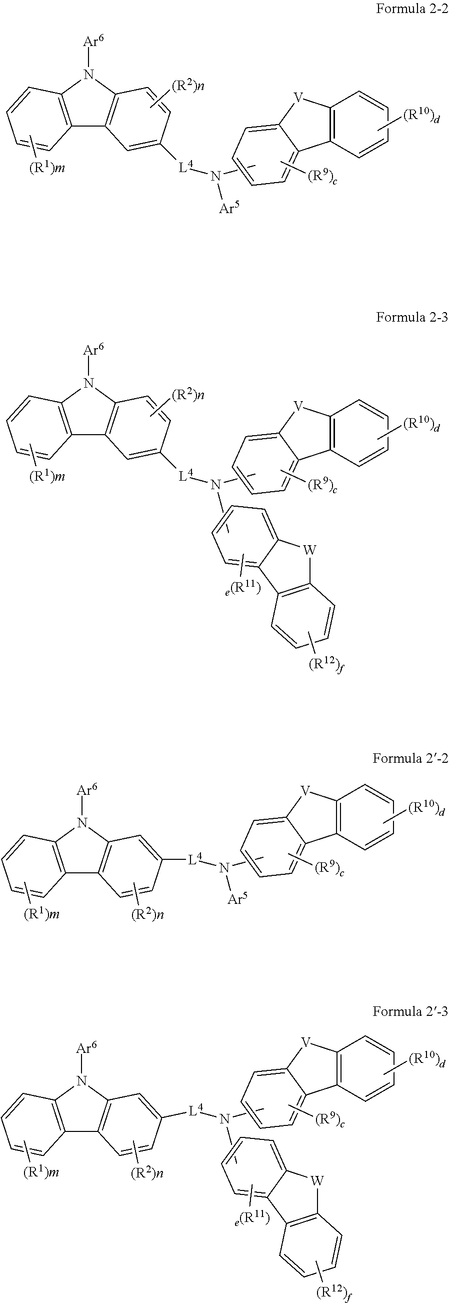
In Formulae 2-2, 2-3, 2′-2, 2′-3 above,
1) R¹, R², Ar⁵, Ar⁶, L⁴, m and n are the same as defined in claim 1,
2) V and W are each independently S, O, or CR′R″,
3) R′ and R″ are each independently selected from the group consisting of a C₆-C₂₄ aryl group; C₁-C₂₀ alkyl group; C₂-C₂₀ alkenyl group; and C₁-C₂₀ alkoxy group, and R′ and R″ may combine each other and form a spiro,
4) R⁹, R¹⁰, R¹¹, and R¹² are each independently selected from the group consisting of deuterium; tritium; a cyano group; nitro group; halogen; aryl group; alkenyl group; alkylene group; alkoxy group; and hetrocyclic group, and when R⁹, R¹⁰, R¹¹, and/or R¹² are in plural a plurality of R⁹ or plurality of R¹⁰ or plurality of R¹¹ or plurality of R¹² may combine to each other to form a ring,
5) c and e are an integer of 0 to 3, and d and f are an integer of 0 to 4.

4. The organic electric element according to claim 1, wherein the compound of Formula 1 is represented by a compound of the following:

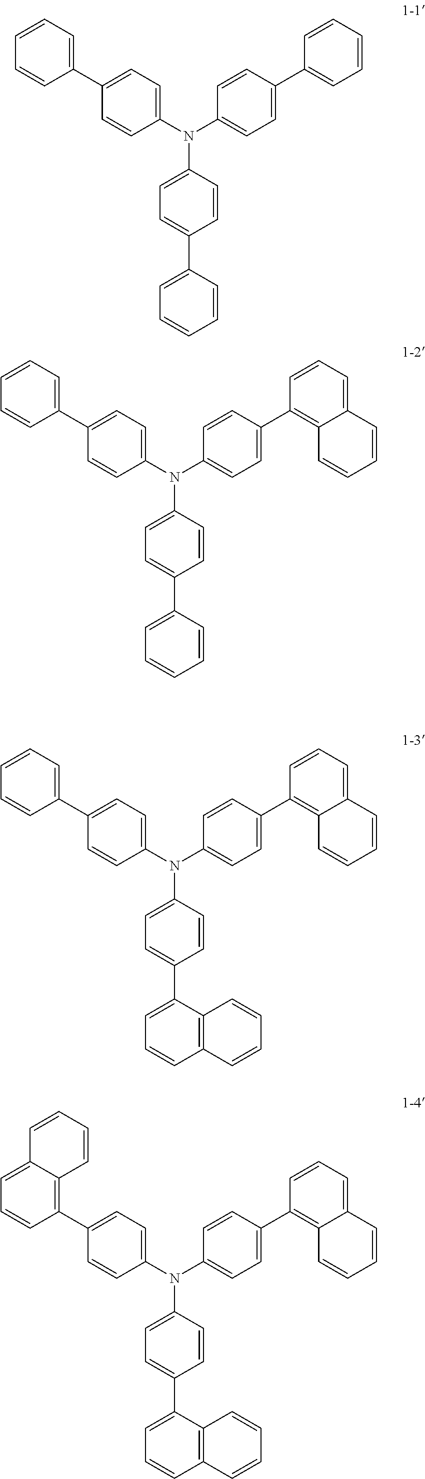 [SMILES: c1ccc(-c2ccc(N(c3ccc(-c4ccccc4)cc3)c3ccc(-c4ccccc4)cc3)cc2)cc1 ; c1ccc(-c2ccc(N(c3ccc(-c4ccccc4)cc3)c3ccc(-c4cccc5ccccc45)cc3)cc2)cc1 ; c1ccc(-c2ccc(N(c3ccc(-c4cccc5ccccc45)cc3)c3ccc(-c4cccc5ccccc45)cc3)cc2)cc1 ; c1ccc2c(-c3ccc(N(c4ccc(-c5cccc6ccccc56)cc4)c4ccc(-c5cccc6ccccc56)cc4)cc3)cccc2c1]

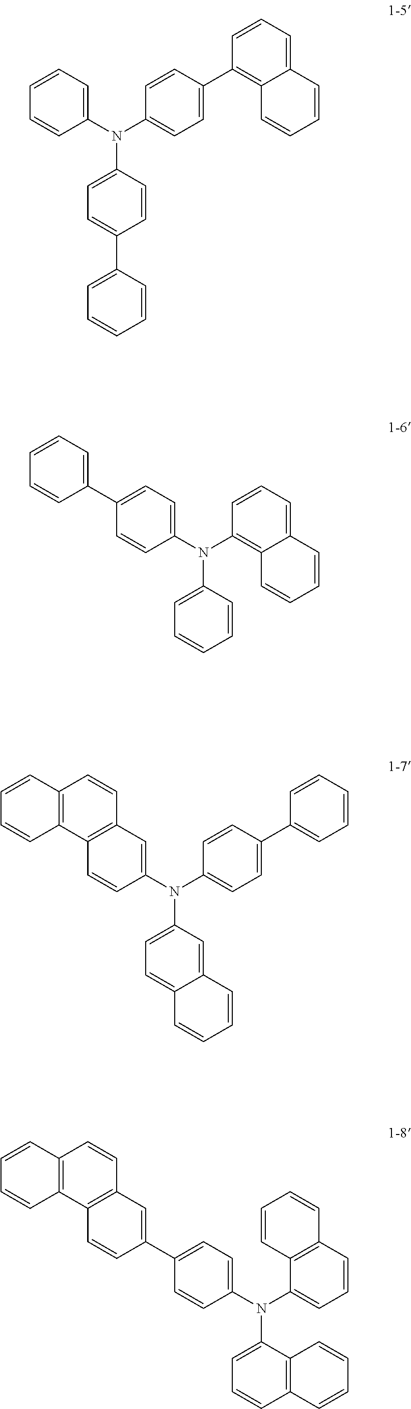 [SMILES: c1ccc(-c2ccc(N(c3ccccc3)c3ccc(-c4cccc5ccccc45)cc3)cc2)cc1 ; c1ccc(-c2ccc(N(c3ccccc3)c3cccc4ccccc34)cc2)cc1 ; c1ccc(-c2ccc(N(c3ccc4ccccc4c3)c3ccc4c(ccc5ccccc54)c3)cc2)cc1 ; c1ccc2c(N(c3ccc(-c4ccc5c(ccc6ccccc65)c4)cc3)c3cccc4ccccc34)cccc2c1]

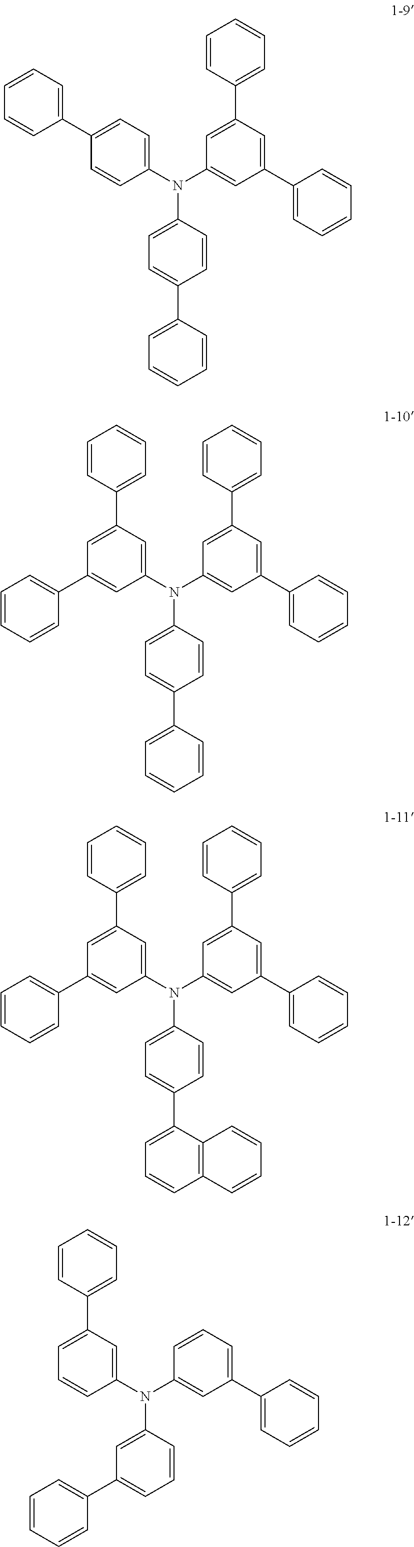 [SMILES: c1ccc(-c2ccc(N(c3ccc(-c4ccccc4)cc3)c3cc(-c4ccccc4)cc(-c4ccccc4)c3)cc2)cc1 ; c1ccc(-c2ccc(N(c3cc(-c4ccccc4)cc(-c4ccccc4)c3)c3cc(-c4ccccc4)cc(-c4ccccc4)c3)cc2)cc1 ; c1ccc(-c2cc(-c3ccccc3)cc(N(c3ccc(-c4cccc5ccccc45)cc3)c3cc(-c4ccccc4)cc(-c4ccccc4)c3)c2)cc1 ; c1ccc(-c2cccc(N(c3cccc(-c4ccccc4)c3)c3cccc(-c4ccccc4)c3)c2)cc1]

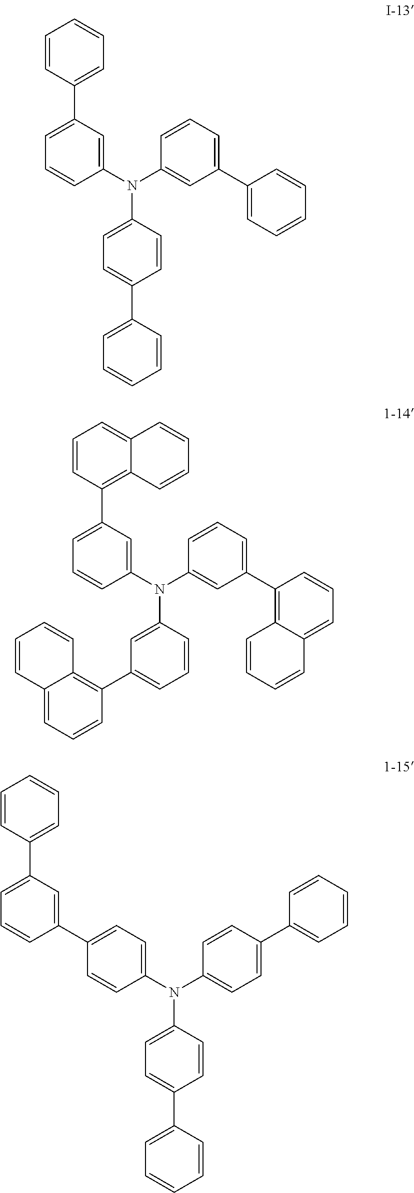

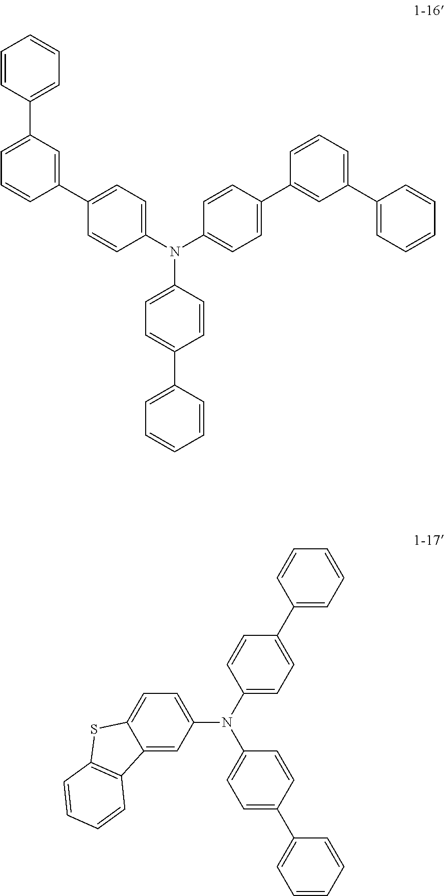 [SMILES: c1ccc(-c2ccc(N(c3ccc(-c4cccc(-c5ccccc5)c4)cc3)c3ccc(-c4cccc(-c5ccccc5)c4)cc3)cc2)cc1 ; c1ccc(-c2ccc(N(c3ccc(-c4ccccc4)cc3)c3ccc4sc5ccccc5c4c3)cc2)cc1]

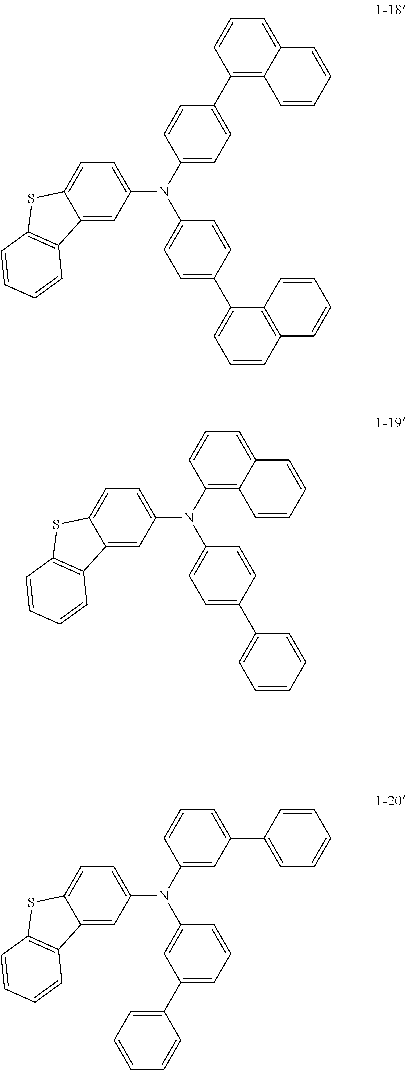 [SMILES: c1ccc2c(-c3ccc(N(c4ccc(-c5cccc6ccccc56)cc4)c4ccc5sc6ccccc6c5c4)cc3)cccc2c1 ; c1ccc(-c2ccc(N(c3ccc4sc5ccccc5c4c3)c3cccc4ccccc34)cc2)cc1 ; c1ccc(-c2cccc(N(c3cccc(-c4ccccc4)c3)c3ccc4sc5ccccc5c4c3)c2)cc1]

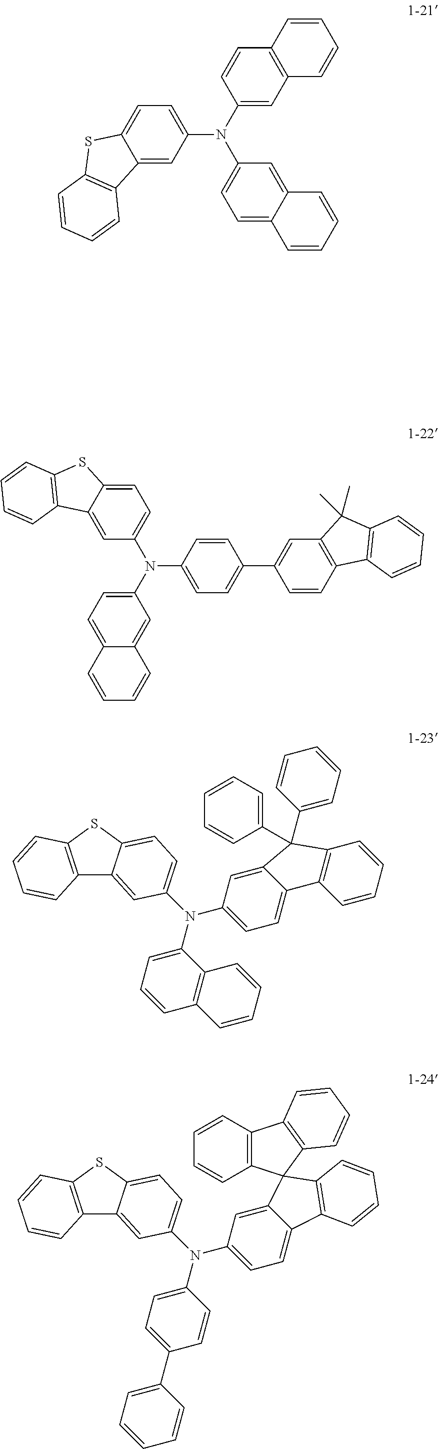 [SMILES: c1ccc2cc(N(c3ccc4ccccc4c3)c3ccc4sc5ccccc5c4c3)ccc2c1 ; CC1(C)c2ccccc2-c2ccc(-c3ccc(N(c4ccc5ccccc5c4)c4ccc5sc6ccccc6c5c4)cc3)cc21 ; c1ccc(C2(c3ccccc3)c3ccccc3-c3ccc(N(c4ccc5sc6ccccc6c5c4)c4cccc5ccccc45)cc32)cc1 ; c1ccc(-c2ccc(N(c3ccc4c(c3)C3(c5ccccc5-c5ccccc53)c3ccccc3-4)c3ccc4sc5ccccc5c4c3)cc2)cc1]

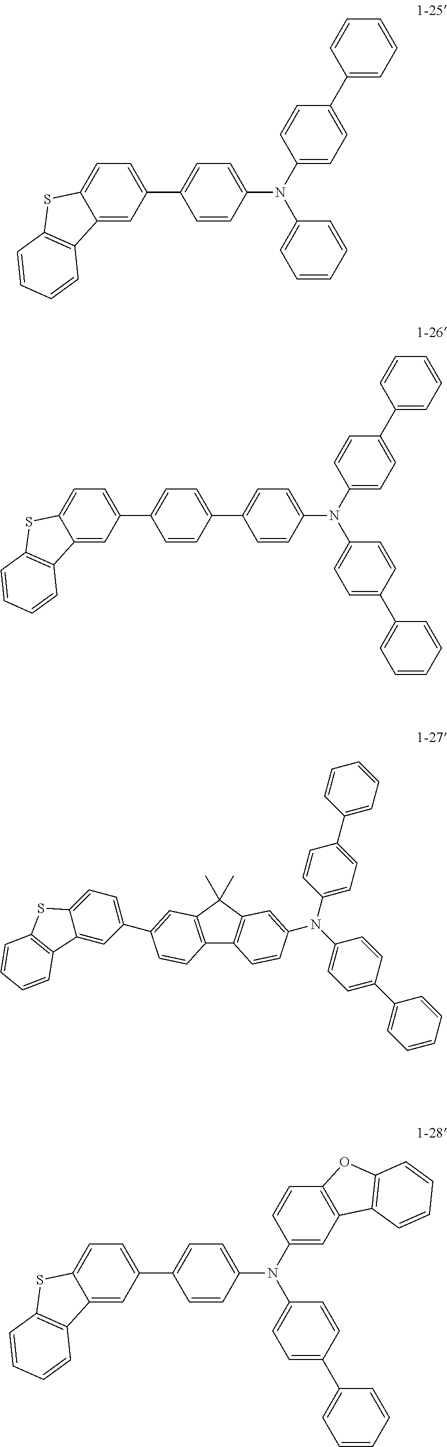 [SMILES: c1ccc(-c2ccc(N(c3ccccc3)c3ccc(-c4ccc5sc6ccccc6c5c4)cc3)cc2)cc1 ; c1ccc(-c2ccc(N(c3ccc(-c4ccccc4)cc3)c3ccc(-c4ccc(-c5ccc6sc7ccccc7c6c5)cc4)cc3)cc2)cc1 ; CC1(C)c2cc(-c3ccc4sc5ccccc5c4c3)ccc2-c2ccc(N(c3ccc(-c4ccccc4)cc3)c3ccc(-c4ccccc4)cc3)cc21 ; c1ccc(-c2ccc(N(c3ccc(-c4ccc5sc6ccccc6c5c4)cc3)c3ccc4oc5ccccc5c4c3)cc2)cc1]

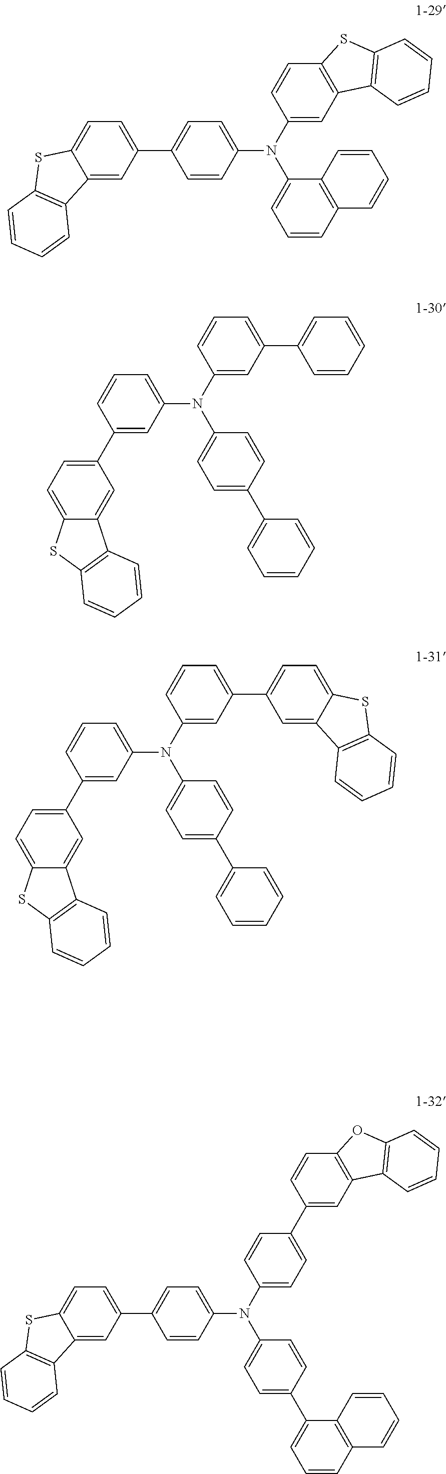 [SMILES: c1ccc2c(N(c3ccc(-c4ccc5sc6ccccc6c5c4)cc3)c3ccc4sc5ccccc5c4c3)cccc2c1 ; c1ccc(-c2ccc(N(c3cccc(-c4ccccc4)c3)c3cccc(-c4ccc5sc6ccccc6c5c4)c3)cc2)cc1 ; c1ccc(-c2ccc(N(c3cccc(-c4ccc5sc6ccccc6c5c4)c3)c3cccc(-c4ccc5sc6ccccc6c5c4)c3)cc2)cc1 ; c1ccc2c(-c3ccc(N(c4ccc(-c5ccc6oc7ccccc7c6c5)cc4)c4ccc(-c5ccc6sc7ccccc7c6c5)cc4)cc3)cccc2c1]

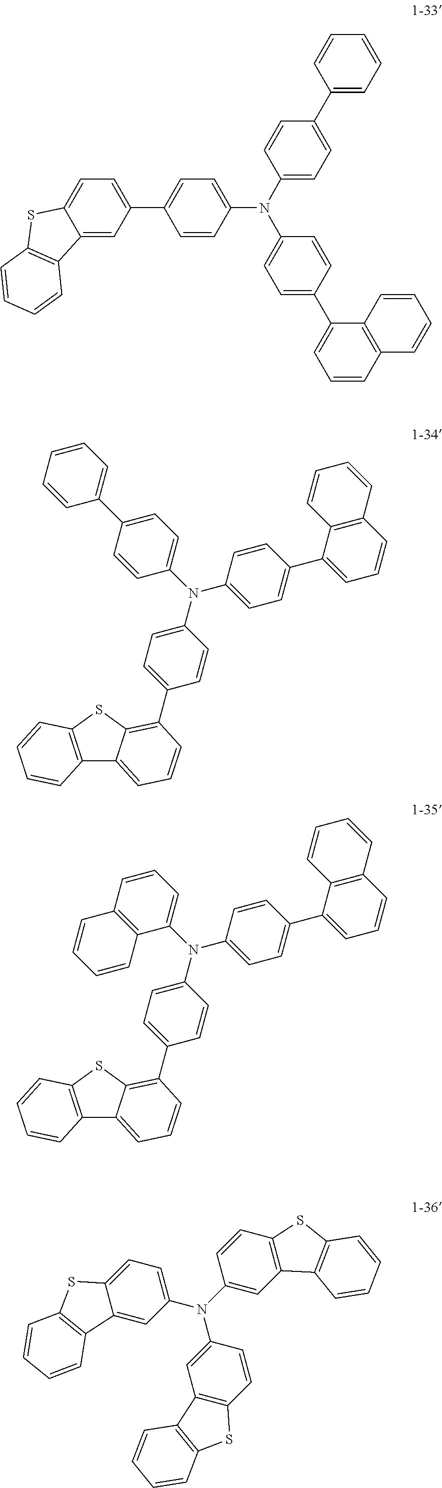 [SMILES: c1ccc(-c2ccc(N(c3ccc(-c4ccc5sc6ccccc6c5c4)cc3)c3ccc(-c4cccc5ccccc45)cc3)cc2)cc1 ; c1ccc(-c2ccc(N(c3ccc(-c4cccc5ccccc45)cc3)c3ccc(-c4cccc5c4sc4ccccc45)cc3)cc2)cc1 ; c1ccc2c(-c3ccc(N(c4ccc(-c5cccc6c5sc5ccccc56)cc4)c4cccc5ccccc45)cc3)cccc2c1 ; c1ccc2c(c1)sc1ccc(N(c3ccc4sc5ccccc5c4c3)c3ccc4sc5ccccc5c4c3)cc12]

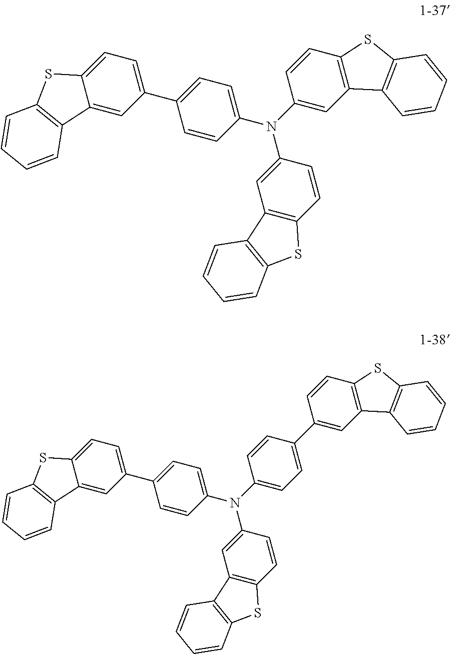 [SMILES: c1ccc2c(c1)sc1ccc(-c3ccc(N(c4ccc5sc6ccccc6c5c4)c4ccc5sc6ccccc6c5c4)cc3)cc12 ; c1ccc2c(c1)sc1ccc(-c3ccc(N(c4ccc(-c5ccc6sc7ccccc7c6c5)cc4)c4ccc5sc6ccccc6c5c4)cc3)cc12]

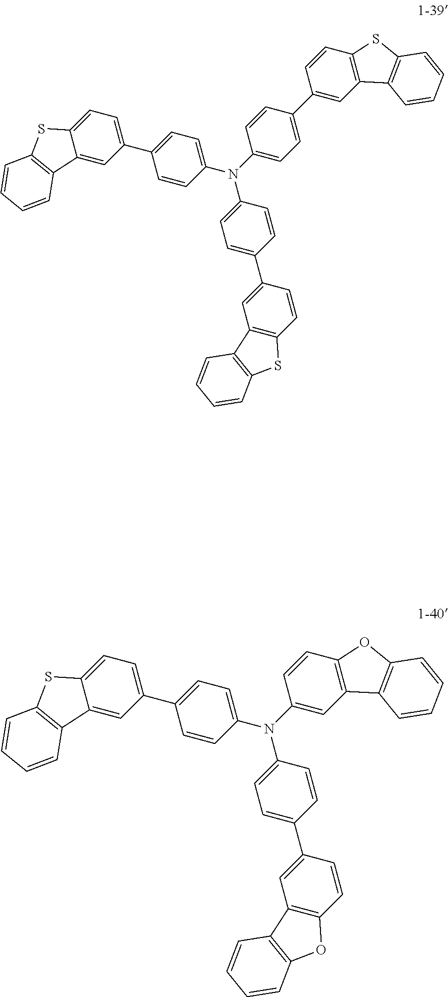 [SMILES: c1ccc2c(c1)sc1ccc(-c3ccc(N(c4ccc(-c5ccc6sc7ccccc7c6c5)cc4)c4ccc(-c5ccc6sc7ccccc7c6c5)cc4)cc3)cc12 ; c1ccc2c(c1)oc1ccc(-c3ccc(N(c4ccc(-c5ccc6sc7ccccc7c6c5)cc4)c4ccc5oc6ccccc6c5c4)cc3)cc12]

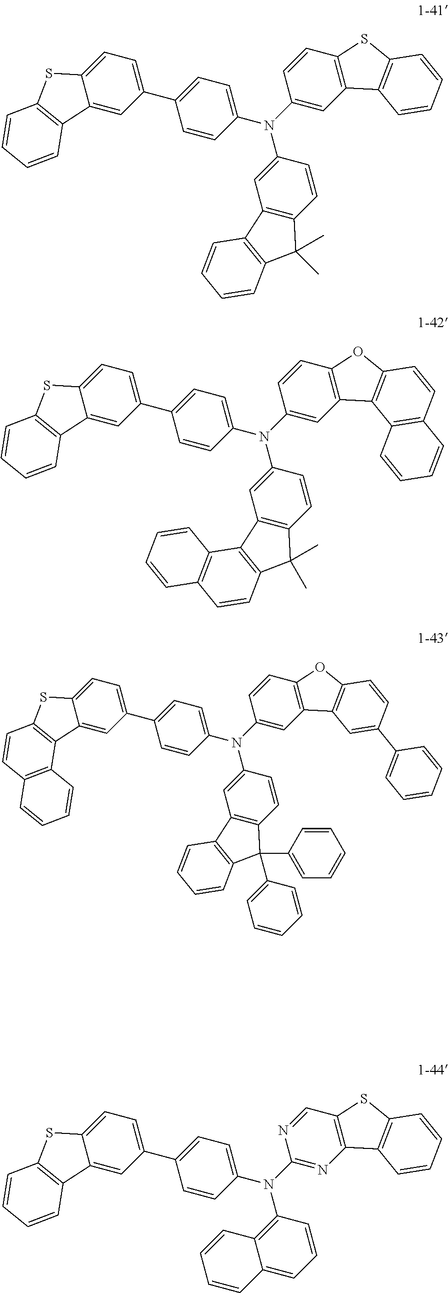 [SMILES: CC1(C)c2ccccc2-c2cc(N(c3ccc(-c4ccc5sc6ccccc6c5c4)cc3)c3ccc4sc5ccccc5c4c3)ccc21 ; CC1(C)c2ccc(N(c3ccc(-c4ccc5sc6ccccc6c5c4)cc3)c3ccc4oc5ccc6ccccc6c5c4c3)cc2-c2c1ccc1ccccc21 ; c1ccc(-c2ccc3oc4ccc(N(c5ccc(-c6ccc7sc8ccc9ccccc9c8c7c6)cc5)c5ccc6c(c5)-c5ccccc5C6(c5ccccc5)c5ccccc5)cc4c3c2)cc1 ; c1ccc2c(N(c3ccc(-c4ccc5sc6ccccc6c5c4)cc3)c3ncc4sc5ccccc5c4n3)cccc2c1]

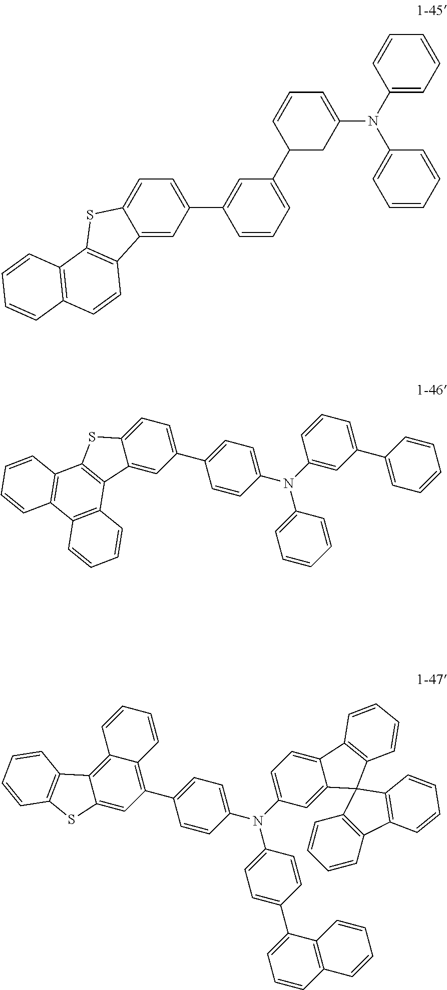 [SMILES: C1=CC(c2cccc(-c3ccc4sc5c6ccccc6ccc5c4c3)c2)CC(N(c2ccccc2)c2ccccc2)=C1 ; c1ccc(-c2cccc(N(c3ccccc3)c3ccc(-c4ccc5sc6c7ccccc7c7ccccc7c6c5c4)cc3)c2)cc1 ; c1ccc2c(c1)-c1ccccc1C21c2ccccc2-c2ccc(N(c3ccc(-c4cccc5ccccc45)cc3)c3ccc(-c4cc5sc6ccccc6c5c5ccccc45)cc3)cc21]

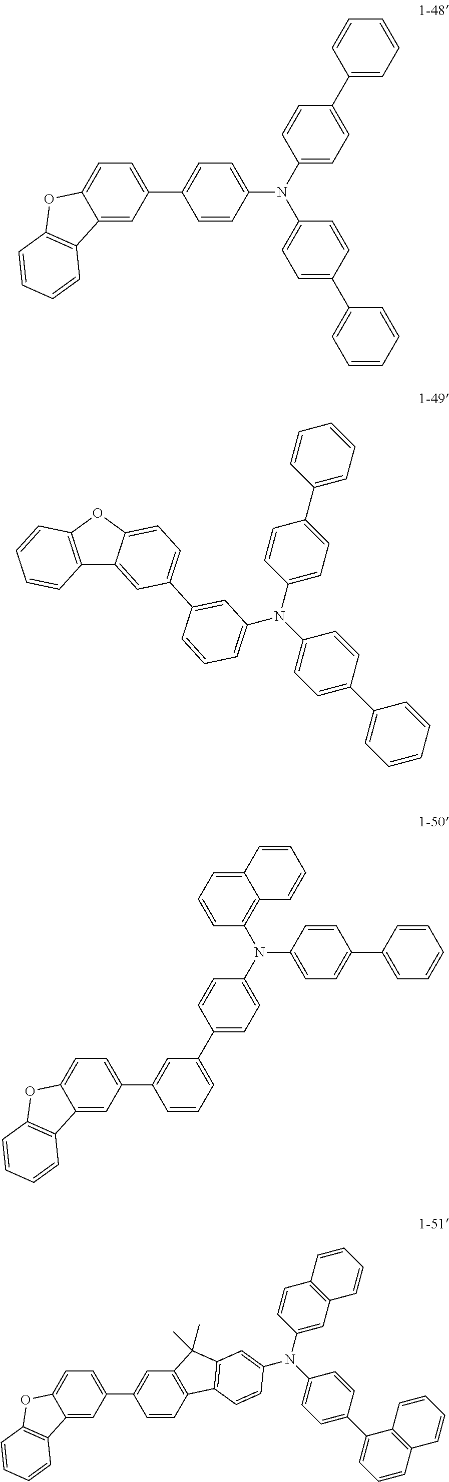 [SMILES: c1ccc(-c2ccc(N(c3ccc(-c4ccccc4)cc3)c3ccc(-c4ccc5oc6ccccc6c5c4)cc3)cc2)cc1 ; c1ccc(-c2ccc(N(c3ccc(-c4ccccc4)cc3)c3cccc(-c4ccc5oc6ccccc6c5c4)c3)cc2)cc1 ; c1ccc(-c2ccc(N(c3ccc(-c4cccc(-c5ccc6oc7ccccc7c6c5)c4)cc3)c3cccc4ccccc34)cc2)cc1 ; CC1(C)c2cc(-c3ccc4oc5ccccc5c4c3)ccc2-c2ccc(N(c3ccc(-c4cccc5ccccc45)cc3)c3ccc4ccccc4c3)cc21]

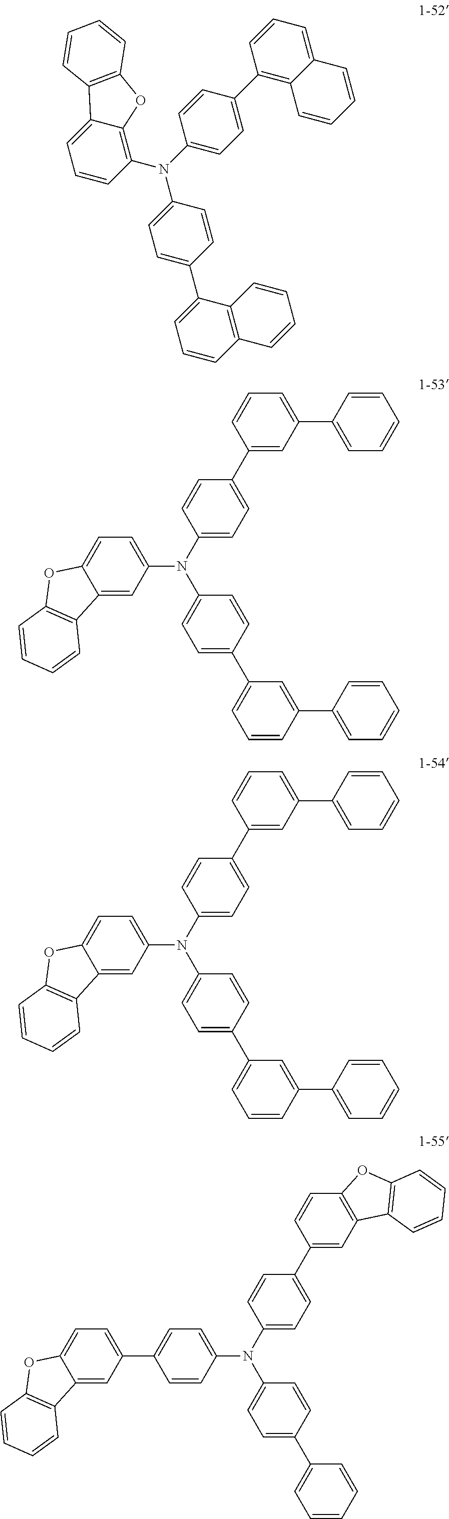

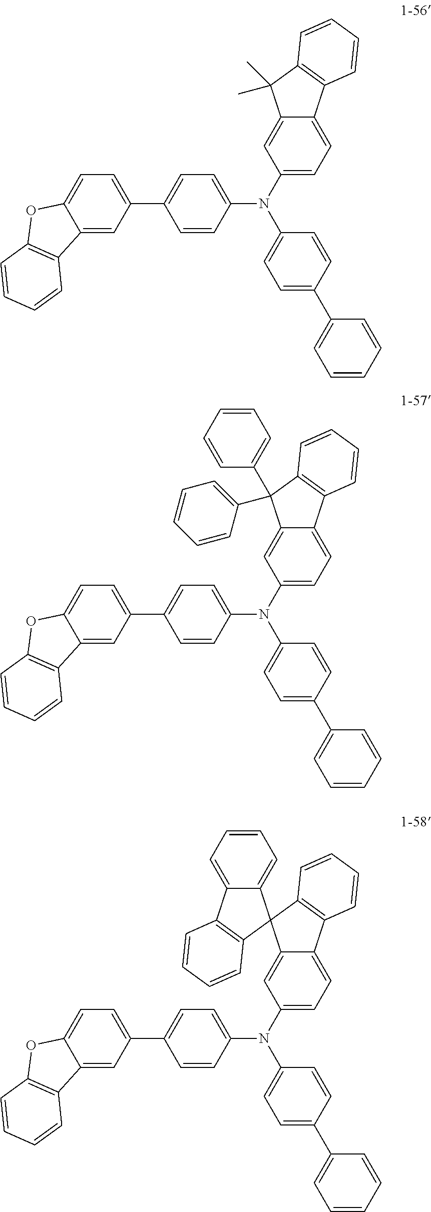 [SMILES: CC1(C)c2ccccc2-c2ccc(N(c3ccc(-c4ccccc4)cc3)c3ccc(-c4ccc5oc6ccccc6c5c4)cc3)cc21 ; c1ccc(-c2ccc(N(c3ccc(-c4ccc5oc6ccccc6c5c4)cc3)c3ccc4c(c3)C(c3ccccc3)(c3ccccc3)c3ccccc3-4)cc2)cc1 ; c1ccc(-c2ccc(N(c3ccc(-c4ccc5oc6ccccc6c5c4)cc3)c3ccc4c(c3)C3(c5ccccc5-c5ccccc53)c3ccccc3-4)cc2)cc1]

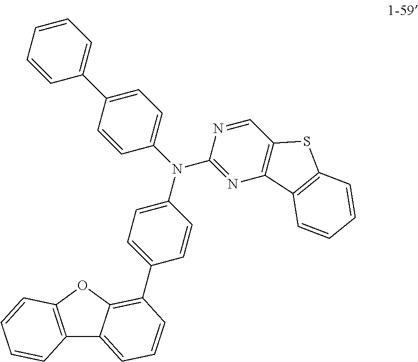 [SMILES: c1ccc(-c2ccc(N(c3ccc(-c4cccc5c4oc4ccccc45)cc3)c3ncc4sc5ccccc5c4n3)cc2)cc1]

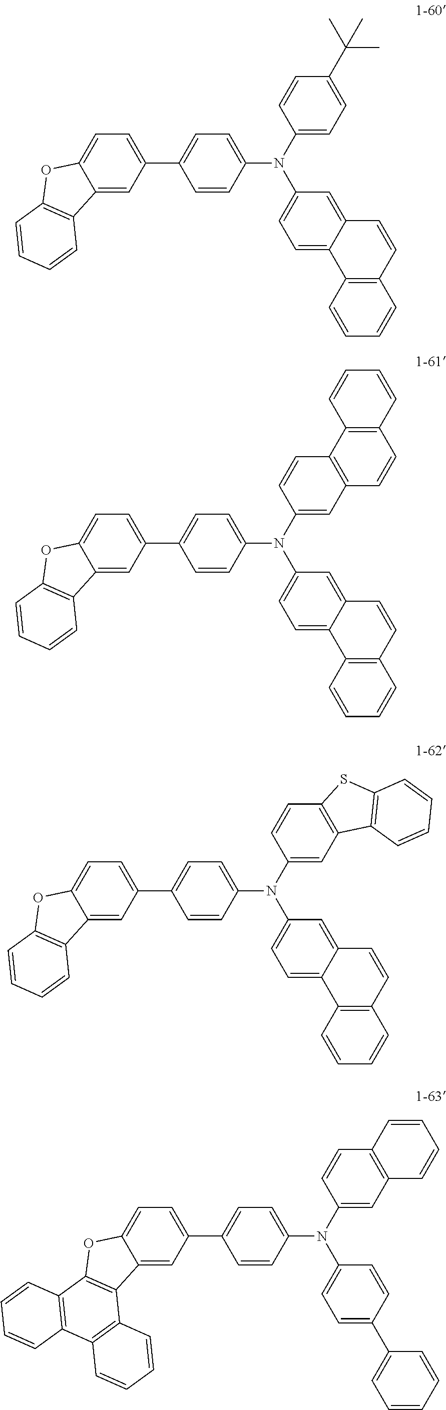

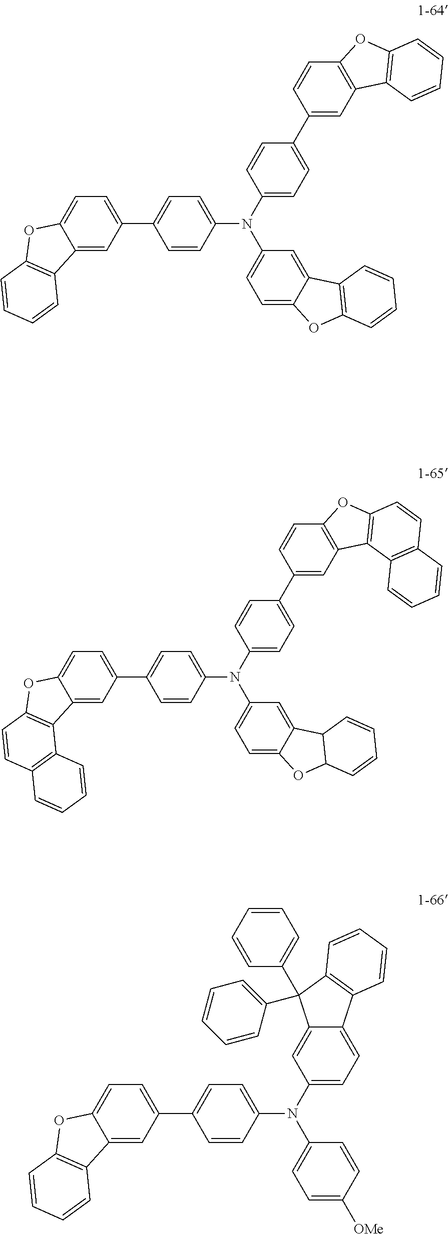 [SMILES: c1ccc2c(c1)oc1ccc(-c3ccc(N(c4ccc(-c5ccc6oc7ccccc7c6c5)cc4)c4ccc5oc6ccccc6c5c4)cc3)cc12 ; C1=CC2Oc3ccc(N(c4ccc(-c5ccc6oc7ccc8ccccc8c7c6c5)cc4)c4ccc(-c5ccc6oc7ccc8ccccc8c7c6c5)cc4)cc3C2C=C1 ; COc1ccc(N(c2ccc(-c3ccc4oc5ccccc5c4c3)cc2)c2ccc3c(c2)C(c2ccccc2)(c2ccccc2)c2ccccc2-3)cc1]

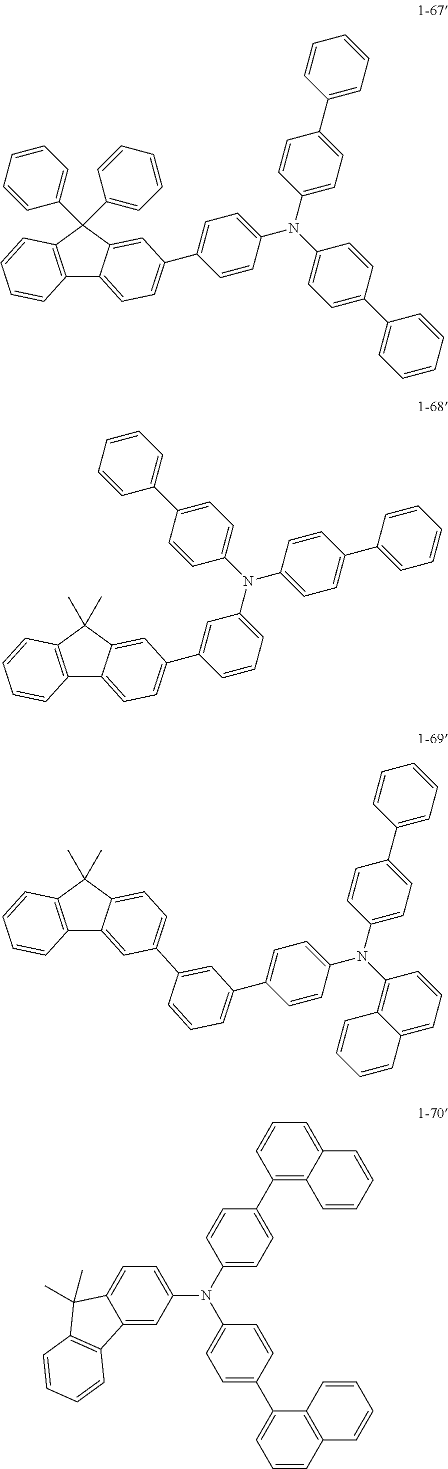

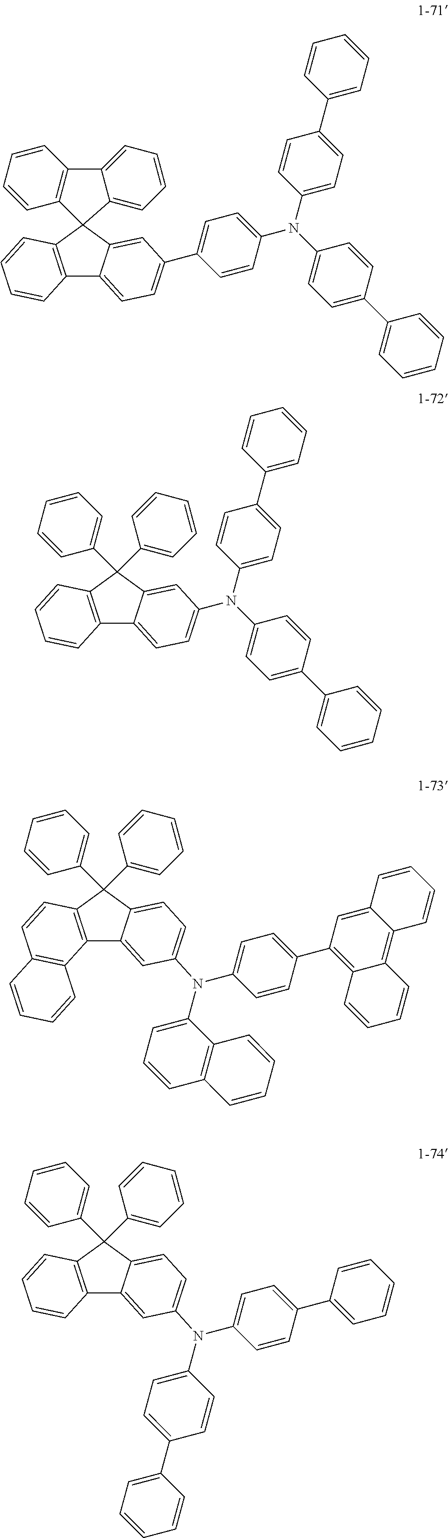 [SMILES: c1ccc(-c2ccc(N(c3ccc(-c4ccccc4)cc3)c3ccc(-c4ccc5c(c4)C4(c6ccccc6-c6ccccc64)c4ccccc4-5)cc3)cc2)cc1 ; c1ccc(-c2ccc(N(c3ccc(-c4ccccc4)cc3)c3ccc4c(c3)C(c3ccccc3)(c3ccccc3)c3ccccc3-4)cc2)cc1 ; c1ccc(C2(c3ccccc3)c3ccc(N(c4ccc(-c5cc6ccccc6c6ccccc56)cc4)c4cccc5ccccc45)cc3-c3c2ccc2ccccc32)cc1 ; c1ccc(-c2ccc(N(c3ccc(-c4ccccc4)cc3)c3ccc4c(c3)-c3ccccc3C4(c3ccccc3)c3ccccc3)cc2)cc1]

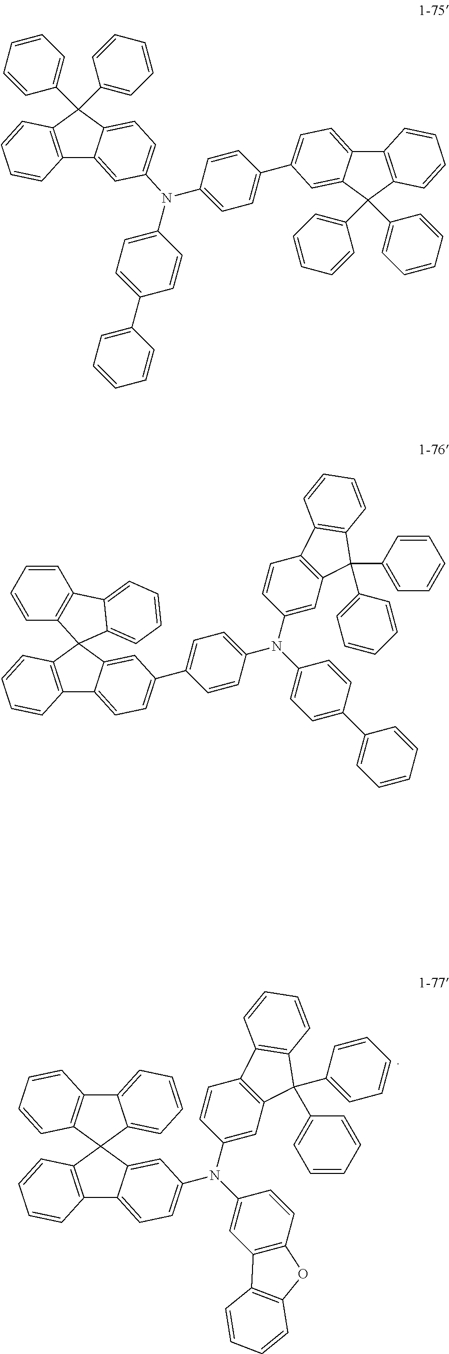 [SMILES: c1ccc(-c2ccc(N(c3ccc(-c4ccc5c(c4)C(c4ccccc4)(c4ccccc4)c4ccccc4-5)cc3)c3ccc4c(c3)-c3ccccc3C4(c3ccccc3)c3ccccc3)cc2)cc1 ; c1ccc(-c2ccc(N(c3ccc(-c4ccc5c(c4)C4(c6ccccc6-c6ccccc64)c4ccccc4-5)cc3)c3ccc4c(c3)C(c3ccccc3)(c3ccccc3)c3ccccc3-4)cc2)cc1 ; c1ccc(C2(c3ccccc3)c3ccccc3-c3ccc(N(c4ccc5c(c4)C4(c6ccccc6-c6ccccc64)c4ccccc4-5)c4ccc5oc6ccccc6c5c4)cc32)cc1]

5. The organic electric element according to claim 1, wherein the compound of Formula 2 or 2′ is represented by a compound of the following:

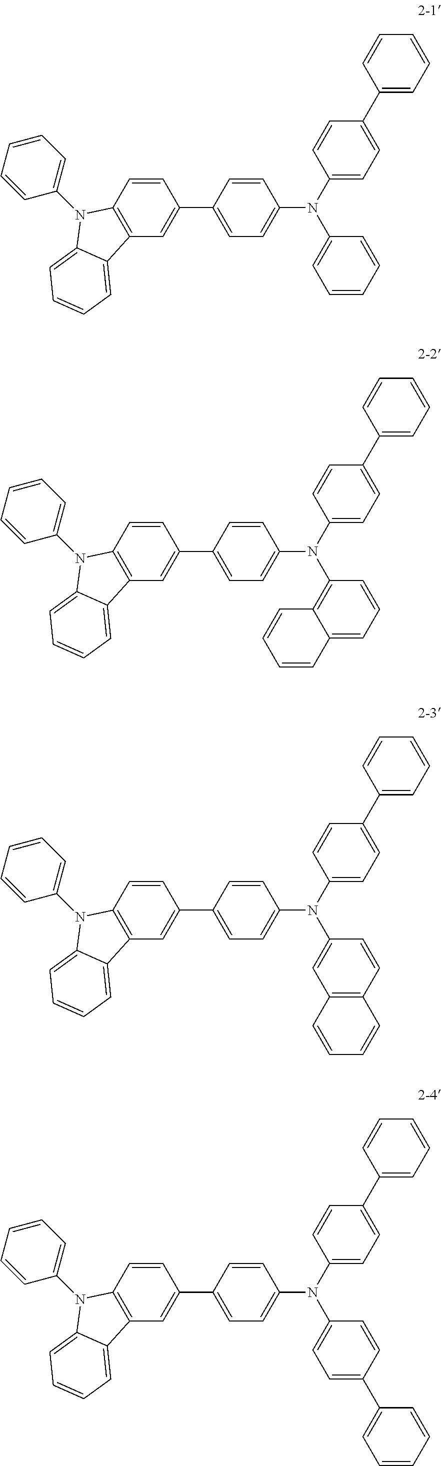 [SMILES: c1ccc(-c2ccc(N(c3ccccc3)c3ccc(-c4ccc5c(c4)c4ccccc4n5-c4ccccc4)cc3)cc2)cc1 ; c1ccc(-c2ccc(N(c3ccc(-c4ccc5c(c4)c4ccccc4n5-c4ccccc4)cc3)c3cccc4ccccc34)cc2)cc1 ; c1ccc(-c2ccc(N(c3ccc(-c4ccc5c(c4)c4ccccc4n5-c4ccccc4)cc3)c3ccc4ccccc4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1]

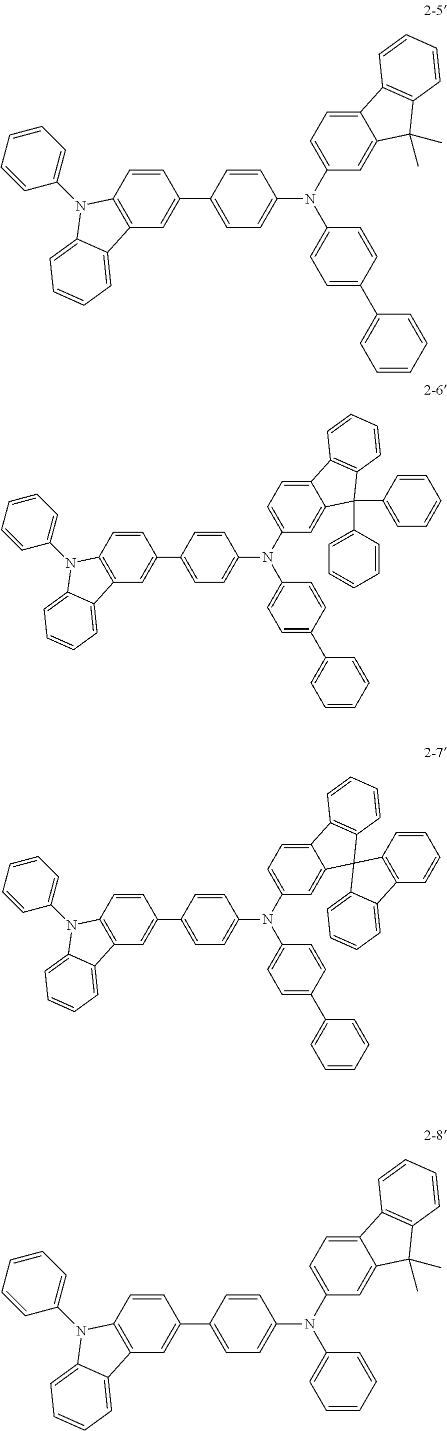 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; CC1(C)c2ccccc2-c2ccc(N(c3ccccc3)c3ccc(-c4ccc5c(c4)c4ccccc4n5-c4ccccc4)cc3)cc21]

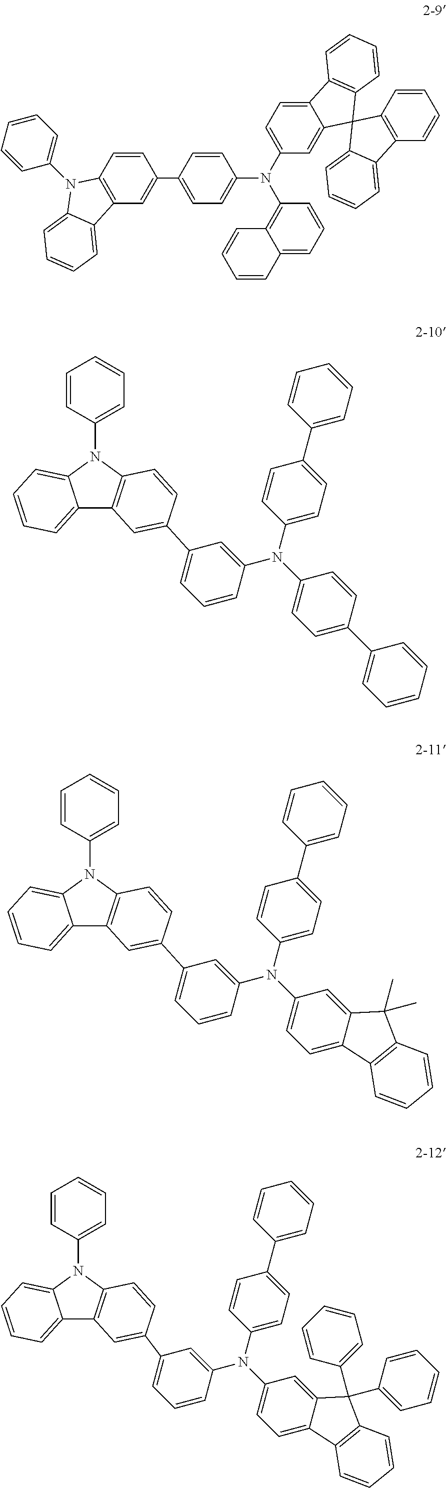 [SMILES: c1ccc(-n2c3ccccc3c3cc(-c4ccc(N(c5ccc6c(c5)C5(c7ccccc7-c7ccccc75)c5ccccc5-6)c5cccc6ccccc56)cc4)ccc32)cc1 ; c1ccc(-c2ccc(N(c3ccc(-c4ccccc4)cc3)c3cccc(-c4ccc5c(c4)c4ccccc4n5-c4ccccc4)c3)cc2)cc1 ; CC1(C)c2ccccc2-c2ccc(N(c3ccc(-c4ccccc4)cc3)c3cccc(-c4ccc5c(c4)c4ccccc4n5-c4ccccc4)c3)cc21 ; c1ccc(-c2ccc(N(c3cccc(-c4ccc5c(c4)c4ccccc4n5-c4ccccc4)c3)c3ccc4c(c3)C(c3ccccc3)(c3ccccc3)c3ccccc3-4)cc2)cc1]

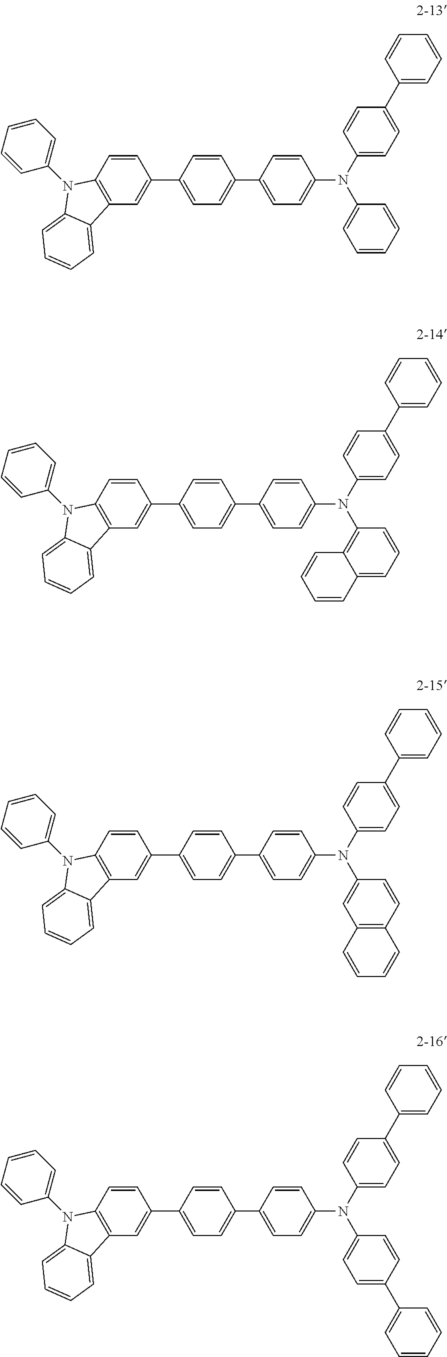 [SMILES: c1ccc(-c2ccc(N(c3ccccc3)c3ccc(-c4ccc(-c5ccc6c(c5)c5ccccc5n6-c5ccccc5)cc4)cc3)cc2)cc1 ; c1ccc(-c2ccc(N(c3ccc(-c4ccc(-c5ccc6c(c5)c5ccccc5n6-c5ccccc5)cc4)cc3)c3cccc4ccccc34)cc2)cc1 ; c1ccc(-c2ccc(N(c3ccc(-c4ccc(-c5ccc6c(c5)c5ccccc5n6-c5ccccc5)cc4)cc3)c3ccc4ccccc4c3)cc2)cc1 ; c1ccc(-c2ccc(N(c3ccc(-c4ccccc4)cc3)c3ccc(-c4ccc(-c5ccc6c(c5)c5ccccc5n6-c5ccccc5)cc4)cc3)cc2)cc1]

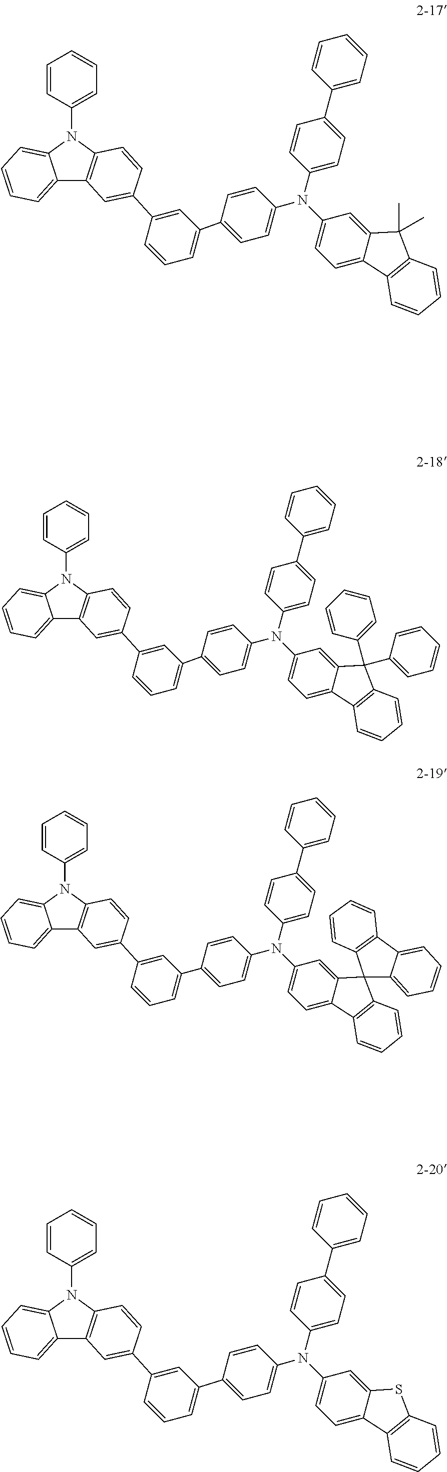 [SMILES: CC1(C)c2ccccc2-c2ccc(N(c3ccc(-c4ccccc4)cc3)c3ccc(-c4cccc(-c5ccc6c(c5)c5ccccc5n6-c5ccccc5)c4)cc3)cc21 ; c1ccc(-c2ccc(N(c3ccc(-c4cccc(-c5ccc6c(c5)c5ccccc5n6-c5ccccc5)c4)cc3)c3ccc4c(c3)C(c3ccccc3)(c3ccccc3)c3ccccc3-4)cc2)cc1 ; c1ccc(-c2ccc(N(c3ccc(-c4cccc(-c5ccc6c(c5)c5ccccc5n6-c5ccccc5)c4)cc3)c3ccc4c(c3)C3(c5ccccc5-c5ccccc53)c3ccccc3-4)cc2)cc1 ; c1ccc(-c2ccc(N(c3ccc(-c4cccc(-c5ccc6c(c5)c5ccccc5n6-c5ccccc5)c4)cc3)c3ccc4c(c3)sc3ccccc34)cc2)cc1]

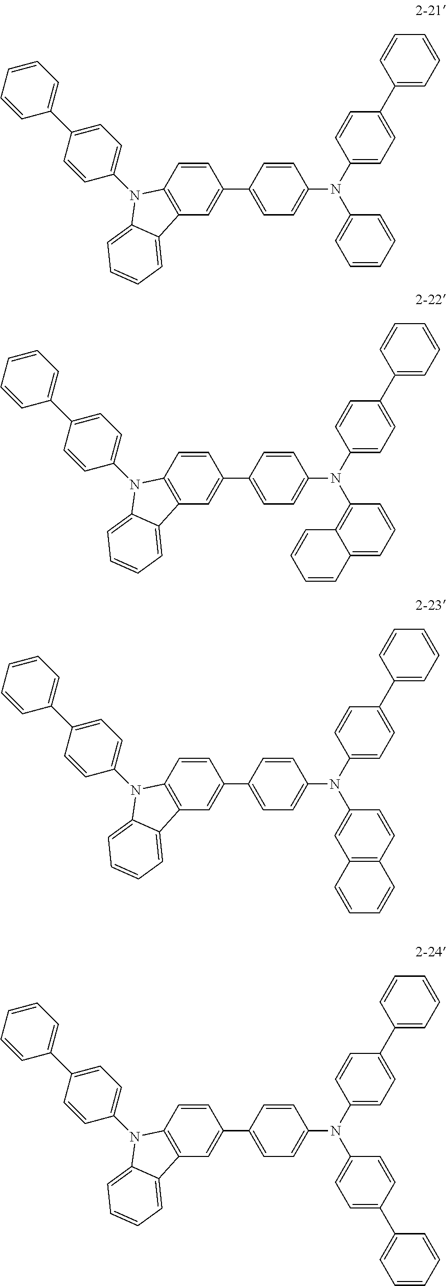 [SMILES: c1ccc(-c2ccc(N(c3ccccc3)c3ccc(-c4ccc5c(c4)c4ccccc4n5-c4ccc(-c5ccccc5)cc4)cc3)cc2)cc1 ; c1ccc(-c2ccc(N(c3ccc(-c4ccc5c(c4)c4ccccc4n5-c4ccc(-c5ccccc5)cc4)cc3)c3cccc4ccccc34)cc2)cc1 ; c1ccc(-c2ccc(N(c3ccc(-c4ccc5c(c4)c4ccccc4n5-c4ccc(-c5ccccc5)cc4)cc3)c3ccc4ccccc4c3)cc2)cc1 ; c1ccc(-c2ccc(N(c3ccc(-c4ccccc4)cc3)c3ccc(-c4ccc5c(c4)c4ccccc4n5-c4ccc(-c5ccccc5)cc4)cc3)cc2)cc1]

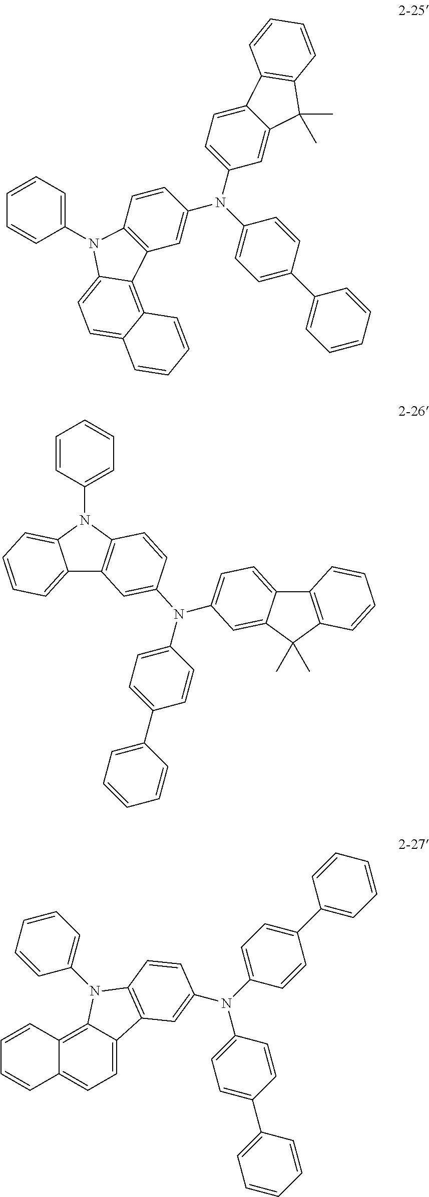 [SMILES: CC1(C)c2ccccc2-c2ccc(N(c3ccc(-c4ccccc4)cc3)c3ccc4c(c3)c3c5ccccc5ccc3n4-c3ccccc3)cc21 ; CC1(C)c2ccccc2-c2ccc(N(c3ccc(-c4ccccc4)cc3)c3ccc4c(c3)c3ccccc3n4-c3ccccc3)cc21 ; c1ccc(-c2ccc(N(c3ccc(-c4ccccc4)cc3)c3ccc4c(c3)c3ccc5ccccc5c3n4-c3ccccc3)cc2)cc1]

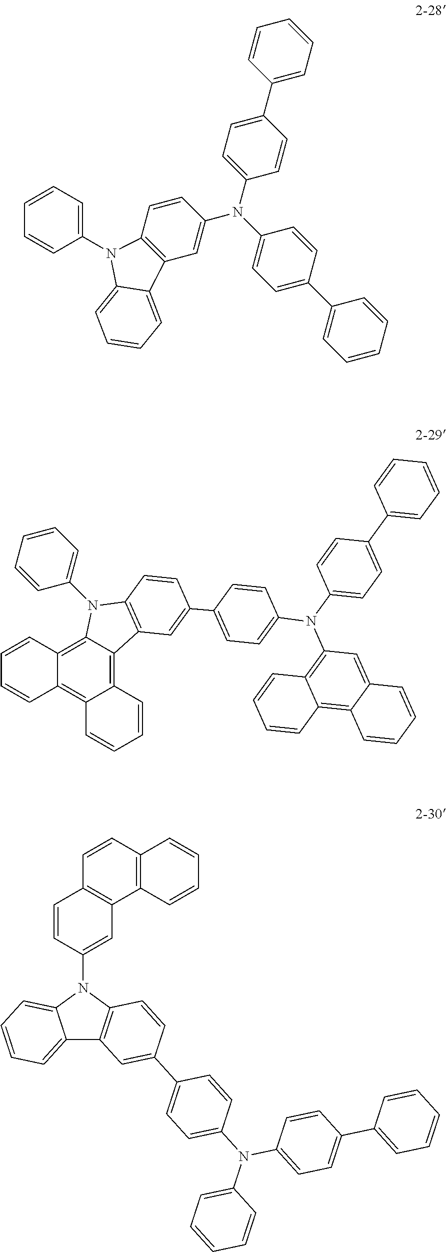 [SMILES: c1ccc(-c2ccc(N(c3ccc(-c4ccccc4)cc3)c3ccc4c(c3)c3ccccc3n4-c3ccccc3)cc2)cc1 ; c1ccc(-c2ccc(N(c3ccc(-c4ccc5c(c4)c4c6ccccc6c6ccccc6c4n5-c4ccccc4)cc3)c3cc4ccccc4c4ccccc34)cc2)cc1 ; c1ccc(-c2ccc(N(c3ccccc3)c3ccc(-c4ccc5c(c4)c4ccccc4n5-c4ccc5ccc6ccccc6c5c4)cc3)cc2)cc1]

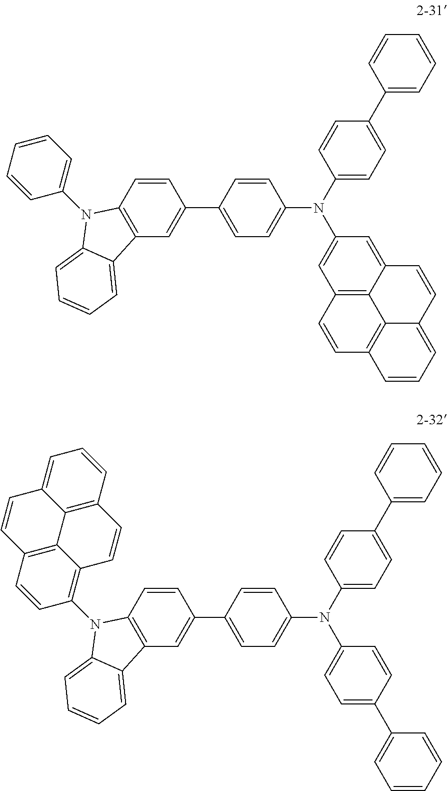 [SMILES: c1ccc(-c2ccc(N(c3ccc(-c4ccc5c(c4)c4ccccc4n5-c4ccccc4)cc3)c3cc4ccc5cccc6ccc(c3)c4c56)cc2)cc1 ; c1ccc(-c2ccc(N(c3ccc(-c4ccccc4)cc3)c3ccc(-c4ccc5c(c4)c4ccccc4n5-c4ccc5ccc6cccc7ccc4c5c67)cc3)cc2)cc1]

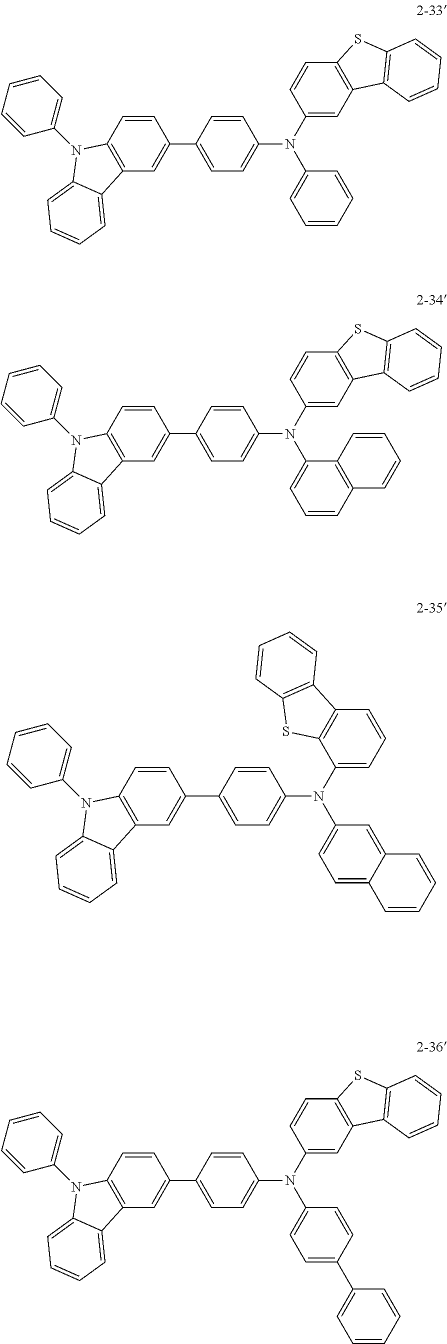 [SMILES: c1ccc(N(c2ccc(-c3ccc4c(c3)c3ccccc3n4-c3ccccc3)cc2)c2ccc3sc4ccccc4c3c2)cc1 ; c1ccc(-n2c3ccccc3c3cc(-c4ccc(N(c5ccc6sc7ccccc7c6c5)c5cccc6ccccc56)cc4)ccc32)cc1 ; c1ccc(-n2c3ccccc3c3cc(-c4ccc(N(c5ccc6ccccc6c5)c5cccc6c5sc5ccccc56)cc4)ccc32)cc1 ; c1ccc(-c2ccc(N(c3ccc(-c4ccc5c(c4)c4ccccc4n5-c4ccccc4)cc3)c3ccc4sc5ccccc5c4c3)cc2)cc1]

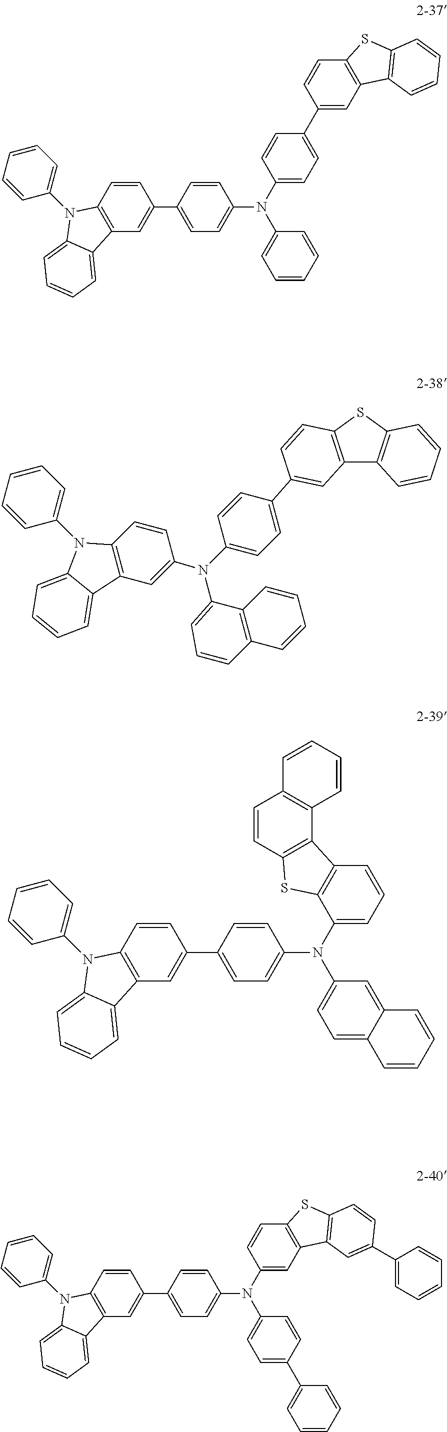 [SMILES: c1ccc(N(c2ccc(-c3ccc4sc5ccccc5c4c3)cc2)c2ccc(-c3ccc4c(c3)c3ccccc3n4-c3ccccc3)cc2)cc1 ; c1ccc(-n2c3ccccc3c3cc(N(c4ccc(-c5ccc6sc7ccccc7c6c5)cc4)c4cccc5ccccc45)ccc32)cc1 ; c1ccc(-n2c3ccccc3c3cc(-c4ccc(N(c5ccc6ccccc6c5)c5cccc6c5sc5ccc7ccccc7c56)cc4)ccc32)cc1 ; c1ccc(-c2ccc(N(c3ccc(-c4ccc5c(c4)c4ccccc4n5-c4ccccc4)cc3)c3ccc4sc5ccc(-c6ccccc6)cc5c4c3)cc2)cc1]

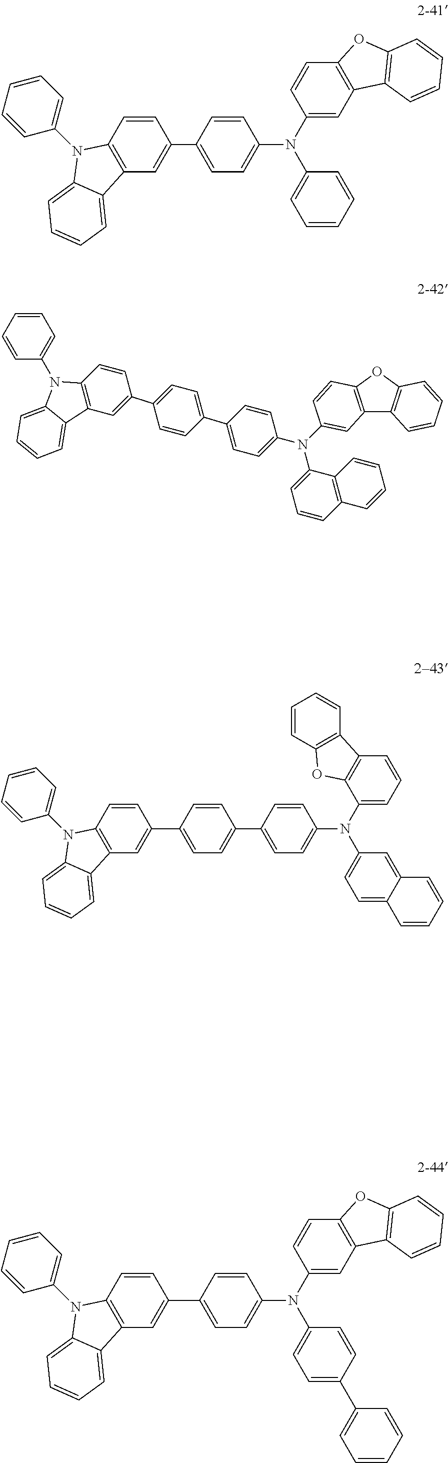 [SMILES: c1ccc(N(c2ccc(-c3ccc4c(c3)c3ccccc3n4-c3ccccc3)cc2)c2ccc3oc4ccccc4c3c2)cc1 ; c1ccc(-n2c3ccccc3c3cc(-c4ccc(-c5ccc(N(c6ccc7oc8ccccc8c7c6)c6cccc7ccccc67)cc5)cc4)ccc32)cc1 ; c1ccc(-n2c3ccccc3c3cc(-c4ccc(-c5ccc(N(c6ccc7ccccc7c6)c6cccc7c6oc6ccccc67)cc5)cc4)ccc32)cc1 ; c1ccc(-c2ccc(N(c3ccc(-c4ccc5c(c4)c4ccccc4n5-c4ccccc4)cc3)c3ccc4oc5ccccc5c4c3)cc2)cc1]

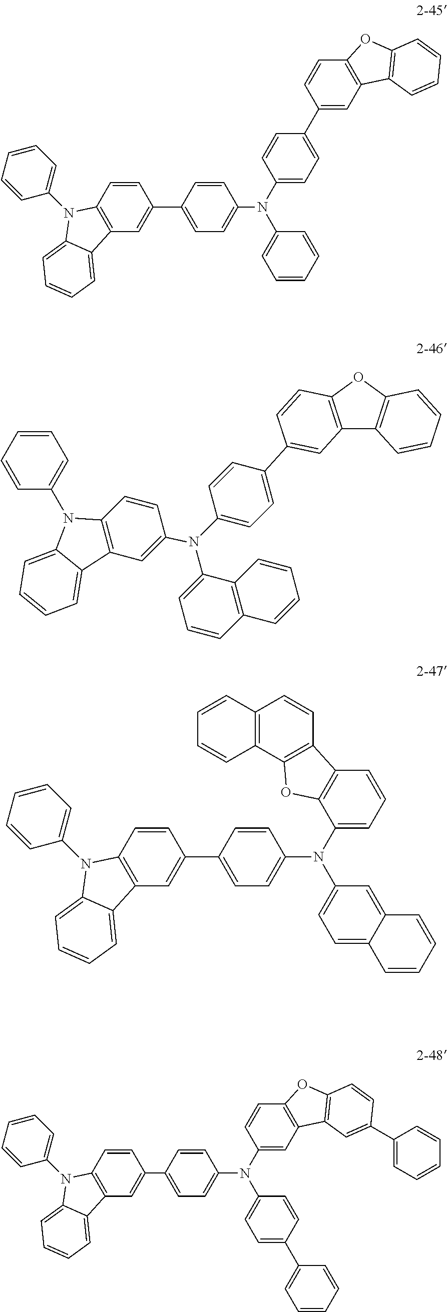 [SMILES: c1ccc(N(c2ccc(-c3ccc4oc5ccccc5c4c3)cc2)c2ccc(-c3ccc4c(c3)c3ccccc3n4-c3ccccc3)cc2)cc1 ; c1ccc(-n2c3ccccc3c3cc(N(c4ccc(-c5ccc6oc7ccccc7c6c5)cc4)c4cccc5ccccc45)ccc32)cc1 ; c1ccc(-n2c3ccccc3c3cc(-c4ccc(N(c5ccc6ccccc6c5)c5cccc6c5oc5c7ccccc7ccc65)cc4)ccc32)cc1 ; c1ccc(-c2ccc(N(c3ccc(-c4ccc5c(c4)c4ccccc4n5-c4ccccc4)cc3)c3ccc4oc5ccc(-c6ccccc6)cc5c4c3)cc2)cc1]

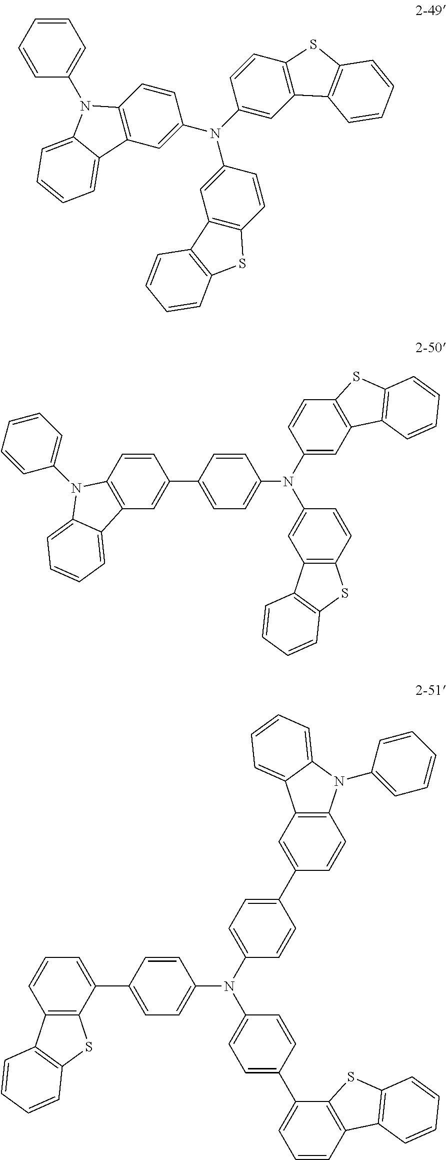 [SMILES: c1ccc(-n2c3ccccc3c3cc(N(c4ccc5sc6ccccc6c5c4)c4ccc5sc6ccccc6c5c4)ccc32)cc1 ; c1ccc(-n2c3ccccc3c3cc(-c4ccc(N(c5ccc6sc7ccccc7c6c5)c5ccc6sc7ccccc7c6c5)cc4)ccc32)cc1 ; c1ccc(-n2c3ccccc3c3cc(-c4ccc(N(c5ccc(-c6cccc7c6sc6ccccc67)cc5)c5ccc(-c6cccc7c6sc6ccccc67)cc5)cc4)ccc32)cc1]

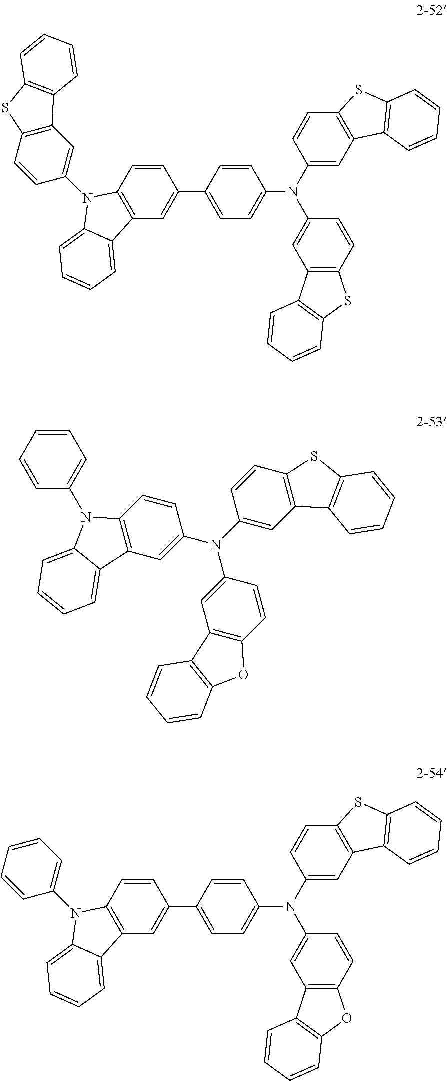 [SMILES: c1ccc2c(c1)sc1ccc(N(c3ccc(-c4ccc5c(c4)c4ccccc4n5-c4ccc5sc6ccccc6c5c4)cc3)c3ccc4sc5ccccc5c4c3)cc12 ; c1ccc(-n2c3ccccc3c3cc(N(c4ccc5oc6ccccc6c5c4)c4ccc5sc6ccccc6c5c4)ccc32)cc1 ; c1ccc(-n2c3ccccc3c3cc(-c4ccc(N(c5ccc6oc7ccccc7c6c5)c5ccc6sc7ccccc7c6c5)cc4)ccc32)cc1]

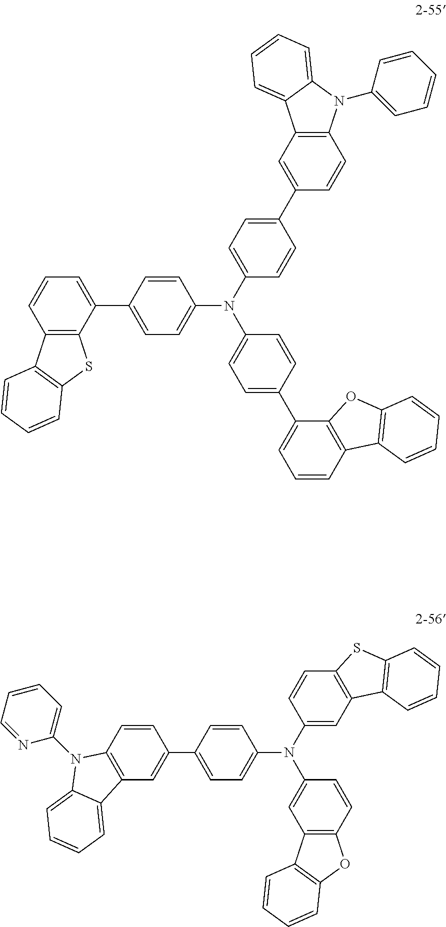 [SMILES: c1ccc(-n2c3ccccc3c3cc(-c4ccc(N(c5ccc(-c6cccc7c6oc6ccccc67)cc5)c5ccc(-c6cccc7c6sc6ccccc67)cc5)cc4)ccc32)cc1 ; c1ccc(-n2c3ccccc3c3cc(-c4ccc(N(c5ccc6oc7ccccc7c6c5)c5ccc6sc7ccccc7c6c5)cc4)ccc32)nc1]

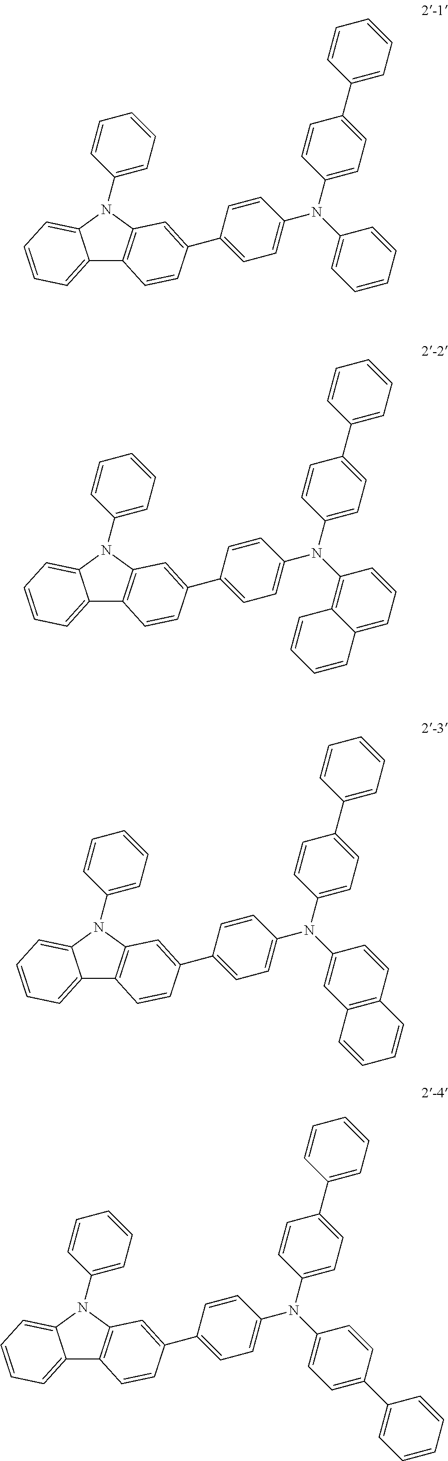 [SMILES: c1ccc(-c2ccc(N(c3ccccc3)c3ccc(-c4ccc5c6ccccc6n(-c6ccccc6)c5c4)cc3)cc2)cc1 ; c1ccc(-c2ccc(N(c3ccc(-c4ccc5c6ccccc6n(-c6ccccc6)c5c4)cc3)c3cccc4ccccc34)cc2)cc1 ; c1ccc(-c2ccc(N(c3ccc(-c4ccc5c6ccccc6n(-c6ccccc6)c5c4)cc3)c3ccc4ccccc4c3)cc2)cc1 ; c1ccc(-c2ccc(N(c3ccc(-c4ccccc4)cc3)c3ccc(-c4ccc5c6ccccc6n(-c6ccccc6)c5c4)cc3)cc2)cc1]

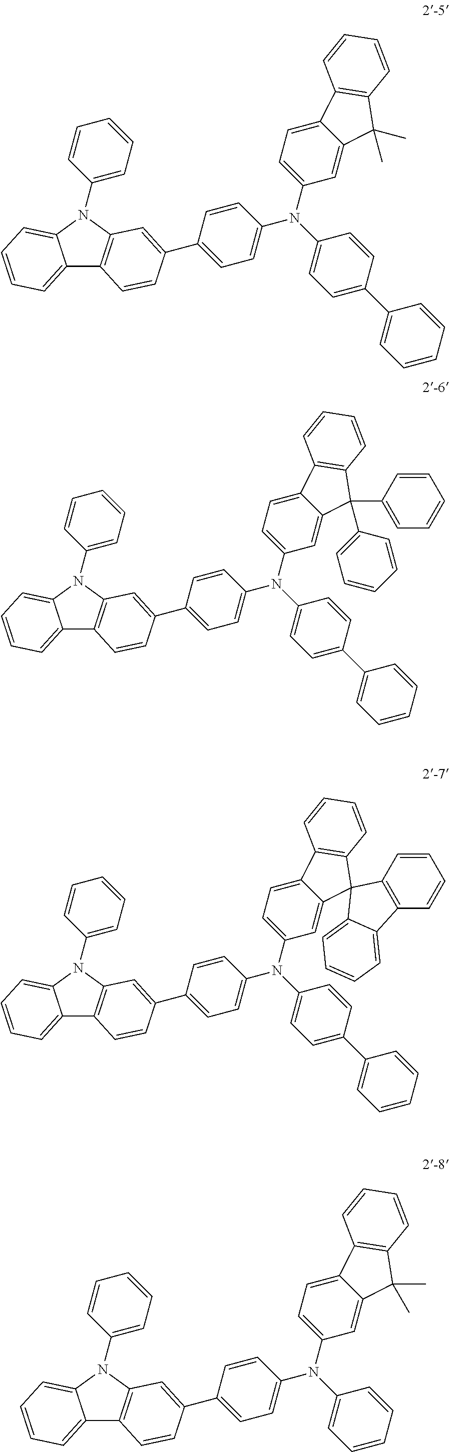 [SMILES: CC1(C)c2ccccc2-c2ccc(N(c3ccc(-c4ccccc4)cc3)c3ccc(-c4ccc5c6ccccc6n(-c6ccccc6)c5c4)cc3)cc21 ; c1ccc(-c2ccc(N(c3ccc(-c4ccc5c6ccccc6n(-c6ccccc6)c5c4)cc3)c3ccc4c(c3)C(c3ccccc3)(c3ccccc3)c3ccccc3-4)cc2)cc1 ; c1ccc(-c2ccc(N(c3ccc(-c4ccc5c6ccccc6n(-c6ccccc6)c5c4)cc3)c3ccc4c(c3)C3(c5ccccc5-c5ccccc53)c3ccccc3-4)cc2)cc1 ; CC1(C)c2ccccc2-c2ccc(N(c3ccccc3)c3ccc(-c4ccc5c6ccccc6n(-c6ccccc6)c5c4)cc3)cc21]

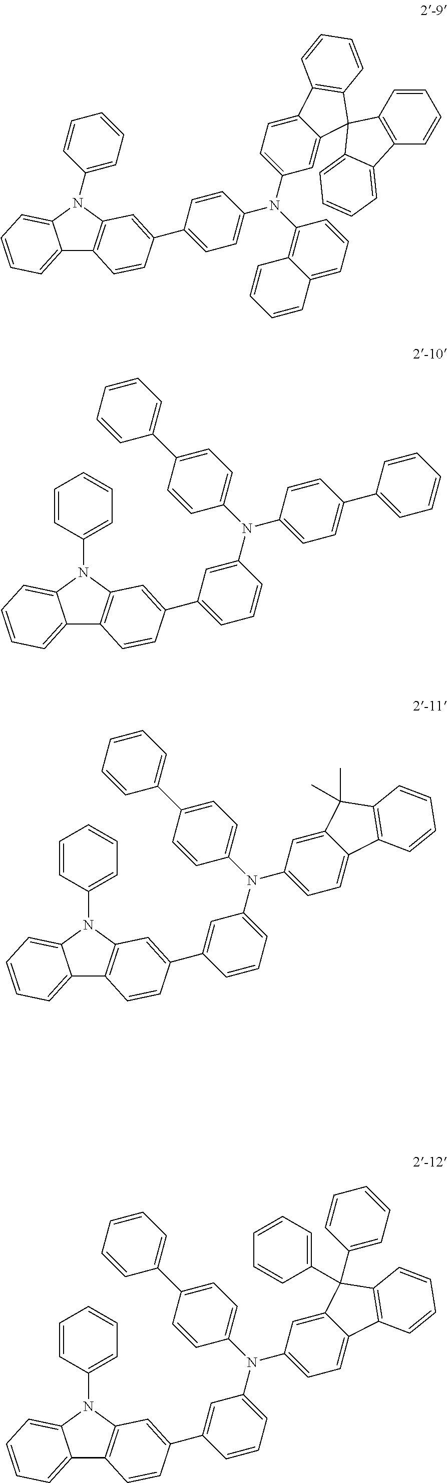 [SMILES: c1ccc(-n2c3ccccc3c3ccc(-c4ccc(N(c5ccc6c(c5)C5(c7ccccc7-c7ccccc75)c5ccccc5-6)c5cccc6ccccc56)cc4)cc32)cc1 ; c1ccc(-c2ccc(N(c3ccc(-c4ccccc4)cc3)c3cccc(-c4ccc5c6ccccc6n(-c6ccccc6)c5c4)c3)cc2)cc1 ; CC1(C)c2ccccc2-c2ccc(N(c3ccc(-c4ccccc4)cc3)c3cccc(-c4ccc5c6ccccc6n(-c6ccccc6)c5c4)c3)cc21 ; c1ccc(-c2ccc(N(c3cccc(-c4ccc5c6ccccc6n(-c6ccccc6)c5c4)c3)c3ccc4c(c3)C(c3ccccc3)(c3ccccc3)c3ccccc3-4)cc2)cc1]

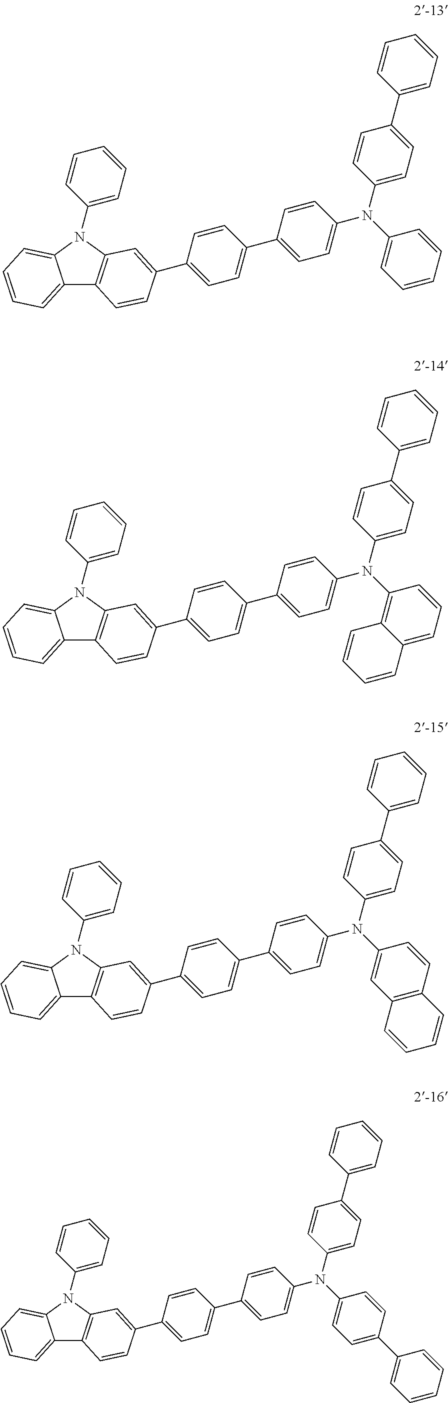 [SMILES: c1ccc(-c2ccc(N(c3ccccc3)c3ccc(-c4ccc(-c5ccc6c7ccccc7n(-c7ccccc7)c6c5)cc4)cc3)cc2)cc1 ; c1ccc(-c2ccc(N(c3ccc(-c4ccc(-c5ccc6c7ccccc7n(-c7ccccc7)c6c5)cc4)cc3)c3cccc4ccccc34)cc2)cc1 ; c1ccc(-c2ccc(N(c3ccc(-c4ccc(-c5ccc6c7ccccc7n(-c7ccccc7)c6c5)cc4)cc3)c3ccc4ccccc4c3)cc2)cc1 ; c1ccc(-c2ccc(N(c3ccc(-c4ccccc4)cc3)c3ccc(-c4ccc(-c5ccc6c7ccccc7n(-c7ccccc7)c6c5)cc4)cc3)cc2)cc1]

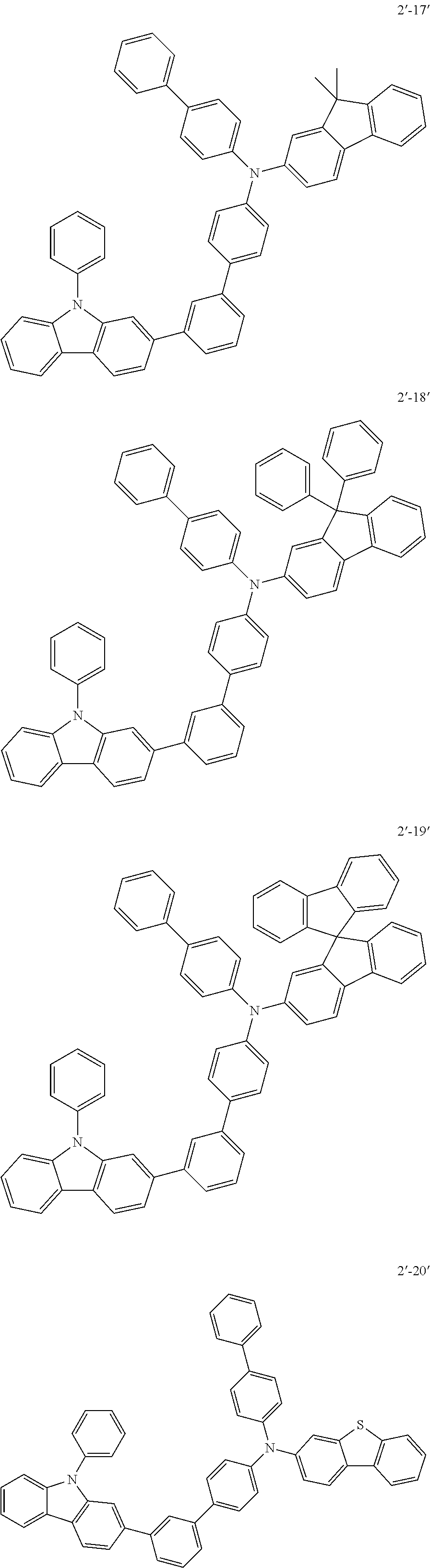 [SMILES: CC1(C)c2ccccc2-c2ccc(N(c3ccc(-c4ccccc4)cc3)c3ccc(-c4cccc(-c5ccc6c7ccccc7n(-c7ccccc7)c6c5)c4)cc3)cc21 ; c1ccc(-c2ccc(N(c3ccc(-c4cccc(-c5ccc6c7ccccc7n(-c7ccccc7)c6c5)c4)cc3)c3ccc4c(c3)C(c3ccccc3)(c3ccccc3)c3ccccc3-4)cc2)cc1 ; c1ccc(-c2ccc(N(c3ccc(-c4cccc(-c5ccc6c7ccccc7n(-c7ccccc7)c6c5)c4)cc3)c3ccc4c(c3)C3(c5ccccc5-c5ccccc53)c3ccccc3-4)cc2)cc1 ; c1ccc(-c2ccc(N(c3ccc(-c4cccc(-c5ccc6c7ccccc7n(-c7ccccc7)c6c5)c4)cc3)c3ccc4c(c3)sc3ccccc34)cc2)cc1]

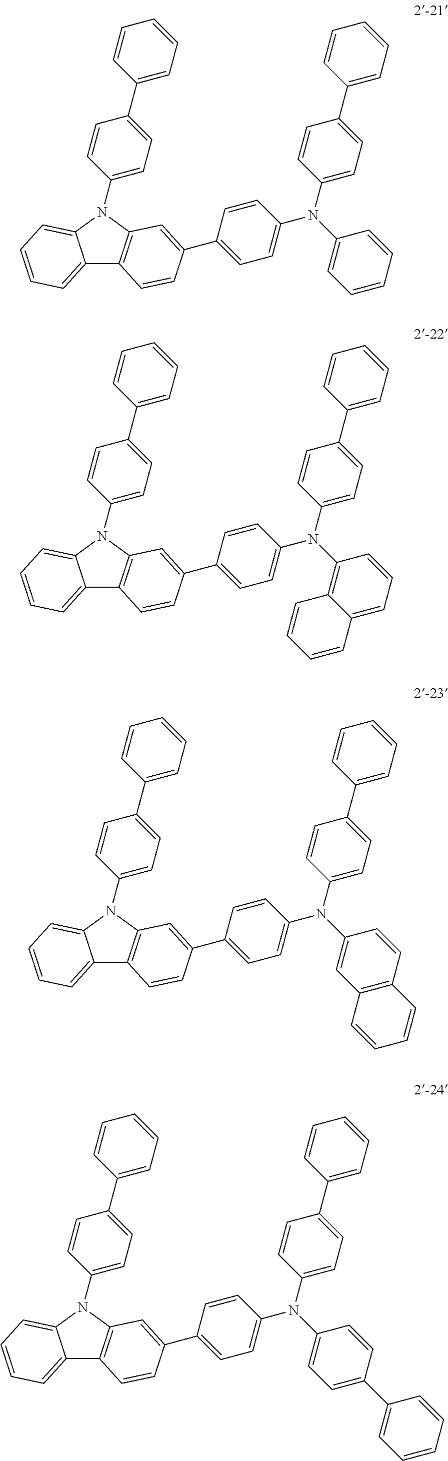 [SMILES: c1ccc(-c2ccc(N(c3ccccc3)c3ccc(-c4ccc5c6ccccc6n(-c6ccc(-c7ccccc7)cc6)c5c4)cc3)cc2)cc1 ; c1ccc(-c2ccc(N(c3ccc(-c4ccc5c6ccccc6n(-c6ccc(-c7ccccc7)cc6)c5c4)cc3)c3cccc4ccccc34)cc2)cc1 ; c1ccc(-c2ccc(N(c3ccc(-c4ccc5c6ccccc6n(-c6ccc(-c7ccccc7)cc6)c5c4)cc3)c3ccc4ccccc4c3)cc2)cc1 ; c1ccc(-c2ccc(N(c3ccc(-c4ccccc4)cc3)c3ccc(-c4ccc5c6ccccc6n(-c6ccc(-c7ccccc7)cc6)c5c4)cc3)cc2)cc1]

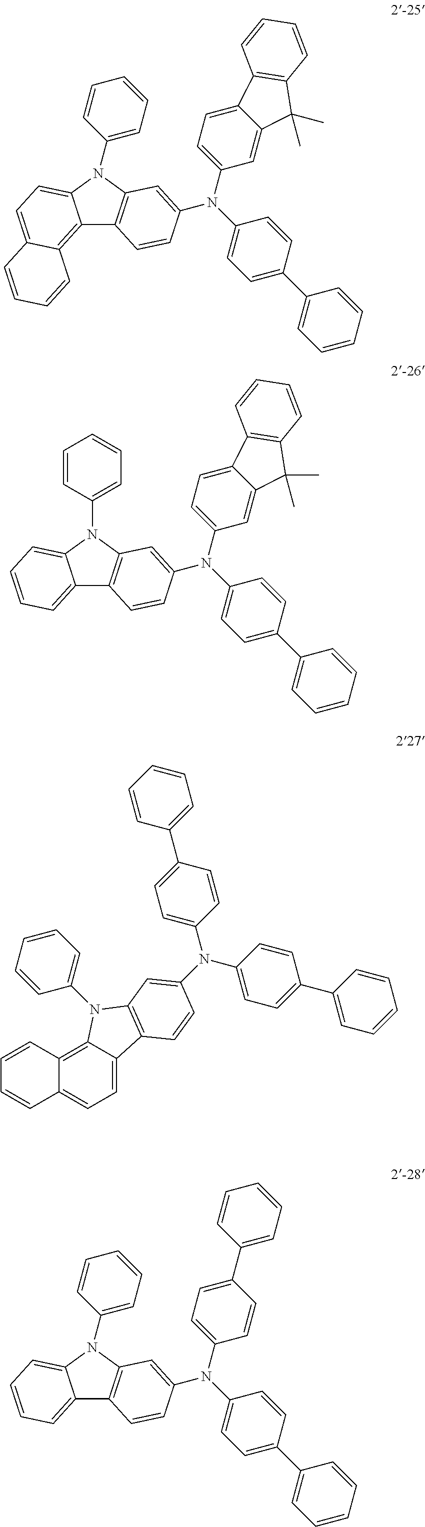

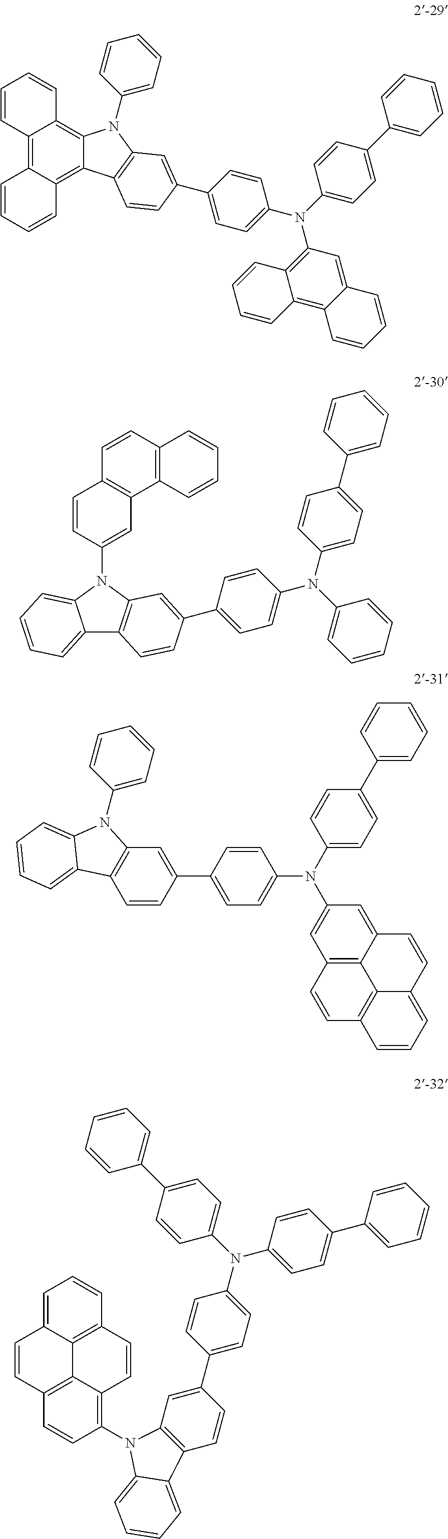

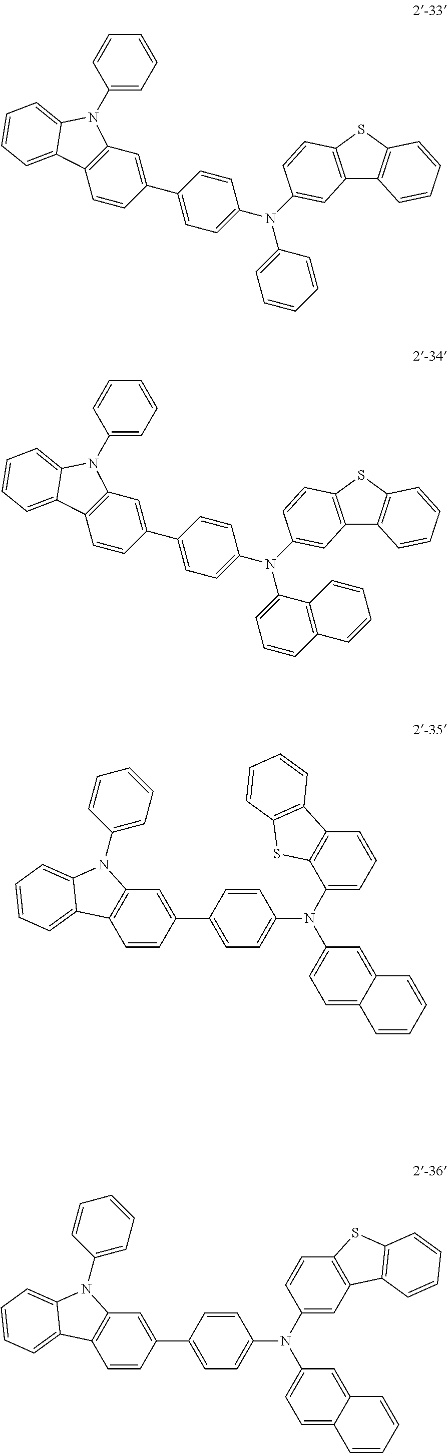 [SMILES: c1ccc(N(c2ccc(-c3ccc4c5ccccc5n(-c5ccccc5)c4c3)cc2)c2ccc3sc4ccccc4c3c2)cc1 ; c1ccc(-n2c3ccccc3c3ccc(-c4ccc(N(c5ccc6sc7ccccc7c6c5)c5cccc6ccccc56)cc4)cc32)cc1 ; c1ccc(-n2c3ccccc3c3ccc(-c4ccc(N(c5ccc6ccccc6c5)c5cccc6c5sc5ccccc56)cc4)cc32)cc1 ; c1ccc(-n2c3ccccc3c3ccc(-c4ccc(N(c5ccc6ccccc6c5)c5ccc6sc7ccccc7c6c5)cc4)cc32)cc1]

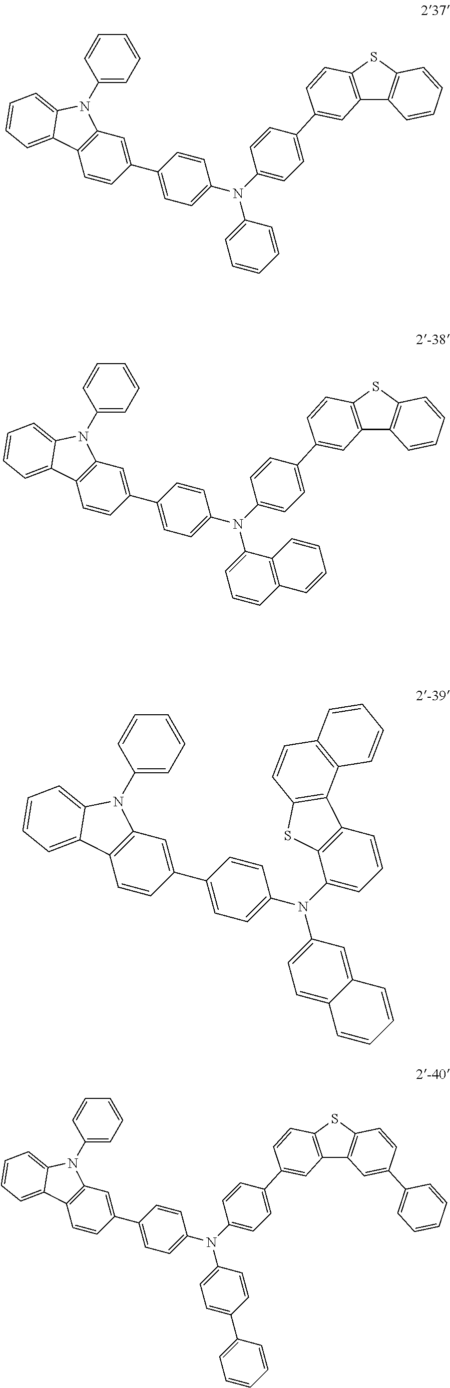 [SMILES: c1ccc(N(c2ccc(-c3ccc4sc5ccccc5c4c3)cc2)c2ccc(-c3ccc4c5ccccc5n(-c5ccccc5)c4c3)cc2)cc1 ; c1ccc(-n2c3ccccc3c3ccc(-c4ccc(N(c5ccc(-c6ccc7sc8ccccc8c7c6)cc5)c5cccc6ccccc56)cc4)cc32)cc1 ; c1ccc(-n2c3ccccc3c3ccc(-c4ccc(N(c5ccc6ccccc6c5)c5cccc6c5sc5ccc7ccccc7c56)cc4)cc32)cc1 ; c1ccc(-c2ccc(N(c3ccc(-c4ccc5sc6ccc(-c7ccccc7)cc6c5c4)cc3)c3ccc(-c4ccc5c6ccccc6n(-c6ccccc6)c5c4)cc3)cc2)cc1]

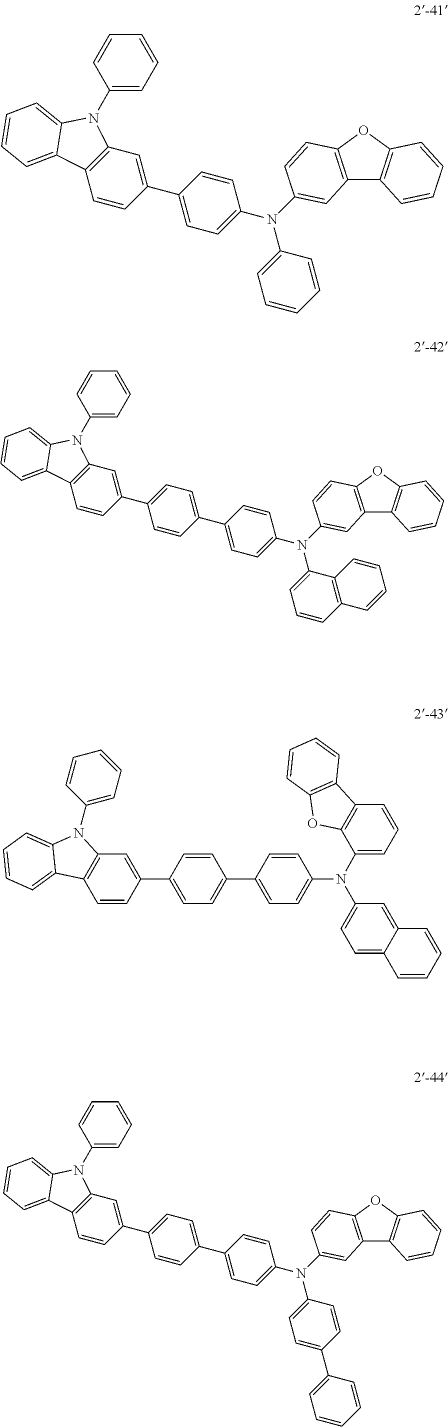 [SMILES: c1ccc(N(c2ccc(-c3ccc4c5ccccc5n(-c5ccccc5)c4c3)cc2)c2ccc3oc4ccccc4c3c2)cc1 ; c1ccc(-n2c3ccccc3c3ccc(-c4ccc(-c5ccc(N(c6ccc7oc8ccccc8c7c6)c6cccc7ccccc67)cc5)cc4)cc32)cc1 ; c1ccc(-n2c3ccccc3c3ccc(-c4ccc(-c5ccc(N(c6ccc7ccccc7c6)c6cccc7c6oc6ccccc67)cc5)cc4)cc32)cc1 ; c1ccc(-c2ccc(N(c3ccc(-c4ccc(-c5ccc6c7ccccc7n(-c7ccccc7)c6c5)cc4)cc3)c3ccc4oc5ccccc5c4c3)cc2)cc1]

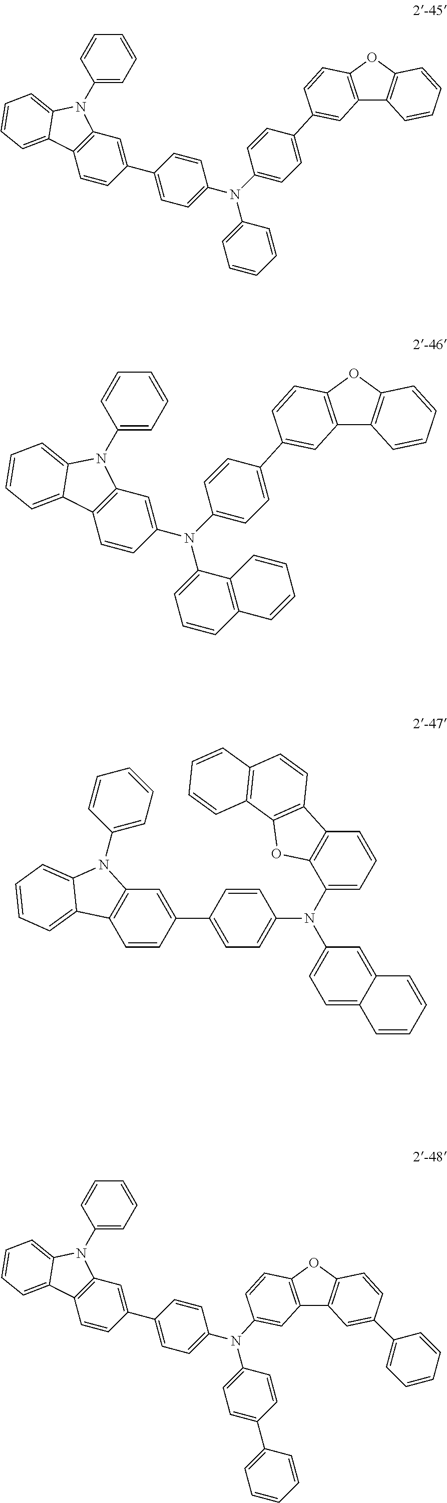 [SMILES: c1ccc(N(c2ccc(-c3ccc4oc5ccccc5c4c3)cc2)c2ccc(-c3ccc4c5ccccc5n(-c5ccccc5)c4c3)cc2)cc1 ; c1ccc(-n2c3ccccc3c3ccc(N(c4ccc(-c5ccc6oc7ccccc7c6c5)cc4)c4cccc5ccccc45)cc32)cc1 ; c1ccc(-n2c3ccccc3c3ccc(-c4ccc(N(c5ccc6ccccc6c5)c5cccc6c5oc5c7ccccc7ccc65)cc4)cc32)cc1 ; c1ccc(-c2ccc(N(c3ccc(-c4ccc5c6ccccc6n(-c6ccccc6)c5c4)cc3)c3ccc4oc5ccc(-c6ccccc6)cc5c4c3)cc2)cc1]

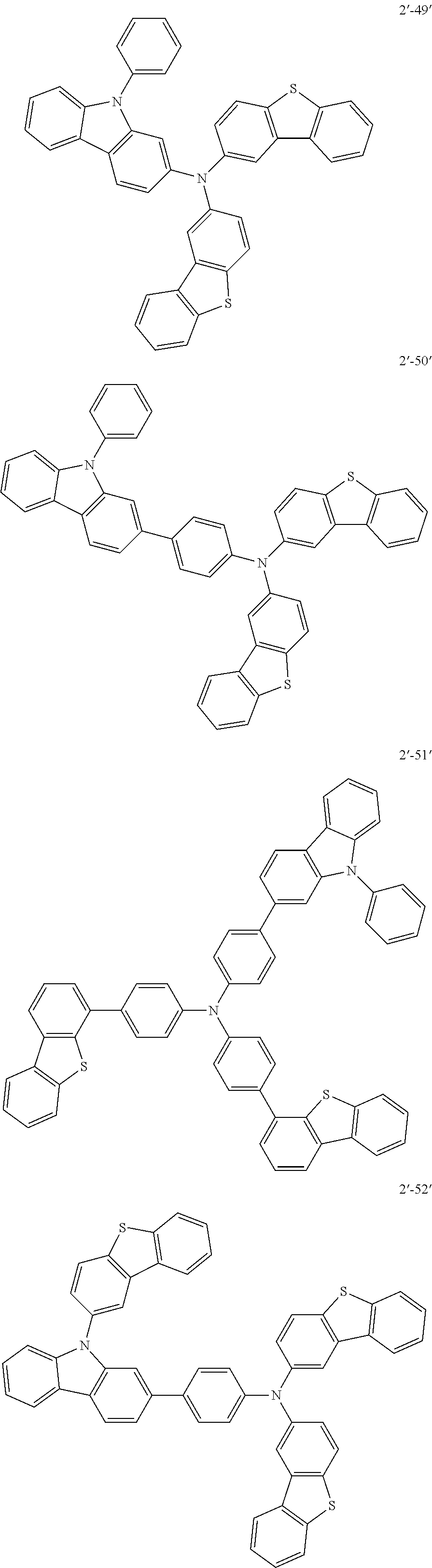

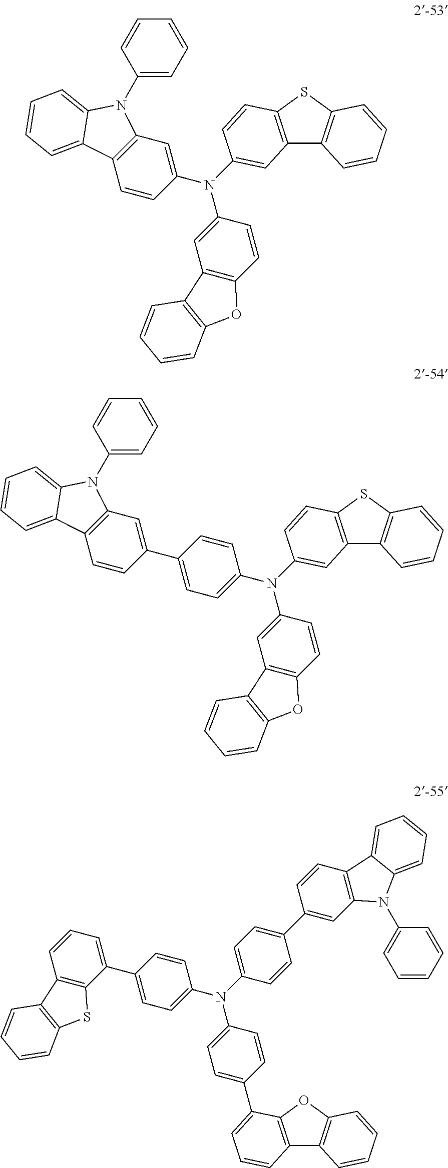

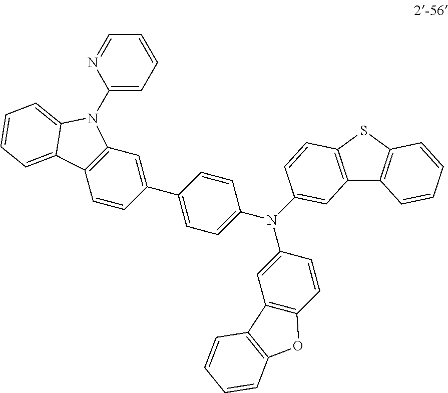 [SMILES: c1ccc(-n2c3ccccc3c3ccc(-c4ccc(N(c5ccc6oc7ccccc7c6c5)c5ccc6sc7ccccc7c6c5)cc4)cc32)nc1]

6. The organic electric element according to claim 1, wherein Ar¹, Ar² and Ar³ of the compounds represented by Formula 1 are each a C₆-C₂₄ aryl group.

7. The organic electric element according to claim 1, wherein Ar⁴ and Ar⁵ of the compounds represented by Formula 2 or 2′ are each a C₆-C₂₄ aryl group.

8. The organic electric element according to claim 1, wherein Ar¹, Ar² and Ar³ of the compounds represented by Formula 1 are each a C₆-C₂₄ aryl group; and at least one of Ar⁴ and Ar⁵ of the compound represented by Formula 2 or 2′ is dibenzothiophene or dibenzofuran.

9. The organic electric element according to claim 1, wherein at least one of Ar¹, Ar² and Ar³ of the compounds represented by Formula 1 is dibenzothiophene or dibenzofuran; Ar⁴ and Ar⁵ of the compound represented by Formula 2 or 2′ are each a C₆-C₂₄ aryl group.

10. The organic electric element according to claim 1, wherein at least one of Ar¹, Ar² and Ar³ of the compounds represented by Formula 1 is dibenzothiophene or dibenzofuran; and at least one of Ar⁴ and Ar⁵ of the compound represented by Formula 2 or 2′ is dibenzothiophene or dibenzofuran.

11. The organic electric element according to claim 1, wherein in the mixing ratio of the compound represented by Formula 1 and the compound represented by Formula 2 or 2′, the compound represented by Formula 1 is 10%˜90%.

12. The organic electric element according to claim 1, wherein in the mixing ratio of the compound represented by Formula 1 and the compound represented by Formula 2 or 2′ is at least one of 5:5, 6:4, 7:3, 8:2, and 9:1.

13. The organic electric element according to claim 1, wherein in the mixture of the compound represented by Formula 1 and the compound represented by Formula 2 or 2′ further comprises one or more of the compounds represented by Formula 1.

14. The organic electric element according to claim 1, wherein a compound represented by Formula 1 is used as an emitting auxiliary layer, between the emitting layer and the hole transport layer composed of the mixture of the compound represented by Formula 1 and the compound represented by Formula 2 or 2′.

15. The organic electric element according to claim 1, further comprising a light efficiency improving layer formed on at least one side opposite to the organic material layer, among one side of the first electrode and the second electrode.

16. The organic electric element according to claim 1, wherein the organic material layer is formed by one of a spin coating process, a nozzle printing process, an inkjet printing process, a slot coating process, a dip coating process, and a roll-to-roll process.

17. An electronic device comprising a display apparatus comprising the organic electric element according to claim 1; and a driving part configured to drive the display apparatus.

18. The electronic device according to claim 17, wherein the organic electric element is one of an organic light emitting diode (OLED), an organic solar cell, an organic photo conductor, an organic transistor, and a device for monochromic or white illumination.
